SEQUENCE LISTING (45 sequences)

SEQ ID NO: 1 - protein, 301 aa, Unknown
  Ruminococcaceae UCG13
KIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSS

SEQ ID NO: 2 - protein, 890 aa, artificial
  Synthetic
MEEAAADAQNAEINYNRSVPLEVKGNKIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSSSPKMLYSWDYKTTAYNGTHIKNRLLSYNTAPEKLDGVYSTDFSTDDVFRSYTAPSGKASIKYSDESGNVAITPAAANWYATLNFPFDWDLNGIQTITMDISAATAGSVNIGLYGSDMEVWTKAVDVNTEVQTVTIGINELVKQGNPQTDGKLDAALSGIYFGAATADTGSITIDNVKIVKLATPVYTANTYPHKDMGEESYIDIDTTGFKKQTTAWNSKFTGTTMQITDANVLNINGETTKTKCVTYTRDATDTEGCRAKFDLNTVPSMDAKYFTIDIKGNGIAQKLTVSLSGLAYITVNMAEGDTDWHQYIYSLEGNVEYPEDITYVQISADTRTTAEFYIDNIGFSNTKSERLIPYPEKTFVYDFATYNKNTTKYEAAISTESGSEGDTIVATKEEGGLGFDSKALEVKYSRNGNTPSKAKVVYSPNDFFKGNVNDDERTANRATLKADMEYMTDFVFYGKSTSGKNEKINVGVIDTASAMTTYTDTKEFTLTTEWKQFRVPFDEFKILDGGSNLDCARVRGFIFSSAENSGEGSFMIDNITHTSIKGDIEWGLPHHHHHH

SEQ ID NO: 3 - protein, 2149 aa, Unknown
  Ruminococcaceae UCG13
MERVIFMKKFLSLVTAIVMTVSLCIMPVYAQTYEEAAADAQNAEINYNRSVPLEVKGNKIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSSSPKMLYSWDYKTTAYNGTHIKNRLLSYNTAPEKLDGVYSTDFSTDDVFRSYTAPSGKASIKYSDESGNVAITPAAANWYATLNFPFDWDLNGIQTITMDISAATAGSVNIGLYGSDMEVWTKAVDVNTEVQTVTIGINELVKQGNPQTDGKLDAALSGIYFGAATADTGSITIDNVKIVKLATPVYTANTYPHKDMGEESYIDIDTTGFKKQTTAWNSKFTGTTMQITDANVLNINGETTKTKCVTYTRDATDTEGCRAKFDLNTVPSMDAKYFTIDIKGNGIAQKLTVSLSGLAYITVNMAEGDTDWHQYIYSLEGNVEYPEDITYVQISADTRTTAEFYIDNIGFSNTKPERLIPYPEKTFVYDFATYNKNTTKYEAAISTESGSEGDTIVATKEEGGLGFDSKALEVKYSRNGNTPSKAKVVYSPNDFFKGNVNDDERTANRATLKADMEYMTDFVFYGKSTSGKNEKINVGVIDTASAMTTYTDTKEFTLTTEWKQFRVPFDEFKILDGGSNLDCARVRGFIFSSAENSGEGSFMIDNITHTSIKGDIEWGLPTPTPEPTPTPLPDPVTVTTAEQLAAITSTEGNIILGADIDLGTTGFTTKSVTHLDLNGHTLTSSGPFVVDPRHEITIVDTGSTKGAIINTGTTQTSYGIRGTTEAATINIDGAEIDAGGQAILINVAGRKCNIKDAVINGGSYAINVGTNGGEINIDNALINNKADYKGYALYLQGGIAIIDDGTFGYNGTTNTLLVARSSELTINGGTFTNPNSGRGAIVTDKQFVGTVTINGGVFENTNAGGYSILDSNEGYQSIDAETSEIIASPVININDGTFKSAIGKTKSTNSSATEISIKGGQFAADPTVLYPNCIDTDIYSITKVAEGKYVVTKKGVEPTPEPTPEPVAKIVSSIEEINTLTASDDYVKLGADIDLGTSSIKTKCAMRLDLNGHTLSGGGSTVIEAMYNLTVVDTGTTKGTIKNVNTSTSYGIKFAVKDAVLTIDGAKVEAMSQAIMLSGTGSILHLKDSVINGNSYAVNLSNGIINIENTVINDDSEYKGYALSVANGTAVINSGIFNYNGNMSSITFSGSSEITINGGTFKNSVSKRGAINTVKGFSGTLTINGGTFENTAENNGYSILDGDEATTETVPVINITGGTFKSTIGATKPANTTTVITISGGTYSFDPTSYVTDTETYRVIDNGDGTYKVAPNSQVYSVTLNACGGSEVMVEDFKEENIPDNGIELPIPTKAGYKFDGWYTEENNGSQVNGITKDNLSDIFRNEATVTLYAHWTLLNYTITYEGLNDATNTNPSNYTVETEAITLAAPGTRKGYTFGGWYTDVEYQNKIEIIEQGTTGNKILYAKWDEIASGSITASFVSTGTIPSDIVQGTINVTEKAYENDEVSFMVTLPKGYTLENVLCTADGENLNTITEENGSYTFIMPGKNVTITVNVRPIQYTINLDLQEGTGTTTTIYGSVENLPVLPNDNPKKQGYNFKGWFDAPTKGTVITMDNLNTASNMLALFGNNTELTIYAQYTEVGNFVVIYSAVGADEETIPTDNTQYNIAETSIIKIPNQEPKKLGYTFEGWKTGTDDTVYKYGTQNDTYTVPNDINGAITFIAQWSINEYEITYELNGGINAENAPVSYTIETDTITLPVPTKDGYNFEGWYTDAAFENAVAAIAKGSVGDMVLYAKWSEKDMAVYKINNYEKGNVSVRKRTDTDDSSSVVIVAFYKTLNNNSVLIKTSIAEIGAIEKGDDISKTVEEPEDYSYAKVFMWNDLNGMMPRCNSPKMDK

SEQ ID NO: 4 - protein, 727 aa, Unknown
  Ruminococcaceae UCG13
MILLIHIKMGGMIMTDFNILRKRYSDVLCGRGYNGKKTADCILQSDERTEQRLVQLGGRIEKAITSNEPGVINATLKGILDISISFSQNNSQFYHNKNIKNEIFNALNTLEKVYNDTTVPKGNWWYWEIGIPLSINSIFTLMYDYTDKSQLKRYMAAEKHFNDRIKLTGANRIWESVIFAVRGILLSDNDSIKNAISGIQDVMVITDSGDGFYKDGSFIQHDNIPYNCGYGRSLIQELAPMLYIFKDTEFENKNTDIINTWIEKSYLPFIYNGRAMDMVRGREISRYYEQSDLACTHILSAMLILSEMPEFNELKGTIKTQITDNFFEYASVFTAELAEHLQEDNNIKPKEIKPYFMAFNSMDRVVKHGNGYTIGLAMHSERTAAYESINDENQNAHHTSDGMMYIYKKNEPKSDFFWQTIDLQRLPGTTVLRGSTVKPNINAAGDFTGGCGIGENGVCTMKLISNENSLKANKSWFFFDKEVVCLGSCINSEEESEVETIIENRLVTDNSRFTVHGNEESEGYIIKGAYLDGSHDVGYCFPEEQEVNIFREIRSGDWNNMSIKSDGKSYKGRYLTMWIKHGRKVKDVSYEYIVIPKCHEEEINDYYRKSGIRIIENSDSIQCVKKNGTTGVVFLKDKTHSAGGISCDRRCIVMTTQTCGTLELSISDITQKQDKIYIELDYSAQEIISKSERINIIQLVPYVCMEIDTCAARGEEQHIKFGGVKNV

SEQ ID NO: 5 - protein, 815 aa, Unknown
  Ruminococcaceae UCG13
MENLLVRRTNMKYGYFDDLNKEYVIETPRTPLPWINYLGTNGFFSLISNTSGGYCFYKDAKHRRILRYRYNNIPADNGGRYFYINDNGDCWTPSYMPMKKELDFYECRHGMGYTKITGERNGVRVEQTAFVPVDDNCEIHRIKVTNTSGEAKNINLFSFVEFCLWNAQDDMLNYQRNLNTGEVEIDGSAIYHKTEYRERRNHYAFYSVNTEISGFDTDRDTFLGAFNGLDTPDRVINGKSGNSVASGWYPIASHQIDVSLDAGESREYIFVLGYIENEKDEKFESLNVINKTKAKEMIARYESSAQCDAELDKLKLYWDNLLSVFTLESNDEKLNRMVNIWNPYQCMVTFNMSRSASYYESGIGRGMGFRDSNQDLLGFVHQIPERARERIIDIASTQFEDGSAYHQYQPLTKQGNNEIGGGFNDDPLWLILGTVAYIKETGDYGILDEQVPFDCDKNNTATLLEHLNRSFGHVTNNLGPHGLPLIGRADWNDCLNLNCFSEIPDESFQTTGDDDGRVAESVLIAGMFVYIGREFARLYKTLNNDEMYKYISDEVEKMTEAVLEYGYDGEWFIRAYDANGNKVGSDECDEGKIFIESNGFCVMAGIGKEDGRAQKALDSVKKYLECEYGIVLNYPPYSGYRLELGEISSYPPGYKENAGEFCHNNPWVIIGETVMGNGERAFELYKKIAPAYLEEISEIHKTEPYVYSQMIAGRDAVRAGEAKNSWLTGTAAWNYYTVSQYLLGIRPDFDGLVIEPCISKDISEFKVTRKFRGKTYNILVKNTGEGTVKITADNGTVNGTTVSSDAEICNVEVVM

SEQ ID NO: 6 - protein, 928 aa, Unknown
  Ruminococcaceae UCG13
MILIYNSDIMYNKYIKPKFIIWYKKEFQMSKNVHIISHSHWDREWYLPFEQHRMRLVELIDKCMEVFEKDDSFKSFFLDGQTIVLDDYLEIRPENKEKLIKYTKEGKFIIGPWYILQDEFYTSGEANIRNLLVGMKEAEKYGAMCKMGYFPDAFGNAGQMPQLLKQAGMDTVTFGRGVRPVGFDNEVQENGNYESPYSEMMWESPDGTKIFGILFANWYNNGNEVPTDKKIAKEYWDDRLKKVATFASTDEYLLMNGCDHQPVQADLGKAIEVASELYPDINFKHSNFPEYIKAIKEKVPNDLAVVKGELTSQDTDGWSTLMNCASSHIYLKQMNRKCESALENGAEPVRVLSSVLGQNYPSDELEYSWKKLMQNHPHDSICCCSVDEVQDEMATRFNKSKQVADYLVSEGKRYIADKINTKEYEKYKNALPFVVFNTAGRERTSVVSVEIDVTRKSGWLKKCAYDLDEINVPNYKLIDSDGNSIPFKIEDLGVKFGYDLPKDKFRQPYMARRVRVTFEAENISAVGYKTYALVEGDTEKVTDTLVSSENCMENDAIRVEINKNGSLNVTDKASGRTYKGVAYYEETGDLGNEYMYKMPEGSKAITTQDTVAKIELAEDEPYRAMYKITNTITVPKSGDDNFEDEKSHMVFFKERVGGRSNDTVEMKIETFVSLDKNGKGVKIKTRFDNEVKDHRVRIMVPTGINSDVHKADSVFEVVTRNNRHNAGWNNPSACEHEQGFVSIDDGEKGIAVANIGLYEYEMLPDLDNTIAVTILRAVGEMGDWGVFPTPKAQCLGISETEIEIVPFKGDLISSGAYEECYQFRTDIITADTDCHDGVMPLDYSMINWQGNGLILTGIKQKGNGEDIIIRWVNVSDKTTTLTIQKSDVIDNLYISNIIEKKIKKIDSNNNYFNIEVKPYEIMTVGIAK

SEQ ID NO: 7 - protein, 950 aa, Unknown
  Ruminococcaceae UCG13
MERKNIKCHFISNTHWDREWKFSAQRTRHMLVTAIDMLLDIFEKEPDYKHFHLDSQTLPIQDYLEINPEKKEILKKYISEGKLAVGPWFCLPDEFCVGGESLIRNLLLGHKIANEFGKVSKTGYSPFGWGQISQMPQLYHGFGIDFASFYRGLNTYMAPKSEFYWEGADGTTIYASRLGQRPRYNMWYIMQRPVFYGKRDGDNRRVSWGAGDGIFRFADPARCEYEYQYSHRKYEYHDEYIAEKTEQALSEQDDEWTTPNRFWSNGHDSSIPDMRESRLIKDANAVYEGVDVFHSTVYDFEQSVIRDFDKNSPVLKGEMRYPFTKGSVSALFGWVLSARIKVKQENFETERLLTSYAEPMAVFASVCGAVYPQAFINKAYNYMLQNHGHDSIGACGRDVVYKDVEYRFRQSREIATCVLERALMDLSGDIDFAGWDKNDMALVMFNPAPFKRSLTVPCELEIPLEWECDSFEIVDAEGNVCPHQNISSINPMYQIVQDLADAVDVLPVSRHTIRIFVKDIPSMGYKTLKVVPKYHTRATTPVNMLCGINTMENEYLKVKINSNGTLKVTEKETGREYDNIGYFKDTGENGSPWEHKTPELDEEYTTVNERAIVSLVYSGELETKYRIVLNWAIPENIVDGGKKRSSRLAPYRIETLVTLRKGARWVEFETKINNNVPNHYLQAAFPTDVDAEFVYAQGQFDVVKRPIAKPDYSKYDEIPMTEQPMNSFVDICNENEGAAILNTGLKAYESDDDYNHTVYLSLLRCFELRIYVTPEEQNYSRIENGSQSFGEHTFRYAFMPHKGDWEDAQVWKAAEDFNMEILIGQTAPTEHGKNPLEKSFIELENENLHISAVKRSEDGLGCVVRLFNPSSETVKNRIRFNGGIAEISDKQSPIERQVHSFELPCTENRKWASVKKVTLEELSETELSVDTNGWCDVEVTPKQIYTLKYE

SEQ ID NO: 8 - protein, 367 aa, Unknown
  Ruminococcaceae UCG13
VNIDKAITYAESIVRKSLNYFYDCFPTEQSENLVFKKFENVSWTTGFYEGILWLMYELTGDKAFYNSAKHHSEMFHKRLVDRVELEHHDMGFLFTLSSVADYRITGDEQAKQDGIEAAEWLLKRYQPKGKFIQAWDAMDDSQSYRFIVDCMLNIPLLFWASEVTGYKKYYDAAYNHMQTSIANIIRPDASSYHTFFFDPVTNKPLRGETHQGFSDDSSWARGQSWAVYGLALCYHYTKEKSILPLFERVTHYFIDHLPEDSVPYWDLIFSDGSDEPRDTSAAVVAVCGILEMEKYYHNQEFLDAAEKMMTSLSEKYTTVDYPQSNGIIKDGMYSRKHGHEPECTSWGDYFYLEALMRMKKSDWKIYW

SEQ ID NO: 9 - protein, 1604 aa, Unknown
  Ruminococcaceae UCG13
MKKIISLMLAVTMICASIGLTAFAATTTTVEAEADGVSAYTLPSSDKSNSKILKNTVSSKESVTYYIQANNTPRATMFKLAQVNTGDKINVDINFTYLDTATMELEYCLFVSDSEITLTSHSQDLVKEELEKHTDESNIKNWSTNKSNMKYSLPNGITASKDGFVYLYIGCGDLSEDKTQVTKKIQWSIDSFDVNIDSDGGGETEPDTTPTPTTTINPDVTPTPTPTASPTPTPTLEPELTLNAVYSSNMVLQRKEPITITGTGKSGNTVSVNFNGADEQTTIEHGLWEITLPAMEAVKSATMTVSSGDNMITLDNVAVGDVIFCTGQSNMFNRLETFPTLMNEELSEAYEDVRYMNSFDEISEWKVATMENSKQFSALGFLIGKRMIKKDSDVPIGLISSSLGGSSIMQWIPTYSVNWDSQAKRMMAGASSKGGLYTQRLLPLKNLKASAVVWYQGEANTTFESGTVYEQALTSLINNWRKTFNDEDLPFVVIQLPTANFAKIYSTIRIGTGVRAGQWNVSQRMDNVKTVVSNDTGTTNNVHPNDKGPIADRAVAYIEDFINNTQSNVESPSFDYMERSGDKLILHFKNTYGSLSTDDGGVPLGFELKDDDGIYKDVTPTINGDTIEIDVTDITNPQVKYAWSDTPGIAKDLVEAQTDTPAVINTFNAAGRPIAPFMTDLTEKYASKAVNKELSTTEFYNYAPYISKVEQSGDDIVISAYDTDGVVSKVEVYIDEGEIKAGDAKQRDDGKWVFTPDVTSGVHSVYAIATDNDNINSLTCVDYTTYNIIRPTRYDYVKGYTESPSSVEYNNGDDMLAKATNDVNGTTTTVTSAIPTGETTKSLKLSATGNKATANATIPISKADNPQKTLTIEYDTMFESADDAIGASRGMYAKTKEGNELWLTYFTASSLRTAITNTGGNWCYEQAMSIKNNQWHHIKLELHPNTGIFSIWLDGTMLQDNVSFVKEGSSFDTCKGAFDTLKEGITDLRFYHTASNNIENATYIDNVKVTEVSYSEEEIIPPAKIQEATPQISIDYINETLTGFESQEPYTIKVGEGNAKDITLGEGVTTISLDDEKIGYAGKLLSIEIVKKARNTETYTDSDVQQLTVKARPKAPTTVQGVNATEIGGKGKLTGMNGMQYKLKRTDEWSSTQLVDTVEVDAGEYNVRKAATDTDFASEKTTITVETFIAEKEMTPEIAIDYTTEELINFVEDGTYTINGLDVTLTDNKLSLANYITNEQITLSIVKKGNNVTTVASEAQTLIVKARPAAPTKSEIIVTQPSVIGGKGTIAGIADTMEYSTNNGINWTTGDGDDIGDIEPGTTYKIRYKAVSADEEAERQFKSAEYSVTIIAYDAMPETQPTISINYVNEKLTGFTEGCDYIIKIDDGVATDKDNVTEDIDIDNTYFGHTLKIVKKDDGIKTSNSEAFELSIPKRSSAPNVAAVEEQTYQGNDGKITGVDTTMEYKSLSEPTFTWMQCVGTEITNLAPGSYIVRVAAVADESFASEVMSVTINAAAKDEPTEPTVNITYDDKNGNVNAIFTNITEEGMVYVAEYNENGTLLSIKSDEISDSVIIPFTCVNKSKVKVFIWKNDMKPLFNKVFTLN

SEQ ID NO: 10 - protein, 266 aa, Unknown
  Ruminococcaceae UCG13
MFNKKFNLLKEATEYGFMPYMETYILDGKKRPIVVIFPGGGYGMVSEREAERIAMAYNAAGFHAAVVYYCVEPHTHPLPIQNAANAVAMLRENAEKWNIDTDKVIVCGFSAGGHLAASLSALWNDSEIFSEREIELAMHKPNAQILSYPVITSGEFAHKDSFKNLTGTDDESNHLWSSLSLERRITDIIPPTFLWHTYEDICVPVENTLMYAAGLRRVGVPFELHIFEKGEHGLSRVSDELIWSKRKFEREYPWLSLSVDWLNQLF

SEQ ID NO: 11 - protein, 532 aa, Bacteroides intestinalis
MKKRHIIGSFLLGLLLTVNTGCEDFLDQKDTSGINENSLFLKPEDGYSLVTGVYSTFHFSVDYMLKGIWFTANFPTQDFHNDGSDTFWNTYEVPTDFDALNTFWVGNYIGISRANAAIPILQRMKDNGVLSEKEANTLIGECYFLRGVFYYYLAVDFGGVPLELETVKDEGLHPRNSQDEVFASVVSDMNIAAGLLPWKAEQGSADRGRATREAALAYQGDALMWLKQYKEAVEVFNQLDSKCQLEENFLNIHEIANRNGKESIFEVQFTEYGSMNWGAFGVNNHWISSFGMPVAISGFAYAYADKKMYDSFENGDLRRHATVIGPGDEHPSPLIDLQDYPKLKDFATKGNGNIPASFYQDEEGNVLNTCGTVENPWLDGTRSGYYGVKYWRNPEVCGTRGAGWFMSPDNIMMMRYAQVLLSKAECLYRLNDSNGAMAIVQKVRDRAFGKLQNSAVEVPAPANTDVLKVIMDEYRHELTGETSLWFLLRRTGEHANYIKEKYGITIPTGKDLMPIPQTQIGLNQNLKQNPGY

SEQ ID NO: 12 - protein, 1047 aa, Bacteroides intestinalis
MKTKFIATFFLLICGSVMFAQTRTVKGKVVDKANEPLIGVAVNIKNTSQGSITDFEGNYSIQVNTENAVLVFSYIGYDKQEIKVGARNVIDVVMHEASIALDQVVVVGYGTSKRGDVTGSISSIDAAEIKKVPVVNVGQALQGRMSGVQVTNNDGTPGAGVQVLIRGVGSFGDNSPLYVVDGYPGASISNLNPSDIQSIDVLKDASAAAIYGNRAANGVVIITTKRGNADKMQLSVDATVSVQFKPSTFDVLNAQDFASLATEISKKENAPVLDAWANPSGLRTIDWQDLMYRAGLKQNYNLSLRGGSEKVQTSISLGLTNQEGVVRFSDYKRYNIALTQDYKPLKWLKSSTSLRYAYTDNKTVFGSGQGGVGRLAKLIPTMTGNPLTDEVENANGVFGFYDKNANAVRDNENVYARSKSNDQKNISHNLIANTSLEINPFKGLVFKTNFGISYGASSGYDFNPYDDRVPTTRLATYRQYASNSFEYLWENTLNYSNTFGKHSIDVLGGVSIQENTARNMSVYGEGLSSDGLRNLGSLQTMRDISGNQQTWSLASQFARLTYKFAERYILTGTVRRDGSSRFMRGNRWGVFPSVSAAWRIKEESFLKDVDFISNLKLRASYGEAGNQNIGLFQYQSSYTTGKRSSNYGYVFGQDKTYIDGMVQAFLPNPNLKWETSKQTDIGIDLGFFNNKLMLTADYYIKKSSDFLLEIQMPAQTGFTKATRNVGSVKNNGFEFSVDYRDNSHDFKYGVNVNLTTVKNKIERLSPGKDAVANLQSLGFPTTGNTSWAVFSMSKVGGSIGEFYGFQTDGIIQNQAEIDALNANAHRLNQDDNVWYIASGTAPGDRKFIDQNGDGVITDADRVSLGSPLPKFYGGINLSGEYKGFDFNLFFNYSVGNKILNFVKRNLISMGGEGSIGLQNVGKEFYDNRWTETNPTNKYPRAVWSDVSGNSRVSDAFVEDGSYLRLKNIEVGYTLPANILKKASISKLRIFASVQNLFTITGYSGMDPEIGQSMSSSTGVAGGVTASGVDVGIYPYSRFFTMGFNLEF

SEQ ID NO: 13 - protein, 783 aa, Bacteroides intestinalis
MKTFILSFLIYAGCSLPLTAQQIKPAIPSDPEIEAKINKLLQKLTLEEKIGQMCEITIDVITDFSDKENGFRLSESMLDTVIGKYKVGSILNTPFSIAQEKEVWADLITRIQKKSMEEIGIPCIYGVDQIHGTTYTRGGTFFPQSINMAAAFNRQLTRRGAEISAYETKACCIPWNYAPVMDLGRDPRWPRMWESYGEDCYVNAEMGVQAVKGLQGENPNHIGENNVAACIKHFMGYGVPVSGKDRTPSSISRTDLREKHFAPFLASIQAGALSLMVNSGVDNGVPFHANKELLTGWLKEELNWDGMIVTDWADINNLCLRDHIAETKKEAIQIAINAGIDMSMVPYEVSFCTYLKELVEEGKVSMARIDDAVSRVLRLKYRLGLFDNPYWDIRKYDQFASPEFASVALQAAEESEVLLKNEDDILPLAKGKKILLTGPNANSMRCLNGGWSYSWQGDKADECAQAYNTIYEAFCNEYGKESVIYEPGVTYKTSADALWWEENTPRIAQAVSAAEKADVIIACIGENSYCETPGNLTDLNLSTNQKDLVKALAATGKPIILVLNEGRPRIIHDIVPLAKAVVHIMLLGNYGADALVNLVSGKANFSGKLPFTYPHLINSLATYDYKPCENMGQMGGNYNYDAVMDVQWPFGFGLSYTTYSYSNLKVNRTSFDADNELVFTVDVTNTGKMAGKESVLLYSRDLVASITPDNIRLRNFEKVDLQPGETKTVTMKLKGSDLAFVGADGKWRLEKGAFRMTCGTQKLEVHCTTTKIWQTPNISKSGI

SEQ ID NO: 14 - protein, 564 aa, Bacteroides intestinalis
MKNTVLPLILFLCMLCLGSHLYAGHSMHPLNQISYVKKKIKEQQEPYFTAYRQLMHYADSIQEVSQNALVDFAVPGFYDKPEEHRANSLALQRDAFAAYCSALAYQLSGEERYGQKACYFLNAWSSTNKKYSEHDGVLVMSYSGSALLMAAELMMDTPIWNPQDKDAFKTWVSQVYQKAVNEIRVHKNNWADWGRFGSLLAASLLDDKEEVARNVQLIKSDLFVKIAEDGHMPEEVVRGNNGIWYTYFSLAPMTAACWLVYNLTGENLFVWEHNDASLKKALDYMFYFHQHPSEWKWDTRPNLGAHETWPDNLLEAMAGIYNDASYLQYVESSRPHIYPLHHFAWSFPTLMPVSLKGYDLTDNNTWANYNRYEVANKTVKKPVAIFMGNSITEGWNRSHPDFFTQNGYVGRGISGQVTAQMLARFRADVLDLKPQVVCILAGTNDIAQNCMYMSVENIAGNIFSMAELAKANGIKVVICSVLPATRYSWRPTVQNPAGQIIQLNKLLQKYAQKNKIPYVDFHSMMKDEQNGLPQKYSKDGVHPTKEGFSMMEPIIKEAIDKLLK

SEQ ID NO: 15 - protein, 345 aa, Bacteroides intestinalis
MKNIYYILILCCLCLFSCDSHPDTKSSLPFGVNLAGAEFFHKKMDGVGQFGIDYHYPTTREFDYWKSKGLTLIRLPFKWERIQRELYGELNREEIDYIKYLLDEAGARDMKILIDMHNYGRRKDNGKDRIIGDSVSIDHFASVWKQIAGELKEHSALYGYGLINEPHDMLDSVPWFKIAQAAIEESRKVDLKTAIVVGGNHWSSAARWQEISDDLKHLHDPSDNLIFEGHCYFDEDGSGIYRRSYDEEKAYPTIGIDRTRPFVEWLKTNNLRGFIGEYGVPGDDERWLVCLDNFLDYLSKENINGTYWAAGAQWNKYILSIHPDDNYQTDKIQLGVLTKYLETKN

SEQ ID NO: 16 - protein, 400 aa, Bacteroides intestinalis
MRKQLSLLLVSISLGWVGCAPDKQADTIHLDRQLEYCDAQIRRTLSEADQDSCLMPRSMEANQTNWNMSNIYDWTSGFWPGILWYDYEATGDEEIKAQAIRYTECLLPLVTPAHGADHDIGFQIFCSFGNAYRITGNEEYKTVILKGAQKLAKLYNPKVGTILSWPGMVKRMGWPHNTIMDNMMNLEILFWAARNGGGQELYDIAVKHAQTTMKYSFREDGGNYHVAVYDTIDGHFIKGVTNQGYGDSSLWARGQAWAIYGYMMVYRETQDKTFLRFAEKVTELYLENLPEDYIPYWDFDAPDMIKQPKDASAAAITASALIELSELEDTPSLASRYLNAATRMLGELSSERYQCRDIKPAFLMHSTGNQPGGYEIDASINYADYYYLQALLKYKKAMGL

SEQ ID NO: 17 - protein, 704 aa, Bacteroides intestinalis
MKTRTLGICLFLLMNVSFIKGQSLADKVDMWMGTYGAGHCVVGPQLPHGSVNPSPQTAYGGHAGYVPDQPIRGFGQLHVSGIGWGRYGQIFLSPQVGFNPGETDHDSPKQGEEATPYYYKVMLSRYDIQVEISPTHHCVAYRFTFPETDQGNILLDIAHNIPQHIVPEVKGLFHGGEINYNPEQQTLTGWGEYSGGFGSTDAYKVYFAMKTDTPLKEVKITDQGDKALYACLALNKNPGVVHLNVGISLKSIENASLFLSEEIADNSFNTVKENAKAIWDNTLSSIKIKSENEAEERLFYTTLYHSFVMPRDRTGDNPHWDSESAHMDDHYCVWDTWRTKYPLMVLLRESYVAQTINSFIDRFAHNGVCNPTFTSSLDWTSKQGGDDVDNIIADAIVKNVKGFDYEKAYALMKWNAYHARSKDYLRLGWEPETGGIMSCSAGIEYAYNDFCTSEIAGIMHDENTQKELYERSGNWSQLFNPLQESHTYKGFIVPRKANGEWVAIDPAKAYGSWVEYFYEGNSWTYTLFVPHQFDRLIEYCGGKANMIKRLSYGFENNLISLNNEPGFLSPFIFTHCGRPDLTARYVSQIRKDNFSLLKGYSDNEDSGAMGSWYIFTSIGLFPNAGQDFYYLLPPAFTDVELTMENGKKISIKVLKDTPDACYIKSVSINGKVLDKGWIYHREIAEGATLVYELTNKENAWHINE

SEQ ID NO: 18 - protein, 314 aa, Unknown
  Ruminococcaceae UCG13
MKYYIGIDLGGTNIAAGIVDKTGKIIAKDSVPTLNTRPIEAIMLDMTKLCKTLLDKSQMDINKIEAVGIGCPGTVDNKNGIISYSNNIPMKNVPMRKFMEKQLNISVNLENDANAAALGEYTANGHNASSYILITLGTGIGGGAVINSKIYRGFNGVGIEPGHMTLINGGERCTCGKHGCWETYGSVTALINQTKLKMTDNPDSLMHKISGKFGEVNGRVAFEAAKAGDKAGLEVVEKYTEYVADGITSVINIFEPEILVIGGGISKEGEYLLNPIRKFVEINEFNKYRPKTKIEIASLNNDAGIIGAALSANR

SEQ ID NO: 19 - protein, 738 aa, Unknown
  Ruminococcaceae UCG13
MKKLVSLIIAMSIFFSINCAIFATNVSYMADFESADAKFGNSTTYSGTKNTAGDYSDFVKPEWVADGGKENSTGLRITYKAATWYAGEVFFPIPVAWQNGADAEYLNFDYNGKGIVNISLSTGSAATDTLTKGTKYSYKLNADTNGEWQSISIPLSEFKNNGNPVTIANIGCVTFQAGENGGLSNSASETKAMTAAELEAKARNGSIIFDNMELSNVGENVLNPNATPEPTEKPDNTTRTIDFDTYTLSHKQTWAGFNNNDKTYSDSIKSEITENGKEGCALELTYKAATWYAGEIFMSIPKEWAINKNSECLEFDAKGQGKIKISLETGEVVNGIRYGHTVTINTNDEWQKISVPLSEFVNNGNEVPLTDVVGMAFSAAESGNLDNNAEETKMMSADELEEKAVTGCVVIDNITLAEQDTTSPTAAPEATTQPTEISYVADFETADTKFASGKTWGGFKNKSNDYQDYIKAKWLQDGGVDGSTAFCVYYQSATYYAGEIFVPAPAVWTNNGAKGAEYLNFDYKGKGAVKISFSTGNTVDGTLTSGTRYTRRFELDSHGDWAKISVPLSEFVNGENIVNMTEIGTVTFQAAENANLDNNSDDTKAMSADELKEIARTGEIIFDNMTLSETEGKTTLFSSVKVTAEIDGKEITNLTNGDIKIKAIASDIEKDTNMVMIVAVYKENGVIDTVRMAGQKIIGDGELMLDLNVTDAEHQTMKVFIFDDFTNLHPIINVTNFL

SEQ ID NO: 20 - protein, 335 aa, Unknown
  Ruminococcaceae UCG13
BMPTIRFVYTYSLLWWAERLCGKMYYIGIDLGGTNIAAGIVTEEGKIVVKDSVPTLSERPTDEIVTDMANLSKKLVQSIGIEMNEIKGIGIGCPGTIDFETGEIVYSNNIKINHYPLADKFKEHIPLPVKVDNDANCAALGEYKINRHCASVFALVTLGTGVGGGVIINGKVFRGFNGAAGELGHMTIVSGGKMCTCGKEGCLESYASATALISQTKDALETHKDTIMHGIVKKEGKISGRTAFEAAKQGDEVAKKVVSNYERYLADGIVSIENIFQPEIIAIGGGISKEGDYLIEPIREYVYNTGFNKHMTKTKIVAAQLFNDAGIIGAAMLAI

SEQ ID NO: 21 - protein, 588 aa, Unknown
  Ruminococcaceae UCG13
MSEKFNNMSFRTKLLLSYIAVIILCIIIFGLTVFSSISRRFENEITDNNAQITGLAVNNMTNTMNNIEQILYSVQANSTIEKMLTASNPPSPYEEIAAIEQELSKIDPLKATVSRLSLYIENRTSYPSPFDSNVTASVYSKNEVWYKNTKELNGSTYWCVMDSSDANGLLCVARAFIDTRTHKILGIIRADVNLSQFTNDIAHISMNNTGKLFLVYENHIINTWNDSYINNFVNENEFFKAISADSDKPQLVQINKEKHIINHSRLKDSSLILVRASKLDDFNSDIHIIEKSMITTGIIALLVALIFIFIFTRWLTAPITKLIKHMERFENNYERIPIEITSHDEMGKLGESYNSMLNTIDSLITDVEDLYKKQKIFELKALQAQINPHFLYNTLDSIHWMARAHHAPDISKMVSALGTFFRHSLNKGNEYTTIENELNQISSYVSIQKIRFEDKFDVVYDIDENLLHCTIVKLTIQPLVENSIIHGFDEIEEGGMITIRIYPEDDYIFIDVIDNGSGADTNELNKAITHELDYNEPIEKYGLTNVNLRIQLYFDKTCGLSFKTNETGGVTATIKIKRKEPEYKTIDL

SEQ ID NO: 22 - protein, 337 aa, Unknown
  Ruminococcaceae UCG13
MQCRGGNVMNFNIPDLGIIDGSSGFRNLPSTTDGRFTSGEDGVKHIVCTGDGKVEFVAFENQTLAYVNSALGYGAYYPLHPVNRNGKIKAVLMDLDGTSVRSEEFWIWIIEKTTASMLDDESFKLEESDIPFVSGHSVSEHLQYCIDKYCPGESLDKARNFYFDHVNREMKEIMEGRGRKNAFVPQEGLKEFLLALKAKGIKIGLVTSGLYEKAMPEILSAFRALDMGEPTDFYDAIISAGYPLRKGSVGTLGELSPKPHPWLYAETCAVGLGVGFDERGSVIAIEDSGAGVCSARIAGYTTIGLAGGNIKESGTMPMCSRYCNNLAEILDYIEEEA

SEQ ID NO: 23 - protein, 334 aa, Unknown
  Ruminococcaceae UCG13
MFFSVLHMAIINIKGVKIVSELYPVRLIPVFKDYLWGGTKLKTVFNKKSELNILAESWELSANKDGQSIIANGKYQGYGLKEYIDIVGKEIVGTKGLALDDFPILIKFIDAKKNLSVQVHPDDEYATCHDGANAKTEMWYILDCNVGAYLYYGFKKDITKQEYQDAIRSNTITDVLNKVPVHKGDVFFIPAGTVHAIGAGILICEIQQNSNTTYRVYDYDRRDKDGNKRELHIREALESSNLKKSTYSNSVLDGDDIILTQCDYFTVRRLKVQNRVQLRIDKTSFHSLIITDGSGELYMGGEILKLNKGDSIFIPAQNNEYTVSGPCEIILSFL

SEQ ID NO: 24 - protein, 524 aa, Unknown
  Ruminococcaceae UCG13
MNVKLLICDDEKIIREGLASLDWNTRGIEVVGTAKNGEVAFELFQKMLPDIVISDIKMPTKDGIWLSEQIHKISPNTKIIFLTGYNDFEYAQSAINNGVCQYLLKPIDEFELYEIVDKLTKEIHLEQQKAEKEIELRKTLRNSRYFLLNYLFNRAQYGILDFELFEISKKAAAMTTFVIRLDTDSTNYGMNFMIFEALIEHLPKTINFIPFFSNSDLVFICCFNEPEGESEQKLFSCCENLGDFIDTEFNVNYNIGIGIFTSEISELEASYTSALQALDYSDRLGQGNIIYINDIEPKSQLSAYQSKLIETYIKALKNNDEKQSKKSVKELFDVMERSDMNLYNQQRRCMSLILSISDALYDIDCDPTILFKNTDAWSLIRKTQSPAELKTFVENITDVVISYIESVQKQKAANIITQVKALVEKNYARDASLETVASQVFISPCYLSVIFKKETNITFKNYLIQTRIEKAKELLEKTDLKIYDIAEKVGYNNTRYFSELFQRICGKTPSQYRASHNPSMPQDI

SEQ ID NO: 25 - protein, 385 aa, Unknown
  Ruminococcaceae UCG13
MSDKKPLYKQIMDKLKERIKSGDFEYDAPFVTEDRITKEYGVSRITAIRALEELEHDGLINRKRGSGSFVSKNAMSILGKDKEDNAAVTIHKKNRDISLVALVMPFDIKLGNMFKCFDGINSVLNKENCFVSIYNANRSVENEEKILRSLLEQGIDGVICYPVRGGRNFEVYNQFLVKKIPLVLIDNYIENMPMSYIVSDNSGGGKALCEYALEHGHKKIGFFCRGRVNETISIRDRYMGYAAALEEKGLGVNLDYVYANIDDKYEMLTEEERQQYGNVENYLKTIVNRMHEQGISCVLCQNDWVAIQVYNCCKALDISVPNEMCIMGFDNISELDEMDGGNKIITVEQNFFELGVKAGETVLREINGEMPGIKYIVPVKIAVRN

SEQ ID NO: 26 - protein, 269 aa, Unknown
  Ruminococcaceae UCG13
AMLVVLGASFTSESAISEFGFHAIPKEWSLDAYRYIITSKETILRAYGVTIFVTIVGTLMSTLVVALYAYPLSRKDFKYRKLFTFIAFFTMLFSGGTVAGYMVTTGILNLKNSIWVLIFPYVMNAWHVIVMRSFYSMSIPTAIIEAAKIDGANEYQIYFKIVLHISLPGLATIALFATLTYWNDWWLPLLYITEPQKYNLQYLLQSMISNIQNLTENSAQMGSANLLANVPKEGARMALCIIATLPILFVYPFFQKYFIQGLTVGSVKE

SEQ ID NO: 27 - protein, 294 aa, Unknown
  Ruminococcaceae UCG13
MLSMCIPGLIFFILFNYLPMFGIIIAFKQYRYDLGIWASPWNGLKNFEFMFSSPDAWVITRNTIAYNLLFIFGGLVFNVAMAIGLSELRNKAVSKLCQTVVIMPHFLSYVIVSFLVLAFLHVENGLINRSLIPALGLEGVDWYSNPKYWPWILVIVNFWKTTGYGSVVYLAGIAGIDTSLYEAAKVDGASRWQQIRYITLPALVPLMVVLTILNVGKIFNSDFGLFYQVPLNTGALYPATNVISTYVYNMLMSAGTGSVGMASAAAFYQSIVGFILVMTTNFIVKKISPENALF

SEQ ID NO: 28 - protein, 515 aa, Unknown
  Ruminococcaceae UCG13
MIMGKDETSEPLSKKKGDKIMRKKIAALLAMLMLGGVLTGCGGGNKVATGGEDPNVVPEDTYEINWYMQGMPQEDVASVEAAVNDYLKDKINATLKMHRLESNQYSKQLNTMIAAGEYFDIAWTTPGVLTYTANARNGAWLALDDYIDTYIPKTIEQLGEIADNARVDGKLYAIPTYKEMADSRGWTYRKDIAEKYNINMDNIKTFDELLPVLKMIKENEPNMQYPIDWGSDRTPEALMKYEEIAGTAVIFYDTDKYDGKVVNLVETPEYLEACKWDNKLYNEGLVKKDIMTATDFEQRLKDGKTFCYVDFLKPGKAKETSAKFDFELDQSTVSDIWQDNGAGTGSMLAVSRTSKNPERVLRFLELLNTDATLSNLINYGIEGKHYTKIDDNTITIPDDTSYTLQGYQWMQGNVFLNYLTEGESPDKVEALKAFNAEAKKPIDYGFKFDNTAVEAEIAACQTVKSEYRKQVIMGSMDPEPIMKEYAAKLKAAGIDKIIEEAQKQYDEFLANKNKQ

SEQ ID NO: 29 - protein, 385 aa, Unknown
  Ruminococcaceae UCG13
MKKLLILFLLASVMLSMCSGCTVEKTVESAQAVTVLKVIKPNYISDFTQNIAEFNEANPDIQVKFIDAPTSTEKRHQLYVSALSGKDSSIDIYWINDEWTKEFVEQKYIKALDGEILLDNSRYIIDAQERFSVNDSFYAMPVGMDTDVIFYRSDKIHNVPETWDGIINLCRNSDFGLPIKLGLTTSDIQDMMYNIIEIKEAIGISYAETLNLYKEFIEEYKDIENYTDTIAAFKIGSAAMLMGNSSLWKKLNGDTSAVKGNIMVASLPNKNQFVRSYALAINSNSKNQEAAIRFLDFMNGKEQQRRLSRDTSLIPIIRELYDDEMILDANPHVKGIKQSVQNSSSFATVSINGENLKKLEEALIKFFNNEETSMNTGKIFEDLMQ

SEQ ID NO: 30 - protein, 1324 aa, Bacteroides intestinalis
MKQLITTLFIFIFLQPSWASLYRNYQVEDGLSHNSVWAVMQDKQGFLWFGTVDGLNRFDGNSFKIYKKLQGDSLSIGNNFIHCLKEDSHGHFLVGTKQGFYLFNRESETFSHVRLDNRSRGGDDTSINYIMEDPDGNIWLGCYGQGIYVLGPDLQVRKHYINKGNPGDIASNHIWCMVQDYNGVIWIGTDGGGLIRLDPKDERFTSIMHEKDLNLTDPTIYSLYCDMDNTIWVGTSISGLYRCNFRTGKVTNIVYPHRKILNIKAITAYSNNELVMGSDAGLIKVDCIQETISFINEGPAFDNITDKSIFSIAHDMEGGLWIGTYFGGVNYYSPYANKFAYYPGSSEEVSKSIISYFTEESSDKIWVGTKNEGLLLFNPAKISFETTHLQIDYHDIQALMMDNDKLWISVYGKGVSMVDVHSNTLLKRYSNDVGGPDLLTSNIVNVIFKSSKGQIFFGTPEGVDCLDAETKKINRLERTKGIPVKAIMEDYNGSIWFAAHMHGLLHLSADGTWESFTHMPEDSTSLMSNNVNCIHQDARYRIWVGSEGEGMGLFNPKTKKFEYILTENLGLPSNIIYAIQEDADGNIWVSTGGGLARIEPETRSICTFRYIEDLIKIRYNLNCALRGRDNHLYFGGTNGFIAFNPKDIQNNEYKPPICLTGFQISGNEVVPGIEGSPLKKSISMTQKIELESNQAAFSFDFVCLSYLSPAQNKYAYKLEGFDTDWHYVANGNNKAIYMNIPSGKYTFYVKGTNNDGVWCDTPIKVTVIVKRHFWLSNMMLLVYAILAISAFTLLIRRYNKRLDSINQDKMYKYKVEKEKEIYETKINFFTNMAHEIRTPLSLIVAPLENIISSGDGSQQTKSNLEIMKRNANRLLELVNQLLDFRKIEEDMFRLCFSKQNISEIVRNIHKRYVQYAKLKDIDIRLVEPEKDIACVVDKEAMEKVIGNLLSNAVKYANSLITINISTDNNLLTISVKDDGPGIKSEFIDKIFESFFQIENNAQRTGSGLGLALSKSLVTKHKGNIAASSDYGHGCTLTFTIPMDLPISISQLTEEYPEKEDISVQQTALSPVEGKLRIVLAEDNQELRSFLSNYLSDYLDVYEAQNGLEALQLVENENIDIIVSDILMPEMDGLELCKALKSNPAYSHLPFILLSARTDTATKIEGLNTGADVYMEKPFSSEQLRAQINSIINNRNSIRENFIKSPLDYYKQKSAEPNGNTEFIEKLNIIILDNLTNEKFSIDNLSEMFLMSRSNLHKKIKNIVGMTPNDYIKLIRLNQSAQLLATGKYKINEVCYLVGFNTPSYFSKCFYEHFGKLPKDFIVIE

SEQ ID NO: 31 - DNA, 55290 bp, Unknown
  Ruminococcaceae UCG13
catttatctatattttatgtacaaatattaatatttgcttctatactatatattatttatctattcgcacttaaggcagcacctataattcctgcatcattattcaaagatgcaatttcaatttttgtctttggtctatatttattgaattcatttatttcaacaaattttctgattggattcaaaagatattccccttctttgcttattccgccaccaataaccaaaatctcaggttcaaaaatatttatgacacttgttataccgtcagcaacatattctgtatatttctcaaccacttccaaccctgccttatcacctgcttttgccgcctcaaaagccactctgccatttacttcaccgaatttccccgaaattttatgcattaagctgtccggattgtcagtcatttttaatttagtctgatttatgagagcagttacagaaccatatgtttcccagcagccgtgtttcccacaagtacacctttcaccaccgtttataagtgtcatatgtcccggttctattcctacaccgttaaatcctctataaattttactgttaataactgcaccaccgcctatacctgtaccaagtgttattagaatatagcttgaagcattatgtccatttgccgtatattcgcccaaggcagctgcatttgcgtcattttcaagattcactgaaatattaagttgtttctccataaatttacgcattggcacattcttcatcgggatattatttgaatacgatattataccatttttattgtccaccgttcccggacacccaatgccaactgcttcaatcttattaatgtccatctgcgacttatctaaaagtgttttacacaatttagtcatatcaagcattatcgcttctatcggacgtgtattcaaagtaggaacactatcctttgcaataatttttcctgttttatcaacaattcctgcagcgatgttagttccacctaaatctattcctatataatacttcatctataaatcactccattccttaagtttgtttaaaattttataaaaatgataatataatttcacaaggtccgctaacagtatattcattattttgagcggggataaatatactatctcccttattcagtttaagaatctctccacccatatacaattccccgcttccgtctgtaattataagtgaatgaaagcttgttttatcaattctaagctgcactctattttgtactttcagtcgacgaacggtgaaataatcacattgagtcaaaataatatcatcaccatcaagcacagaatttgaataagtagattttttcaaatttgaagattcaagagcttctctaatatgtaattctcttttattcccgtccttatcgcgtctatcataatcatatacacgataagtcgtattggaattctgttgtatttcacatataagaattcccgctcctatcgcatgtacagttcccgcaggtatgaaaaatacatctcctttgtgaacaggcaccttattaagtacatctgttattgtattgcttctgattgcatcttgatattcctgctttgtaatatcttttttaaatccgtaatacagatatgcaccaacattgcaatcgagtatgtaccacatttctgtcttagcatttgcaccgtcatggcaagtggcatactcgtcatcgggatgaacctgcacagacaaattcttttttgcatctataaactttataagtattggaaaatcgtcaagggcaagaccttttgtacctacaatttcctttccaactatatctatgtactctttcaagccatatccttgatatttaccattggctattatactttgaccgtccttattagccgatagttcccaactttctgccagtatattcagttctgattttttattaaacacagtttttaattttgttccaccccagagataatccttaaaaacaggaataaggcgaacaggataaagttctgacactattttcactcctttgatatttataatagccatgtgcaaaacactgaaaaacatagagtttatacacttttacggatatggctaatccgttttgtcactataaattatattgggtatatagaaaaaccactctgatttggtataatatttgcacgtttttaatttattttataataaataacaaacagaacatacaaacgacacaaaattccatttagtttgacatgggtaacgttttttaagataagaatttacagtcggttatatgttctgtttataaattaatatttagatgttttgttatattatttatccatcttcggcgaattacaacgtggcatcattccatttaagtcattccacataaatacttttgcataagaatagtcctccggttcttccactgtctttgatatatcatcacctttttcaatagctcctatttcggctattgacgtcttaatcaagacagaattattgtttaatgttttataaaatgcaactattacaacgcttgagctatcatctgtgtctgtacgctttcttacgcttacatttcctttttcatagttgtttatcttatatacagccatatctttttccgaccattttgcatataaaaccatatcaccaacggaacccttggcaatagctgcaactgcattttcaaatgcagcatctgtataccatccctcgaagttatagccatcctttgtcggaactggcaatgttatcgtatctgtttcaatagtatacgatacaggagcattttcggcatttattccgccattgagctcatatgtaatctcatattcatttattgaccattgtgcaataaatgtaatagcaccgtttatatcatttgggactgtatatgtgtcattctgtgtaccatatttataaaccgtgtcatcagtgcctgtcttccagccttcaaatgtatatcctaatttttttggttcttgattaggaattttaattatcgatgtttccgcaatattatattgggtgttatcagtaggaattgtttcctcatccgctcctaccgcagagtatataactacaaaattacccacttctgtatactgagcataaattgtaagctctgtattatttccgaacagtgcaagcatatttgaagcggtattaagattatccatcgtaattacagttccttttgtcggtgcatcaaaccagcccttgaagttatatccttgttttttcggattgtcattcggtaaaacgggtaaattttcaacactgccgtatatagtcgttgttgtacctgtaccctcttgcaaatccagattaattgtatactgtatcggacgtacattcactgttatcgtaacatttttacctggcattatgaatgtatagctgccattttcttctgtaatagtgtttaaattttctccgtcagcagtacacaaaacattttccaacgtatatcccttaggaagcgtaaccataaatgaaacttcatcattttcatatgccttttctgtcacatttatcgtaccttgtactatatctgacggaattgttcccgttgatacaaagcttgcggtaatacttcctgacgctatttcatcccattttgcatataatattttatttcccgtcgtgccttgttcgattatttctatcttgttttggtattcaacatcagtgtaccaaccgccaaatgtatatccctttcttgtaccgggagcggcaagagttattgcctcagtttcaaccgtatagtttgacggattggtgtttgttgcatcattaagtccctcgtaagttatcgtataatttaacagagtccagtgtgcatataatgtgactgttgcttcgtttctaaagatgtcagacagattgtcttttgttatcccattaacctgactgccattgttttcttcagtgtaccagccgtcaaatttgtatccggcttttgttggtataggaagttcaataccattatcagggatattttcttccttaaaatcctcaaccattacttcacttccgccgcacgcattaagtgtgacagaatacacctgtgaattcggagctactttgtatgtaccatcgccgttatcaataactctgtatgtttccgtgtcggtaacatagcttgtcgggtcaaatgaatatgttccgccggaaatagtgatgaccgttgttgtgttagcgggctttgtcgcaccaatagtagatttaaacgttccgcccgtgatatttattaccggcactgtttctgtagtggcttcatcaccatcaagaatactgtaaccgttattctctgcggtattttcaaaagtgcctccgtttattgtaagagttccggaaaagccctttacagtgttaatcgcacctcttttactaacagaattcttaaaagttccgccgtttattgtaatttcgcttgaacccgagaaggtaatactgctcatattcccgttataattgaatattccactatttatcaccgcggtaccattggcaactgacagagcatagcccttatattcagaatcatcatttataacggtattttcaatatttataataccgtttgaaaggtttaccgcatagctatttccgttaattacagaatcttttaaatgaagtattgaacccgtaccgcttaacattatcgcttgactcattgcctcaacttttgctccgtcaatggtaagaactgcatcttttactgcaaattttataccataagaagttgatgtattaacattcttgattgtgccttttgttgtacctgtatcaacaactgtaaggttatacattgcttctatgaccgttgagccgcctccactcaatgtatgtccattaaggtcaagacgcattgcacactttgtctttatacttgatgttcccaaatcaatatctgcacccaactttacataatcatcagacgctgtaagagtgttaatttcctcaatactcgatacaatttttgctaccggctccggtgtcggttccggtgttggttccaccccttttttcgtcacaacatacttgccttcggcaacctttgttatactatatatatctgtatctatacaatttggatacagcacagtcgggtctgcggcaaactgcccaccttttatagatatttcagttgctgatgagtttgttgactttgtttttcctattgccgatttgaatgttccatcattaatatttattaccggtgacgctataatctcacttgtttctgcgtctatagactgatagccctcattgctatcgaggatactgtaccctccggcatttgtattctcaaaaacacctccattaatcgtaacagtacctacaaattgtttatctgtaactatagcacctcttccactgtttggatttgtgaatgtgccgccattaattgtcaactcacttgaacgtgctacaagaagagtgtttgtggttccgttatagccaaatgttccgtcatctataatggcaataccgccctgcaaataaagtgcgtagcctttataatccgctttattatttataagagcattatcaatattaatctcaccgccgttcgttcctacgtttatggcatagctgcccccgttaattactgcgtcctttatattgcactttcgtcctgcaacattaatcaatatcgcttgacctcccgcatcaatttctgcaccgtcaatatttattgtcgctgcttctgttgtacctcttattccataagaggtttgcgttgtacctgtatttataatggcacctttggtgcttcctgtgtcaacaatcgttatttcgtgtcttgggtccactacgaacggtcccgaagatgtcaatgtatgaccgttaaggtcaagatgtgtcacactttttgtcgtaaagcctgttgtgccaaggtctatatctgctccaagaattatatttccctcagtgcttgtaatcgctgcaagctgctctgccgttgtaacagttacagggtccggtagcggagtaggcgtcggttcaggagtaggagttggtaagccccattctatatcaccttttatgcttgtatgagttatattatcaatcatgaatgaaccttccccactgttttcagcagaagaaaatataaaacctcttacgcgtgcacaatcaagattgcttcctccatcaagtatcttaaactcatcaaacggaactcgaaattgtttccactccgtcgtcaaagtgaattccttagtgtctgtataagtagtcatcgcacttgctgtgtcaatcaccccaacatttatcttttcattcttgccgcttgtagattttccataaaacacaaagtctgtcatatattccatatcagcttttaaagtagcacggttagcagtacgctcatcgtcatttacatttcctttaaagaaatcattcggtgaataaaccacctttgcctttgacggtgtgtttccgtttcttgaatatttcacctcaagtgcttttgaatcaaatccaagcccgccttcttctttagtcgcaacgattgtatctccctcacttccggattctgtggatattgctgcctcatattttgtggtgttcttattataagtagcaaaatcataaacaaatgtcttttccggatacggaatcagacgttccggctttgtgtttgagaagccaatattatcaatgtaaaattcggcagttgttcgcgtgtctgctgaaatctgaacatatgttatatcttcagggtattcaacattaccttctaaactgtatatatattgatgccaatcggtatcgccctctgccatatttacagtaatatatgccagtccgcttaaacttactgtcagcttctgtgcaataccattacctttgatatcgatagtaaaatacttggcatccatagacggaacagtattaaggtcaaattttgctctgcaaccctcagtatcagttgcatctcttgtatatgtcacacattttgtttttgttgtctcaccatttatgtttaagacattcgcatccgtaatttgcattgttgttcccgtaaatttggaattccacgccgtagtctgttttttaaagccagtcgtatctatatcaatatagctttcctctcccatatccttatgaggatatgtattggcagtataaacaggtgtagcgagttttacaatttttacattatcaatagttatagatcccgtatctgccgtagcggctccaaaatatatgccggaaagagcggcgtcaagcttaccgtcagtctgcggattaccctgtttcacaagttcatttatgcctattgttactgtttgaacttcagtgtttacatctacagctttagtccatacttccatatcagaaccataaagacctatatttacacttccagcagttgctgcggaaatatccattgttattgtctgaattccatttaaatcccaatcaaacgggaaattcagtgttgcataccaattcgccgccgcaggtgttatcgctacatttccgctttcatcagaatatttaatcgaagccttacccgatggagcagtatagcttctgaatacatcgtctgtgctgaaatctgttgaatatacaccgtcaagcttttccggtgcagtgttgtatgaaagtaagcgattctttatatgagtaccgttatatgccgtagtcttataatcccatgaatagagcattttcggcgatgaactcatatgcagattccatgctgtccagtttacaggaattccgtcatactgattgtcagtatcgtccatccagttcataatctgattcatccatatatcgcttgtgcagtctccgcccgatatgttcttgtcggatgagtcccatccccattcacctataatcaccggtgcaacacgtcttaccggacctattatcgtatcccatgcggtttttgcacccttaaccggatatgcgtgagagtcatacataacgccatggccttccgccgtatctattagtctatatccatttgggcgttcattatagccatcagcaaaaccgcttatatcaaatgcccagttcagaccgccggctatacagatattatttgcaccttgtttgcgaatttcgtttagaagttgttgatgacctatagcagttacttcttcaccgccaactataatttgtccgccattataccacacgtcccattgttccactgtagttggtttttcaactcctaccggttttatatcatgtggttcattcaaaagaccgaaaagtaccgcactgttattgccgtacttaacagcaagttctttccacatatcaagatcgtcttgttgtggcataacatatctgtgacaatcaagaattatatacttacctctcgcctgtgcagctttaaccatatcatcaatatatttctgatattgttcctttgttaagtttttttcgtcccatacgctgccgtttttccaatatttaggatttattggcaaacgtatcagattagctccccagctatcatagaccattgtcatagactcaaatagatgttctgccataccccagtccatactcggaacatttacaccacgaagaactaccatttcatctgttccctgcttaactattttatttcccttgacctcaagaggcacagaacgattatagttaatttccgcattttgagcgtctgctgcagcttcctcatatgtctgtgcataaacaggcataatgcaaagcgacactgtcattacaatagccgttacaagacttaagaattttttcatgaatatcaccctttcgatttaattatataaaaaggactgtttttttgtagggagaaagagagagagaatattggaaggtatccattgtccaataaataaaacctattctaaacagtcctttactatattaacattataaaagaaaagttcacctcaaaaaacgaggtaaactttttctcactaccgattaattatgttttcggcacattattcatattttaatgtataaatttgttttggagtaacttcaacatcgcaccagccatttgtatccactgaaagctctgtctctgaaagttcctcaagcgtgacttttttaacacttgcccacttcctattttccgtacacggaagttcaaacgagtgtacctgtctttctattggcgattgtttatctgatatctccgctatgccaccattaaatctaattctatttttaactgtttcacttgacggattaaataatctcaccacacaccctaagccgtcctcactgcgttttacggcacttatgtgtagattttcattttcaagttcaataaacgacttttcaagagggttcttgccatgttctgttggtgctgtctgtccaattagtatttccatattaaaatcttcagcagctttccatacttgagcatcttcccaatcacccttatgaggcataaaagcatatcggaaagtatgttcaccaaaagactgtgaaccgttctctattctcgaatagttctgctcctcgggagttacatatatgcgaagctcaaagcagcgcaataatgacaaatacacagtatggttataatcatcatccgattcatacgctttaagtcccgtattcaaaatcgccgcaccttcattttcattgcatatatcaacaaacgagttcatcggctgctctgtcatcggaatttcgtcatacttcgaataatccggtttcgcaatcggacgctttaccacatcaaactgtccctgagcgtatacaaactctgcatccacgtctgtggggaaagcagcctgaagatagtgattaggaacattgttattaattttcgtttcaaattcaacccatcttgcgccttttctgagtgttacaagcgtttctattctgtaaggcgcgaggcgggaacttcttttcttgccgccgtcaactatattttcgggaattgcccaattaagaactattctgtacttagtttcaagctcaccgctgtatacaaggcttacaatcgcccgttcattaacagttgtatattcctcatcaagctccggtgtcttgtgttcccacgggctgccattttcaccggtgtccttaaaatatccgatattatcgtattccctgccggtttccttttcagtaaccttaagcgtaccgtttgaatttatcttaaccttcagatattcattctccatagtatttatgccgcaaagcatattaaccggcgttgtagcccgtgtatgatacttaggtacaaccttgagtgttttatagcccatcgacggaatatccttaacaaatattcttattgtatgtcttgacacaggaagaacatcgaccgcgtctgccaaatcctgaacaatttgatacataggattaattgacgatatgttctggtgcgggcatacattaccttcagcatcgacaatctcaaagctgtcacactcccactcaagaggaatttcaagctcacacggaaccgtcagacttcttttaaacggagccggattaaacattacaagcgccatatcattcttatcccagcccgcaaagtcaatgtcgccagacaaatccataagagctctttcaagcacgcaggttgcaatctctcttgactgcctgaaccgatattctacgtccttatatacaacatctctgccgcacgcgccaatcgaatcatgcccgtggttttgaagcatatagttataggctttattaataaacgcctgcggatatacagctccgcatacagacgcaaaaaccgccatcggctcagcatatgatgtgagcaatcgttctgtttcaaagttctcctgcttaaccttaattcttgcagaaagaacccagccaaacagcgcacttacgctgccttttgtaaacggatagcgcatttcgccctttaaaacaggtgaatttttatcaaaatctctgatcacgctctgctcaaagtcataaactgtactgtgaaaaacatcaacaccttcataaacagcattagcatccttgataagccttgattctctcatatccggtattgatgaatcatgaccgttcgaccagaagcggttaggcgttgtccactcatcgtcctgttcagagagtgcctgctccgtcttttcagctatatattcgtcatgatactcatattttctatgcgaatactgatattcatattcacatcttgccggatccgcaaaacggaatattccatctccagctccccatgaaacacgacgattgtcaccgtcacgcttgccataaaacacagggcgctgcattatataccacatattatatctcggtctctgccccagtcttgaagcataaattgttgtaccgtcggcgccctcccagtagaactccgatttcggtgccatatatgtattaagccctctgtaaaaggacgcaaaatctattccgaagccatgatatagctgtggcatttgtgatatttgtccccagccaaagggcgaatagcctgtctttgaaactttgccaaattcatttgcaattttatgtcccaggagaagatttcttataagagactctccacccacgcaaaactcatccggcaaacaaaaccacggacccacagcaagctttccctcactgatatactttttcagaatttccttcttttcagggtttatttcgagataatcctgaatgggaagtgtctgcgagtcaagatgaaaatgtttgtaatccggctctttctcaaaaatatcgagcagcatatcaatcgcagtcacaagcatatgtcttgttctttgagcgctgaatttccactccctgtcccagtgggtattggatataaaatgacattttatattttttcgctccatttacgcttcctcctctatgtaatcaagtatttccgcaagattattgcagtagcggctacacataggcatcgttccgctttcttttatattgcctcccgcaagtcctattgttgtatatccggcaatccttgccgagcatactcctgcgccgctgtcctcaatagcgattacgctaccgcgctcatcaaaaccaactccgagtccaaccgcgcaggtttcagcataaagccacggatgaggtttcggcgatagctcaccgagcgttccaacgctacccttgcggagcgggtatcctgctgaaattattgcgtcgtaaaaatccgtaggctcgcccatatcaagagctctgaacgccgaaagtatttccggcatcgccttttcatataatcccgatgttacaagtcctatcttaatccctttggcttttagtgcgagcaaaaattcttttaatccctcctgcggaacaaaagcattttttctgcctctgccctccataatttctttcatttcacgattaacgtggtcaaaatagaaattccgcgctttgtcaagcgattcacctggacagtatttatctatacaatactgtaaatgctccgatacgctatgacctgatacaaacggtatatcgctctcttcaagcttgaagctttcatcatcaagcatactggcggttgttttttcgattatccaaatccagaactcctcgcttcttaccgatgttccatccaaatccataagcacagccttgattttaccgtttctgttcacaggatgaagcggatagtacgcaccatagccaagagctgaattaacataggcaagagtctgattttcaaaagccacaaattcaacctttccgtctcctgtgcatactatatgttttactccgtcctcaccggaggtaaatcttccgtctgtagtggagggaaggttgcgaaatcctgagctgccgtctataattcccaaatcgggtatattgaaattcattacattaccacctctacattgcatatttccgcatccgatgaaacggttgtaccgtttaccgttccgttgtcggctgttattttaacagttccctctcctgtgttctttactaaaatattatatgtttttcctctgaattttcttgttaccttaaattcagatatatcttttgaaatacacggctctattacaagtccgtcaaaatcaggacgtatacccaaaagatattgtgacaccgtataataattccacgccgccgtgcctgtaagccatgaattcttcgcttctcccgctcttaccgcatcacgtccagcaatcatctgtgaatatacatacggctcggttttgtgaatttcacttatctcctctaaatatgccggcgcaatctttttatatagttcaaacgctctttcgccgttacccattaccgtttcaccgattattacccatgggttattatggcagaactctcccgcattttccttataccccggcggatacgacgagatttcgccaagctcaagtcggtatcccgaatacggcggataattgagtacaatgccatattcacattcaagatattttttaacgctgtcaagcgctttttgcgccctgccgtcctccttgcctatcccagccattacacagaagccgttcgattcaataaagattttaccctcgtcacattcgtcagagccaaccttattgccgttagcgtcgtaagctcttatgaaccactcaccgtcatagccatactctaaaactgcttcggtcattttttcaacctcatccgaaatatatttatacatttcatcattgttgagcgtcttataaagccttgcaaattctctgccaatatacacgaacattcctgcaataagcacagactctgctactctgccgtcatcgtctcccgtagtctggaaagattcgtcaggaatttcggaaaagcagttcaaattcaagcaatcgttccagtccgcacgtccgataagcggaagcccgtgagggccaaggttgtttgtaacgtgtccgaatgagcgattaagatgttctaaaagcgttgccgtattgtttttgtcacagtcaaacggcacctgctcgtctaaaataccgtagtcgcctgtttccttgatgtacgcaaccgtgcctaaaatcagccacagcggatcatcattaaatccgcctccgatttcattattgccctgctttgtaagcggctgatactgatgatacgcgcttccgtcctcaaactgcgttgaggcaatgtcaataatcctctctctcgccctttcgggtatttgatgtacaaagccaagcaaatcctgatttgaatcgcgaaatcccattccccggccgataccgctttcgtaatatgatgcacttcttgacatattaaaggtcaccatacactgatacggattccatatgtttaccatgcggttaagcttttcatcgtttgactcaagagtaaacaccgaaaggagattatcccagtacaatttaagtttatcaagctccgcgtcgcattgtgctgatgattcatatcttgcaatcatttcctttgcctttgtcttattgattacattaaggctttcaaatttctcgtccttttcattctcaatatatccaagaacaaatatgtattcgcggctttctcccgcgtcaagtgacacgtcaatctgatgagatgcgataggataccagcccgaagctactgaattgccgctcttgccatttattactctgtcaggagtgtcaagaccgttaaaggctccaaggaaagtgtctctgtctgtgtcaaaaccacttatttcggtatttactgaatagaaagcgtaatggtttcttctctcacggtattctgttttatggtatatagcgctgccgtcaatttccacctcgcccgtattgaggttacgctgatagttaagcatatcgtcctgagcgttccaaagacagaattcaacgaatgaaaacagatttatgtttttagcctcaccgcttgtatttgtcaccttaattctatgtatttcgcagttatcgtccacaggaacaaaagctgtctgctcaacgcggacgccgtttcgctcgccggtgatttttgtgtaacccataccgtgacgacactcataaaaatcaagctcttttttcatcggcatatacgagggtgtccagcaatcgccattatcgtttatgtaaaaatagcgtccaccgttatccgcagggatattgttgtatcggtatctcagaattcttctgtgctttgcgtccttgtagaagcaatagccgccggaggtgtttgaaattaatgagaaaaatccgtttgttccaagataatttatccatggaagcggcgttctcggagtctctattacatattccttattcaaatcgtcaaaatatccatatttcatatttgttctccttaccaataaattttccaatcgctttttttcatgcgcattaatgcttcaagatagaagtagtctccccaacttgtacattccggctcatgaccgtgttttctgctgtacataccgtcttttataattccgttgctttgaggataatctactgtcgtgtacttttccgaaagacttgtcatcatcttttccgccgcatcaaggaattcctgattgtgataatatttttccatttcaagaattccgcacaccgcaaccactgctgcggaggtatcccgtggttcatcgctaccatcggagaaaatcaaatcccaatacggaacagaatcctccggaagatggtcaataaaataatgcgtaacccgttcaaacaacggcaaaatcgacttctctttagtatagtggtaacatagagcaagaccatatactgcccatgattgtcctctcgcccaactgctgtcatcggaaaatccctgatgagtttctccccgaagcggcttattggttacaggatcaaagaaaaaggtgtgatatgaagacgcatcaggacggataatatttgcgattgatgtctgcatatgattataggcggcgtcataatactttttgtagcctgtcacttcgctcgcccagaatagaagcggaatatttaacatacagtcaacaataaatctataactttgcgaatcatccatagcatcccacgcctgaataaatttaccttttggctgataacgcttcaaaagccattctgccgcctcaattccatcctgcttcgcctgttcatctcctgttattcgataatcggcgacgcttgaaagtgtaaacaaaaagcccatatcatgatgctccagttcaactcgatcaaccaacctcttatgaaacatttcactgtgatgctttgccgaattataaaaagctttatcacctgtaagctcatacataagccataatatcccctcataaaatccggtagtccatgaaacgttctcaaattttttgaacacaagattttcactttgttctgtaggaaaacaatcatagaaataatttaagctttttcttacgatgctctccgcatatgtaatcgctttatcaatattcactttttatcactcctcctactataataaatttatgcgtaaatcacacgacagtcattcgtgtatatatccatcttgttgcttaaactgatataatgaaagtgcaatttctgacgtatcacgcctcagattttctgtctgtagataagtcagtctctcatttgttgtgttcgatttatgcagtagataaaaggagcttttatccagccctttttattataattagttgacgctgtttaccgccttcggtatgaaggattacctttgatgctgcctctcctgcactctgctctgcgagcaaatcatcctctgtgggaacatatattctatttgtagtctcatcaataaacaacacattatatcccgagaaatcagctatttcaattttgtctgtctgtcccagagcagtatcaacctttgttttcagaacaagatttgttccgtttctgtaaagcaacgtaccatataccaatctatacattgcaccgtaattatagctaccaatggcattgtatatatgtgatgtattatggccattttcagcatactttccgtctgacacagcaatcattgttttatttagtgttgacgtctcaacagtttttccttcttcattccctgtttttacataatcaggattgtcttcatccttcaaagagaataccttaacataatccacaagctcgcctttactgtttgtaacataacgaattatatcaccatgctttacttccgacataatttttgaaccgttaggtccattatagtaacgtttcagatagacatcttctgcaaggtcaacctgcatttgagcattacactgccagccgattattcgagttacactctcatcattatcattaactgcctttactacttcgtccactactgtaaggggtacttttttatctatctcaggtattacgtcacttgtataatatactattacacctgctgtccgagattcatccacgttatatgcttcaatgcgattttcggttgtgtattgtgtcccggtctgcggataatatacccaatcatcgaaataggttaaatttgtcttaatatactgtgatgcatctccgatatcccctctatcgtttgaaacgggaactacaaaaacctttgtctttgaatttatcgccacattatttccgttatcactattaaatgcaagtgtatttctaagatacatgcctcccggttttatggaagatgtccttttagaaaaatcgttatacattgtcatagaattgtcatggaatacatcattccactccggattttcattgtcgctatgatatggagtatcgatatatttaatctctccctcatcgttgagcttatacataattgggactcttacatacacataactgtcatcactataatcagccattatcttatatgacgaatccagtgttgcaagcgtagacctctgataatccgctgctatttttaatgcagttattattttttctgcatctttgcaggttaaaccatcaatttttgtacttcttgcaagtttgaattcattcatatcaccagtataaggaagtagtttggctataacatcgctcctgttatcctcaatccaagcctgtattaagaatccatattgtatatcgtcagttttaagcaaatcataaccggctatcctacctcggtaatcaagatacaccgtatatttatttccatttttaattgcttttgcgtctttatagtattcagcatttgttgaatacttatatgtcctgtcctcaaatttaattttagtgccattagtgctctttactacaccggtatacatcaaattgcttactataatctttgtcttgtttgtttcaatatctttagcaactgatagaattgaatcctctgttatataataagcatcaacaggattgcccatacggtcagtcatgtcgcagtcagcaaaatttacctttaatgacccgtttgggtcatcattgtacagaccataaatcattccctgcgactctacaatacgttttacaaccattatgtcatatttgtatacaaaaacaacatcgtacttaccattgttatcgttatccactaatctgatataatcgcagttataaaaatcttcctcgtctaacaattttggcgaaaatccgttaagtatctctttcgctgatgaattaagagatactttttttgttttattgtcgctatagtatcgtatcatgttattttcaacacttttaatatcttcgccgtctatcgttattatgctgttatttttgtgataagagatatataaaagagtatttcctccggaatcttcctttcgataatatgcctccaccttacacgcaaggtaatcatataaatcactgcggtcactggcaagaagataatctcctattctaatacctttatatattgtatcaccactcgttattgcatgctctgaaacagattctacaactccggtgattttataagcatccttgaaatactctaaagtattaatgcttttatgaccgttatttccatatgaaatgtatacatcggcctttatagaatcattaagaagagatgccgcatcagttcttctgatagaagcacttcggttatccagaaagttctttgttatatcataatttgcggcaatttgtatatacgcattatcattgcctccatacatataagcaatctgcttataccccaaagcatttacaagtgttctgagtgcattatcaaaacttatttcatccgtacaatctatattgaaatctacatatcccatacttttaagcatatcatgggctttttcccacatgtcttctttgtttgcatcttcgtcataaaatgagtcacagtttatgtagcggcacactgacagaaaaaattcacctgccttgattggctgaagatatgaaccatcatcaataggaacccacatttcaaaagctgtaagcttttttactgtttcatcatattcattcttacccataatgtacgtatcatattcaatatcctctatgtatgcgtcatcggggttatccagttcatgcttagggtgcgagtcttgaatgagaacattttgttcctcatcgcttagtcccgcattattatctctgctgccgtcatcatcaaagttttcagctgaaaacactgatattgccgctgtaaatattacactcagagaaagcatcaacgcaattatttttcttaatttattcatgttacttccccctttatcgtattactattgccgcaatcgatggcacatcgttatgtcttgaaataatcactctcgaagcttttgcctctcctgcaagattagatggaattatatcatcaagacttcccaaatgaactccttcgcgactgtcctccaaaatatatacacgattatttttaagattgaagtatctttcgcatttcagactgttgcttcctgctgttcccggatatgtctgtataatcattgatgaacctgttattgattttacaatgccaaattccgttgaatattgaacaccggtgaaccagtatggatttttgccttgccatatacgtcccgatacaacatcggtatcaggattagcagccatgtcatcaaatgcgataagtgtctttgcagaaccgatatatgactttgtttctccgtattcattgcctctgattaccacatcgggattatcattgttatcaaaatcaaatattttatgatagtcaacaatttcattattagcattcgttgcaatccttataaggtctccctcgtcaatggttgatgtaacaggtccttcgctgctattatactgtctttcaagttctactccctcagcggtcacaaattcctgttctgtaccattgtataaaagagttagtaaataacgctcgtcatcatcaacagaagtgagcgaaacattcttaaccgccgccataacagaactgtaagtgagctcatttcctcctgcattatccgaacgtattacggcaattcccgctaccatgtccttagacatattatatagttccaccgtaccaaaagttgcagacttcaaatctttaaattgctttatctcataatattgggagtcgtccattaatgatttgctcgccggaacatacattatttttgtcttctccgaaagtcttgcataacccgggaaactcctatagtttgcattatagtatgtgtcagaaaggtctgtgctgcgggtgaatacacttgccgttgtataaagctcgtcagcatctacatattgtgcagtttttactgactgaatttcaccattattattcagcttgtatcttataagctgattaactgaatccgggtctgtatttacatccttgaatagcttttcaatgtctgtatattcatttacttttttgttatttacctttgcattccgtgcgaactctaatgtttccatatttccggatgtagtatatacttttaggtatggtccatcgctatgctctgccggaaaagcttgtgcaagatatgcaaagttatttacctcatcattgtcatcaaattcggcatatgctattctgccacgatggtcaagcaatacactgacagcatttcctactgacaataatgaatatgttgtattttgtgccgcaagaatatctgtcaagtcatactccgcatcatcaatactaactgttaaattcccgttgtttcgagcagtcctcttaacaactccgtcagcttcttccctcgatatgtaaattacagcattttctttttgtttatcctcgataactgaaagaatatcataaatctttaaatttcctatcgatgtaaacttttcgtcagtgtcatatacaattacagtttccaaatcattaagttttatcgacggctgattataataatcatataatgtattttcatacggtgtaagctgatttatacagtatattgcttctctgattacatttactgtatcatacattccatcattgttactgtctatcagaataacctcatcagcgtttttaatgtcttctgtatcatattcagcaacataattgtaattatacaagcgatttattgtcttcggaagtgtttctttcttctccgagtttgttgtttcatttttataatatttcaatacagaaccatcaaagtctgaaatcaagtcctgctctaatgaaaggacattatttttctggttcggctcaataaattttagagtttcatcatcttcaacgtaaaaagcgtttactctgtaaccaaggtatctgtatacatcttttatatccgtactgaaactataagaacctattctcacttcatctgcactaagacctccacttccgtctaatgtgcgagttccacatacatatactatatcatcaagagtaagtattctatgataataatacaaaggtgttatatcagaaatagagtatattgacgatttatccaaatcatccacaaccatatatgccttggacgccgaatcaaatatctccaatatatccataaaagaaagcgtatcatcaatagtctttctcaaattcggaataatatcgttgcttaccgctacactgtagtatgaactgatattaccgccgttctcgatagcataaacatcatatcccagtacacggcacatgataatagcaacctcacccagagttatcggattgtcagccccaaatactgagttgttcttatcaatataaccgttatcataaagaaatcgaatttcattgtaatatgtacttccgggcaccacatcactgaatatggtcttgtcatcgcttttctcataatttttagcattcacaaaacctgccagatattttgcaaactgacctttcgttacaataccgttttcctcgttaggaaaaggaattatatccagtgccataaactttccggcaagaagcatatatctgtcactgcctgtcaaattcgtatacgatatgtttcttgatttcataaacagagatacagagtaaagtatctgagcagtctgtgctcttgttgcatttgccctcggcataaagcttccatcacccataccgtttataagtccaagattttttagtgcatacacgctatctttagcataatcagaaatatcaccatcatctgtaaagctatctatcgcagcaccgttcaattcacagttttcttgcttaagatatcgtactataagaactgacatatcttgacgtgtaattttttctcccacaccgaatatatcttcctcaattccacttacgatacccatatcggcagcagtatttacatatggagcataccacttattctcatccacatcactgaacttattatcaacattttccgtttcctttccgaaaattccaagaagcatcttaacaaactcctcacgggttacggaattatctgtgccaaagcttccgtctccgcgtccgctgataactccaagattttttaatgaatttatcgcagtatacgcccaatggtccgtatttacatctgtaaatattttattgtctttttcagatgacacagtatcatttccggtcacttgcgaaactatcatagatgtactgccttttcccgaaccgtttcctgatactgagcctatcccgttcgttttatttggatttttactgatagaattcatgtactcatcaagcaatgtccctaaagatgacaacgactccactcttttttcagttacatatttataatatcctgtacgattactggcactaagtccctcaaggctgctataactgatataaggtgcaagtatattcttattttctgaaataagattgccaatctcaccataaccatttacgcatccaaaagccacaagcagagcattttcattaaatgcacttctcaacgattctatagaatcataatcattttttgccgtagccgaaagaacattgctgagtaaactatcagatgttatatactttttaaaataatcggttcctccatcaagttcagccaagtcattatatgaggaatatatctcttttaaagtttgaattgaatttgttttgtttaaaagcttaactacaagttcttcaccgatgttttttcgtatatcgcttactgctgatacgtttctgtcggcaaccgacacggcttttgcaatttcagacaatgttaattcctgtgacaaataattgtcaagatttattattgccttatatttttcaaaataatttgcacaatcagattcggtagtgccgagagttttatactcgcttatacatgagtttatttctgtctgcgaggcattataaaattttctcgatactgtatttccatacgataaaaccgcatatatttgtccatatgccattccaccggtatgaatagcactgtttattgtattattttcagaattaatagtatacgcacctatatacttatcatcttttttaaacacaacaaatgcagttccatctcctgtgacggttccgtttatgtttatagtttcaccaccgtcagaagctattatatgactttcaaaaatcgttccggaaggtgcagtgtattgcattactccgcttcccacttctataggcgggcataagagtttataatcggtttcattattaaaaatgtatatctctaccgaactatcactttcttttttgatgttaaaattaaatgttgttgtatcacccgcaccaatactgcgttgctcagcaaaagacgcacttgtacatactccatcgttgttttttacaactgcgagagcacatgcttcaacggaaacactcgatgattcatttgtcaccggtacacttatattatttccgttttcttcaatatcgctcacttccggttgtataccgcttctggcagtgaagtttacattatatgttttctttacttttctgctgcctgaagtgattgtaatcattccgtttccgggaacagactcaggagcggttatttcatatgatgcacccgtaagttcttggcgtgtattggtctttggaatagtaggtgtctcaacatctacggttacatttttcttaatatcctccgcagtatatgacgccggcacaggaatattatttgatgacgcgtttttgtcaaagtcctcgacttttactccctttacaaaaacttctgaaatatttgcattttcaaaatacgcttcttctccggttgacggcggggttattgcctttacttcatctgtagaacggtcaaaaagtatttcgtcaatcttcaatggttcttcccacggtgtttcaagtgtatctgaagtcggattagcaccaaaataatctttatacgggaaaattattgtcatttgattaagtgccgtataaatatcgctctgtattccttggtccatcgttactgacccggacttaaacgcagatgtcggtattgttatgtattcccattcgccgttgtttggaagttggaacttttttgaatacttcttgttcccctctgtacttgaatactccatggcgatttcaagaacacgatgctctgctgcaccattcccgtggtcaactgtctgaggtgtatgtacccacatggaaatattttttgtatctctcataaggtcaagcattgaaatactttcattagcaagttctatcggctcgctcttaaactgcattacaaagccattgtatctttttgacgggtcggaaatctcatgtcccggatatgttatctgcatagcatttccatacttacctgatacatagctttttttagtgcttatagtacctccgttataactgtagaccatacgaaagtcagagtcactttccattgaaagcttatagttatatttgatttgtgattctgtatcctgtgcggttacaacaatgtttacaccaccaagagcaattacagcggcacatataaccgaacagatttttttaatcatcctcattctctgcctccattctattttactatcagtttataggtctgcggctcaataccgttattcgatacatccgatacggttattgtatattctccctttttatccttaggaatggtaaatttaaagttactatacgaataatttctccacggccaatgcggatatgaaacccatttcggataaaccttgtaaagtgcattatcaacattgtcaacaggcaatccctcaacctttagttcaccatttatctcatgctccgttttattcataatatgaattgaaatatgttcttcaattcctgcctcaacctcaaatgtctttggaatttgaattgctttgtttgcaggatattttgtaaccagttcacctatcggttccgatttgaccggcgttgcatgtcttactgattgtcctgtgagtttatcaccaatattcgtaaaagcaaaatcgtcaatccaaagtcgtcccgaagagtttagtgtcatattaaaaagactgatatatcttaccttgttaagatgaagcatatcgctcataccgaggtcgcttatcctaaatttatactgtttccattccgtgctgttgacatcgaatgcgatacaaaatctttcaaactcacgtttcttgaaatttgaacgctgttcattttgatttgtacctttttcctgttcaaaacgcagtataaagcgttgcttagaaccatcgccctttgcccaaaatgtaaaatattcagcatcgcctatatcccaagacttagggattgttgccctcgcctgccctccatacgaaccttcagggcaattatagctaaacataacaggattcgagccttgtcccctgctttcttcaactgcattagtaacagtaaacgagtctgtttctgtgttttcatcgtcttcccatgccttccatgtcatactttccttttgggtaacgctcgataccgtaaaaggctttatttcttcgtatggcagtcctcccacttttatattatcaattataaaactgccgggaccgtcatcaagagatgtaaagagtacagacttaatttgtgttgtatcaagtatcccttgctcattcttaaacaagcttaatggaaaaaaccagtccttccagttatcatgtaaatccatctgcatataagttgtaaataatctaccatctttcaactcaagtccaattccgattttacgcatatcaccgtcgccttttattctcacggcaagccattttgcaccgctcaaatcagtatctttatcaaaatcagcaatcgcagcactgtcccaactatcaacacagcttctgtcaaaatcaataagctgacctctgccttcataaccactatctgttcgttctgtccttacttctttgccatacactttaaagtccacggtattatcgtcaaaatcaataatatgttcccaagtcgaaagtggtgatgggtcgacatactgtctatctttttcctccggacgtgtttcaggctcttgaagcattttatatttcatatatatacccgaataatttgatggtgtaaatgtgtcatagtccataaacatatttggatttgcacccgtgtgaaaagaccatgccgtatagttaagttcattacggtcaataaaatcatggatttcgttcatccatacatgtggatattcacaaagataattgtcataccaatcaaaaagtctttctccccagtgtccatattcacccacaagtataggagcttcatctacaagacactctacggctttttcaaccggattatattccggcttcattggataaatgtgtgtgtcatagattacaccgttacccgttttatcctctaatttatagccatgttccatgccgttatatccatcgcacagaccgtcaaaatagtatgaccaatcaagaccgcccgcaattacaatattttttgcacccttgttgcgtatcatatccagaatttcttgatgaccatatacacgctgtgttttctttccatagctgtcggtagtttcaagcataccgccgttacgccacatttcccagtcaatatcatgaggttcattgagcaaaccgaatataactcccggattatttccataaacctctactgcatcattccagaaatccttctgctgttctgtaatagcataaaattcatgcagattaagtattacatactttccgcgtgatgtaatttgattaattacattatcaaccattttgcggtaatcaccataactcttaccctgataccattcttgtccaaaccaaaatttagaatgaacgcacaaacgaacacaatttgcattccaactgtcacaaagaagtcctactctcttcataaggtcattatctccgcctgtggcccactgcaaatccggaatattcgctccggtaagcctaaccttttcgccttcagcattatatatgtatctgccttcaacatgaagctccgagggtaatatcttttgtaccgttccaattttagcttgtgctgattcctgtacaagcaacaatgacatgctaaaaactgcaacaagtaagcaactgataactctttttattttcatagtaaactccttatccgaataaatataaaattattcttttactgaacctaccgtcaatccttgaataaaatatttctggaaaaacggataaacaaacaatataggtaaagtcgctattatacaaagtgccattctcgcaccttccttaggaacattagcgagaagatttgccgaacccatctgagctgaattttcggtcaaattctgaatatttgaaatcatactttgtagaagatactggagattatatttttgaggctctgttatataaagaagcggaagccaccagtcattccagtaagtaagtgttgcaaacagtgcaatcgttgccaatccaggtaaagatatgtgcagaacaatcttaaaatatatctgatactcatttgccccgtctatcttcgccgcctctattattgcagtcggtattgacattgaatagaatgacctcattacaattacgtgccatgcattcataacataaggaaaaataagtacccatatactgttcttcagattaagaatgcctgttgtcaccatatatcctgccactgttccaccactgaaaagcattgtaaaaaacgctataaatgtaaacagttttctgtatttaaaatcctttctggaaagcggatatgcatatagtgcaaccaccaatgtactcataagagttcctacaatcgtaacaaaaatagttacaccatatgctcttaaaatcgtttctttagatgtaattatatatctataagcatcaagactccattctttaggaatagcatgaaaaccaaactctgaaattgccgattcacttgtaaacgatgctccaagaaccacgagcaaaggataaacacaagctactacaataagaaaaaatataaaataaagaataatgtccgaaactttgattttacgtctttttttcttagccggagctttaacttccggctgtttgatactatctatgatattaatttcttgtttcttcactgttatacctccttttagaataatgcgttttcggggcttattttcttcacaataaaattagttgtcataacaagtataaatccaacaattgattgataaaaagctgccgctgaagccattcctacacttcccgtacccgctgacatcaacatattatatacgtatgtactgataacatttgtagccggataaagggcaccggtatttaacggcacttgatagaataaaccaaaatcggaattgaatatctttccgacatttaaaattgtaagaactaccataagaggaacaagtgccggcaatgttatgtatcgtatttgctgccatcgagatgctccgtcaaccttagcggcttcataaagcgaggtgtcaattcctgctatacctgcaagatatacaacactgccatatcccgttgttttccaaaagtttactataacaagaatccatggccagtatttcggatttgagtaccagtctacaccttctaatccaagtgccggaataagacttctatttatcagaccgttctcaacgtgaagaaatgccagcacaagaaagcttacaattacgtatgataagaaatgcggcataattactactgtctggcacagctttgaaactgctttattcctcaattcagaaagtccgattgccattgccacattaaatacaagaccgccgaaaataaacaacaaattatatgctatcgtatttctcgtaataacccatgcgtccggactgctaaacatgaactcaaaatttttgagtccattccacgggcttgcccatattccaagatcataacgatactgcttaaacgcaattataataccaaacattggcaaataattaaacagtataaaaaatattaatcctggaatgcacattgagagcaaggaaccattttcttttaaatctcttataaggcttttcttttttttcacgtctcatccactccttaaaaacttaataattgcaaggattaaagagatatataaatctctttatcttgcaattattataacatgtactatttttcattaaaatgaggtaaactttaagctaggtggtttaatttataaaaagtttgtcacattgattatcgggtgaagatttgtaaaatcatcaaatataaacactttcatagtttgatgctcagcatctgtaacattcaagtcaagcatcaattcaccgtctccgattattttctgcccagccatacgaactgtatcaatcacaccgttttctttatacacagctacaatcattaccatatttgtatctttttcaatatcagatgctatcgcttttatttttatatctccatttgtaagattagtgatttcctttccgtcaatttccgctgttactttaacagatgaaaacagagttgtttttccttctgtttccgacagtgtcatattatcaaagataatctcaccagttctcgctatctctttgagttcatctgcactcattgcctttgtgtcgtctgaattattatcaagattggcattctcagccgcttgaaatgtaacagtgcctatctccgtcatattaacaatattttcaccatttacaaactccgaaagcggaacactgattttcgcccaatcgccatgagaatcaagttcaaatctcctcgtataacgtgtaccacttgtaagcgttccatcgacagtgttgcccgttgaaaaacttatttttacagctcctttacccttataatcaaagtttaggtactcagccccctttgcaccattatttgtccacactgcaggagccggcacaaaaatttcgccggcataataagttgccgattgatagtaaacacagaaagccgtacttccatcaactccgccgtcctgcagccattttgcttttatatagtcttgataatcattcgacttatttttgaatcctccccatgtcttaccgcttgcgaacttagtatccgctgtttcaaaatccgcaacatacgatatttctgtcggttgagttgtcgcttccggagcagctgtcggggaagttgtatcctgttccgcaagcgtgatgttatctattactacacatcctgttacagctttttcttcaagttcatccgcactcatcatctttgtttcttcagcattgttatcgaggtttccactttctgccgcagaaaaagccattcctaccacatctgtcaacggcacttcattaccgttatttacaaattcagaaagtggcacactgattttttgccattcatcatttgtgtttatagtcactgtatgaccatatcgtattccgtttacaacctcgcccgtttcaagagatatttttatttttccttgtcccttagcatcaaattcaagacactctgaatttttatttattgcccattcctttggtattgacataaatatctctccggcataccatgtggcagctttgtaagtcagttcaagagcacaaccctctttaccgttttcagttatttctgactttatactgtcactgtaggtcttatcattgttattaaatcctgcccaggtctgcttatgactgagagtataagtatcaaaatctatggttcttgttgtattatccggtttctctgtaggttccggtgttgcatttggattaagaacattctctccgacattggacaactccatattgtcaaagattatacttccgtttctcgctttcgcttcaagctccgctgccgtcattgctttcgtttcagaggcactgttactcaaaccgccgttttcgcccgcttggaatgtcacgcagcctatattggctattgttacaggatttccgttatttttaaattccgaaagtggaatacttatactttgccattcaccgtttgtatcggcattaagtttataactgtactttgtacctttcgtaagagtgtctgttgcggcacttcctgttgacaggctgatatttacaatacctttgccattgtaatcaaaattaaggtactcagcatccgcaccgttttgccaagctacaggaattgggaagaaaacctcgcctgcataccatgtcgcagctttgtaggttatgcgaagtcctgttgaattttcctttccaccatcggctacccattctggtttgacaaaatcactataatcgccggctgtgtttttagttccgctatatgttgtgctattaccaaatttcgcatctgcagactcaaaatcagccatatatgacacgtttgtcgcaaagatagcacagtttattgaaaaaaatattgacattgcgataattaatgaaactaatttcttcataactaaatctcctaaatatccctcatataagtatagcggtcaaagaccgctatacttatacctaattatttgttcttgttattgcttgttcttgttcgcaagaaattcatcatactgtttctgtgcttcttcaataattttgtcaatgccggcagccttaagtttagcggcatattctttcataatcggctcagggtccattgaacccataattacctgtttacgatactcgctcttaactgtctgacatgccgctatttccgcttctaccgcagtattatcgaatttaaagccataatcgataggtttcttagcctctgcgttaaatgctttcagagcctcaaccttatcagggctttctccttctgtaagataatttaggaatacatttccctgcatccactgataaccttgtaacgtatacgatgtatcatccggaattgttatagtattatcatcaatcttggtgtaatgttttccttcaataccatagttgataaggttgctgagagttgcatcagtgttaagtagttcaaggaaacgaagaactctttccggattctttgatgttcttgaaacagcaagcatcgaacctgttccggctccattatcctgccatatatccgatactgttgattggtcaagttcaaaatcaaacttagcggaagtttccttggccttacctggttttaagaagtccacataacagaaagtttttccgtcctttaatctctgctcaaaatccgtagcagtcataatgtctttctttacaagaccttcgttataaagcttattatcccatttgcatgcttcaagatattccggagtttctacaagatttacaaccttgccgtcatatttgtctgtatcgtagaaaataacggctgttcctgcgatttcctcgtacttcataagagcttcaggtgttctgtcactgccccagtcaatcggatattgcatatttggttcattttccttaatcattttaagcaccggtaacagttcgtcaaatgtcttaatattatccatattgatattgtatttttcggcaatatctttgcgatatgtccagcctcttgaatctgccatttccttatatgtcggtatcgcataaagcttgccgtccactcttgcattgtcggctatttctccaagctgctcaattgtctttggaatatatgtgtcaatgtaatcatcaagtgcaagccatgcaccgtttctcgcatttgccgtataagtaagaactccgggtgttgtccatgcaatatcaaaatattcacccgccgctatcattgtgtttagctgcttgctgtactgatttgattccagtctgtgcattttcagtgtagcgttaattttatccttaagataatcattaacagcagcctctacggaagcaacatcctcctgtggcataccttgcatataccagttgatttcatatgtatcctccggtacaacattaggatcttcaccgcctgttgcaaccttgtttcctccgccacagcctgtaagcacacctccaagcattagcatggccaataatgccgcaattttctttctcataattttatctcctttcttttttgataacggttcagacgtttcgtcctttcccattatcagaacattttatattggtctttctttatatttaacattatacaccatagtatttcaaatataaatagcgataaactttaaaatgtgcatatttttttaataaaatttatatcatttcctacttgaaataatataaaaatatgttaaaatgttatatagtttaatataaagataagataggatggagaattatgaatgtaaagttgttaatttgtgacgatgagaagataatacgtgaaggacttgcttcactggattggaataccagaggtattgaggtagtaggaacagcaaaaaatggtgaggtagcatttgagctttttcagaaaatgcttcccgatattgttatatcagatataaaaatgccaacaaaagacggcatatggctttcagaacaaattcataaaatttcacctaatacaaaaattatatttcttacaggatacaatgattttgaatatgcacaaagtgctattaataacggcgtatgtcaatatcttttaaaaccgatagatgaatttgaactttatgaaatagttgacaaattaacaaaagaaatacaccttgagcaacaaaaagcagaaaaagaaattgaattacgcaaaacgcttagaaatagccgttattttctattgaattatttatttaatcgtgcacagtacggtattcttgattttgaactatttgagatatctaaaaaagctgcggcaatgacgacatttgtaatacgtcttgatacagacagtaccaactacggaatgaattttatgatttttgaggcactaatcgaacatcttcctaaaacaataaactttattcccttttttagtaattcggacctcgtatttatttgctgctttaacgaaccggaaggagaatccgagcaaaaacttttctcttgttgtgagaatttgggagactttattgatactgaatttaacgttaattataatataggaataggtatctttacttcggaaatatccgaacttgaggcaagctatacctctgcattgcaagcactcgattacagtgacagacttggacaaggaaacattatttatattaatgatattgaaccaaaatcacagctttcggcatatcagtcaaaactgatagaaacctacataaaagcacttaaaaacaacgacgaaaagcaaagcaaaaaaagcgtcaaggaacttttcgacgttatggaacgttctgatatgaatctttataatcagcagcgacgctgtatgtcacttattctttcaatttcagatgcactctatgatattgactgcgatccaacaatcctctttaaaaatacggatgcgtggtcattaatcagaaaaactcaatctcctgcagaacttaaaacctttgttgaaaatattacagatgttgtaatatcatatattgaaagtgttcaaaaacaaaaggctgcaaatataatcactcaagtaaaggctctggtcgaaaaaaattatgcaagggatgcctcgcttgaaacggttgcttcgcaagtgtttatttcaccttgctatttaagcgttatatttaaaaaagaaactaatataactttcaaaaattatctcatacagacaagaattgaaaaggccaaagaacttcttgaaaaaactgatttaaaaatatatgatattgccgaaaaagtaggatacaacaacacccgctatttcagcgagttatttcagcgtatctgtggtaaaactccatcgcagtatagagcgagtcacaatccatctatgcctcaagacatataggaaaaacaccaactacgttcaatgtcattaatttaagtcaaacaaaaagaattaagtttagaaatatacttgaatttaaaataattaaaaaagtgcttgactgaggtgaactcgaatcaatggtgtggtcgaactattttattatgataatctaagactgtaaccacaatcactatttaagtatccgtacaacttttaacttaaattcataactatattttgcaacaaaaaaaccgacctccaaaagttagatttgtggtctaacttttggaaatcggttcaatacctacatgtttccaaaccattttctcattttgtgccaagcaatatcatattaactaatgacattgcggctatggtggttttcctgttaaatatcacattagtttattcttattgcatcaaatcctcaaatatttttcctgtattcattgacgtttcttcattattaaagaatttaataagtgcttcctccagcttttttagattctctccattaatacttactgttgcaaaagagcttgaattttgtacactttgctttattcccttcacatgtggatttgcatctaatatcatttcatcgtcataaagttctcttattatcgggataagcgatgtatctcttgatagtcttctctgctgctcttttccattcataaaatcaagaaaccttattgctgcctcttgattttttgaatttgagtttattgcaagggcataacttcttacaaattgatttttattaggcagacttgctaccattatatttcccttaactgccgaagtatcaccattaagctttttccataaagagctgtttcccatcaacatcgctgcactgccaattttaaaggcggctattgtatcagtgtaattttcaatatctttgtattcttcaataaattccttatatagattaagcgtttctgcatagcttatgcctatcgcctcttttatttctataatattatacatcatatcttgaatatctgaggtagtcagtcccagtttaataggtagtccgaaatcagaattacggcaaagatttataattccatcccatgtttcaggaacgttatgaatcttatcgctcctataaaatataacatcagtatccattcccacaggcatagcataaaaagaatcattaacagaaaatctttcctgtgcgtcaattatatatctgctattatctaataaaatctctccatcaagagcctttatgtatttctgctcaacaaactccttagtccactcatcattaatccagtatatatcgatcgaactgtctttaccacttaacgccgaaacatatagctgatgcctcttttctgttgaagtgggtgcatcaataaattttacttgaatatctgggtttgcttcattaaattcagctatattttgcgtgaaatctgagatatagtttggttttattacttttaaaacagtaacagcctgtgcggattccactgttttctccactgtgcatcctgaacacattgataacatcacagaagcaagcaaaaacaaaataagtaattttttcattaaaaacacttcctttataagtcttgctaataatattataccctaaatactttatttagtaaatagcggaaaactttaaatagtgcatatttttttgtattactctgccaaatatttttatgacttgaaatttaatatggttatggtataattatatcatagaagtgtttttatatataccaatttaactttataatgataaaacggattagctatatccataaaaattcataaactttatattttcagtattttgtaatggctattatattataaaaaggaggtgtttgtcatgtctgaaaagttcaataatatgtcatttcgtacaaaattgttgctttcatatattgcggttattatattatgtatcattatttttggtttaaccgtattttcgagcatttcaagaagatttgaaaatgaaataactgacaataacgcacagataacaggcctcgctgtcaataatatgacaaataccatgaataatattgagcaaattctgtatagtgttcaagccaattcgacaattgaaaaaatgctgactgcgtccaatcctccgtctccttatgaagaaattgccgccatagaacaagaactttcaaaaatagaccccttaaaagcaacagtatcacgtcttagtctatatattgaaaaccgtacatcatacccatctccgtttgattcgaatgtgaccgcttccgtttattccaaaaatgaggtatggtataaaaacacaaaggaactgaacggaagcacatactggtgcgttatggattcctctgatgccaatggcttgttgtgcgttgctcgtgcgtttatagatacgagaacccataaaatactcggaataatccgtgcagatgttaatctttcgcaatttacaaatgatattgcacatataagtatgaacaatacaggtaagctttttttggtatatgaaaatcacataataaacacgtggaatgatagctacataaacaactttgtaaacgaaaatgaattttttaaagcaataagtgctgattccgataagcctcagcttgttcagataaacaaagaaaaacatattataaaccatagccggctcaaggacagctctcttatccttgtacgtgcttcaaaacttgatgattttaacagtgatatacatataatcgaaaaatcaatgataactacaggaattatagcattacttgtcgctctaatattcatttttatttttacacgttggcttacagctcctataacaaagcttataaagcatatggaacgctttgaaaataactatgagcgtataccgatagaaataacctctcatgacgagatgggcaaactgggtgagtcctacaactctatgctcaacaccatagattctctcataaccgatgttgaagatttatataaaaaacaaaagatatttgaacttaaggctttgcaagctcaaattaaccctcactttttatacaatacccttgactcaattcattggatggcacgtgctcatcatgcaccggatataagtaaaatggtgtccgcattgggaacttttttccgtcattctcttaataaaggcaacgaatatactacaatagaaaacgaattaaatcaaatatcaagctatgtatctatacaaaagatacgctttgaggataaatttgacgttgtatatgacattgacgaaaatcttctgcactgtacaatcgtaaaattaacaattcagcctcttgttgaaaattctatcatccatggttttgatgaaattgaagaaggcggtatgataacaattcgtatctatcccgaagatgattatatatttattgatgttattgataatggtagcggcgcagacactaatgagttaaataaagctattactcatgaattggactacaacgaaccaatcgaaaaatatggacttacaaatgtaaatctgagaattcagttatattttgataaaacctgcggcttatcatttaaaaccaatgaaaccggcggtgtaacagccacaataaaaataaagcgaaaagaaccggaatataaaactattgatttataattttgtatatgaggattgactgcaattcaaataccgaaaataatataactcaatactgccgcaaacttaatgcttattcagctttgcacactgttatgaagaatctttcataacagtgctatctttgccatgttccggcaataccgagattatattcaacagaatgtaacgaatgtcaaaaataattgaccgttcattatatttatataaaagcaccttgctttttaagttcttttcttaattcttcaatgtcaatgtcttgcacgtttatattatttacagcacatattccggcggcaacacctccgccatgacctatagctcctgcaataggtgatacacgtattgccgcttgtgcctcgaaatttgcactgatgcagcgtccgactgtaatcaaatttttcacattgtcagaaataagcgaacggtacggaatatgatatatatccccatattctaacgaaagttttgttttttcatacatttgggtatcatttccctccggagcgtgaatatctattggataaccgccgcaagcaatggtatcatcaaaatctacacaggatataatatcctcagctgtaagcacatagctacctttcagctgacgtgaacctcgtatgcctataaatggacccgtaaactcaagctctgcattttcaaagccctgtacctctgttttaagcaattcaagcacttcccaagcctgctttcttccgagaatttcagcacgagttaaatccttcggttcggtggggtctgcattaattatgcgtgtggtatttacaataaactctcctaccctgtctgtttcaaaaaacagaatatcctccctctgaaatgaaatcttgcctgtcttttgtgcttttttcaatgtatttacatacccgcctatagatagctttggagcacgggtaactttgcttaaatcactcttaagccgaggaaattcatcattattattcattatatattccttgactctatccgtatcaacattaataaccttaaaattcattgtcataggctggcatttcccatctgcctcacgacctttgtttgtttcaagccctgcaagaaaagcaaggtcacaatctccgcttgcatcaacaaacattctagactctatacatcgtttaccgtttttcccatatacctcaacagagtttattacggcattttcgtattcaacatcacagaccacactatgatagagaagttcaacaccggcttctgccatcatatcctcaagttcaagtttcattccttcagccgaaaacggagtaactgtatatgtatagcccgtagtatcaaaaatatgccccggagagaaccctttattcattaatctttctataagttcattcgttacaccccgaacaacttgaacatcaccggcatgaaacgtcatcataggtcccgtaccacatgctgtaagtgtaccgcctaaaaagccatatttctcaatcaatataacacttgctccacatcttgcagctgctattgctgccatacaaccggatattccacctccgattaccgctacatcatacattttttacacctccaaattttatatgttgttcctcgccccttgcagcacaagtatcaatctccatacatacatacggtacaagttgtattatattaattcgttcgctcttacttattatttcttgtgcactgtagtcaagctctatataaattttatcttgtttctgtgtaatatcagatatagaaagttcaagcgttccacaagtctgcgtggtcatcacaatacaacgcctatcacaacttatcccccctgcactgtgcgtcttatctttcaaaaacaccactccggttgttccattcttttttacacactgtattgaatcagaattctcaattatccttattccactttttctgtaataatcatttatttcttcttcatggcattttggaataactatatattcgtaagaaacgtcttttacctttcttccgtgtttaatccacattgtaagataccttcccttgtaactcttaccatctgattttatgctcatattattccaatctccgcttcgtatctctcgaaaaatatttacctcctgttcctccgggaaacagtagcccacatcatgactaccatcaagataagctccttttataatataaccttcactttcttcattaccatgtactgtaaatctcgaattatctgttacaagtcgattttcaattatcgtttctacttcactttcttcttcactgtttatacaacttccaagacaaaccacttctttatcgaagaaaaaccacgatttattcgctttcagactattttcatttgaaatcaacttcattgtgcatacaccgttttcccctatgccgcatcctcctgtaaaatcacctgcggcattaatattaggttttactgtcgagcctcgtaaaacagttgtccctggtaggcgttgtaaatctattgtttgccaaaaaaagtccgactttggctcattttttttataaatgtacatcataccgtccgaggtatgatgagcattttgattttcatcgttaattgattcatatgctgcagtccgttctgaatgcatggcaagacctatcgtataaccatttccgtgtttaacaactctatccatactgttaaatgccataaaatatggcttgatttctttcggtttaatattattatcttcttgtaaatgttccgcaagttctgccgtaaatactgaagcatattcaaaaaaattatctgtaatctgtgtcttgatcgtacccttcaattcattaaactcaggcatttcacttaatataagcattgccgaaagtatgtgcgtacaagcaaggtcactctgctcataataacgtgaaatttctctgccacgtaccatatccatagctctgccattatatatgaaaggaagataggatttttcaatccatgtattaattatatctgtatttttattttcaaattctgtatctttaaaaatatataacatcggtgcaagttcttgtataagcgagcgtccgtaaccacaattatatggtatattatcatgttgaataaatgaaccatccttataaaaaccgtcaccgctgtctgttataaccattacatcctgtataccggatatagcattctttatactatcgttgtctgaaagtaatattccacgaacagcaaaaataactgactcccaaattctgttagcaccggtaagctttatcctatcattaaaatgtttttccgccgccatatatcgtttgagttgtgatttgtcagtataatcatacatcaaggtaaaaatactgttgatactaagagggatacctatttcccagtaccaccagttacctttaggcacagtagtgtcattatatactttttctaaagtatttagtgcattaaatatttcattttttatatttttattatgatagaattgtgagttattttgagaaaacgatatagaaatgtccagtattcctttcagtgttgcatttataactcccggctcatttgatgtaattgccttttctatacgcccaccaagctgtacaagcctttgttctgtgcgttcatctgattgtagtatgcaatcagccgtttttttgccgttgtatcctctaccgcataaaacatcagaatatcgctttcttagtatgttaaaatccgtcataatcattcctcccatttttatatgtatcaataatatcatatatgtctatgcatattaaatgggagaatctttaattaatgcgttaaattttttatatttcaatttaaaaggacgctttaaaatagagcgtccctttaaattttataacagttcccattcttttattcattctctttttataggctcatctcttgcctaatcaatatacacgttttaaaacaactgatttagccaatcaactgacagactcaaccacggatactctctttcaaactttcgtttggaccatattaactcatcactcacccttgacaaaccatgctcgcctttctcaaatatatgtaattcaaacggtactccaaccctcctcaagccagctgcatacatcagtgtattttcaacaggaacacatatatcctcatatgtatgccataaaaacgttggtggaattatatccgtaatacgcctctcaagcgacagactgctccatagatgatttgactcgtcatctgtacctgtaagatttttaaatgaatctttatgagcaaattcacccgatgttattaccggataacttaaaatttgagcatttggcttatgcattgctaactcaatctcgcgctcgctgaatatttcagaatcattccacaaggcacttagtgatgctgcaagatgtccaccggcagaaaatccgcatacaattaccttatccgtatctatattccatttttctgcattttctctaagcatggcaactgcattagctgcattttgtataggaagcggatgtgtgtgcggttcaacacaataatacactactgccgcatggaatcctgctgcgttatatgccatagcaatacgctctgcctcacgctcagataccattccatatccacctccgggaaatatcacaactattggacgtttcttaccgtccagaatatacgtttccatatacggcataaatccatattctgtcgcttctttcaacaagttaaatttcttattaaacataacattcactccgtcttaatttttgtcagttaaacgggcatatcctatcacagaatatgcccgatatgctcatcttaattcaatgtaaatactttattaaataatggtttcatatcatttttccatataaagaccttcaccttactcttatttacgcaagtaaatggaataattacactgtctgaaatttcatctgattttatcgaaagcaatgtaccattctcattatattctgcaacataaaccatgccttcttctgttatattggtaaatatagcattaacatttccatttttatcatcatacgttatattcactgtcggttctgtaggctcatcctttgctgccgcattaattgtcacactcataacttcactggcaaaggattcatctgcaactgccgccacacgaacaatgtaagaccccggtgcaagatttgtgatttccgtgcctacacactgcatccatgtgaatgtaggttctgacaatgatttatattccattgtagtgtcaacacctgtgattttgccgtcattgccttgataagtctgttcttcaaccgctgctacatttggtgcggatgaacgtttaggaatagaaagttcaaacgcttcactgttgcttgtttttatgccatcatctttttttacgatttttagggtatgaccgaaatatgtgttgtcaatgtcgatatcctctgttacattatctttatccgtcgctacgccatcatctatttttattatatagtcacagccttccgtaaagcctgttagcttttcgtttacatagttaatcgagattgtaggctgcgtttccggcattgcatcatacgcaataattgtaaccgaatattctgcactcttaaactgcctttcggcttcctcgtctgcacttacagctttataacggattttatatgtggttccaggctcaatatcacctatatcatctccatcgcccgtagtccaattaataccattattcgtactatattccatagtgtctgcgatacctgcaatagtacctttgccgccaatcacactcggttgtgttactatgatttccgattttgtcggtgctgcaggacgtgccttaactattagagtctgtgcttcactcgcaaccgttgtcacattattacccttctttactatcgaaagcgttatctgttcatttgttatataattagctaatgataacttgttatctgttaatgtaacatctaaaccattaattgtatatgttccatcctcaacaaagtttataagttcctctgttgtataatcaattgcaatttcaggcgtcatttccttttcagctataaacgtttcaactgtaattgttgtcttctcacttgcaaagtctgtatctgttgcagctttgcggacattgtattcgcctgcatcaacctcaactgtatcaaccaactgcgtactactccattcatctgttctcttgagtttgtactgcatgccattcatacctgtgagtttacctttgccgcctatttccgtagcatttacaccttgaacggttgtaggtgccttaggacgtgctttaactgtcaactgttgcacgtcactgtcggtatatgtttcggtattgcgtgctttttttacaatttcaatagacaataatttgccggcataaccaattttttcatcatcaagcgatattgtcgtcacgccctcgccaagcgttatatctttagcattaccttcacccacctttatcgtatacggttcctgtgactcaaagccggtaagtgtttcatttatataatcaatgcttatctgtggagttgcttcctgtatttttgccggtggtataatttcctcctccgaatatgatacttctgttactttaacgttatctatatacgtcgcattttcaatattatttgaagctgtatgatagaatcgcaagtctgttattccttcctttaaagtgtcaaaggctcctttacatgtatcaaatgaacttccttcttttacaaaactcacattatcttgaagcattgttccatctagccatattgaaaatattcctgtgtttggatgaagttcaagttttatatggtgccattggttattctttatagacattgcttgttcatagcaccagtttcctcccgtattagtaatagctgttctgagtgacgatgcggtgaaatatgtaagccatagttcatttccttcctttgttttagcatacatacctcttgatgcaccaatcgcatcatcagcactttcaaacatcgtgtcgtattctattgttaatgttttctgcggattatctgccttacttataggaatagttgcattagccgttgccttattgccggtggcagacaacttcaacgactttgttgtttctcctgtaggtattgccgacgttaccgtcgttgttgtcccattaacgtcattcgttgcctttgccagcatatcatcgccgttgttatactcaacagatgatggactttctgtatatcccttcacataatcatatcttgtaggtcttataatattataagttgtatagtcaacacaggtaagactgttaatgttatcattgtctgttgcaattgcataaacactgtgcacacccgatgtaacatcaggtgtaaatacccatttaccgtcgtcacgttgtttagcatcacccgccttgatttctccctcgtcaatatatacctcaactttactgacaacaccatctgtgtcataagcacttattacaatgtcgtcgcctgactgctctacttttgatatatacggtgcataattatagaactctgtcgttgacaactctttatttacagctttagaagcatatttctctgtcaaatcagtcataaacggtgctataggtctgcctgcagcattaaatgtattaatcacggctggggtgtcagtttgtgcttcaactaaatcctttgcaatacccggagtatctgaccatgcatacttaacctgtggatttgttatatctgttacatctatttctatagtatcgccgttaattgtcggtgtaacatctttgtatattccgtcatcatctttaagttcaaagcccaacggcacacctccatcatcggttgaaagagagccgtatgtattcttgaaatgaagaataagtttatcgccactgcgttccatataatcaaaagatgggctttcaacatttgattgtgtattgttaataaaatcttctatataagcaactgctctgtcagcaataggtcctttatcatttgggtgaacattatttgtagttcctgtatcattgctcacaactgtttttacgttatccattctttgtgatacattccattgtcccgctcttactccggtgccaatgcgtattgtcgaataaatttttgcaaaattcgcagtaggaagttgaatgactacaaatggtaagtcctcatcattaaatgtttttctccagttgttaataagcgaagtcaatgcttgctcatacactgtgccgctttcaaaggtagtatttgcctctccttgataccatacaacagcagatgctttcaaattctttagcggcagaagtctttgtgtatatagtccgccttttgagcttgctcccgccatcattctctttgcttgtgaatcccaatttactgaatatgtcggaatccactgcattattgacgacccacccaaacttgaacttataagacctataggaacatcagaatctttttttatcatcctcttacctatcaagaatccaagtgcagaaaattgctttgaattttccatagtagcaaccttccactctgaaatctcgtcaaatgaattcatatatctcacgtcttcgtaagcttcacttaattcttcgttcatcaacgttggaaaagtctcaaggcgattaaacatatttgattgtcccgtacagaatataacatcaccgacagccacattatcgagagtaatcatgttatcaccgcttgatactgtcatagttgctgattttacagcttccatagcaggaagggttatctcccataaaccatgttctattgttgtttgctcatcagccccattaaaatttactgaaaccgtattccccgattttcctgtacctgtaatagtaataggttcttttctctggagtaccatgtttgaggagtacaccgcattcaaagttaactccggttcaagagtaggcgttggggttggacttgccgttggggttggcgttggtgtcacatccggattgattgttgtggttggcgttggtgttgtatccggctctgtttcgccacctccatcactgtctatattcacatcaaatgagtcaatagaccactgtattttcttagtgacttgagttttatcctcacttaaatctccacagcctatatataaatatacgaatccatcctttgatgccgtaattccgttaggcagggaatacttcatatttgatttgtttgttgaccagttttttatattactttcatctgtatgcttttcaagttcctcttttaccaaatcctgcgagtgtgatgtaagtgttatttcgctatcagaaacaaaaagacaatattccaattccattgttgcagtatcaagatatgtaaaattaatatcgacattaatcttgtcgccggtatttacttgtgccaacttaaacattgtcgctcttggtgtattgtttgcttgtatataataagtgacactttctttactgcttacagtattttttaatattttactattacttttgtctgaacttggcagtgtatacgctgatactccgtccgcttccgcttctactgtagttgtagtagccgcaaatgccgtcaaaccgatagatgcacatatcatcgtcacagctaacatcaatgaaataatttttttcatttttttcccctctttccgtttttatatacttttactcataataactatatcttgttattaatcttaccacgggaatttttttattaaaatgcggtaaactttaagctgtgcggttaaatttattaaaaccaaccatgtttttcggtacaaaagtcatataaatcgtaaaatatgctgttaaaagcttgacttgccataacattatccataaccctattgacgcaaaaaaacagtgccattagagttgattttcaaaccaacctaacgacactgttttattataaagtataaataatttcttctaaaatttaatcgctatatcattccaaaatgtttgagtgcgtattctatgccattatcgagcaaatcttttgtcacaaaagatgccgactttttaagttcttcactgccattacccataacgatactgttaggcactgccgtaagcataggcaaatcattcatactgtcgcctattgcatacgcattatcaatcggaatattatggtattcaagaactttttttattcctgtaccctttgaaaatccttttaccgtcatctcacaaaagccgtcaccacgcacaataaaatcaaaatctttttcgatttctcttttaaatcgttctatattgcttttttcatcataccaagccaagaatttgtcaaaagcaaaaccatcgtcgctgacatcaggtgacaaatctttaccttgcatttcaaaacgtcttttcagttttacaaaaccgtctaaatttctactgcgtttatcacaataaaatgacttagaatgttcatacattggtgtcatattacattcaaacacaagtttagcaacatttttgcaaagctccgatgacaatgtatgctgatatatcactttgccatcacattctatatacattccgcaaccacatatatatccgtcaaagcctatatctcttattctctgttcaacgttcataacggttctgcccgtatcaacatacatcaaatgtccgtttttctgtgccttatgtattgcatttaccgcactgtcgggtattatatatgcctcgtcatcagatataagtgtaccgtccaaatcaaaaaatactatcataaaaatcactccaataagtatattatatatggtgcaaaaagatttttcaagtcggacatatttaatttatatcgcaagcattgccgcacctataattcccgcatcattgaaaagctgagccgcaacaatctttgtttttgtcatatgcttattaaagcccgtattatacacatattcacgaatcggctcaataagataatcgccctctttgcttattcctccgccgattgcaataatttcgggttgaaaaatattttctatactgacaattccgtctgccaaatatctctcataattgcttacaactttttttgcgacctcgtcaccttgcttagccgcctcgaatgccgttctgcccgaaattttgccctcttttttcactataccatgcataatagtatctttatgagtttcaagtgcatctttagtctgcgatataagagcagtcgcagacgcatatgattccaagcacccctctttaccgcaggtacacattttacctccgctgacaatagtcatatgaccgagttcaccggcagcaccgttaaaacctctgaaaactttaccgttgattattacaccgccacccactccggtacctaaagtaaccaaagcaaacacacttgcacagtgtctgtttatcttatattcacccaatgccgcacaatttgcgtcattatccactttaacaggcaaaggtatatgttctttgaacttatcggcaagaggataatgatttatttttatattatttgaatacactatctctcctgtttcaaaatcaattgtaccgggacagcctatgccaatacccttaatctcgttcatttcaataccaatactttggacaagtttttttgacaaattcgccatatcggtaactatttcatctgtcggacgttctgacaaagtaggtacagaatcttttacaacaatctttccctcctctgtcacaatacctgccgcaatatttgttccgccaaggtctattcctatataatacatttttccacatagcctttctgcccaccaaagcaatgaatatgtatatacaaatcttatcgtgggcagataaggttataatacattattttgcaattcctacggtcattatctcatacggttttacctcaatattgaaataattgttattactgtcaattttcttaatcttcttttctatgatattactgatgtaaagattatcgataacatcactcttttgaattgtcaacgttgtagttttatcgcttacatttacccaacgaataattatatcttcgccgttgcctttttgcttgatacccgtaagaatcaagccgttgccttgccaattaatcatagaataatcaagcggcataactccgtcatgacaatctgtatcggctgttataatatctgttctgaactgatagcactcctcataagctccacttgaaatcaaatcacccttaaacggtacaatttcaatttccgtttcgcttatacccaagcattgagccttaggagtcgggaacacgccccaatcgcccatttcaccgactgcacgaagaattgtaactgcgattgtattatctaaatccggtaacatttcatattcgtacaagcctatatttgcgaccgctatacccttttcaccgtcatcaatactcacaaatccctgctcgtgttcacacgcactcggattattccaacctgcattatgacgattatttcttgtaacaacctcaaacacggaatcagccttatgtacatcggaatttatacctgtcggcaccataattctaactctatggtcttttacttcattatcaaatctcgttttgatttttacacccttaccgtttttgtcaagtgaaacaaatgtttctattttcatttcaacggtatcgttacttcttccgccgactctttctttaaagaataccatatgactcttttcgtcctcgaaattatcatcgcccgacttcggaactgtaattgtgtttgtgattttatacattgctctgtacggctcatcttccgcaagttcaatcttcgcaactgtatcttgcgttgttatcgccttacttccctcaggcattttatacatatattcattgcccaagtcacctgtttcttcgtaataagcgacgcctttatatgttctgccgcttgctttgtctgttacattaagcgagccgttcttgtttatttctacacgaattgcatcgttttccatacaattctcactgctgacaagtgtatctgttactttttccgtgtcaccctcaacaagggcatatgttttatatccgacagcggatatattttcagcctcaaatgttacacgaacacgtcttgccatgtacggttgtctgaacttatccttaggcaaatcgtagccgaatttaactcccaaatcttcgattttaaacggtattgaatttccgtctgaatctataagcttatagttcggcacatttatttcgtctaaatcatatgcacattttttaagccagcccgatttacgagttacgtcaatttccaccgatacgaccgatgtgcgctctctgcctgccgtgttaaacacaacaaacggaagtgcatttttgtacttttcgtattcctttgtattaatcttatccgcaatatatctttttccctctgatacaagatagtcggcaacttgcttactcttattaaaacgtgttgccatttcgtcttgtacctcatcaacactgcaacagcagatactgtcgtgagggtgattttgcataagtttcttccatgaatattcaagttcgtccgacggataattttgtcccaaaacagatgataaaactctgacaggctctgcaccgttttcaagtgccgattcacattttctgttcatctgctttaagtaaatatgcgatgacgcacaattcataagcgttgaccaaccgtctgtatcctggcttgtaagttcgccttttacaactgccaaatcatttggtactttctctttaattgccttaatatattccgggaaatttgaatgtttaaaattaatgtcgggataaagttccgacgcaacttctattgccttgcctaaatcagcctgtacaggctgatggtcacaaccgttcatcaacaaatattcatcagttgacgcaaaagtcgcaacttttttcaatctgtcgtcccaatattcttttgcaattttcttgtctgtcggaacttcgttgccgttattataccaattcgcaaacagaataccgaaaatcttcgtaccgtccggagattcccacatcatttctgaatacggagattcatagtttccattttcttgaacttcgttatcaaacccgacaggacgtacacctcgtccgaaagtcactgtatccattcccgcttgttttaaaagttggggcatttgccccgcattaccgaatgcgtccgggaaatatcccattttacacatagctccgtacttttcagcctctttcataccgacaagcaaatttctgatatttgcctcaccgctcgtataaaattcgtcttgcaagatataccaaggaccgattataaatttaccctccttggtatacttaataagtttttctttattttccggtcttatttcaagataatcgtcaagcacaatagtctgaccgtcaaggaaaaagcttttgaatgaatcgtccttttcaaaaacctccatacatttatctatcaattcaacaagtcgcattctgtgttgttcaaacggaagataccactctctgtcccaatgagagtgtgatattatatgtacatttttgctcatttgaaattccttcttataccatataataaatttgggtttgatatatttgttatacattatatcgctattatatatcaaaatcaatactcaagtaaaatttcgttgcacctgttgcactattattaccgtcataaagttcaacctttgatgttgcaatagattgattatcacgaataagttttatttgaataccgttagtgcttccctccaatataaacaccattgcgttttacaatgatttgtaccacgagtatagaactactctacactgtctcatttgtatacgaatagacttcttcatgatatattttaacttcaaattatctccgtccattccaattatatatcaattttttatttgttatgtataattatattacacaatatatcaaagttcagtatttttctgtttttacataatctaaaatttaattttaacaaaaaaaataaaccgttagattgtttcaaacggtctatttttaaaattcaattgcgaacagcaattttaacaggcacaatgtatttaattccgggcatttcgccgtttatttctctaagaacggtttcacctgctttcacacccagttcaaagaagttttgctccacggttattatcttgtttccgccatccatttcgtcaagctcgcttatattatcaaaacccattatgcacatttcatttggaaccgatatatcaagtgccttacaacaattatatacttgaattgccacccaatcgttttggcacaaaacacaagaaattccttgttcatgcatacgatttacaatcgttttcaaataattttcaacgttgccgtattgttgtctttcttcttcagtcagcatttcgtacttgtcgtcaatgttcgcatatacataatcaagattaactcctaaacctttttcttcaagtgccgccgcatatcccatatatctatcccttattgatatagtttcattcacacgacctctgcaaaaaaatccaatctttttatgtccatgctctaatgcatattcgcaaagtgcttttccgccgccgctgttatccgacacaatataactcataggcatattttctatataattatcaataagcacaagaggaatttttttaactaaaaactgattatacacttcaaaatttctgccaccacgtacaggataacatataacgccgtctatcccctgttccaaaagtgaacgaagtattttctcctcgttttccacacttctgtttgcattatatatactgacaaagcaattttccttattgagcacactattaattccgtcaaagcatttgaacatattaccaagctttatatcaaacggcataaccaatgccacaagagatatatctctgttttttttgtgtatcgtaactgcggcattgtcttccttgtccttgccaagaatactcatcgcattttttgaaacaaaactaccgctgcctcgttttctgttaatcaatccgtcatgttcaagctcctcaagtgctctgattgcggttatacgactcacaccgtattccttagtaattctgtcctctgtgacaaacggtgcgtcgtattcaaaatctcccgactttatacgctcttttaatttatccataatttgtttgtacaatggttttttatctgacattaacgttattcctccaagaacaatatatcttaattcattttctttcttatatttaatatatcacttttcgagtgacttgtcaaacaatatatcaattaaaacaaacaatttgtttctctctcga

SEQ ID NO: 32 - DNA, 18073 bp, Bacteroides intestinalis
ccttgcaaacaacaagttaagttcttgttgacaaggaatggacttcttgcaaacacctataaatcaaagtcttgtttattccacttacaggcccatagcttttttatatttaagcaatgcctgtaaatagtaataatctgcataatttatagaagcatcgatctcatatccgcctggctgattaccagtactatgcatcaaaaaggcaggttttatgtcccgacactgatatctttcggaagataattctcccaacatccgtgtggctgcatttaaatagcgggaggccaatgatggagtatcttccagttcggataactcaataagcgcagaagccgtaattgctgctgccgaagcatctttaggttgtttgatcatgtccggtgcatcaaaatcccaatagggtatataatcttcgggcaggttttccaaataaagttctgtgactttttcggcaaatcgcagaaatgtcttatcctgagtttcccgataaaccatcatatagccgtagatagcccatgcctgtccgcgtgcccataaactggagtcaccgtatccctgattggttaccccttttatgaaatgtccgtcaatcgtatcatagactgcaacatgataattgcctccatcttcacggaaggagtatttcatagtcgtttgtgcatgtttcacggctatatcatacagttcctgtccgccaccattcctggcagcccaaaagagaatttccagattcatcatattatccataatggtattatggggccaccccattctttttaccattccaggccacgaaagtatggtgcctaccttgggattatataactttgctaacttttgtgcccctttcaggatgaccgttttatattcctcatttccggttatgcgataagcattaccgaaactacagaatatctggaaaccgatatcatggtcggcaccatgtgcaggagttaccaatggcaacaaacattcggtataccgaatggcttgtgcttttatctcttcatcacccgtagcctcataatcataccaaaggataccgggccagaagccactggtccaatcataaatattgctcatgttccaattggtctgattggcttccatgctcctgggcattaaacaggaatcttgatcggcctcgcttaatgtccgccttatctgggcgtcacagtactccaattgtcggtctaaatggatagtatctgcttgtttatcgggcgcacagcccacccatccgaggctgatgctaaccaacaacaaactcagttgctttctcatacttttagctcatttaaattattattctttacaacacactctttacctgcttcagattgccaatggttccgacccaaccgcaaacaacctcttagcagacagagatctttttctttttattcattaatatgccatgcattttccttatttgtcaattcataaacaagggtagctccttctgctatttccctgtgatatatccaccccttatccagtaccttaccattgatagatacagattttatgtaacaggcatcaggcgtatcctttagaaccttgatacttatttttttcccattctccatagtcagttccacgtcagtgaaggcgggtggcaacagataataaaagtcttgtcccgcattagggaataaccctatggaggtaaatatataccaggaccccatcgcaccgctatcttcattatccgaatatcctttgagcagagaaaaattatcttttcgtatctgagacacataacgagctgtcagatccggtcttccgcaatgggtaaatatgaagggagataagaaaccgggttcattatttaaactgatcagattattctcaaatccataagacagccgctttatcatattcgcttttccaccacaatattctatcaatcggtcaaactgatgtggaacaaacaaagtgtaagtccaagagttaccttcataaaaatattctacccacgaaccatatgccttggcagggtctatagctacccattcaccattcgccttacggggtactatgaatcctttataagtatggctttcctgcaaaggattgaataattggctccaattgccggaacgttcgtaaagttccttttgagtattttcatcatgcatgattccggctatttcagaagtacagaaatcattgtaagcatattctattccggcactacacgacatgattccgccagtttccggttcccagcctaatcgcagatagtctttactgcgtgcatgataagcattccacttcataagtgcataagctttttcataatcgaatccctttacatttttcacgatagcatcagctattatattatccacatcgtcacctccctgtttagaagtccaatccaatgaactggtaaaagtgggattgcatacaccattgtgtgcaaaacgatcaataaatgaatttatagtctgagccacatagctctcgcgcaacaaaaccatgagtggatatttggtacgccacgtatcccaaacacaataatggtcgtccatgtgggctgattcactatcccaatgcggattatcgccggtacgatctcgaggcatcacaaaactgtggtaaagcgtggtataaaacagccgttcctcagcttcattctcagatttgatttttatggaagaaagcgtattgtcccatatcgctttagcattctcctttacggtattgaaactgttatccgcaatctcttctgataaaaacagggaggcattttctatacttttcaatgatatacctacgtttagatggactactcccggattcttattcagagccagacaagcgtataaagctttgtcaccctgatctgtgatcttcacttccttcaaaggtgtatctgttttcatcgcaaaatacactttataagcatctgtacttccaaaaccaccgctgtattctccccatcccgtcaaagtttgctgttcgggattgtagtttatctccccgccgtggaataatccttttacctcaggcactatatgctgcggaatattgtgcgcaatatccagtagaatattcccctgatccgtttcaggaaaagtgaaacgataggcaacgcaatgatgtgtaggcgaaatctccacctgaatatcgtatcgacttaacattaccttataatagtaaggtgtggcttcttcaccttgcttgggcgaatcgtgatctgtttcacctggattaaaaccgacttgaggtgacagaaatatctgtccgtaacgcccccagccgattcctgaaacatgcagttgtccaaagccccgtatcggctgatcgggtacataaccggcatgtccaccatatgcagtctggggagatgggttgacggagccatgtggaagttgaggtccgacaacacaatgcccagctccataagttcccatccacatatctactttgtcggctaaagattgtccttttataaaggatacattcattaataagaaaaggcatatgcccaatgttctggtcttcatggaatgttatgtttaaggtgataaatctattattttcattgatacacaaaagtacgcgatagttaccattggcagatacaaaaattctcttaaagtatacaacaataacaagcactacagctttttacaatattcttgcacccaaaaattatatatttgtatgtcggaaataaaaataggcctacttttgggcagttaaaatcactactatgaaacagcttattactaccttatttatctttatattccttcagccatcctgggcttcgctctacagaaactatcaggtggaagacgggctctctcataatagcgtctgggctgttatgcaagacaagcaaggttttctatggtttgggacggtagacggccttaatcgttttgacggtaattccttcaagatctataagaaattgcaaggggattccttatccataggcaataattttatccattgcctgaaagaagattctcacggtcattttctggtaggaaccaagcagggattctatctgttcaaccgcgagagtgaaacattcagccatgtcaggctggacaaccgctcacgtggaggagatgataccagcattaattatataatggaagatcccgacggaaatatatggttaggatgctacggacaaggtatctatgtgttaggcccggacctgcaggtcagaaaacattatatcaataaagggaatccgggtgacattgcttccaatcatatctggtgcatggtgcaggattataatggagtaatctggataggaacagacggaggaggcttaatccgccttgaccccaaggacgaaagatttacttcgattatgcacgaaaaggacttaaacctgacagatcccacgatttacagtttatactgtgatatggataatacgatttgggtaggaacttctatcagtggactctatcgttgtaacttccggacaggaaaggtaaccaatatagtataccctcaccgtaagatattaaatattaaagctattacggcatattccaataatgagttggtgatgggttcggacgcaggactgatcaaagtcgattgcattcaggaaacgatttcctttattaatgaaggaccggcatttgataatataacagacaaaagtatattttccatagcccatgatatggaaggcggcctatggataggaacctactttggaggtgtcaactactattctccatacgccaataaatttgcctattatccaggatccagcgaagaggtttcaaagagtattatcagttattttacggaagaatcttccgacaagatatgggtaggaaccaagaatgaagggctattactattcaatccggcaaaaatatcgtttgagactacccatttacagattgattatcacgacatccaggcattgatgatggacaatgacaaattgtggatcagtgtatatgggaagggagtcagtatggtcgacgtacattccaataccttgctaaagcgctattccaatgacgtaggaggccctgatctgctaacatccaatattgtgaacgtcatatttaagtcgtcgaaaggacagattttctttggaacccctgaaggtgttgattgtctggatgctgaaactaaaaaaatcaaccggctggaacgcacaaagggcataccggtgaaagccataatggaagattataatggttccatctggtttgccgctcacatgcatggactgctccatttatcggctgacggaacctgggaatccttcacccacatgccggaagattcaacctcattaatgagtaacaatgtgaattgcattcatcaggatgccagatatcgcatctgggtaggtagtgaaggagaaggaatgggacttttcaatccgaaaaccaagaagtttgaatacatacttaccgaaaatctgggacttccctcgaatataatctatgccatccaggaagatgcagatggcaatatatgggtaagtaccggtggcggtctggcccggattgaaccggaaacacgttctatctgtactttcagatacattgaagacctgattaagatacgttacaacctgaattgcgccctgcggggtagagataatcatctatatttcggaggaacaaatggcttcattgctttcaatccgaaagatatacagaataacgagtataaaccgcccatctgcctcacgggattccagatttcagggaatgaagttgtccccggtatcgaaggttcaccattgaagaagtctataagcatgacgcagaagatagaacttgaatctaaccaggctgcctttagtttcgactttgtttgcttgagttatctctcgcctgcacagaacaaatatgcgtacaagcttgaaggctttgatacggactggcactatgtggccaatggtaataacaaagccatctatatgaatataccttcgggcaaatatactttttatgtgaaaggaaccaataatgacggagtttggtgtgatacccctataaaggtgactgttatcgtaaaacgccacttctggctatccaatatgatgttactggtttatgccattctcgcaatctccgcatttactttacttatccgcaggtacaacaagcgtctggactctatcaatcaggataagatgtataagtacaaagtagaaaaggaaaaagaaatatatgaaaccaagattaactttttcaccaatatggcccatgaaatacgtactccgttgtcattgattgtagctcctctggagaacatcatttcatcgggcgacggaagtcagcaaaccaaaagcaatctggaaataatgaaacggaatgcaaaccggctgctggaactcgtaaaccaactattggatttccgcaagatagaagaagatatgttccgcttgtgcttcagcaagcagaatatttcagagattgtccgcaatatacataaacggtatgtgcaatatgcaaaactgaaagacatagatataagactggtagaaccggaaaaggacattgcttgtgtggtagataaggaagcgatggaaaaagtcatcggaaacctgctctccaatgccgtaaaatatgccaatagcctgataactatcaacataagtacagacaacaatctattaacaatcagtgtaaaagatgatggtccgggcattaagagtgaatttatagacaagatattcgaatcattttttcagatagagaataatgcgcagagaacaggttcgggattagggttggcattatcaaaatcactggtaacaaaacacaaagggaatattgcagcctcatccgattatgggcatggatgtacattgacattcacaattcctatggatcttccaatcagtatatcacagcttacggaagaatatccggaaaaagaagatatctccgtgcaacaaactgcgctatctcctgtagaagggaagttaagaatagtgttggctgaagacaatcaggaactccggagttttttaagtaattatttaagtgactatctggatgtatatgaagctcaaaatggtttggaagcattacaattggtagagaatgaaaacattgatatcatagtatcggatatccttatgcccgaaatggacggtctggagctttgcaaagccttgaagtctaatccggcttactcgcatctgccgtttatcttattgtccgcccgaacagatacggccaccaagatagaaggactgaacacgggagccgacgtgtatatggaaaagcctttttcgagcgaacagttgcgtgcacagatcaacagtatcatcaataaccgtaacagtatccgtgaaaacttcattaaatcgcctttggattattacaagcagaagagtgccgaacccaatggaaatactgagttcatagagaaactgaatattattattttagataacctcaccaatgagaaattctccatagacaatctctccgagatgtttctgatgagccggtccaatctgcataagaagataaagaatatcgtaggcatgacgcctaatgattatatcaaactgattcgtttgaatcaaagtgcacagctgctggctaccgggaaatataagataaatgaggtgtgctatctggtaggttttaatacaccttcttatttctccaaatgtttttatgagcattttggaaagctgccaaaagactttatcgtgatagaataaatgattactaacccaataattcaagaagggaggcttatgaggaaaagataaaacaggatggtaatcgaagaagaaagcatgaggcatccgacctccccatgctctctgcataataagacttacagcacaggatatttgtttgcaagctgatattatgcccaaagataaccattttcacactgaagagaagtaaccatgagaattttatattggtttttatggtttgtttgtaatttctaatactaatagtatccattaaacaaggattaatagcatgaaaacaaaatttattgccacattctttttgcttatatgtggttccgtcatgtttgctcaaacacgtacggtaaagggcaaggttgtcgataaggcaaatgaaccgctgattggtgtagcagttaatattaagaatacatcacaaggcagtattacagactttgaaggaaattattccatacaagtgaatacggagaatgccgtactggtattttcgtatataggatatgataaacaagaaataaaagtaggtgcacgcaatgtgattgacgtggtaatgcatgaagcttccattgcgctggaccaagtagtggtagtaggctatggaacatccaaaagaggagatgtaaccggctctatcagttccatcgatgcggcagaaataaagaaagtaccggtggtaaatgtaggacaggctttgcaaggccgtatgtcgggtgtgcaggtgaccaataatgacggaacacccggagccggagtgcaggtcctgatacgtggcgtaggatcatttggagataactcaccgctgtatgtagtggatggatatcccggtgcaagcatttccaatctgaatccgagtgacatacaaagcattgacgtactgaaagatgcttcagcagcagccatttatgggaaccgtgctgctaacggcgttgtcatcatcactaccaaaagaggaaatgcggataaaatgcagttgtcggtagacgcaactgtttccgtacagtttaagccttctacttttgacgtactgaatgcacaggattttgcatctttggctacggaaataagtaaaaaggaaaatgctccggtactggatgcatgggctaatccttccgggttgcgcaccatcgactggcaggatctgatgtatcgtgccggattgaagcagaactacaatttaagtctgcggggaggttctgaaaaggtacagacttccatctctctgggattaaccaatcaggagggtgtagtgcggttctctgattacaaacgctataacatagcattaacacaggattacaagccgttgaaatggttgaaatcttctaccagcctgcgctatgcatatacggacaataagactgtattcggttccggccagggcggcgtaggaagattggccaagctgattccgaccatgacgggtaatccactcaccgatgaagtggaaaatgcaaatggagtattcggcttctatgacaagaatgccaatgccgtaagagataacgagaacgtatatgcacgttccaaatcgaacgaccagaaaaacatatcccataatctgatagccaatacctcattggaaatcaaccctttcaaaggcttggtattcaagactaattttggtatcagctacggtgcttcttccggttacgacttcaatccttacgacgaccgtgttcccaccacacgccttgccacttacagacagtatgccagcaatagttttgagtatttgtgggaaaacaccctgaattactctaacacattcggcaaacatagcatcgacgtattgggtggtgtatctattcaggagaacacagcacgcaacatgagtgtgtatggtgaaggattatcgagtgacggtctgagaaacctgggctctctgcaaacgatgcgtgatatcagtggcaaccagcaaacctggtctctggcttcacaatttgcccgtctgacctacaaatttgccgaacgttacatcctgacaggaacagttcgtcgcgacggttcatcccgttttatgcgcggaaaccgctggggtgtattcccttccgtatcagcagcatggcgtattaaggaagaaagtttcctgaaagatgtggatttcatcagtaacctgaagttgagagcaagttatggtgaagcaggtaaccagaatatcggtctgttccaataccagtcatcttacactaccggtaagcgcagcagcaattatggatatgtattcggacaagacaaaacctatatcgacggtatggttcaggccttcttgccgaaccctaacctgaaatgggaaacttccaaacagacggatataggtatagacctgggattcttcaataataagctgatgcttacagccgattattacatcaagaaatcaagtgacttcctactggaaatccagatgcctgcacaaaccggttttactaaagccacacgtaatgtaggtagcgttaaaaacaatggttttgaattcagcgtggattaccgcgacaacagtcacgactttaagtatggtgtaaatgtgaatttaactaccgtaaagaacaagattgaaagattgtcaccgggaaaagatgccgttgcgaatcttcaatcattaggcttcccaactacgggtaacacatcctgggccgtattcagtatgtcgaaggtaggtggttctatcggagaattttatggattccagacagacggtatcattcagaatcaggcagaaattgacgccttgaatgcgaatgcccacagattgaatcaagacgacaatgtgtggtacatcgcttccggaacagctcccggagaccgcaagtttatagaccagaacggtgatggcgtaattaccgatgccgaccgtgtttccctgggtagcccgcttccgaagttttatggaggtatcaacctctccggtgagtataaaggctttgatttcaatttattcttcaactactccgttggaaataagatattgaacttcgttaagcgcaatttgataagtatgggaggtgaaggcagtatcggtttgcagaatgtcggcaaagaattctacgataaccgctggactgaaacgaatccgaccaacaaatacccgcgtgccgtatggtctgacgttagtggaaacagccgtgtgtcggatgctttcgtggaagacggttcttatcttcgcctgaagaatattgaagtaggatatacattgccggcaaacatcctgaagaaagccagtatttctaagctgagaatctttgccagcgtacagaacctcttcactattaccggctattcaggtatggacccggaaataggtcagagcatgagcagttcaaccggagttgccggtggagttaccgcctcgggagttgatgttggcatttatccttactcacgctttttcaccatgggattcaatcttgaattctaaggagagacatttctgtatgacaaattataaatttacaatacaatgaaaaaaagacatataatcggttcattcctgctcggattgcttttaacggtaaataccggctgtgaagattttcttgatcagaaagatacatcgggtatcaatgagaattctcttttcttaaaacctgaagacggttactctttagtcacaggcgtatactctactttccacttcagtgtagactatatgctgaaaggaatctggtttaccgccaacttccctactcaggattttcacaatgacggttcggatacgttctggaatacgtatgaagtaccgactgattttgatgcattaaacacgttctgggttggaaactatatcggaatttccagagccaatgctgctattcctattttacagcgcatgaaagacaacggtgtactgagtgaaaaagaagctaacacactgattggcgaatgttatttcctgagaggtgtattctattattatctcgctgttgattttggaggtgtacctctggaacttgaaacagtaaaagacgaaggtttacatccgcgcaattcacaggatgaagtatttgcatcggtagtctcggatatgaacatagcagcaggcttgctcccgtggaaagcggaacaaggcagtgcagacagaggacgtgctacccgagaagcggccttggcgtatcagggagatgctttgatgtggctaaagcaatataaagaggcagtagaggtattcaatcaactggacagcaaatgccaactggaagaaaacttcctgaatatccacgaaattgccaacagaaacggaaaagaatctattttcgaagtgcagtttacagaatatggttctatgaactggggcgcttttggtgtaaacaaccattggatcagttcgttcggcatgccggttgccatttccggttttgcttatgcatatgccgacaaaaagatgtacgactctttcgagaatggtgacttacgtagacacgccaccgttatcggaccgggtgatgaacatccgtcaccattgattgacctgcaggattatccgaagctgaaagatttcgcaacgaaagggaatgggaatatcccggcttctttttatcaggatgaggaaggtaatgtgctgaatacctgcggaacagtagaaaacccctggttagacggtacacgttccggatattatggagtaaaatactggcgtaatccggaagtttgcggaaccagaggtgcaggttggtttatgagtccggacaacattatgatgatgcgttatgcccaggtacttttaagtaaagcggaatgtttgtatcgcctgaatgacagtaatggtgcaatggctattgtacaaaaagtcagagaccgtgcttttggtaaattacagaattccgcagtagaggtaccggcacctgccaacacagacgtacttaaagtaatcatggatgaatatcgtcatgaactcaccggtgaaacgtctctttggttcttgctgagaaggacgggagagcatgccaattacatcaaagagaaatatggcataacgatacctaccggaaaggatttgatgccaatacctcagacacaaattggtttgaaccagaatttgaaacaaaatcccgggtattaattcttaagtaggtaagaagattgtatttttgtggtgggttgccatgtgaaagccggcaacccaccctatttcaatactaataaaaaaagatgtaggatatgaagaacactgttttaccgttgatactgtttttatgtatgctttgtttgggttcacacttgtatgccggccacagtatgcatcctctgaatcagatatcttacgtaaagaagaaaataaaagaacagcaagagccttattttacggcttatcgccagttaatgcattatgcagattcgatacaggaggtttcacagaatgccttggtcgattttgcggttccggggttctatgataaaccggaagaacaccgggctaattctctggctttacagcgtgatgcttttgcggcctattgctcggcattggcttaccagttatccggtgaagaacgctatgggcaaaaggcatgttactttctgaatgcctggtcttctaccaataaaaaatattcggaacatgatggtgtcctggtaatgagttattcaggctccgccttgctgatggcggcagagttaatgatggatacgccgatatggaatcctcaggataaagatgcttttaaaacttgggtatcccaagtgtatcagaaagctgtgaatgagattcgcgttcataagaataattgggcggactggggacgtttcggttctttgctggcagcttcccttctggatgataaagaagaagtggcccgtaacgtgcagttgataaagtccgatttatttgtgaagattgcagaagacggacacatgccggaagaagtggtccggggaaataatggaatatggtatacctatttttcattggctccgatgactgccgcctgctggttggtttacaacctgaccggtgagaacttgtttgtatgggaacataacgatgcgtcattaaagaaagctctggactacatgttttacttccaccagcacccttcggagtggaaatgggatacacggccaaatttaggagcccatgagacctggcctgataatttactggaagcaatggcaggaatctataatgatgcttcatatcttcagtatgtagaaagcagtcgcccgcacatatatccattgcatcatttcgcctggtcttttcctactttgatgccagtgtcgcttaaaggctatgacttaacggataataatacatgggctaattataatcgctatgaagtggcaaataaaacagtgaagaagcctgtagctattttcatgggaaattccataacggaaggctggaaccgcagtcacccggacttctttacacagaacggatatgtgggacgtggcatttcaggacaggtcacagcacaaatgctggcccgcttccgtgcggatgttctggatttgaagcctcaggtagtatgtatcttagccggtacaaacgatattgcccaaaactgcatgtatatgtcggttgagaatatagccggcaatatcttttctatggcggaactggccaaagccaatggaataaaagtcgttatctgttccgtactgcctgctacccgttattcatggcgtcctactgttcagaatcctgccggtcagattattcaattaaacaagctactgcaaaagtatgctcaaaagaataagattccttatgtcgattttcattccatgatgaaagacgaacagaacggactgcctcaaaaatactccaaagatggagtacatccaaccaaagaaggcttcagcatgatggaacccatcataaaagaagcaattgacaaactgctgaaataaattcagcgcacgtaaacctttatagaatgaagaatatatattatatcctgatactttgttgtttatgcttattctcatgtgactcacaccctgatactaaaagctcactgcctttcggcgtgaacctggccggtgcggaatttttccataagaaaatggacggagtgggacagtttggaatagattaccattatccaaccaccagagagttcgattattggaagtcaaaaggcctgaccttaatacgtcttcccttcaaatgggaacgtattcaacgcgaactctacggcgaattgaatcgcgaagaaattgattatataaagtatctgctggatgaagccggagcacgcgatatgaaaatcctgatagatatgcacaactacggacgccggaaagataatggcaaagaccgtatcataggtgacagtgtttctatcgatcattttgcatcggtctggaagcaaattgccggtgagcttaaagaacatagtgccctatacggatatggtctgataaatgagccgcatgatatgttagattccgtgccctggttcaagattgcccaggctgcaattgaggagagcagaaaagtagatttaaagacagcgattgtcgtaggtggtaatcattggagttccgctgcccgctggcaggagattagcgatgatttgaaacacttacatgatccttcggacaatttgatttttgaaggccattgctattttgacgaggatggttcgggtatttatcggcgctcctatgatgaagagaaggcatatcctactattgggattgatcgtacccgccccttcgtagagtggttgaaaacaaataatctacggggattcatcggagaatacggagttccaggagatgacgaacgttggctggtatgtctggataattttctggattacctgagtaaggaaaatataaacggtacttactgggcagccggtgcacaatggaataaatatatattatcaatccatccggatgataactatcaaacagataagatacagttaggagttctgacaaagtatttagaaaccaagaattaaatcaaacagattcaacaatgaaaacattcatattatcttttttaatatatgccggctgttcattacccttgacggcgcaacagataaaacccgctattccttctgatccggaaatagaggcaaagattaacaagctcttacagaaactgacgcttgaggaaaagatcgggcaaatgtgcgagatcacaattgatgtaatcacagatttctcggacaaagaaaacggattcagattgagtgagagcatgctcgataccgtaatcggtaaatataaagtaggttctattctgaacactccctttagtatagctcaggaaaaagaagtctgggcagacctgattacaagaatccagaaaaagtcaatggaagaaataggtattccctgtatatatggagtcgatcagattcatggcaccacctacactcgcggaggaactttctttcctcaaagcatcaacatggctgccgccttcaaccggcaactcacgcgacgtggagctgaaatctcggcttatgaaaccaaagcatgctgtattccctggaattacgctccggttatggatctgggacgtgatccccgctggccgcgtatgtgggagagctacggcgaagactgctatgtaaatgcagaaatgggcgtacaggcagtgaaaggtttgcagggagaaaatccgaaccatatcggtgagaataatgtggctgcctgcatcaagcacttcatgggttatggcgtacctgtttcaggaaaagaccgcactccttcctctatctcccgcacggatttacgcgagaagcattttgctcctttcctggcatccatccaggccggtgctttatccctaatggttaattcaggcgtagacaacggcgtaccttttcatgcaaacaaagaattgctgaccggctggctaaaggaagaactgaactgggacggcatgattgtaacagactgggccgatatcaataacctttgcctgcgtgatcatattgccgaaacgaagaaggaggcaattcaaatagccattaatgccggcattgacatgtctatggttccctatgaagtaagtttctgcacttatctgaaagaattggtagaagagggtaaagtatcgatggctcgtattgacgacgctgtatctcgtgtactccggttgaaatatcgcctgggactctttgataatccttattgggacatcaggaaatacgatcaatttgcatcaccggaattcgcaagtgtagcattgcaggcagcggaagagtcggaagttctactgaagaacgaagacgatattcttcctttggcgaaaggaaaaaagatattactgaccggtcctaacgcaaactccatgcgttgcctgaatggaggatggtcttattcctggcagggagacaaagcggatgaatgtgcacaagcatacaatacaatctacgaagctttctgtaacgagtatggaaaagaatctgttatctatgaaccgggagtcacttataagacttctgccgatgctttatggtgggaagaaaatactccccggatagcccaagccgtatcggcagcagaaaaagctgatgttattatagcctgtataggtgagaattcgtattgcgaaactccggggaatctgacagacctgaatttatcaacgaatcagaaagatttagtgaaagcgctggcagcaacaggtaagccgatcattttggttttaaatgaaggacgcccacgaattatccatgatatcgttcctttggcgaaagccgttgtacacatcatgttactcggcaactatggagctgatgcattggtcaatctggtatcagggaaagcgaacttcagtggaaaacttccttttacgtacccgcatctcataaattcattggctacttacgattataaaccttgtgaaaacatggggcagatgggtggtaactacaattatgatgctgtaatggatgtgcaatggccgtttggcttcggactgagttataccacttacagttatagtaatttgaaagtgaatcgtacttctttcgatgctgataatgagttagtatttactgtggacgtaactaatacgggaaaaatggcaggaaaagaaagcgtactattgtactcacgtgatttagtggcaagcatcactcctgataatatccgcctgcgtaactttgagaaagtggatcttcaacccggtgaaacaaaaactgttaccatgaaattaaagggaagtgacttggcctttgtcggtgctgatggaaagtggaggctggaaaaaggtgctttccgtatgacatgcggaacacaaaagctggaggtacattgtactacaacaaagatatggcagacgcctaatattagtaaatccggaatttgaaaagacctgtacttaaataagaaaatataaaagaaagagtaaagttatcctttagggagactttactctttcttttatataaccag

SEQ ID NO: 33 - protein, 977 aa, artificial
  Synthetic
GSAEINYNRSVPLEVKGNKIVKQGTDEMVVLRGVNVPSMDWGMAENLYESMTMVYDCWGANLIRLPIHPKYWKDGSIWDGKNLTKEQYQKYIDDMVKAAQARGKYIILDCHRYVMPQQDDLDMLKELAVKYGNNSAVLFGLLNEPHDIKPTDIEKPTMEDQWEVWYNGGQIIVGGEEVTAIGHQQLLNEIRALGANNICIAGGLSWAFDISGLADGYNGRENGYRLIDTAEGHGVMYDSHAYPVKGTKSSWDTIIGPVRRVAPILIGEWGWDSSDNNISGGDCTSDIWMNQIMNWMDDTDNQYDGIPLNWTAWNLHMKSSPRMISSWDYKTTAYNGTYIKNRLQSYGNLPETQDGVYSTDFSTNDVFRGYKAPSGAASVSYSEANENIVVSHKPADWYATLNFPFDWDLNGIQTITMDISADTAETLNIGLYGSDMEEWTAPVKVDSTVKNITLSIDQLVRQGNQQTDGILNGAVSGIYIGSSTTETANNTKVVTMADEDNTAEYKINNYENGKVSVRKRTDAEDSASTVIVAFYDKNSVLTGISTANIRADEKGDEIIKAVNEPASYSCAEVFMWDSLNGMVPRCNPISNKVNITIDNIKIVKLAEPIYTATEYPHTDIGAESYIDVDNTDFASQSTTKGAASTSYFTCENAEVVGADGENTQAKYITYDRREGLYGGTVQFDLETVPSMDTKYFTISLKGSGTAQTINVNLGSEVSYNIALAEGDTDWHQYIFDISYGAQYPEDIAFVKLASNTKIESYFYADDFGFSKTKPERVIPNPEKTFIYDFATYNRNTAKYEAVISTMPGSNDDEIRAEKVDGGLDFETQALEITYSRNGNIPSKTMVVYSPSDFFKGNSNDDERTANRATLKADMEYMTDLVFYGKSTSDKNEKINVGVIDAANSMMTYTDTKEFTLTSQWQQFRVPFDEFKVLDGGSELDCSRVRGFVFSSAENSGEGSFMIDNITHTSVADIEWAE

SEQ ID NO: 34 - protein, 575 aa, Bacteroides salyersiae
MRHRVILFICVLQTLFAYAVGAETHFMLTLNEQWKFSTGDSSAWATTEFDDNQWGTISSRQYWEEQGYDGYDGYGWYRQHFMISEDWKPIVTNAGGLYIRYEFADDVDEVFVNGVSVGRMGEFPPEYKVIYGGMRKYKISPGLLRFGEENLIAIRVYDNGGAGGLKTENILLQSITPMDDLMLDIRCDDSDWVFENTETIDFRVRPKQPLAAGGEFNLVCSVTTDTYLPVDSFVYRVKGDFEQPVSFVPPAPGFYRITLYGEQQGVKSDFLKFNMGYCPEQIISPVDVEPDFDQFWETTLKELSEVVPDYRMTLLEEKSQGAKNIYRVEMYSLGNVRIEGYYAVPKQKGKFPSVISFLGYGSGGGFPRPDNLPGFCEFILSTRGQGIQLPVNTYGKWIVHGLEDKSQYYYRGAFMDLVRGIDFLCSRPEVDTEKIFAEGGSQGGAFTLAACALDRRICAAAPYIPFLSDFEDYFKIAPWPRSVFEEYLRSHEESSWDEIYRLLSYFDSKNLAPRITCPIIMGVGLQDNICPPHINFSGYNQVKSPKRYYIYYDKEHTVGKSWWTIRNNFFRSFCN

SEQ ID NO: 35 - protein, 737 aa, Bacteroides salyersiae
MKKLFKLFAFTCLAMSATAQNKTPIYLDETKPIEQRVEDALQRMTLEEKIKLCHAQSKFSSHGVPRLGIPELWMTDGPHGIREEVLWDEWKGAAWTSDSCIAFPALTCLAATWDLDMSALYGKSIGEEARFRGKDVLLGPGVNIYRTPLNGRNFEYMGEDPYLAAKMVVPYIKGVQQNGVAACVKHFALNNQEMYRGHINVEVSDRALHEIYLPAFKAAVLEGGTWSIMGAYNQYKGQHCCHNQYLLNDILKKDWNFDGTVISDWGGVHDTYQSAYYGLDLEMGTWTDGLSWGKTNAYNNYYMALPLLEKIKNGEIEENTVNDKVRRLLRMMFRTSMNTQKPWGSFGTEEHALAGRTIAENGIVLLKNENGLLPVDLSQIKKIAVIGENATKVMTLGGGSSSLKVKYEVSPLEGLKKRVGNAVELVYAPGYASPLTDKRDPRYIVLEGYRLPDAEKLTKEALEAAKNADIVLFFGGLNKNEHQDSEGTDRLNYHLPYGQDELIAQLSKVNKNIAVILISGNAVAMPWIKEVPSVLEAWFSGTESGNAIASVLVGDVNPSGKLPMTFAVRLEDYPAHTVGEYPGDSINVKYNEGIFVGYRWTDKHKIRSLFPFGHGLSYTTFQYGKALLSSSEMNEKEILTVTIPIKNTGKVKGKEIVQLYIGDEKSSLERPVKELKGFQKIELNPGEEKVVEFNITSNDLKFYDEAIQDWKAEQGKFNIFIGSSSTDIRAKTKFNLK

SEQ ID NO: 36 - protein, 756 aa, Bacteroides salyersiae
MGVSVFAADDGGALYLDAGRPVEQRVKDLMSRMTLEEKVGQMCQWVGLEHMRTASQDLTVDELSNNTARGFYPGITEEDVRQMTIDGKVGSFLHVLTVKEANQLQELAMKSRLKIPLIIGIDAIHGNAQVVGTTAYPTSIGQASMFDVGLVEEICRQTALEMRATGSQWTFNPNVEVARDPRWGRVGETFGEDPYLVSLLGVASVRGYQGDGFGKAENVLACAKHFIGGSQPINGTNGSPTDISERTLREVFLPPFKATVDAGVYSFMTAHNELNGIPCHANPWLMEDILRKEWGFDGFIVSDWMDIEHIHDLHRTAVDNKDAFYQSVDAGMDMHMHGPEFYEKVIELVKEGKLTEARIDESCRKILAAKFRLGLFEKSFTDEKAAKSVLFNEKHQATALEAARKSIVLLTNDGILPLDEAKYKNVFVTGMNADNQTILGDWALTQPDENVITVLEGLKLVSPDTKFSFVDLGWNIREMDKNKVEQAAKQAAKADLAIVAVGEYSLRTNWYDKTCGEDCDRSDINLAGLQQELVESILATGVPTVVVLVNGRQLGVEWIAGHANALVEAWEPGSLGGQAIAEILYGKVNPSGKLPVTVPRHVGQIQMIYNHKPSMYFHPYAIGESTPLFYFGYGLSYTEYAYSDLTVSSAQMSGDGSVEVSVKVTNTGTTDGEEIVQLYIRDLYSSATRPVKELKDFRRVPLRVGETKTVSFILPAGKLAFYDKKMDYTVEPGDYEIMVGASSRDEDLMKRIVNVK

SEQ ID NO: 37 - protein, 1045 aa, Bacteroides salyersiae
MEKKTKRIAFVLATMLCGWQMMLAQPVSPAPTPTRAANDVKAMFSDAYPEKFGKFQIDYDDWNSDKFLTTKTIVTPFGAADEVLKIEGLSTGSLQHNAQIALGTCNLSDMEYLHMDVYSPSENGIGEFSFYLVSGWSKTVSCNVWYNFDTKQEYDQWISIDIPMSTFKNGGLNLAEINVLRIARGKQGAPGTIVYVDNVYAYGKAVEPESDVKIVANGNANLTTDVPLISAPTPKVAAANVFNFFSDHYGDGKFDYAQSDYGDQKTVKSLITINDTEDQVFKIDNIVNGSKANVSIGSPNLSGVDMLHLDIFSPGNDQGIGEFDFALTDFGGNGNDAGIWLNITDKGWHGQWISIDIPLSKWTGAANMIRFRRGGKGSTGKLLYVDNVYAYKSESDDPKPVPDPTTVPVLTKDKSDVISIFCEQYEEPGYQDEFGIVSAGNWGQNAKQKDEFVEIVAGNQTLKLTSWDLFPFKVHKNSDVMDLSQMDYLHLSIYQNGALDENNKPVSVCIWINDKDNKVAQAPLLEVKQGEWTSVSFGMDYFKNKIDLSRVYVIRLKVGGYPTQDIYVDNIFGYKGDPIRPGQVTEPYVDECDQKIQDSTPGTLPPMEQAYLGVNLASASGGSNPGTFGHDYLYPKFEDLYYFKAKGIRLLRIPFRAPRLQHEVGGELDYDAGNTSDIKALAAVVKEAERLGMWVMLDMHDYCERNIDGVLYEYGVAGRKVWDSAKNTWGDWEAMDEVVLTKEHFADLWKKIATEFKDYTNIWGYDLMNEPKGININTLFDNYQAAIHAIREVDTKAQIVIEGKNYANAAGWEGSSDILKDLVDPVNKIVYQAHTYFDKNNTGTYKNSYDQEIGGNVEVYKQRIDPFIAWLEKNNKKGMLGEYGVPYNGHAQGDERYMDLIDDVFAYLKEKQLTSTYWCGGSMYDAYTLTVQPAKDYCTEKSTMKVMEKYIKDFDTSIPSSLVETNADGNAIVLYPNPVKDNLKITSESGIEQVIVFNMIGQKVSERNEKGTNIELNLEALGKGTYLVTVRLEDGNVVNRKIVKM

SEQ ID NO: 38 - protein, 413 aa, Bacteroides salyersiae
MSCVLVCAGVLLLLSGLRETDVVGTKKQLSYCDTQIKKTLDAIEGSGLMPRCIDTDATDWYKIDIYDWTSGFWPGILWYDYENTQNEEIRKAAIHYTESLVPLLDPEHPGDHDLGFQFYCSFGNAYRLTKDDKYKQVLLKGADKLAGFYDPRVGTILSWPGMVTEMNWPHNTIMDNMMNLELLFWAAKNGGNREYYGMAVSHAKVTKENQFRPDGSCYHVAVYDTIDGRFLKGVTNQGYSDSSLWARGQAWAIYGYTLVYRETGDKEYLRFAEKITDIYLKRLPEDYVPYWDFDDPAIPDAPRDASAAAIVASGLLELVQLEDNTEKAEEYRDAAVNMLLSLSSDAYQSGIKKPSFLLHCTGNLPGGYEIDASINYADYYYIEALTRYKKMQAGRDIVEKYPQATQKQVTIAM

SEQ ID NO: 39 - protein, 1029 aa, Bacteroides salyersiae
MKSHPLLILLIIIPTCLFAGNPDKVSLVDMFMGVKNSSNCVIGPQLPHGSVNPAPQTPNGGHNGYDENDVIRGFGQLHVSGIGWGRYGQVFISPQVGFKPGETEHDSPKSDEVATPYYYKVNLDRYKIKTEITPTHHSVYYRFTYPKSGNKNILLDMKHNIPQHIVPIVKGTFLGGNIEYDKASGLLTGWGEYAGGFGSAAPYKVFFAMRPDVKLKEVKVTDKGTKALYARLSLPEEAETVHLGIGVSLRSVENACKYLEQEIGARSFDEVKRVAKSAWEDVFATIDVKGGTQEEQRLFYTAMYHSFVMPRDRTGDNPRWTSGQPHLDDHFCVWDTWRTKYPLMMLVNESFVAKTVNSFIDRFAHDGECTPTFTSSLEWEMKQGGDDVDNIIADAFVKNLKGFDRQKAYELVKWNAFHARDSLYLKKGWIPETGARMSCSYTMEYAYNDDCGARIARIMKDDETADYLENRSQQWVNLFNPNLESHGFNGFVGPRKENGEWIGIDPALRYGPWVEYFYEGNSWVYTLFAPHQFSRLIRLCGGKEAMADRLTYGFEKELIELDNEPGFLSPFIFSHCDRPGQTAKYVDFIRKNHFSRATGYPENEDSGAMGAWYIFTSIGFFPNAGQDFYYLLPPAFSEVTLTMENGKKIDIKTVKSTPEVNYIESVSLNGKLLDRTWIRHAEIAEGATIVYHLTDKPGQWSISPFEASRREPQPFGVNLAGAEFFHKKMEGVGRFNKDYHYPTTDELDYWKSKGLTLIRLPFKWERIQRKLYGELNREEMDYIKFLLAEADKRDMQILIDMHNYGRRKDDGKDRIIGDSLSIDHFASAWGSISRELKDCKGLYGYGLINEPHDMLASTPWVGIAQAAIDSIRKNDAKNAIVVGGNHWSSAERWKLVSDDLKNLRDPSRNLIFEAHCYFDEDGSGIYRRSYEEEKAHPYIGVERMRPFVEWLKENDFRGLVGEYGVPADDERWLECLDNFLAYLSAEGVNGTYWAAGARWNRYILSVHPENDYRKDKPQMKVLMKYLRTQ

SEQ ID NO: 40 - protein, 1311 aa, Bacteroides salyersiae
MKHTILVLLGLALSFFPARAYHFRSYQVEDGLSHNSVWAVMQDSKGFMWFGTNDGLNRFDGKKIKVYRKIQGDSLSIGNNFIHCLKEDSRGRFLIGTKQGLYLFDDKLEKFRHIDLDKNIKDDVSINAIMEDPSGNIWLACHGYGLYVLTPELTTKKHYLSGSDPYSLPSNYIWSIVQDYYGNIWLGTVGKGLVHFDPKEEKFTQMTQAKELGIDDPVIYSLYCDIDNNIWIGTATSGLIRYTPRSQKATHYINHVFNIKSIIEYSDHELIMGSDKGLVKFDRTLESFDLINDDTSFDNMTDKSIFSIARDKEGSFWIGTYFGGVNYYSPAINRFQYCYNSPHNSSKKNIISGFAENENGDIWIGTHNDGLYLFNPKSLSFKKPYDIGYHDVQSILSDQDKLYASLYGKGIHILNIKNGQVSASANDIGINHTINSIAKTSKGQILFTSEGGVISMDASGTLKTLDYLTNTPVKDIAEDYDGSIWFATHSKGLIRLTSDNRWEVFVNNPDNPKSLPGNNVNCVFQDSKFHIWAGTEGEGLVRFNAKEQNFEPILNDQSGLPSNIIYSILDDSDGNLWVSTGGGLVKISSDLKNIKTFAYIGDIQRIQYNLNCALRASDNRLYFGGTNGFITFNPKEITDNPNKPVVMVTGFQIASKEITLSESSPLKETISATKEITLRHDQSTFSFDFVALSYLSPEQNRYAYILEGFDKEWHYTSDNKAMYMNIPPGTYVFRVKGTNNDGVWSDETADITVKIKPPFWLSNLMIGLYIVLAIGIILYFIRRYHRFIERKNQEKIFKYQTAKEKEMYESKINFFTNIAHEIRTPLSLIAAPLEKIILSGDGNEQTRNNLGMIERNANRLLELINQLLDFRKIEEDMFHFKFKRQNVVKIVEKVYKQYYQTAKFNKLEISLEAEKNDIECNVDSEAIYKIVSNLIANAIKYAKSQILITVKERSGNLEIKIKDDGTGIEKQYMEKIFEPFFQIQDKNNAVRTGSGLGLSLSQSLAMKHNGKISIESEYGKNCNFTLTIPIADGTEEEVQETEAAIPEKSEMPEQSVVEAGTRIIIVEDNTDMRTFLCESLNDNYTVFEAENGVQALEMVEKENIDIIISDIMMPEMDGLELCNRLKSDPAYSHLPLVLLSAKTDTSTKIEGLNQGADVYMEKPFSIEQLKAQISSIIENRNNLRKNFIKSPLQYFKQNTENNESADFVKKLNTIILENMSDEDFSIDSLSSQFAISRSNLHKKIKNITGMTPNDYIKLIRLNESARMLSTGKYKINEVCFLVGFNTPSYFSKCFFEQFKKLPKDFIQITNE

SEQ ID NO: 41 - protein, 723 aa, Bacteroides salyersiae
MKKQFSTLIALLIVGAAPLLGQETDPLNDPTNIDADLYLHAGFSQDSIRPDYSHTYYDNTNHKLVKGEDGIYSITVPLKKEQIVNKNMEVGIYTYAYSVIYGGKVNGSGNDAVKGSVGPVIADEPRLFELAEDRDVTFYAKKLNTGTADAPWYRTMFICDAQPLYLDGTELPLPGEDGVTRYVVDRGETSRRWEYKLSPIGRWSKTQDFMEDVIPAKWKSNEAYAFLPNGGWWLGGRFLLAYDYKKLSLEVGKLVDELQTPLFTVNGESIPENLGIVDELLLNGSVITFLKGYYANGGKDSYDPAFNTSIATVKLCWQIDELPAASFPLTNGEVVRDDNYNKTTEWTVSEADLFEGTTLPAGIHTLKVWYESEYLGDVLTSEVQSTSFEIEEIVVIPLENKGTAVDLILEGDWNPETFRTIIEEQAVRITTIDLTGVAGLTELPEMEGLNPNCLVYVNPDVVIAEGVDNVVVFDNEEGRAANILLTEGSDFNNVRLFTADRISYSHNFTADVWSTICLPFSADKGDVTVEEFTGADGEKVIFTGTSAIEANVPYLAKTSNSEVKTFTATDVQMSVTAEPAPVVPENGYAFHAGYRAVEGDAVVGLHLMNDVGTAFVKVADGNPEAAGVSAFHAYMQATVDELLTIVHGDDNPTGLGSTEDTGRLTIISHNGSVEIKTGKAQMIGLYALDGRLVKMVELSQGSNFVNGLDKGIYIMDCQKVVVK

SEQ ID NO: 42 - protein, 357 aa, Bacteroides salyersiae
MKKIITIAFLSFLYFVYGYASNHSMHPLKQIDYVLKQVKAQQEPYYSAYQQLIHDADSILKVSHHALVDFAVPGFYDKPEEHRANSLALQRDAYAAYCSALAYTLSGQQEYGEKACYFLNAWASTNEKYSEHDGVLVMTYSGSAFLMAAELMADDPLWSNKEKKDFRKWVKRIYQHAANTIRVHQNNWADWGRFGSLLAASFLNEKKEVAENVRLIKSDLFHKIATDGSMPEETRRGGNGIWYTYFSLAPMTGACWLVYNLTGENLFALEQDGTSIKKALDYMAYYNKHPKEWKWDKNPNTGKNEVWPENLLEAMANLYNDNSYVEYVKGKRPIIYRNHHFCWTFPTLMPTSFENYQ

SEQ ID NO: 43 - protein, 1025 aa, Bacteroides salyersiae
MISKDENIKRRIIGVLFFLCALSPALWAQSRIIKGEVLDPNGEPLIGVGVMIKNTTAGTITDVDGRYSIQVPDNNAVLSFSYVGYKRKEVKVGSQSVINISLEEESVLMDQVVIVGYGSQKKVNLTGAVAAISVDESLAGRSVANVSSALQGLMPGLSVSQSSGMAGNNSAKLLIRGLGTINSADPLIVVDDMPDADINRLNMNDIESITVLKDATASSVYGSRAANGVILVKTKSGKGLEKTQITFSGSYGWEKPTNTYDFISNYPRALTLQQISSSTNPGKNGENQNFKDGTIDQWLALGMIDDKRYPNTDWWDYIMRTGSIQNYNVSATGGSEKSNFYASVGYMKQEGLQINNDYDRYNARFNFDYKVMKNVNTGFRFDGNWSNFTYALDNGFTSDSNLDMQSAIAGIYPYDPVLDVYGGVMAYGEDPQAFNPLSFFTNQLKKKDRQELNASFYLDWEPVKGLVARVDYGLKYYNQFYKEADIPNRSYNFQTNSYGIREYVTENAGVTNQTSTGYKTLLNARLNYHTVFATHHDLNAMFVYSEEYWHDRYQMSYRQDRIHPSLSEIDAALSGTQSTSGNSSAEGLRSYIGRINYSAYGKYLLELNFRVDGSSKFQPGHQYGFFPSAALGWRFSEESFVKPYIGKWLASGKLRASYGKLGNNSGIGRYQQQEVLYQNNYMLDGSIAKGFVYSKMLNPDLTWESTGVFNLGLDLMFFDGKLAAEFDYYDRLTTGMLQKSQMSILLTGAYEAPMANLGTLRNRGFEANLTWRDRIADFTYSANFNISYNRTNLEKWGEFLDKGYVYIDMPYHFVYSQPDRGLAQTWTDSYNATPQGVAPGDVIRLDTNGDGRIDGNDKVAYTNFQRDMPTTNFALNLQMGWKGIDVSLLFQGSAGRKDFWNNKYTEINLPDKRYTSNWDQWNKPWSWENRGGEWPRLGGLVTNKTETDFWLQNMTYLRMKNLMIGYTFPKKWTRKCFIENLRIYGTAENLLTITGYKGLDPEKAANSQDLYPITKSYSIGVNLSF

SEQ ID NO: 44 - protein, 613 aa, Bacteroides salyersiae
MKRVYIKYIGLIAGMMMLFSSCADLLNQEPTVDLPATNYWKTESDAESALNGLVSDIRWLFNRDYYLDGMGEFVRVRGNSFLSDKGRDGRAYRGLWEINPVGYGGGWSEMYRYCYGGINRVNYVIDNVEKMIANASSEKTIKNLEGIIGECKLMRALVYFRLIMMWGDVPYIDWRVYDNSEVENLPRTPLAEVKDHILDDLLDAFKKLPEKATVEGRFSQPAALALRGKVLLYWASWNHYGWPELDTFTPSEEEARKAYKAAAEDFRTVIDDYGLTLFRNGEPGECDEPGKADKLPNYYDLFLPTANGDAEFVLAFNHGGTNTGQGDQLMRDLAGRSVENSQCWVSPRFEIADKYQSTITGDFCVPLVKLNPSSVPDARTRPNSAVNPESYKDRDYRMKASIMWDYEICQGLMSKKVTGWVPFIYKMWGSEVVINGETYMSYNTDGTNSGYVFRKFVRNYPGEERADGDFNWPVIRLADVFLMYAEADNAVNGPQPYAIELVNRVRHRGNLPVLASSKTSTPEAFFEAIKQERIVELLGEGQRAFDTRRWREIETVWCEPGGRGVKMYDTYGAQVAEFYVNQNNLAYERCYIFQIPESERNRNPNLTQNKPYR

SEQ ID NO: 45 - DNA, 29818 bp, Bacteroides salyersiae
ttattttagattaaacttggtttttgcgcgtatgtcagtagatgagctgcctatgaagatattgaatttgccttgttcggctttccagtcctgtatggcttcatcgtagaatttcaggtcattggaggtgatgttgaactctaccaccttttcttcgcccggattcagttctattttttggaaccctttcaactctttgacaggacgttccaatgagcttttttcatcaccgatatagagttgcacaatctcttttccttttacttttccggtgttttttatagggatagtaacggtcagtatctccttttcgttcatttcggaggatgaaagaagggcttttccatattggaaagtggtgtagcttaaaccgtgtccgaacgggaacaagctccggattttatgtttgtccgtccaacggtagcctacgaatatgccttcgttgtatttcacgttgatgctgtcaccgggatactctccgacggtatgtgccggatagtcctccaggcggacagcgaaagtcatcggtaacttgccggaagggttaacatctccaaccaatacggaagcaatggcattaccggattccgtaccgctgaaccaggcttccaaaacagacggcacctctttgatccacggcattgcgactgcatttcccgaaataagaataacggctatgttcttatttactttgctgagttgggctatcagttcgtcctgtccgtaaggcaaatgatagttgagccggtccgtgccttcgctgtcctggtgttcgttcttattcagacctccgaagaacagtacaatatccgcatttttggcagcttcaagagcctctttcgttagtttctccgcatcgggaagccggtatccttcgagaacgatgtaccggggatctctcttatcggtgagcgggcttgcatatccgggagcgtaaaccagttcgacggcattgcctacccgtttcttcagcccttcgagcggagaaacttcgtatttcactttcaatgaagaagagccacctcccaatgtcatgactttcgtggcattttcgccgataacggctattttctttatttgggaaagatcgacaggcagcaatccgttttcgttcttgagtagcacgatgccgttttcggcaatggtgcgtcctgccagtgcatgttcttccgtgccgaatgatccccatggcttttgagtgttcatgcttgtccggaacatcatgcgaagcagcctgcgcactttgtcattcacggtgttttcttcgatttctccgtttttgatcttttccagtagcggcaatgccatgtagtagttgttgtaggcattggtttttccccagctcaatccgtctgtccatgttcccatttccagatcgagtccgtaataggccgattggtaagtgtcgtgtacacctccccagtcggaaatcacagttccgtcgaaattccagtcttttttcagtatatcgttcagcaaatactggttgtggcagcaatgttggcctttgtattggttgtaagcccccataatggaccatgttcccccttccagtacggccgctttgaaagcaggcaggtaaatttcgtgcagggcgcggtcactgacctccacattgatgtgtccgcgatacatttcctgattgttcaatgcaaaatgcttgacacaggcggccaccccgttttgttgtacccctttgatgtatggcactaccattttagcggccagataaggatcttctcccatgtactcaaagtttcgtccgttgagtggtgtgcggtatatgttgacacccggacccagcagaacatctttcccccggaagcgggcttcttcgcctatcgacttgccatacagggccgacatatcaagatcccatgtggcggcaaggcaggtcagtgcagggaaagcgatgcaggaatcactggtccaggcagctcctttccattcatcccacagcacctcttcccggataccgtgtggtccgtcggtcatccaaagttccggtatgccgagacggggcacgccatgggaactgaatttagattgtgcatggcacaattttattttttcttccagagtcattcgttgaagtgcatcttccacgcgttgctctatcggttttgtttcgtccagatagatcggagttttgttttgcgccgtggcagacattgccaggcatgtgaatgcgaataatttaaaaagctttttcatgttgtaagataatctttattgataattttcaaaagaagtgggcatcagcgtggggaatgtccagcagaagtgatggttgcggtaaatgattggacgcttcccttttacatattccacataagagttgtcattgtataggtttgccattgcttcgagaaggttctcgggccagacttcatttttcccggtattcgggttcttgtcccatttccattctttgggatgtttattatagtaggccatatagtccagcgctttttttatggaggttccgtcttgttccagagcgaacagattctctccggtgagattgtacactagccagcacgctccggtcatcggtgccagtgagaaataggtgtaccatatgccgttgccgcctctccgggtctcttcgggcatgctgccgtctgttgctattttatggaataagtccgattttatcaggcgcacgttttccgcaacttcttttttctcatttaggaatgaagccgccagcagcgagccgaaacgtccccaatccgcccagttgttctgatgaacccggatggtattggcggcgtgctggtagatgcgtttcacccatttccggaaatcctttttttctttattgctccaaagcggatcatctgccatcagttccgctgccatcaggaatgctgaaccggaataggtcatcaccaacactccgtcgtgttccgaatatttctcgttggtagatgcccatgcattcaggaaatagcaggctttttctccatattcctgttggccggatagggtgtaggccaatgccgaacagtaggcggcatatgcatcccgctgcaatgccagggagttggcacggtgttcctcgggtttatcatagaatcccggtacggcaaaatctaccagtgcatggtgcgacactttcaagatggagtcggcatcgtgaatcaattgctgataggcagaataatagggctcttgctgtgccttgacctgttttagtacatagtcgatttgctttaacggatgcatgctgtgattgctggcgtatccgtagacgaaatataaaaaagagagaaatgctatcgttataattttcttcatttttaggatttatttattcgtttgttatttgtataaaatctttaggaagtttcttgaactgttcgaagaaacattttgagaaataagacggtgtgttgaaacctaccaggaagcatacttcgtttattttgtatttgccggtggacaacatccgtgcgctttcattcagcctgatcagcttgatgtagtcgttgggggtcattcctgtgatgtttttaatctttttgtgcaggtttgaacggcttatggcaaactggctggaaaggctgtcgatggagaagtcctcatccgacatgttttccaatatgatggtattcagcttcttcacgaaatctgcgctttcgttgttttccgtgttctgtttgaaatactgcaacggagatttgatgaagttctttcgcaggttgttcctgttttcaataatgctgcttatttgcgcttttagttgttcgatggagaatggtttttccatgtaaacgtcggctccctggttcagaccctctattttagtggaagtatccgttttggcagatagcaacaccaaaggcaggtgagagtaggcgggatcgcttttcagccggttacataattccaacccgtccatttccggcatcataatatcggatatgatgatgtctatgttctctttttccaccatttccagtgcctgtactccgttctctgcttcaaagacggtgtagttgtcgtttaggctttcgcaaaggaaagtccgcatatccgtgttgtcttccacgatgatgatcctcgtacccgcttccacgaccgattgttcgggcatttcactcttttcgggtatggcagcttccgtttcctgtacctcttcctctgttccgtcggcaatggggattgtcagtgtaaaattacagttctttccgtattccgattcgatggaaatttttccgttgtgtttcatggccagcgattgcgatagcgataaccccaggccggagcctgtccgcacagcgttgttcttatcctgtatctggaagaaaggttcgaatattttttccatatactgcttttcaatgccggtaccatcgtctttaatctttatttccaggtttccgcttctctcttttacggttatcaggatttggcttttggcatatttaatggcgttggcaatcaggttgctgacaatcttatagatggcttcggagtcgacattgcattctatatcgttcttttccgcttccaaagagatttccagtttgttgaatttggccgtctggtaatattgcttatacactttttccacaatcttgacgacattctgccgcttgaatttgaagtggaacatgtcctcttctattttacggaagtccagcagttggttgatcagttcgagcagcctgttggcattgcgttctatcatccccaggttgttcctggtctgttcgtttccgtctccggacaaaattattttttccaatggtgcggcaatcagcgagagcggtgtacgtatttcatgggcaatgttcgtgaagaaattgattttcgattcgtacatctctttttctttggccgtctggtatttgaatatcttttcctggtttttacgttcgataaagcggtggtatctccggataaaataaaggataatgccgatggcaaggacaatatacaggccgatcatgaggttggacaaccagaacgggggctttattttcaccgtaatgtctgccgtttcatcgctccatactccatcattattcgtgcctttcacacggaatacataagttccgggcgggatgttcatgtacatggccttattgtcggaggtgtaatgccactctttgtcgaagccttcgaggatgtaggcatatctgttttgttccggcgaaagatagctcaatgctacaaagtcgaagctgaaagtggactggtcgtgccgcaacgttatctctttggttgcgctgatggtctcttttagtggcgacgattcggaaagtgttatctctttgctggcaatctggaaacctgtgaccatgacgaccggtttgttggggttatctgtaatctctttcggattgaatgtgatgaatccgttggttccgccaaagtaaagtcggttgtcggaagctctcaatgcgcagttcagattgtattgtatccgttgtatatcgccgatataggcaaatgttttaatgtttttcaagtcggaggatattttaaccaaccctccgcctgtgcttacccacagattgccgtccgaatcgtccagtatggaatagatgatgttggaaggtaggcccgactggtcgtttaagattggttcgaagttttgctctttggcgttgaaccgtaccagcccttctccttccgtccctgcccagatgtggaatttggagtcttgaaatacgcagttgacattatttcccggcaaggatttcggattatcgggattatttacgaatacttcccatctattgtctgaggtgagccgtatcagccctttggaatgggtggcaaaccaaatggagccgtcataatcttctgcaatgtcttttaccggggtgttggtcaggtaatcgagggtcttgagcgtgccggatgcatccatcgagatcactccgccttcggaggtgaagagtatctgccctttggaggttttggctatggagttgatggtatgattaattcctatgtcgttggcggaggcgctgacctgtccgttctttatgttcaggatatggatgcctttgccgtaaaggcttgcataaagtttgtcctggtccgacagaatgctctgtacatcgtggtaaccgatgtcgtatggcttcttgaagctcaggctcttcggattgaaaaggtatagtccgtcgttgtgcgttccgatccatatgtccccattttcattctcggcgaatccgctgatgatattttttttggaagagttgtgtggagagttatagcaatactggaaacggttgatggcaggcgaataataatttacgcccccaaagtaagttccgatccagaaagacccttccttgtcacgtgcaatggagaaaatggatttatccgtcatgttgtcaaaagaagtatcgtcgttgatcaggtcgaaactctccagcgtacggtcgaatttcaccagtcctttgtccgatcccatgatgagctcgtggtcggaatattcgatgatggatttgatgttgaatacatgatttatataatgtgtggctttctgtgatctgggggtatagcgtatcaatccgcttgtggccgtccctatccagatgttattgtctatgtcgcaatacaggctgtaaatcacgggatcgtcgatgcccaactctttagcctgtgtcatttgcgtgaacttttcctctttaggatcaaagtgtacaaggcctttgcccactgtgcccaaccatatatttccgtaataatcctgaacgatgctccagatgtaattggagggcaacgaatagggatcgctaccggatagataatgtttcttggtcgttaattcgggagtaaggacatacaggccatatccgtggcaggccagccatatatttccggaagggtcttccataatagcattgatgctcacgtcgtcttttatgtttttgtccaggtcgatgtgtctgaacttctctaacttatcgtcgaaaagatagagcccttgtttggttccgatgaggaatcttcctcgcgaatcctctttcaggcagtggataaagttattgccgatagataaagagtcgccctgtattttgcggtacactttgattttcttgccatcgaaacggttgaggccgtcgttggtcccgaaccacatgaagcctttgctgtcctgcataaccgcccagacactgttatgcgacaatccgtcttccacctgatagctcctgaagtgataggcgcgtgcaggaaaaaaagataaagccaaacctaataaaactaaaatcgtatgtttcatagcctgatgaaattaagatgttcaaatatagggctttgctctctttggcgatgcaaatatcttcttaaaacctataaaaatatggtataattgtgagaatgcagtgtatttatatctttgaaaagtatatttctatccactttgttttatcagttctacatttgtgtcattcatattagtaattaaagtctaatctttagaaacatgaataagttagtcagtacttttattatttcatcctttactgctgctatgggcgtatcggtttttgctgctgatgatggcggtgcgttatatctggatgcgggccggcctgtcgagcagagggtgaaagatttgatgtcgcgcatgactctggaggagaaagtggggcagatgtgtcaatgggtcggcttggagcatatgcgaaccgcttcacaggatttgacggtagacgaattgagtaataacacggcgcgggggttctatcccggcatcacggaagaagacgtgagacaaatgacgatagacgggaaggtgggctctttcttgcatgtactcacagtcaaggaggccaatcagttgcaggagctggcaatgaaaagccgtctcaaaatccctttgattataggcatcgatgccattcacggcaatgcgcaggtagtgggtactacggcgtatccgacgagcatcgggcaggcatccatgttcgatgtcggcctggttgaagagatttgccggcaaacggctttggagatgcgtgctacaggttcgcagtggacattcaatcccaatgtagaggtcgcccgcgacccgcgttgggggcgtgtcggcgaaactttcggcgaagatccctacttggtatctttattgggcgtggcttccgtgcgcgggtatcagggagacgggtttggaaaggcggaaaatgtgttggcttgtgccaagcattttattggaggcagccaaccgataaacggaacgaacggctctcccacagacatttcggaacggacactccgggaggtattcctgcccccctttaaggcgaccgtagatgccggtgtatatagctttatgacagctcataatgaactgaacggcattccctgtcatgccaatccatggctgatggaagatattcttcgcaaagaatggggattcgatggtttcatagtcagtgattggatggacatcgagcatatacacgacttgcatcgcacggcagtggataataaagatgctttctaccagtcggtagatgccggaatggatatgcacatgcatggaccggagttttacgaaaaggtgattgaactggtgaaggagggaaaactcacggaagcccggatcgatgagtcttgccggaaaatattggctgcgaaattccggttaggactgttcgagaaatcttttaccgatgagaaagcggcgaaaagcgtcctgttcaatgaaaagcatcaggccacggcattggaagcggcgcgtaagtccattgtgctattgaccaatgacggcatacttccgctggatgaagcaaaatataaaaatgtattcgtaaccggaatgaatgccgacaatcagacgattctcggtgattgggctttgacacagccggatgagaatgtgattacagtgctcgaagggctgaaactggtatctcccgacactaaattttcatttgtggatttgggatggaacatccgggaaatggataaaaacaaagtggaacaggccgcaaagcaggctgccaaagccgatttggcaattgtggcggtgggagaatattccttgcggaccaactggtacgacaaaacttgtggcgaagactgcgaccgttcggatatcaatctggcagggttacagcaggaacttgtggagtccattctggcaacgggagttcctaccgttgtggttttagtaaacgggcgtcagttgggggtggaatggattgccggtcatgccaatgctttagtcgaagcgtgggagccgggtagtctcggaggacaggccattgccgaaatattatatggaaaagtaaacccttccggcaaactgccggtgacggttccgcgccatgtgggacagatacagatgatttataaccataagccgtccatgtattttcatccgtatgccatcggagagagtacgcctttgttctattttggatacggcctgagttatacggaatatgcgtattcggatctcacggtttcctcggcgcagatgtcgggggacggcagtgtggaagtgtccgtgaaagtgacgaatacgggaacaacggatggggaggagattgtgcagttgtatatccgcgacctctattccagtgcgacgcgtccggtgaaagagttgaaggacttcaggcgcgtgccccttcgtgtaggcgaaaccaagacagtttctttcatcttaccggcagggaaacttgctttctatgataagaagatggactatacggtggaacctggagactatgaaatcatggtgggagcttcgtcgagggatgaagatttaatgaagagaattgtaaatgtaaaataatagttgggatgaaaagattgatgagctgtgtgttggtttgcgcaggagtattgcttttgctgtcgggactgagagaaacagatgtagtcggaacaaaaaagcaattatcgtattgtgacacgcagataaagaaaacactggatgccatcgaaggttccggattgatgccccgttgcatcgatacggatgccacagactggtataaaatcgatatttatgattggacgagcggtttctggcccggcatcttgtggtacgattatgagaacacccaaaatgaagagatcaggaaagcagccattcactatacggaatcgcttgtgcctttgctcgatccggagcatccgggcgaccatgatctgggattccagttttattgcagctttggcaatgcctatcgactgacaaaggacgacaaatacaagcaggtattgctgaaaggtgccgataaactggccggattttatgacccccgggtggggacaatcctctcgtggccgggtatggtgacggagatgaactggccacacaataccatcatggacaacatgatgaatcttgaactgctgttttgggcggccaagaatggcggcaacagggaatactatggcatggcggtgagccatgcaaaggtgacaaaagagaatcagtttcgtcccgacggttcttgctaccatgtagcggtgtacgataccatcgacgggaggttcttgaaaggcgttacgaatcaaggatatagtgatagctccctgtgggcgcgcggacaggcatgggccatttatgggtatacgttggtttacagggaaaccggtgataaggaatacctccgttttgccgagaaaataacggatatatacctcaaacgtttgccggaagattatgttccgtattgggatttcgacgatccggctatcccggacgctccgagagacgcatctgcagcggccattgtagcttccggattgctggagctggtgcaattggaagataatacggagaaagccgaagagtatagagatgcggctgttaatatgctgctcagtctgtcgtctgatgcttaccagagtggtatcaaaaaaccgtctttcctgctccattgcacgggcaatttaccgggagggtatgagatcgacgcatccattaattatgctgactattattacattgaagcgctgacacgttacaaaaaaatgcaggctgggcgtgatattgttgaaaagtacccacaagctacgcagaaacaggtcactattgctatgtaaacaggattttggtagtaataaataatattgttgtatttgtttatcgcttgtcgggctacttttgtgcagaacagattgtttaaacttaaaaatattgtattatgaaaaaacagttttctactttgattgcattacttattgtcggagctgctccccttttggggcaagaaaccgaccctctgaacgatccgactaatattgatgcggatctctatcttcacgccggattttctcaggattccatccggccggattattcccatacttattatgataacaccaaccataaactggtaaaaggggaggatggcatatattccattacggttcctttgaagaaagagcagattgtgaataaaaacatggaggttggtatttatacctatgcttactctgttatttatggaggaaaagtgaacggttcaggcaatgatgccgttaagggaagtgtaggaccggttattgccgatgaacccagactctttgaactggccgaagaccgggatgtcactttttatgcaaagaaactgaatacaggaacggcggatgctccgtggtacagaactatgttcatctgcgatgcacaaccgctatatctggacggaacggagctgccgttgccgggcgaagatggagtgacgagatacgtagtggatagaggtgaaaccagcagacggtgggagtataaactcagccctatcgggcgttggagcaaaacgcaggattttatggaagatgtgataccggccaaatggaaatctaacgaagcatacgcttttctgcccaatggcggctggtggctcggagggcgttttctgttggcgtatgactataagaagttgagtctggaggtcggcaaattggttgatgaactgcaaactcccttgtttacggtgaatggagaaagtattccggagaatttgggaatagtcgatgaattgttgctgaatggttctgtgattacattcctgaaaggatattatgccaatggcggcaaagactcttatgatccggcatttaatacaagcatcgccaccgtgaaattgtgttggcagatagacgaattgcctgctgcctctttccctttgacaaacggtgaggtggtcagagacgataattataataaaacgaccgagtggacggttagtgaagcggatcttttcgaaggaacaactttgccggcgggaatacatacgctgaaagtatggtacgagtcagaatatttaggggatgtacttacttctgaagtacaatcgacgtccttcgagatcgaagagattgtggttattcctcttgaaaataaaggaacggctgtcgatcttattctggagggagactggaatccggaaacgttccgtacgattatcgaagaacaagccgttaggattactacgattgaccttaccggagtggccggcctgacggaacttcccgaaatggaaggtttaaatccgaactgcctggtttatgtgaatccggatgttgttatcgcagagggcgttgataacgtggttgtatttgataacgaagagggtagagcagccaatatacttctgacggaaggttccgatttcaataacgtgagattatttacggccgaccggatctcctactcccataactttactgctgatgtttggtctaccatctgcttgcctttcagtgcggataagggagatgtaaccgtagaagagtttacgggtgccgatggtgagaaagtcatctttacgggaacatccgccatcgaagccaatgttccctatttggctaaaacaagtaattcggaggttaagacctttacggcaacagatgtacagatgagcgttacggcagaaccagctccggtagttccggaaaatggttacgcattccatgccggttaccgtgcggtagaaggagatgctgtcgtaggactccatttgatgaacgatgtggggactgctttcgtaaaagtagccgatggaaatccggaagctgcgggagtttctgcttttcatgcttacatgcaggcaactgttgatgaactgttgacaatcgtccatggtgacgataaccctaccggattgggttcgacggaagataccggccggttgacgattatctcccataacggttctgtcgaaattaagacgggcaaggcgcagatgataggtttgtatgcattggatggccgtttggtgaagatggttgaactgagccagggcagtaattttgtcaatggattggataaaggtatttatattatggattgccaaaaggtagtagtgaagtaaaagaagtctccgtgtcttgtcccttgtacaagccggtagaatcagaataaagaaaaatttgaatggataataaataaaagaggtattgttttttttatgcagattcaagataataagttcattgtatcactttatcttgaatctgctttttttgaaatgacagcctctccccaaccctctccgtgggagagggagcaaaaaatgacttgtaaacaattgattaacagaactaactttagctccctctcccacggagagggttggggagaggctttataactttataaaaatgagacatcgggttatcctatttatttgtgtgttgcaaaccctgtttgcatatgctgtgggtgcggagactcactttatgctcaccttgaatgagcaatggaaattctcgacgggcgattcatccgcatgggccactacggaattcgacgataaccaatggggcactatctcttccaggcaatactgggaagaacagggttatgacggctatgacggttatggttggtacaggcagcatttcatgatttccgaggattggaaaccgatcgtaacgaatgccggaggtttatatataagatatgaatttgccgatgacgtggatgaggtttttgtcaacggggtctctgtcggtaggatgggagagtttccaccggaatataaagttatttatggcggtatgcgtaaatacaagatcagcccgggactgttgcgattcggtgaagagaatctcattgccatccgggtgtacgacaacggtggtgcaggagggttgaagacagaaaatatactcctgcaatccataactccgatggacgatctgatgctggatattcgttgtgacgatagcgactgggtattcgaaaatacagagacaatcgatttccgtgtacgtccgaaacaaccgcttgcggcgggaggggagtttaatctcgtttgcagcgtgacgacggatacctatctcccggtagactcttttgtgtaccgggtgaaaggagattttgagcaacccgtctctttcgttccgccggctccgggtttttaccggattactttgtatggagaacaacaaggtgtaaaaagcgattttctgaaatttaatatgggatattgcccggaacagattatttctcccgtcgatgtcgaacccgatttcgaccagttctgggaaactacgctgaaagagctttccgaagttgttcccgattaccgcatgactttactggaagagaagtcacaaggagccaaaaacatctaccgggtggaaatgtattcgttaggaaatgtccgtatcgaagggtattacgccgttcccaagcaaaagggcaagtttccgtctgtcatctcttttctgggctatggttccgggggtggttttcctcgtccggataatctgcccggcttttgcgagtttatcctttccaccagagggcaaggcattcagcttcctgtcaacacctatggcaaatggatcgtacacgggctggaagataaatcacaatactattatcggggggcatttatggatttggtgcgtgggatcgacttcctgtgttcacgtccggaggtggacacggagaagatttttgccgaaggcggaagtcagggcggagcttttacgctggcagcctgtgcactggatagacgcatctgtgcggcagcaccttacatccctttcctgtcggattttgaggattattttaagatcgcaccctggccgcgtagtgtgttcgaagagtatctgcgtagccatgaggagagtagttgggacgaaatataccggttgctttcctatttcgacagtaagaatctggcaccgcgtattacgtgtcccatcatcatgggcgtagggttgcaagataatatttgccctccccatatcaatttttccggctacaatcaggtgaagtctcctaagcgttattatatctattacgataaagaacatacggttgggaagagttggtggacaatcagaaataactttttccgtagtttttgcaactgaatctaatttatgtataccaaaatattgttcttgtcatattttggtatacatagattatatttttgcataagcggattcttttttgggcttattttgcttctgtcaagaaagctaaattgtttaattaaagaatctgtgaatacaatgaaaagtcaccctttactcatcttattaataattattcccacttgtcttttcgccggaaatccggataaggtatctctggtagatatgttcatgggggtaaagaacagcagtaattgtgtaattggccctcagttgccgcatggctctgtgaacccggcgccgcaaactcccaacggcggtcacaacggatacgatgaaaacgatgtgattcgcggattcggacagctgcatgtttccggcattgggtggggacgctacggacaggtgtttatctctccgcaggtcggtttcaaacccggcgagacggaacacgactctcctaagtccgatgaagtggctacgccctattattataaggtaaatttggaccgctataagataaaaaccgaaataacccccactcaccacagtgtgtactaccgcttcacctatccgaaatccggtaacaagaatatccttttggatatgaaacacaacattccgcagcacattgtccccatagtgaaaggtacttttctgggagggaatatcgaatacgacaaggcatcgggcttgctgaccggttggggcgaatacgccggaggtttcggaagcgctgctccctacaaagtgttttttgccatgcgtccggatgtgaaattgaaggaggtgaaagtcaccgataaggggacgaaggctctgtatgcccgtttgagtttgccggaagaggctgaaactgtccatctgggcatcggcgtttcactcagaagtgtggagaatgcatgtaaatatctggaacaggagatcggtgcgcgtagcttcgacgaggtgaagcgtgtggcgaaatctgcttgggaggatgtgtttgccactatcgatgtaaaagggggaacccaagaagagcagcgtctgttctatacagccatgtatcatagttttgtgatgccccgcgatcgtacgggcgacaatccccgttggacgagcggacaacctcatcttgacgatcatttctgcgtgtgggatacatggcgcaccaagtatcctttgatgatgcttgtcaatgagagtttcgtggcaaaaacggtgaattcttttatagaccgtttcgctcacgacggagagtgtactccgacctttaccagctctctggaatgggagatgaaacagggcggagatgacgtggacaatatcatagccgatgctttcgtgaaaaacctgaaaggattcgaccgccagaaggcgtatgaactggtgaaatggaatgcgtttcatgcccgtgacagcctttacctgaaaaagggatggattcctgaaacgggagcaaggatgagttgcagctacactatggagtatgcctacaatgacgattgcggtgcacgtattgcaaggataatgaaggatgatgagacggcggactatctggaaaaccgttcccaacagtgggtgaatttgtttaatccgaatctggaaagtcatggtttcaatggctttgtcggtccgcgcaaagagaacggcgaatggatcggtatcgatccggcgttgcgctacggtccgtgggtggaatatttctacgaaggtaattcttgggtgtacacattgttcgctcctcatcagttcagtcgtctgatccgtctttgcggagggaaagaggcgatggcagacaggcttacttatggattcgaaaaagagttgatcgaactggacaatgaaccgggattcctgtctccctttatcttcagccactgcgaccgtcccggtcaaaccgccaaatatgtagattttatccggaaaaaccacttctcccgggctaccggttatccggagaatgaagatagcggagcaatgggggcatggtacatctttacatcgatcggtttctttcccaatgccggacaggatttctactatttgcttcctccggctttttcggaggtgacgctgacaatggagaatggcaagaaaatagatattaaaaccgttaagtcgactcccgaagtcaattatatagagtctgtcagtctgaacggaaaactgctggaccggacatggatacgccatgccgagattgcggaaggcgctacgattgtctatcacttgacggataaaccgggacagtggagcatctctccttttgaagcaagcagaagagagccgcaaccgttcggggtgaatctggcaggggcggagttcttccacaaaaagatggagggagtggggcgctttaataaagattatcactacccgactacggacgagctggactactggaagtccaaaggactcactttgattcgattacctttcaaatgggaacgcatacagcgtaagttatacggagaattgaaccgggaagagatggattatatcaaattcttattggccgaagcagataagcgcgacatgcagatattgatcgatatgcacaattacggccggcgtaaggacgatggtaaggaccgcatcataggcgacagcctttcgatcgatcattttgcatcggcttggggatcgatctccagagaattgaaagactgcaaaggcctgtacggttacggcctgatcaacgaaccgcatgatatgctggcttctactccgtgggtagggattgcacaggcagccatcgactccattcgcaaaaatgatgcgaagaatgccattgtggtgggtggtaatcattggagttctgccgaacgctggaaactggtcagtgatgatttgaagaacttgcgcgacccgtcacgcaatctgatattcgaagcgcattgctactttgatgaagacggatcgggcatttaccgccgttcgtatgaggaagaaaaagcacatccgtacattggcgtggagcgtatgcggccttttgtggagtggctgaaagagaatgattttcgcgggcttgtcggtgaatacggagttccggcagacgatgagcgctggctggaatgtctggacaatttcctggcttatcttagtgcggaaggcgtgaacggtacctattgggcggccggtgccagatggaacaggtatattctttccgttcatccggagaacgattaccggaaagacaaaccgcagatgaaagtattgatgaaatatttgagaactcaataatagattgtaaactaaaattaagtattatggagaaaaaaacaaaaaggattgcatttgtcctggcaaccatgctatgtggatggcaaatgatgctggcccaaccggttagcccggcaccgacgccaacacgggcggcgaatgatgtgaaggcaatgttcagtgacgcttatccggagaagttcggaaagttccagatagactatgatgactggaatagcgataaatttttgactaccaaaacgattgttactcctttcggagctgcggacgaggtgcttaaaatagaaggtctgtccaccggttctttgcagcacaatgcccagatagccttgggtacatgtaatttgagcgatatggagtatcttcatatggatgtatattctccttccgaaaacggaataggcgagtttagcttttatctggtaagcggttggagcaagacagtatcttgcaatgtgtggtacaactttgatacgaagcaggagtacgaccagtggatttcgatagacataccgatgagcacatttaaaaacggaggattgaacctggccgaaatcaatgtgttacgaattgcaagaggaaaacagggagcacccggcacaattgtctatgtggacaatgtttatgcatacggtaaagcggttgaaccggagtcggatgtgaagattgtggccaatggcaatgccaacctgactacggatgttcctttgatctccgctccgacaccgaaggtagctgccgccaatgtattcaacttcttcagcgatcactatggcgacggtaagttcgattatgcacaaagcgattatggcgatcagaaaacagtgaaatccctcattaccattaatgatacggaggatcaggtattcaagatcgataacatcgtgaatggaagtaaggcgaatgtttccatcggctcaccgaatctttcgggagtggacatgctgcatctggatatattttctccgggcaatgatcagggaatcggtgaatttgattttgccctgacggattttggaggaaacggtaatgatgccggtatctggctgaatattacggacaaaggatggcatggacaatggatctccatcgatatacctctcagcaagtggacgggagctgccaatatgatcagattccgccgtggtggtaaaggctcgaccggtaagctgttgtatgtagacaacgtttatgcttacaagagtgaatcggacgatccgaaaccggttcccgatcctactactgttcctgttcttaccaaagataagtccgatgttatttctattttctgcgaacagtacgaagagccgggataccaagatgaatttggcatagtaagtgccggaaactgggggcaaaatgcgaagcagaaagatgaatttgtagaaattgtagcaggtaaccaaacattaaaacttacgtcgtgggatctcttcccgttcaaagtgcataagaacagtgacgtgatggatttatcccaaatggactatttgcacttaagcatatatcagaatggcgctttggatgaaaacaacaaaccggttagcgtttgtatctggatcaacgacaaggataataaggtggcacaagctcctttgttggaagtgaagcaaggcgaatggacttccgtcagtttcgggatggattatttcaaaaacaagatcgatttgagccgtgtatatgtgatccgtttgaaagtgggcggttatcctacccaggatatttacgtagataatatttttggttataagggcgatcctatccgtccgggtcaagtaaccgagccatatgtggacgagtgcgatcagaagattcaggattccacaccgggcactctgccgccgatggaacaggcctatctgggagtgaatttagcttctgcttccggtggaagtaatccgggcacattcggacacgattacttgtatcctaagtttgaggatttgtattatttcaaggcgaaaggcatacgtttgctccgtatcccgttccgtgctccgcgtttgcaacacgaagttggaggagaactggattatgatgccggtaatacgtcggatatcaaggcgttggccgctgttgtgaaagaagcggaaagattaggtatgtgggttatgctggatatgcacgactactgcgaacggaatattgacggtgtattgtatgaatatggagttgccggacgcaaggtatgggactctgccaaaaacacctggggagattgggaagcaatggatgaagtggtgttgaccaaagagcattttgccgacctgtggaagaagattgctactgaatttaaagattatacgaatatctggggatacgacctgatgaacgagcccaaaggcattaacatcaatacgctgtttgataattatcaggctgccattcatgcgattcgtgaggtggatacaaaagcacaaatagtaatcgaaggtaagaattatgccaatgctgccggttgggaaggttcaagcgacatactgaaagatctggtcgatccggtcaataagatcgtttatcaggcacatacctactttgacaagaacaatacgggtacctataaaaattcttacgatcaggagattggcggaaatgtagaggtctataaacaacgtatcgatccttttattgcctggttagaaaagaacaacaaaaaaggtatgttgggtgaatacggagttccttataatggacatgcgcaaggtgacgagagatatatggacttgatcgatgatgtatttgcttatctgaaagagaaacagcttacctctacttattggtgcggtggatcgatgtacgatgcttatacgctgactgtacaacctgccaaggattattgtacagagaaatctaccatgaaggttatggagaaatatatcaaggattttgataccagtattccttcttccctggtggaaaccaatgctgacggcaatgccatcgtgctctatcccaatccggtgaaagataacttgaagattacttctgaaagcggaatcgaacaggtgattgtcttcaatatgataggccagaaagtaagcgagcgaaatgaaaagggcactaacatcgaattgaacctcgaagcattgggcaagggtacttacttagtaactgtccgcttggaagacggtaatgtggtgaaccgtaagattgtgaaaatgtaattgatgatgaaatgaaatacagccgggcaacggctgtatttccatacttgacagatagacaaaagagacgcagcatcttattgaaaaggtgctgcgtctcttttttaatgaaagattgatagagataggaacgacttattattttttcgacagaagaacaaaagaacatatttcctgcatagcctttataggcggtttatttgttcttttgttcttctgtcgaaaaatagattcgtgacttgttttgagttgaagttgaaccgttttatcgatgatattgaataaaggcagccagtggaatccccatcgtaggataatttttgtagggatgaggctgatagatctgcatgccctcttcgtcatagtaatagccgtcttcgttgtctatcgtgataccgatgtctaccagataatacccttcggattcctttttatcgaaattgtcgatcaggtatttccggaaacgccacatggcaaagttttgcatgacggtgaagttcccgtctttgttgtccatcgtaccgcaggtgctgcaagggatcagtatcacaaacttgccgttgggcaccgctttcagatacgactctttcactattctcagttgtttgtcgaaagtggagaagtcggcgttgatgttgtttctgaaatcgttcagaccgagcatttccgctaagaactggggaggggtaatgttccacatggcaaggtatttgccatagtcgaaattccatgtgaaatcatctttctggacattgacccattggttgccatcgtacatgacgaatgatcttttagcgttgtcatatagtatgtccccttttgcgggcgacttaagatacccgtcttcgttgaacttatacaggcaacttccatatttaccgttggtggcttccaggttgggtctttctcctttctctacaaggaaacagagttgccagaattcggtcgatccccaataacggaaatccccgtccggatgcatgaaaccgtgatagcggttgtttcccgtgaatacctcaaagtaccagctcatgcaggcgccgttgcgtccttcgtcgtattgcccggttgtgtactgcggatcgtcttccgtttcaacctttacgtctcttaatcctacgagcttgaggttcggtacatatccttttcgcaataacgcatctttgtaaaaggcaccttgtgtatagctgtcgccgatgatttgtgccacgacttcggaattaccggtaccttttattcccagtctgattctggaagagtgagtcgccactctggtgaagtttttgagttcgtataagttggcaatgattttcttgtcattttccggtttgtctaccgatactacccgttccaaccggcgtgagtagaaatctccattgaataggacactataatcgaacggataccatctttttatgaacggttctacaaaaatgtcatttctggtatccgacagcatgtacagataactgggcaggcatatttcatttacgtcggacttattgacggccagcgtgatctgtgtaccgtcactgaaactgaaagtcatttgatcgccattggccgagatatttgtaatttggcttccgtcggtgccgtcgattccgttttgcagcacaacggtgctcccatcggagagggtgattgtgtaaccatcgtccgtagtggctacatgggtgatgtagatattgttttgagatgcttcgagcagctgtttctggacgtttagctcgttgcgcaatttttctacttcctctttccagtcgtcgttctgacacgaaggcaggaggagggtacaacataaaagaatggtggtggtgatgaggtttttcataagcgtttttattaaataatgatgagattaaaaatgaaaatatcccgaaactgcttgaatcccgggatattttaggtaatgatggaaactggtcttttttacagttttataatgtgtttgcttactgtttttccactaaccaacttcacggaaataatgtatgaacccgattgcagggccgataggttgatttgattctctcctgccatgttgtagctgccggccaattgaccgctgatggcatacaggttggccgactgaactgcttcttcggaatcgatcgtaatatagtctgttacggcagtaggatagatgtttaaaccgtcgttggctttagcagacttgattcctgtgggcgaacctttataagcgaatatgttggatacataaatattaggtgcatattgcttggagagtggttcgtatataccgtctctgctgccgagtcttaagccgttgatcacataattcttattctccgcagtccagtcgaagttttcgataggaagatcgatagaattccattgattggctttcaagtcgaatatttcgctgtaagcatctgccattgccgggtaattccaggttacgcctaccacgaactggcaatcctgatccggccagaaatcgaaatgcagatagtcataatcggtaacggtggcgccggcattggtatagaatgaggaccattccaggttaatcatatgcagaaccgcatccttattaatataatcgtctacgaaattatccggactaagtccccagtttgtacgtaggatcagtttgtgatctgatgccggttcataagtctttccatagaacgaaatgacgtctgcttccgggtaagtcggagtaggagcggccattgtcggttcttgtgcatttgcaaattgtgtactgcctaataatgccaaggctgcaataaaataagtaatctttctcataatcttaaaattttagagtttaacgatttgttcccttttggtgtgggcaaagtaatggaaaagatcattttggggatgtaataatcttatttttttatagaagaatattgttttaactatttatttttctgaaattcaaccccactaaactaagattattatatccttctataaatatgaaatattcttctatggaacaagctccgaggaagctacttttgtagacaggtaaaagaaaacttagtttgtcaacaaaagaaaggaggacatgtagaagaaacgatgaattcaataaactgcacttgtgatagatgataatcttccgggtcggagagcttgtgatttatttaaaaaagaatctaatactgataattgtatgatttcaaaagacgaaaatataaaaaggcggatcattggtgttttatttttcttatgtgctctaagtcctgcattatgggctcagtcgcgcattataaaaggtgaagtgctcgatcccaacggagaacctctgataggtgtaggggttatgattaaaaatactactgctggaaccatcactgatgtcgatggaagatattccattcaggttcccgataataatgctgttctttccttctcttatgtaggctataaaagaaaagaggtcaaggtgggaagtcaaagcgtgattaatatttctctggaagaggaatccgtattgatggatcaagttgtcattgtgggatatggtagccagaagaaagtcaatctgacgggagccgtagctgcaatttccgttgacgaatcccttgccggccgttcggttgccaatgtctcttccgctttgcaggggttgatgccgggactgtccgtgagccagagctcgggtatggcgggaaataattctgccaaactgttgattcgtggtttaggaacgatcaatagtgccgatccgctgatcgtggtggacgacatgccggatgccgatattaaccggctaaatatgaatgatatagaaagtataaccgtcttgaaggatgcaacggcttcttccgtttacggttctcgtgcagccaacggtgtaatacttgttaaaaccaaatcgggtaaaggtttggaaaagacgcaaataaccttctccggatcgtatggatgggaaaagccgacgaatacttacgattttatatccaattatccacgcgctttgactttacagcaaatttcctcttcgaccaatcccggcaagaatggagaaaatcagaattttaaggatggaacgatcgaccaatggctggcattgggaatgattgacgacaagcggtatccgaacacggactggtgggattacatcatgcgaacgggttccattcaaaattataatgtatcggcaacgggtggaagcgagaaatcgaacttttacgcatctgtgggatatatgaagcaggaaggattacagataaataatgactacgaccgctataacgcccgttttaactttgactataaggtgatgaaaaatgtgaataccggattccgttttgacgggaactggagtaatttcacttatgccttggacaatggtttcacgagcgattctaacctggatatgcagagtgcgattgccggtatctatccttatgatccggttctggatgtttatggcggtgtaatggcgtatggagaagatccacaggctttcaatccgttgagctttttcacaaatcagttgaagaagaaagacagacaggagttgaatgcttctttctatcttgactgggaacccgtaaagggtctggtagcccgcgtggattatggtttgaagtattataaccaattttataaggaagcggacatccccaaccgttcttacaatttccagacgaactcgtatggtatcagggaatatgttacggagaatgccggagttacaaaccagacgagcaccggttacaaaactctgttgaatgcccgtttgaattatcacacggtttttgctacacaccatgatttgaatgccatgttcgtatatagcgaggaatactggcacgaccgttatcagatgtcctataggcaggacagaattcatccgtcactctccgaaatagatgctgccttgtccggaacacagtctacttccggtaattcttcggcagaaggactccgttcttatatcggacgtatcaattattctgcttacggcaaatatttgctggaacttaatttccgtgtcgatggttcgtctaagtttcaaccgggacaccagtacggctttttcccgtcggcagctttgggctggaggtttagcgaagagtcgtttgtgaagccttatatagggaaatggctggcaagcggaaaactccgtgcttcttacggtaagctgggtaacaatagcggtattggcagataccagcagcaagaggtgctttatcagaataactatatgctggacggttcgattgccaaaggttttgtgtattctaaaatgttgaacccggatctgacttgggaatctacgggagtattcaacctgggactggacctgatgtttttcgatggaaaactcgctgcggaatttgattattacgaccgtctgacgaccggtatgttgcaaaagtcgcagatgtccattctgctgaccggtgcttatgaagcgcctatggcaaatctggggacgctccgtaaccggggattcgaagcgaacttaacctggagagaccggattgcagactttacttattctgccaatttcaatatctcttataaccgtacgaaccttgagaagtggggggagttcctggataaaggatatgtttacatagatatgccttatcattttgtatacagccagccggatcgcggattggctcaaacctggaccgattcctataacgctacccctcaaggagtggctccgggagatgtgatccgtctggataccaatggcgacggacgcattgatggcaatgacaaagtggcctatacaaacttccagcgcgatatgccgactaccaacttcgccttgaaccttcagatgggatggaaaggtatcgatgtatctttactgtttcaaggatcggctggtcgtaaagacttctggaacaacaaatatacggaaatcaacctgccggacaagcgttatacctccaactgggatcaatggaataagccttggtcgtgggagaacagaggaggagagtggccgcgtttgggaggattggtgactaacaagacggaaactgatttctggttgcagaacatgacttatttaagaatgaagaacctcatgatcggttatacctttccgaaaaaatggacgagaaagtgtttcatagagaatctccggatttatggaacggcggaaaatctgctgactattaccggttataaaggactcgatccggaaaaagcggctaactcacaagatttgtatcctatcaccaaatcttattctattggcgttaatctgagtttttaataaatgaaaagcggaaattatgaaaagagtttatattaaatatataggtttgattgctgggatgatgatgctattcagttcctgtgccgacttgttgaatcaagaacctacggtggatctgccggctactaattattggaaaacagagtccgatgccgaatcagcattgaacgggctggtatccgatatacgctggctttttaaccgggactactatctcgacggaatgggagaatttgtcagagtgcgcggtaactctttcctgagcgataaaggacgcgacggaagagcttacagggggctttgggaaatcaatccggtaggctacggcggcggatggtccgaaatgtacaggtattgctatgggggcatcaaccgtgtaaactatgtaatcgacaatgtcgagaagatgatagctaatgcaagtagtgaaaaaacgatcaagaacttggaaggcataatcggtgaatgtaagctgatgcgggctttggtttatttcagattgatcatgatgtggggagatgtgccttatatcgactggagagtatacgataattcggaggttgagaacttaccgcgtactccgcttgccgaagtaaaggatcatatcctggatgatttgctggatgcttttaagaaattgcccgaaaaggcgacagttgaaggccgtttttcacaacctgccgcattggctttacgcggaaaggtactgctttattgggcaagctggaaccattacggttggccggaactggatacgtttacaccgagcgaagaggaagctcgaaaagcatataaggcggcagccgaagatttcagaacggtgattgatgactatggtctgactctgttcagaaatggagagccgggagaatgtgacgagccgggaaaagccgacaagctgcccaattactatgacctgtttttgcctacggcaaacggtgatgccgaatttgtactggcatttaatcacggtggcacgaacacagggcagggcgatcagctgatgcgggatttagccggacgaagtgttgaaaactcacaatgttgggtatctccccgtttcgaaattgccgataaatatcagtctacgataaccggtgacttctgtgtaccgttggttaagttgaatccctcttctgtgcccgatgcccgtacccgtcctaattcagccgtgaatccggagagttataaggaccgggattaccgtatgaaagcgtcgatcatgtgggattatgaaatatgccagggactcatgtccaagaaagtgacaggatgggtgcctttcatctacaagatgtggggaagtgaagtagttattaatggtgaaacctatatgtcctacaataccgatggtaccaattccggatatgtattccggaagtttgtgaggaactatcctggtgaagaacgggctgacggagatttcaattggcctgtcatacgtcttgccgatgtgtttttaatgtatgctgaggcggataatgccgtaaacggtcctcagccttatgccatagagctggtgaacagagtgcgtcacagaggtaatcttccggtgttggcatccagtaagacatctactcccgaagcatttttcgaagcgataaagcaggagagaattgtggaactgctgggagagggccagcgtgcatttgatacgcgcaggtggagagagatcgaaacagtctggtgcgaacccggtggcagaggagtaaagatgtatgatacgtatggagcacaggttgccgaattttatgtgaatcagaataacctggcttatgaacgttgctatattttccagataccggagtcggaacgtaaccgtaatccgaatttgactcagaataaaccatacagataa

(19) United States
(12) Patent Application Publication
(10) Pub. No.: US 2024/0026406 A1
(43) Pub. Date: Jan. 25, 2024

(54) ENZYMES AND MICROBES FOR XANTHAN GUM PROCESSING

(71) Applicants: The Regents of the University of Michigan, Ann Arbor, MI (US); Norwegian University of Life Sciences, As (NO)

(72) Inventors: Eric Martens, Ann Arbor, MI (US); Matthew Ostrowski, Ann Arbor, MI (US); Phillip Pope, As (NO); Sabina Leanti La Rosa, As (NO); Benoit Kunath, As (NO)

(21) Appl. No.: 18/245,493

(22) PCT Filed: Sep. 15, 2021
(86) PCT No.: PCT/US21/50494

§ 371 Date: Mar. 15, 2023

Related U.S. Application Data
(60) Provisional application No. 63/079,318, filed on Sep. 16, 2020.
(60) Provisional application No. 63/195,983, filed on Jun. 2, 2021.

(51) Int. Cl. C12P 19/06; C12N 9/42; C12N 15/74; C12N 9/88

(52) U.S. Cl. CPC C12P 19/06; C12N 9/2434; C12N 15/74; C12N 9/88; C12Y 402/02012

(57) ABSTRACT

The present disclosure provides polypeptides having xanthan gum hydrolytic activity, compositions, and uses thereof. The present disclosure also provides, polynucleotides, expression vectors, host cells, and genetically modified bacteria encoding xanthanases or xanthan-utilizing gene loci.

### CROSS-REFERENCE TO RELATED APPLICATIONS

This application claims the benefit of U.S. Provisional Application Nos. 63/079,318, filed Sep. 16, 2020, and 63/195,983, filed Jun. 2, 2021, the contents of which are herein incorporated by reference in their entirety.

### SEQUENCE LISTING STATEMENT

The text of the computer readable sequence listing filed herewith, titled “38573-601_SEQUENCE_LISTING_ST25”, created Sep. 15, 2021, having a file size of 388,374 bytes, is hereby incorporated by reference in its entirety.

### FIELD

The present disclosure provides xanthanase polypeptides, compositions, and uses thereof. The present disclosure also provides polynucleotides, expression vectors, host cells, and genetically modified organisms (e.g., bacteria) encoding xanthanase or xanthan-utilizing gene loci.

### BACKGROUND

Xanthan gum (XG) is an exopolysaccharide produced by Xanthamonas campestris that has been increasingly used as a food additive at concentrations of 0.05-0.5% (w/w) to increase stability, viscosity, and other properties of processed foods. Xanthan gum may also be included in foods as a replacement for gluten at up to gram quantities per serving. The polymer backbone is similar to (mean cellulose, having β-1,4-linked glucose residues, however, xanthan gum contains trisaccharide branches on alternating glucose residues consisting of an α-1,3-mannose, β-1,2-glucuronic acid, and terminal β-1,4-mannose. Xanthan gum has also been used extensively in non-food industries. For example, the oil and gas industry uses xanthan gum in drilling fluid or mud for its rheological properties and in the secondary and tertiary recovery of petroleum.

### SUMMARY

Disclosed herein are polypeptides comprising a truncated xanthanase, wherein the truncated xanthanase comprises a glycoside hydrolase family 5 endoglucanase domain and three carbohydrate binding domains. In some embodiments, the polypeptides comprise an amino acid sequence having at least 70% sequence identity to SEQ ID NO: 2. In some embodiments, the polypeptides comprise an amino acid sequence having at least 70% sequence identity to SEQ ID NO: 33. Also disclosed herein are polynucleotides comprising a nucleic acid sequence encoding the polypeptides, expression vectors comprising the polynucleotides operably linked with a promoter and host cells comprising the polynucleotides or expression vectors.

Further disclosed herein are compositions comprising the polypeptides disclosed herein. In some embodiments the compositions are cleaning compositions. In some embodiments the compositions are wellbore servicing compositions. The compositions may be liquids, gels, powders, granulates, pastes, sprays, bars, or unit doses. Also disclosed are methods comprising contacting an object or a surface with the polypeptide disclosed herein or a composition thereof.

Additionally, methods of making intermediate sized xanthan gums and/or pentasaccharide repeating units of xanthan gum are disclosed. The methods comprise contacting xanthan gum or a composition comprising xanthan gum with the polypeptides disclosed herein or compositions thereof.

Additionally, genetically modified organisms (e.g., bacteria) and compositions thereof are disclosed. In some embodiments, the genetically modified organisms comprise the polypeptides or polynucleotides disclosed herein. In some embodiments the genetically modified organisms comprise a heterologous xanthan-utilization gene or gene locus, wherein the heterologous xanthan-utilization gene or gene locus comprises one or more nucleic acids encoding a xanthan or xanthan oligonucleotide degrading enzyme. In some embodiments, the xanthan or xanthan oligonucleotide degrading enzyme comprises a glycoside hydrolase family 5 enzyme from Ruminococcaceae UCG13. The bacteria, for example, may be in the genus Bacteroides, Parabacteroides, Alistipes, Prevotella, Clostridium, Faecalibacterium, Eubacterium, Ruminococcus, Peptococcus, Peptostreptococcus, Bifidobacteria, Escherichia, or Lactobacillus.

Other aspects and embodiments of the disclosure will be apparent in light of the following detailed description and accompanying figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a representation of xanthan gum structure showing the β-1,4-linked glucose backbone residues (blue circles) with branches of mannose (green circles) and glucuronic acid (blue and white diamond). The inner and outer mannose residues are variably modified by acetylation and pyruvylation, respectively. FIGS. 1B-11D show growth characteristics of the xanthan-degrading cultures. FIG. 1B is growth curves of the original xanthan-degrading culture showing that increases in xanthan gum concentration resulted in increases in culture density. The original culture displayed relatively stable composition over sequential passaging (FIG. 1C). An additional 20 samples (FIG. 1D) were sequentially passaged in xanthan containing media (10×) and analyzed for composition by 16S rRNA sequencing (16 of the most abundant genus are displayed for clarity). All cultures shared an abundant operational taxonomic unit (OTU), classified as Ruminococcaceae uncultured genus 13 (R. UCG13).

FIG. 2 is schematics of putative xanthan utilization loci color-coded and annotated by predicted protein family. The four boxes below each gene are colored to represent expression levels of each gene at timepoints taken throughout the culture's growth on xanthan gum.

FIG. 3A is a schematic showing the annotated domains, signal peptide (SP), three carbohydrate binding modules (CBMs), and multiple Listeria-Bacteroides repeat domains, of the xanthan-degrading GH5 in R. UCG13. FIG. 3B is the extracted ion chromatograms showing various acetylated and pyruvylated penta- and deca-saccharides produced by GH5 degradation of xanthan gum—841 for the pentamer, 883 for the acetylated pentamer, 925 for the di-acetylated pentamer, 953 for the acetylated and pyruvylated pentamer, 1665 for the decamer, 1707 for the decamer with a single acetylation, 1749 for the decamer with two acetylations, 1847 for the decamer with one acetylation and two pyruvylations and 1889 for the decamer with two acetylations and two pyruvylations. Retention times are shown above each extracted peak. FIG. 3C is the proton NMR contrasting tetrameric products obtained from incubating lyase-treated xanthan gum with either R. UCG13 GH5 or P. nanensis GH9. FIG. 3D is a graph showing the kinetics of R. UCG13 GH5 on native and lyase-treated xanthan gum (error bars represent mean and standard deviation, n=4)

FIGS. 4A-4B show that a strain of B. intestinalis cross-feeds on xanthan oligosaccharides. FIG. 4A is a graph of the growth curves of B. intestinalis isolated from the original xanthan-degrading culture. (curves represent mean SEM, n=2) for a variety of feed sources. FIG. 4B shows the fold-change in expression of B. intestinalis genes when grown on xanthan oligosaccharides relative to glucose.

FIG. 5 is a schematic showing that xanthan degrading loci are present in modern human microbiomes but not in the microbiome of hunter-gatherers. Multiple microbiome metagenome datasets were searched for the presence or absence of the R. UCG13 and B. intestinalis xanthan loci. Map colors correspond to where populations were sampled for each dataset displayed on the outside of the figure. Circle segments are sized proportionately to total number of individuals sampled for each dataset. Lines represent presence of either the R. UCG13 xanthan locus (green) or the B. intestinalis xanthan locus (red). Percentages display the total abundance of R. UCG13 or B. intestinalis locus in each dataset.

FIG. 6 is a graph of an extinction dilution series with either XG or an equal amount of its component monosaccharides as growth medium.

FIGS. 7A-7C are metagenomic, metatranscriptomic and monosaccharide analysis of residual polysaccharide of two replicates of the original culture grown in liquid medium with XG. FIG. 7A are growth curves indicating timepoints for residual polysaccharide analysis (FIG. 7B) and metatranscriptomic analysis (FIG. 7C).

FIGS. 8A and 8B show the results from three independent cultures fractionated with a variety of purification methods (FIG. 8A) and the respective proteome analysis (FIG. 8B).

FIG. 9 is a schematic of the Ruminococcacea UCG13 XG PUL and B. intestinalis XG PUL loci in 16 additional XG-degrading identified communities.

FIG. 10 is a graph of the growth curves of the original xanthan-degrading culture showing greater culture density as xanthan gum concentration was increased (n=12, SEM≤3%).

FIG. 11 is extracted ion chromatograms showing various acetylated and pyruvylated penta- and deca-saccharides produced by incubating culture supernatant with XG.

FIG. 12 shows that Xanthan degrading loci are present in modern human microbiomes but not in hunter-gatherers'. Multiple microbiome metagenome datasets were searched for the presence or absence of the R. UCG13 and B. intestinalis xanthan loci. Map colors correspond to where populations were sampled for each dataset displayed on the outside of the figure. Circle segments are sized proportionately to total number of individuals sampled for each dataset. Lines represent presence

FIG. 13 is a schematic of an exemplary cellular model of xanthan degradation.

FIG. 14 is thin layer chromatography of xanthan gum incubated with different fractions of an active xanthan gum culture (supernatant, washed cell pellet, lysed cell pellet, or lysed culture). Negative controls were prepared by heating fractions at 95° C. for 15 minutes prior to initiating with xanthan gum. EDTA was added to a final concentration of ˜50 mM to determine the necessity of divalent cations for enzyme activity. Strong color development in circles at baseline is undigested polysaccharide while bands that migrated with solvent are digested oligosaccharides and monosaccharides.

FIGS. 15A-15G show activity of R. UCG13 GH5 enzymes on various polysaccharides. FIG. 15A is an SDS-PAGE gel of purified GH5 constructs and their resultant activity as assessed by TLC, xanthan gum (FIG. 15B), carboxymethyl cellulose (CMC, FIGS. 15B-15C), hydroxyethyl cellulose (HEC, FIG. 15C), barley β-glucan (FIG. 15D), yeast β-glucan (FIGS. 15D-15E), tamarind xyloglucan (FIG. 15E), xylan (FIG. 15F), and wheat arabinoxylan (FIGS. 15F-15G). Enzymes are 1, RuGH5b (GH5 only); 2, RuGH5b (GH5 with CBM-A); 3, RuGH5b (GH5 with CBM-A/B); 4, RuGH5b (full protein); 5, RuGH5a (GH5 only); 6, RuGH5a (GH5 with CBM-A); 7, RuGH5a (GH5 with CBM-A/B); 8, RuGH5a (GH5 with CBM-A/B/C); 9, RuGH5a (full protein); 10, replicate of 8. Strong color development in circles at baseline is undigested polysaccharide while bands or streaking that migrated with solvent are digested oligosaccharides and monosaccharides. Although minor streaking appears in some substrates due to residual oligosaccharides, comparing untreated substrate with enzyme incubated substrate allows determination of enzyme activity. RuGH5a constructs with all 3 CBMs (8-10) showed clear activity on XG.

FIGS. 16A-16J are LC-MS analysis used to track relative increases and decreases of intermediate oligosaccharides with the addition of enzymes, verifying their abilities to successively cleave XG pentasaccharides to their substituent monosaccharides. Integrated extracted ion counts (n=4, SEM) that correlate with compound abundance are shown for acetylated pentasaccharide (FIG. 16A; M-H ions: 883.26, 953.26, 925.27), deacetylated pentasaccharide (FIG. 16B; M-H ions: 841.25, 911.25), acetylated tetrasaccharide (FIG. 16C; 2M-H ion: 1407.39), tetrasaccharide (FIG. 16D; M−H ion: 661.18), acetylated trisaccharide (FIG. 16E; M+Cl ion: 581.15), trisaccharide (FIG. 16F; M+Cl ion: 539.14), cellobiose (FIG. 16G; M+Cl ion: 377.09), and pyruvylated mannose (FIG. 16H; M−H ion: 249.06). Reactions were carried out using xanthan oligosaccharides produced by the RuGH5a to test activities of the R. UCG13 (A-I) and B. intestinalis (J-O) enzymes. R. UCG13 enzymes were tested in reactions that included (A) no enzyme, (B) R. UCG13 CE-A, (C) R. UCG13 CE-B, (D) R. UCG13 PL8, (E) R. UCG13 PL8 and CE-A, (F) R. UCG13 PL8 and CE-B, (G) R. UCG13 PL8, both CEs, and GH88, (H) R. UCG13 PL8, both CEs, GH88, and GH38-A, (I) R. UCG13 PL8, both CEs, GH88, and GH38-B. B. intestinalis enzymes were tested in reactions that included (J) no enzyme, (K) Bi PL-only, (L) Bi PL-CE, (M) Bi PL-CE and Bacillus PL8, (N) Bi PL-CE and GH88 and Bacillus PL8, (O) Bi PL-CE, GH88, and GH92 and Bacillus PL8. A legend of enzymes included in each reaction is shown in FIG. 16I. FIG. 16J is an SDS-PAGE gel of purified enzymes with 4.5 μg loaded, including (1-2) ladder, (3) B. intestinalis GH3, (4) B. intestinalis GH5, (5) B. intestinalis PL-only, (6) B. intestinalis PL-CE, (7) B. intestinalis GH88, (8) B. intestinalis GH92, (9) R. UCG 13 GH38-A, (10) R. UCG13 GH38-B, (11) R. UCG13 GH94, (12) R. UCG13 PL8, (13) R. UCG13 CE-A. FIG. 16K is an SDS-PAGE gel of purified enzymes with 4.5 μg loaded, including (1) ladder, (2) B. intestinalis PL-only, (3) B. intestinalis PL-CE, (4) B. intestinalis GH88, (5) B. intestinalis GH92, (6) R. UCG13 GH38-A, (7) R. UCG13 GH38-B, (8) R. UCG13 CE-A, (9) R. UCG13 GH88, (10) R. UCG13 CE-B, (11) R. UCG13 PL8. FIG. 16L is TLC analysis of R. UCG13 GH94 and B. intestinalis GH3 activity on cellobiose. From left to right lanes show (A) RuGH5b (full protein), (B) RuGH5a (full protein), (C) B. intestinalis GH3, (D) B. intestinalis GH5, (E) R. UCG13 GH94, (F) odd standards, (G) even standards, (H) cellobiose. Odd and even standards are maltooligosaccharides with 1, 3, 5, and 7 hexoses or 2, 4, and 6 hexoses, respectively. While the B. intestinalis GH3 only produced one product, the R. UCG13 GH94 produced two, one matching the approximate Rf of glucose while the other had a much lower Rf which presumably is phosphorylated glucose (matching the known phosphorylase activity of the GH94 family).

FIG. 17A is traces of RNA-seq expression data from triplicates of the original culture grown on either XG or polygalacturonic acid (PGA), illustrating overexpression of the XG PUL. FIGS. 17B and 17C are growth curves for Bacteroides clarus (FIG. 17B) and Parabacteroides distasonis (FIG. 17C) isolated from the original culture showing a lack of growth on XG oligosaccharides (XGOs). FIG. 17D is growth curves for Bacteroides intestinalis showing lack of growth on tetramer generated with P. nanensis GH9 and PL8 (Psp Tetramer) even in the presence of 1 mg/mL RuGH5a generated XGOs to activate the PUL. Growth on glucose confirmed that the Psp Tetramer was not inherently toxic to cells. All substrates were used at 5 mg/mL unless otherwise noted. Growths are n≥2, error bars show SEM (in most cases, smaller than the marker). FIG. 17E is traces of RNA-seq expression data from triplicates of B. intestinalis grown on either glucose (Glu) or XG oligosaccharides (XGOs), illustrating overexpression of the XGO PUL.

FIG. 18A is a schematic of the metagenomic sequencing of additional 16 cultures (S, human fecal sample) that actively grew on and degraded xanthan gum revealed two architectures of the R. UCG13. The more prevalent locus contained a GH125 insertion. The 10 additional samples with this locus architecture include: S22, S25, S39, S43, S44, S45, S49, S53, S58, and S59. FIG. 18B is a schematic of the B. intestinalis xanthan locus present in 3 additional cultures. FIG. 18C is a schematic of additional members of the Bacteroideceae family harbor a PUL with a GH88, GH92 and GH3 that could potentially enable utilization of XG-oligosaccharides. FIG. 18D is a schematic of the GH125-containing version of the R. UCG13 xanthan locus was detected in two mouse fecal samples (M, mouse fecal sample). FIG. 18E is a comparison of the human and mouse RuGH5a amino acid sequence, showing the annotated signal peptide (SP), GH5 domain, three carbohydrate binding modules (CBMs), and multiple Listeria-Bacteroides repeat domains. FIG. 18F a schematic of the genetic organization and amino acid identity (%) between the B. intestinalis xanthan locus in the original human sample and a PUL detected in a fracking water microbial community (FWMC) using LAST-searches. FIG. 18G is an SDS-PAGE gel of purified enzymes with 4.5 μg loaded, including ladder and the different mouse RuGH5a constructs. A, B, and C are all versions of the GH5 domain alone, D is a construct designed to terminate at a site homologous to the last CBM in the human RuGH5a, and E is a full-length construct of the mouse RuGH5a. FIG. 18H is TLC of each mouse RuGH5a construct incubated with XG and also odd (1, 3, 5, and 7 residues) and even (2, 4, and 6 residues) malto-oligosaccharide standards. The GH5-only constructs did not degrade XG but constructs D and E (with regions homologous to the human RuGH5a CBMs) were able to hydrolyze XG.

FIG. 19 is a graph of B. salyersiae WAL 10018 (DSM 18765=JCM 12988) grown in minimal media with various substrates. All substrates were provided at a final concentration of 5 mg/mL. The monosaccharide mix consisted of 2:2:1 glucose:mannose:glucuronic acid. The xanthan gum tetramer was produced by incubating Megazyme xanthan lyase (E-XANLB) with xanthan gum oligosaccharides produced with RuGH5a.

FIG. 20 is a schematic of the PUL29 identified from B. salyersiae WAL 10018 as the putative locus responsible for catabolizing xanthan gum oligosaccharides.

FIG. 21 is a graph of gene expression analysis of B. salyersiae grown on PL8 treated xanthan oligosaccharides or glucose. qRT-PCR demonstrated overexpression of the identified enzymes PUL29 when grown on PL8 treated xanthan oligosaccharides, providing evidence for these enzymes' role in catabolizing xanthan gum oligosaccharides.

### DETAILED DESCRIPTION

The present disclosure provides a polypeptide comprising a xanthanase (an enzyme capable of degrading xanthan gum) which can hydrolyze xanthan gum in a single step compared to known xanthanase enzymes which typically require two enzymes. The enzyme generates xanthan degradation products, including pentasaccharide repeating units and intermediate sized xanthan gums, poly- and oligo-saccharides of average molecular weight less than native xanthan gum but more than a single pentasaccharide repeating unit. Additionally, two genetic loci from two microbes have been identified as having xanthan-degrading activity which may be introduced alone or with the xanthanase polypeptide to into heterologous bacteria for use as probiotics in subjects who suffer from gastrointestinal or metabolic diseases or inject a larger than average level of xanthan gum.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein” are used interchangeably herein.

A “polysaccharide” or “oligosaccharide” is a linked sequence of two or more monomeric carbohydrates connected by glycosidic bonds. The polysaccharides can be natural, synthetic, or a modification or combination of natural and synthetic. polysaccharide may be modified by the addition of sugars, lipids or other moieties not included in the main chain of the polysaccharide.

An “expression vector,” as used herein, refers to a linear or circular DNA molecule that comprises a polynucleotide encoding a polypeptide and is operably linked to control sequences that provide for its expression. The term “operably linked” means a configuration in which a control sequence (e.g., a promoter) is placed at an appropriate position relative to the coding sequence of a polynucleotide such that the control sequence directs expression of the coding sequence.

The term “bacterial artificial chromosome” or “BAC” as used herein refers to a bacterial DNA vector. BACs, such as those derived from E. coli, may be utilized for introducing, deleting, or replacing DNA sequences of non-human mammalian cells or animals via homologous recombination. E. coli can maintain complex genomic DNA as large as 500 kb or greater in the form of BACs (see Shizuya and Kouros-Mehr, Keio J Med. 2001, 50(1):26-30), with greater DNA stability than cosmids or yeast artificial chromosomes. In addition, BAC libraries of human DNA genomic DNA have more complete and accurate representation of the human genome than libraries in cosmids or yeast artificial chromosomes. BACs are described in further detail in U.S. application Ser. Nos. 10/659,034 and 61/012,701, which are hereby incorporated by reference in their entireties.

The term “host cell,” as used herein, refers to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

As used herein, “genetically modified” refers to an organism (e.g., a bacterium) which has a modification to introduce a nucleic acid that does not naturally occur in the organism or to introduce additional copies or modified forms of nucleic acids that naturally occur in the organism. The nucleic acid can be integrated in one or more copies into a genome or one or more copies of the nucleic acid can remain episomal, e.g., in a plasmid, phagemid or artificial chromosome.

The term “textile,” as used herein, refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir, or manmade cellulosics (e.g., originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabbit and silk or synthetic polymer such as nylon, aramid, polyester, acrylic, polypropylene, and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax/linen, jute, cellulose acetate fibers, lyocell).

A “wellbore,” as used herein, refers to any hole drilled to aid in the exploration and/or recovery of natural resources, including oil, gas, or water. For example, a wellbore may be the hole that forms a well. A wellbore can be encased, for example by materials such as steel and cement, or it may be uncased.

As used herein, “treat,” “treating” and the like means a slowing, stopping, or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing,” “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Xanthanase Polypeptides and Polynucleotides

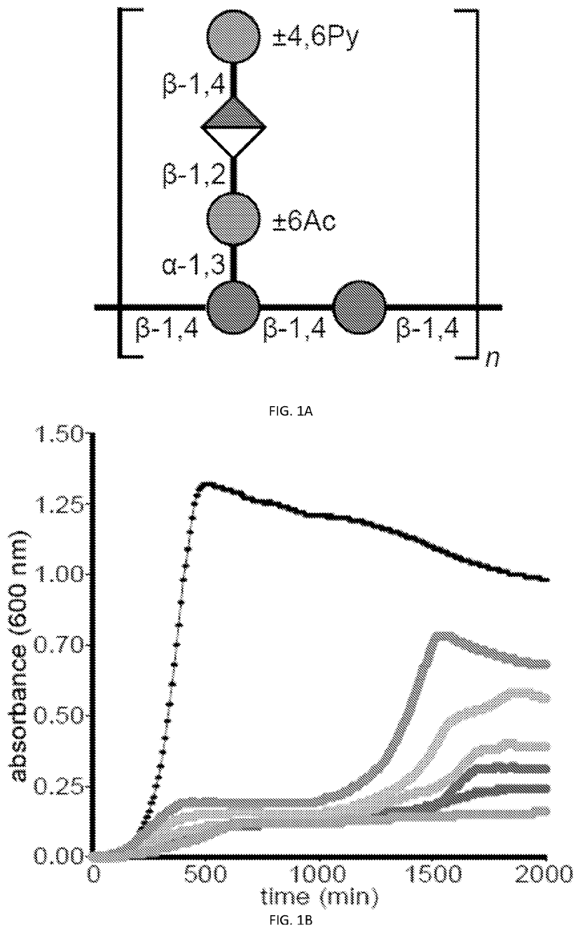

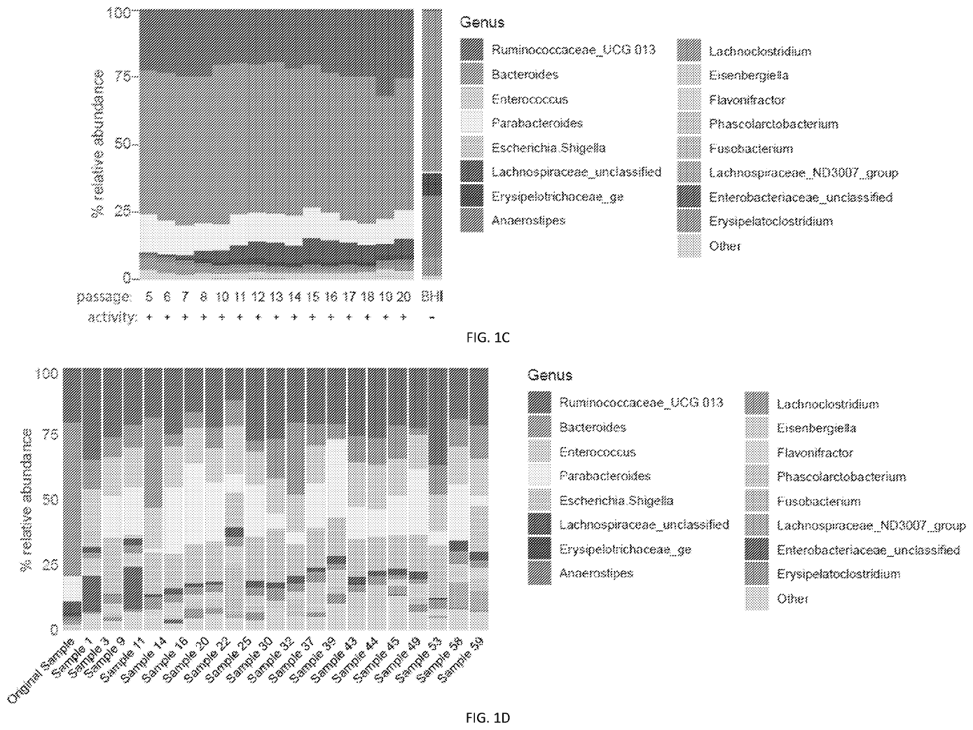

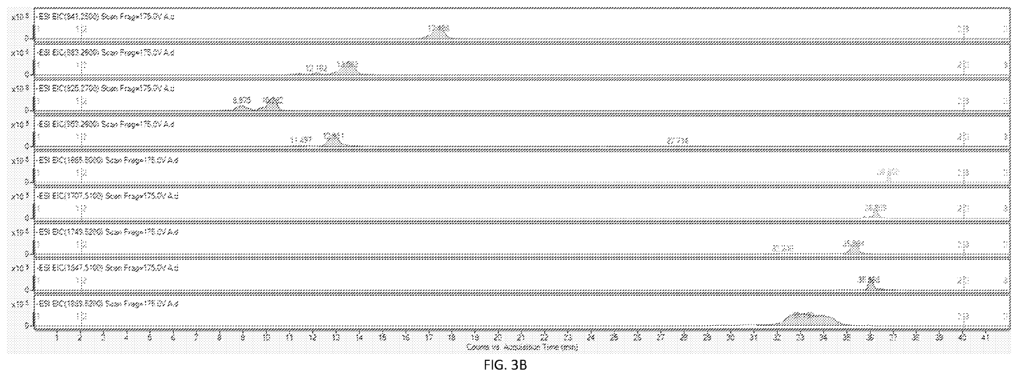

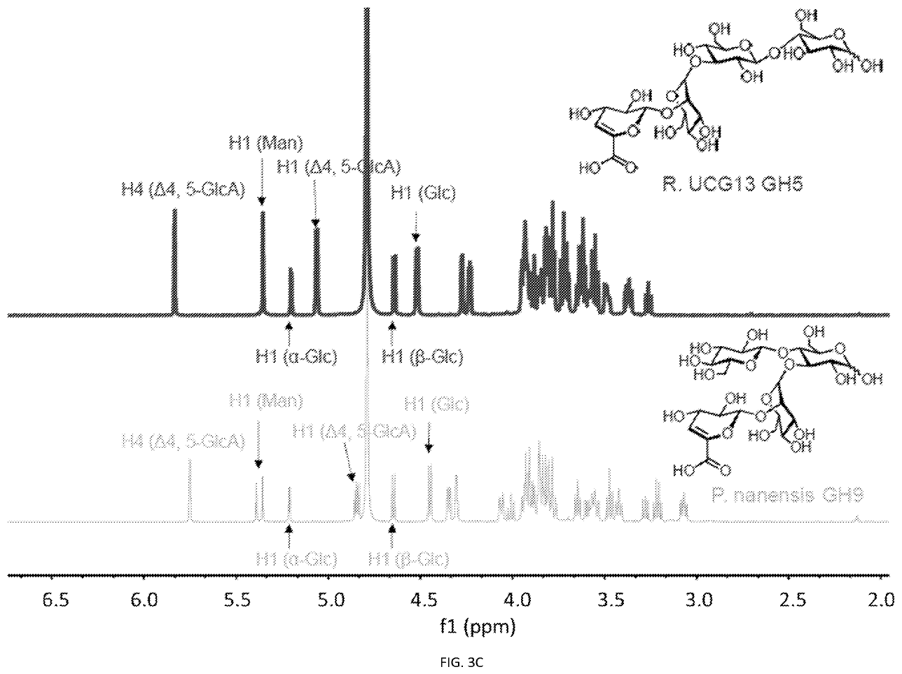

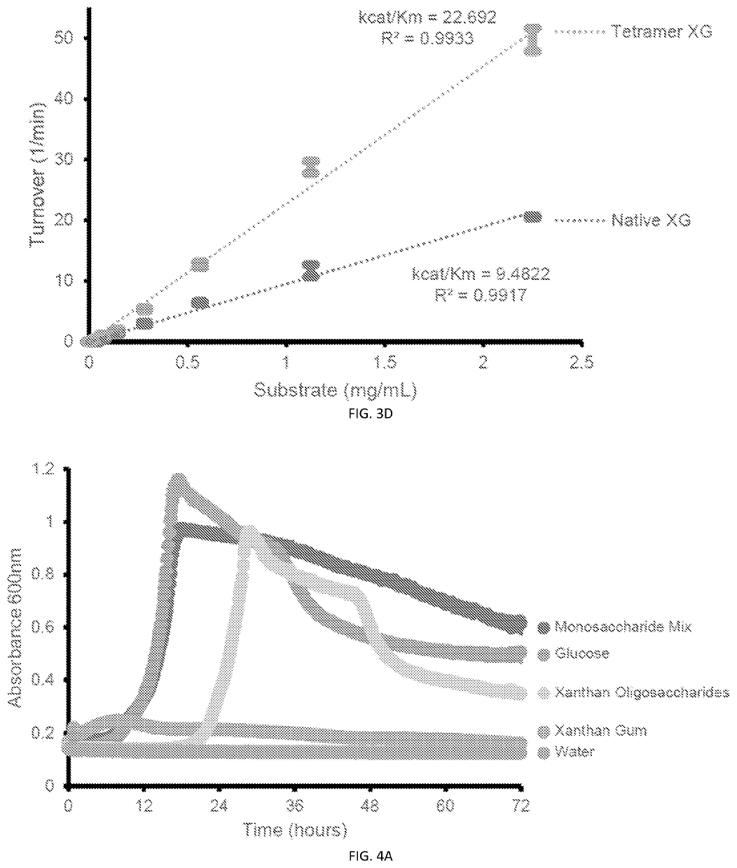

The present disclosure provides a polypeptide comprising a truncated xanthanase. The xanthanase has activity on xanthan gum, both native and lyase-treated xanthan gum. In contrast to other known xanthanases, the truncated xanthanase cleaves the reducing end of the non-branching backbone glucosyl residue of xanthan gum (FIGS. 1A and 3C). The truncated xanthanase does not comprise SEQ ID NO: 3.

The truncated xanthanase may comprise a glycosyl hydrolase 5 endoglucanase domain and three carbohydrate binding domains. The glycosyl hydrolase 5 endoglucanase domain comprises an amino acid sequence having at least 70% (e.g., 70%, 75%, 80%, 85%, 90%, 95%, or 95%) sequence identity to SEQ ID NO: 1. In some embodiments, the polypeptide comprises an amino acid sequence having at least 70% sequence identity to SEQ ID NO: 2. In some embodiments, the polypeptide comprises an amino acid sequence having at least 70% sequence identity to SEQ ID NO: 33.

The present disclosure also provides nucleic acids encoding the polypeptides described herein. In some embodiments, the polynucleotides disclosed herein can be introduced into an expression vector, such that the expression vector comprises a promoter operably linked to the polynucleotides encoding the peptides or polypeptides described herein. The expression vector may allow expression of the peptides or polypeptides in a suitable expression system using techniques well known in the art, followed by isolation or purification of the expressed peptide or polypeptide of interest. A variety of bacterial, yeast, plant, mammalian, and insect expression systems are available in the art and any such expression system can be used. Alternatively, a polynucleotide encoding a peptide of the invention can be translated in a cell-free translation system.

The selection of promoter will depend on the expression system being used. For example, suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus lichemformis penicillinase gene (penP), Bacillus subtilis xylA and xylB genes, and prokaryotic beta-lactamase gene.

The expression vector may contain other control, selectable marker, or tag sequences. Control sequences include additional components necessary for the expression of a polynucleotide, including but not limited to, a leader, a polyadenylation sequence, a propeptide sequence, a signal peptide sequence, and a transcription or translation terminator. The control sequence(s) may be native or foreign to the nucleotide sequence encoding the polypeptide or native or foreign to each other. The control sequences may be provided with linkers for the purpose of introducing specific restriction sites facilitating ligation of the control sequences with the coding region of the nucleotide sequence encoding a polypeptide.

The selectable marker and any other parts of the expression construct may be chosen from those available in the art. In some embodiments, the selectable marker is a gene the product of which provides for biocide or viral resistance, resistance to heavy metals, prototrophy to auxotrophy, and the like and thereby permits easy selection of transformed, transfected, transduced, or the like cells. The selectable markers are primarily dictated by the host cell being used. For example, bacterial selectable markers commonly include markers that confer resistance to antibiotics, for example ampicillin, kanamycin, chloramphenicol, or tetracycline.

Various types of expression vectors are available in the art and include, but are not limited to, bacterial, viral, and yeast vectors. For example, the vector may include a plasmid, cosmid, bacteriophage, p1-derived artificial chromosome (PAC), bacterial artificial chromosome (BAC), yeast artificial chromosome (YAC), or mammalian artificial chromosome (MAC). The various vectors may be selected based on the size of polynucleotide inserted in the construct.

Also provided is a host cell comprising the polynucleotides or the expression vectors described herein. The host cell may be any cell useful in the recombinant production of a polypeptide of the present invention, e.g., a prokaryote or a eukaryote. The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma. The host cell may also be a eukaryote, such as a mammalian, insect, plant, or fungal cell.

In some embodiments, the host cell is a gastrointestinal microbiota (gut flora) microorganism that is modified to express and/or secrete the polypeptides described herein. Such host cells find use in populating gastrointestinal systems of host organisms (e.g., people, livestock, etc.) to regulate (e.g., increase) that ability of the host organism to digest or otherwise process xanthan gum. These host cells find particular use in subject that have a high dietary intake of xanthan gum (e.g., human subject on a low gluten or gluten-free diet). Host cells that find use in such application include, for example, bacteria belonging to the genera Bacteroides, Clostridium, Faecalibacterium, Eubacterium, Ruminococcus, Peptococcus, Peptostreptococcus, and/or Bifidobacterium. Such host cells may be introduced into a subject by any suitable methodology including, but not limited to, administration of probiotics containing the host cells and fecal microbiota transplantation. In some embodiments, endogenous gastrointestinal microbiota are genetically modified.

### 3. Compositions and Methods of Use

The present disclosure further provides compositions comprising the polypeptides described herein and methods of use thereof. The composition may take on any desired form (e.g., liquid, gel, powder, granulate, paste, spray, bar, unit dose, microcapsule, and the like). The compositions and the polypeptides described herein may be used in any application which requires or it is beneficial to degrade or remove xanthan gum.

In some embodiments, the composition is a cleaning composition. The cleaning composition includes, but is not limited to, detergent compositions (e.g., liquid and/or solid laundry detergents and dish washing detergents); hard surface cleaning formulations, such as for glass, wood, ceramic and metal counter tops, floors, tables, walls, and windows; carpet cleaners; oven cleaners; fabric fresheners; fabric softeners; and textile and laundry pre-treaters.

The cleaning compositions may comprise one or more additional enzymes, such as proteases, amylases, lipases, cellulases, endoglucanases, xyloglucanases, pectinases, pectin lyases, peroxidaes, catalases, mannanases, redox enzymes, or any mixture thereof. The cleaning compositions may also comprise one or more components selected from surfactants, builders, chelating agents, bleaching components (e.g., precursors, activators, catalysts), antibacterial agents, antifungal agents, polymers, degreasers, corrosion inhibitors, stabilizers, antioxidants, colorants, fragrances, foaming agents, emulsifiers, moisturizers, abrasives, binders, viscosity controlling agents, and pH controlling agents. One of skill in the art is capable of selecting the additional components based on the desired functionality of the composition.

In some embodiments, the composition is a well treatment composition or a wellbore servicing composition. Xanthan gum is commonly used for increasing the viscosity of drilling fluids (e.g., drilling mud, drill-in fluids, or completion fluids). Compositions comprising a xanthanase, such as those disclosed herein, may be used to decrease viscosity of the fluids and/or clean well bores and wellbore filter cakes. Filter cakes are coatings on the walls of the wellbore that limit drilling fluid losses, preserve the integrity of the drilling fluid, prevent formation damage, and provide a balanced density. To form a filter cake, the drilling fluid is often intentionally modified with a weighting material including barite, iron oxide, or calcium carbonate and some particles of a size slightly smaller than the pore openings of the formation. It is these particles which may contain xanthan gum and improve viscosity and emulsification properties of the drilling fluid.

The well treatment composition or wellbore servicing composition may also comprise one or more additional components selected from chelating agents; converting agents (carbonate, nitrate, chloride, formate, or hydroxide salts); other polymer removal agents (persulfate salt, a perborate salt, a peroxide salt, and other enzymes, for example, amylases, glucanases, mannanases, cellulases, oxidoreductases, hydrolases, lyases); organic solvents; surfactants; binders; an aqueous liquid, which may be water, brine, seawater, or freshwater; fragrances; colorants; dispersants; pH control agents or acidifying agents; water softeners or scale inhibitors; bleaching agents; crosslinking agents; antifouling agents; antifoaming agents; anti-sludge agents; corrosion inhibitors; viscosity modifying agents; friction reducers; freeze point depressants, iron-reducing agents; and biocides. One of skill in the art is capable of selecting the additional components based on the desired functionality of the components. Exemplary compositions and methods of using well treatment or wellbore servicing compositions can be found in U.S. Pat. Nos. 5,881,813, 6,110,875, and 9,890,321 and U.S. Patent Publications 2020/0131432 and 2020/0115609; each incorporated herein by reference in its entirety.

The present disclosure provides methods of cleaning utilizing the polypeptides or compositions disclosed herein. The methods comprise contacting an object or a surface with the polypeptides or compositions disclosed herein. In some embodiments, the methods further comprise at least one or both of rinsing the object or surface and drying the object or surface. In some embodiments, the object or surface comprises a textile, a plate, tile, dishware, silverware, glass, a wellbore, or wellbore filter cake.

The process of contacting can be done in a variety of different ways, depending on the composition and the subject or object being cleaned. For example, the composition can be diluted into water to for a cleaning solution which is then contacting the surface or object as commonly done in dishwashing, laundry, and floor cleaning applications. The composition may be directly applied to the surface or object as a spray, liquid, foam, or solid, as is common for fabric spot treatments and hard surface cleansers. The contacting may be carried out for any period of time and may include a soaking period in which the object or surface remains in contact with the composition for a period of time, for example, for at least about 1 hour, at least about 4 hours, at least about 8 hours, at least about 16 hours, or at least about 24 hours.

For cleaning of a wellbore or wellbore filter cake, the composition can be injected into the wellbore to dissolve the filter cake within, the composition can be injected directly at the site of a filter cake, the composition can circulate in the wellbore for a period of time, or the composition may be left in the wellbore in a static manner to soak the wellbore or filter cake.

The present disclosure provides methods of modifying xanthan gum in a subject (e.g., in a digestive tract of a subject). In some embodiments, polypeptides are provided to the subject. In some embodiments, the polypeptides are provided orally such that they are made available in the digestive tract (e.g., mouth, stomach, small intestine, large intestine, etc.) at a concentration sufficient to digest xanthan gum present in the subject. In some such embodiments, purified polypeptides are provided in a capsule or other carrier that releases the peptides at a desired location in the digestive tract. In some embodiments, polypeptides are made available by expressing them in a host cell in a subject. In some embodiments, the host cell is a gastrointestinal microbiota microorganism. The polypeptide may be transiently or stably expressed in the microorganism. A nucleic sequence encoding the polypeptide may be under the control of a promoter that provides optimized expression (e.g., overexpression) of the polypeptide. In some embodiments, the promoter is an inducible promoter that permits control over the timing and/or level of expression. In some embodiments, the polypeptide is encoded by a nucleic acid sequence that further encodes a signal sequence such that the translated polypeptide contains the signal sequence. Signal sequences find use, for example to increase extracellular secretion of the polypeptide.

The present disclosure also provides methods of making intermediate sized xanthan gums and/or pentasaccharide repeating units of xanthan gum. The methods comprise contacting xanthan gum or a composition comprising xanthan gum with the disclosed truncated xanthanase or compositions thereof. The contacting may be done for various lengths of time or under various conditions which facilitate activity of the xanthanase. One of skill in the art can monitor the reaction and the products produced by using any carbohydrate analysis method known in the art, including but not limited to, liquid chromatography-mass spectrometry (LC-MS), thin layer chromatography (TLC), gas chromatography (GC), high performance liquid chromatography (HPLC), and quantitative size exclusion or molecular sieve chromatography.

The truncated xanthanase cleaves the reducing end of the non-branching backbone glucosyl residue of xanthan gum. The length or molecular weight of the intermediate sized xanthan gums and/or the relative percentage of pentasaccharide repeating units of xanthan gum formed can be regulated by changing the length of time in which the enzyme is in contact with the xanthan gum, the temperature of the reaction, and/or the quantity of the enzyme.

The intermediate sized xanthan gums and/or pentasaccharide repeating units of xanthan gum may be purified and employed in a number of applications or, alternatively, further modified using chemical modifications known in the art for xanthan gum and other starches. The intermediate sized xanthan gums and/or pentasaccharide repeating units of xanthan gum may be utilized in applications in which rheological and viscosity characteristics different from those conferred by native xanthan gum are desired. For example, the intermediate sized xanthan gums and/or pentasaccharide repeating units of xanthan gum may be employed in drilling fluids/muds, cosmetics, water-based paints, construction and building materials, food products, drug delivery compositions, hydrogels, and tissue engineering (See Kumar, A., et al., Carbohydr Polym 180:128-144 (2018) and Ramburrun, et al., Expert Opin. Drug Deliv. 14, 291-306 (2017), both incorporated herein by reference in their entirety).

### 4. Genetically Modified Bacteria

The present disclosure provides genetically modified bacteria. In some embodiments, the genetically modified bacteria comprise the truncated xanthanase polypeptides or polynucleotides disclosed herein. In some embodiments, the genetically modified bacteria comprise a heterologous xanthan-utilization gene or gene locus.

The heterologous xanthan-utilization gene or gene locus may comprise one or more nucleic acids encoding a xanthan or xanthan oligosaccharide degrading enzyme. The xanthan or xanthan oligosaccharide degrading enzyme may comprise a glycoside hydrolase, a xanthan or polysaccharide lyase, a mannanase, or a carbohydrate esterase.

In some embodiments, the xanthan-utilization gene or gene locus comprises a gene encoding a glycoside hydrolase family 5 enzyme from Ruminococcaceae UCG13. In some embodiments, the glycoside hydrolase family 5 enzyme may comprise an amino acid sequence having at least 70% sequence identity to SEQ ID NO: 2 or 3. In some embodiments, the glycoside hydrolase family 5 enzyme may comprise an amino acid sequence having at least 70% sequence identity to SEQ ID NO: 33.

The heterologous xanthan-utilization gene or gene locus may further comprise one or more nucleic acids encoding at least one or all of: one or more carbohydrate uptake proteins; one or more carbohydrate esterases; a polysaccharide lyase family protein (PL); a glycoside hydrolase family 88 (GH88); a glycoside hydrolase family 94 (GH94); and a glycoside hydrolase family 38 (GH38). In some embodiments, the heterologous xanthan-utilization gene or gene locus further comprises one or more nucleic acids encoding each of: one or more carbohydrate uptake proteins; one or more carbohydrate esterases; a polysaccharide lyase family protein (PL); a glycoside hydrolase family 88 (GH88); a glycoside hydrolase family 94 (GH94); and a glycoside hydrolase family 38 (GH38).

Carbohydrate uptake proteins include any proteins or enzymes necessary for the import of carbohydrates, including xanthan oligosaccharides, into the bacterial cell. Carbohydrate uptake proteins may include, but are not limited to, carbohydrate binding proteins and carbohydrate transporters. In some embodiments, the carbohydrate uptake proteins include transporters capable of transporting xanthan oligosaccharides produced by the xanthanase described herein.

Polysaccharide lyases (or eliminases) are a class of enzymes that act to cleave certain activated glycosidic linkages present in polysaccharides. These enzymes act through an eliminase mechanism, rather than through hydrolysis, resulting in unsaturated oligosaccharide products. Polysaccharide lyases are endogenous to various microorganisms, bacteriophages, and some eukaryotes. The polysaccharide lyases have been classified into approximately 40 families available through the Carbohydrate Active enZyme (CAZy) database.

In some embodiments, the polysaccharide lyase family protein comprises a polysaccharide lysase family 8 protein. In some embodiments, the polysaccharide lyase family protein comprises an amino acid sequence having at least 70% identity to SEQ ID NO: 4.

Glycoside hydrolases are enzymes that catalyze the hydrolysis of the glycosidic linkage of glycosides, leading to formation of sugar hemiacetal or hemiketal products. Glycoside hydrolases are also referred to as glycosidases, and sometimes also as glycosyl hydrolases. The glycoside hydrolases have been classified into more than 100 families available through the Carbohydrate Active enZyme database. Each family contains proteins that are related by sequence, and by extension, tertiary structure. A number of glycoside hydrolases may be used in the heterologous xanthan-utilization gene or gene locus disclosed herein.

In some embodiments, the glycoside hydrolase is from the glycoside hydrolase family 88 (GH88). In some embodiments, the glycoside hydrolase family 88 protein comprises an amino acid sequence having at least 70% identity to SEQ ID NO: 8.

In some embodiments, the glycoside hydrolase is from the glycoside hydrolase family 94 (GH94). In some embodiments, the glycoside hydrolase family 94 protein comprises an amino acid sequence having at least 70% identity to SEQ ID NO: 5.

In some embodiments, the glycoside hydrolase is from the glycoside hydrolase family 38 (GH38). In some embodiments, the glycoside hydrolase family 38 protein comprises an amino acid sequence having at least 70% identity to SEQ ID NO: 6 or SEQ ID NO: 7.

Carbohydrate esterases are a group of enzymes which release acyl or alkyl groups attached by ester linkage to carbohydrates. The carbohydrate esterases catalyze deacetylation of both O-linked and N-linked acetylated saccharide residues (esters or amides). The carbohydrate active enzyme database has 16 classified families of carbohydrate esterases. In some embodiments, the carbohydrate esterase used herein is capable of deacetylating xanthan oligosaccharides produced by the xanthanase described herein. The heterologous xanthan-utilization gene or gene locus may include one or more carbohydrate esterases. In some embodiments, the one or more carbohydrate esterases independently comprise an amino acid sequence having at least 70% identity to SEQ ID NO: 9 or SEQ ID NO: 10. In some embodiments, the heterologous xanthan-utilization gene or gene locus includes two carbohydrate esterases, ones having an amino acid sequence having at least 70% identity to SEQ ID NO: 9 and the other having an amino acid sequence having at least 70% identity to SEQ ID NO: 10.

The heterologous xanthan-utilization gene or gene locus may further comprise, in addition or alternatively, one or more nucleic acids encoding at least one or all of: one or more carbohydrate uptake proteins; a polysaccharide lyase family protein (PL); a glycoside hydrolase family 88 (GH88); a glycoside hydrolase family 92 (GH92); and a glycoside hydrolase family 3 (GH3). In some embodiments, the heterologous xanthan-utilization gene or gene locus further comprises two carbohydrate uptake proteins. In some embodiments, the heterologous xanthan-utilization gene or gene locus further comprises each of two carbohydrate uptake proteins and at least one or all of: a polysaccharide lyase family protein (PL); a glycoside hydrolase family 88 (GH88); a glycoside hydrolase family 92 (GH92); and a glycoside hydrolase family 3 (GH3). In some embodiments, the heterologous xanthan-utilization gene or gene locus further comprises each of two carbohydrate uptake proteins, a polysaccharide lyase family protein (PL), a glycoside hydrolase family 88 (GH88), a glycoside hydrolase family 92 (GH92), and a glycoside hydrolase family 3 (GH3).

The carbohydrate uptake proteins may include members of the starch utilization system (Sus) of Bacteroides. The Sus includes the requisite proteins for binding and processing carbohydrates at the surface of the cell and, the subsequent oligosaccharide transport across the membrane for further degradation. All mammalian gut Bacteroidetes possess analogous Sus-like systems that target numerous diverse glycans. The carbohydrate uptake protein may include SusC or SusD or homologs or variants thereof from Bacteroides known in the art (See, for example, Xu, et al., PLoS Biol. 2007 July; 5(7): e156 and Foley, et al., Cell Mol Life Sci. 2016 July; 73(14): 2603-2617, both incorporated by reference herein in their entirety. In some embodiments, the one or more carbohydrate uptake proteins independently comprise an amino acid sequence having at least 70% identity to SEQ ID NO: 11 or SEQ ID NO: 12. In some embodiments, the one or more carbohydrate uptake proteins independently comprise an amino acid sequence having at least 70% identity to SEQ ID NO: 43 or SEQ ID NO: 44.

In some embodiments, the polysaccharide lyase family protein comprises a polysaccharide lysase family 2 protein. In some embodiments, the polysaccharide lyase family protein comprises an amino acid sequence having at least 70% identity to SEQ ID NO: 14. In some embodiments, the polysaccharide lyase family protein comprises an amino acid sequence having at least 70% identity to SEQ ID NO: 42.

In some embodiments, the glycoside hydrolase is from the glycoside hydrolase family 88 (GH88). In some embodiments, the glycoside hydrolase family 88 protein comprises an amino acid sequence having at least 70% identity to SEQ ID NO: 16. In some embodiments, the glycoside hydrolase family 88 protein comprises an amino acid sequence having at least 70% identity to SEQ ID NO: 38.

In some embodiments, the glycoside hydrolase is from the glycoside hydrolase family 92 (GH92). In some embodiments, the glycoside hydrolase family 92 protein comprises an amino acid sequence having at least 70% identity to SEQ ID NO: 17. In some embodiments, the glycoside hydrolase family 92 protein comprises an amino acid sequence having at least 70% identity to SEQ ID NO: 39.

In some embodiments, the glycoside hydrolase is from the glycoside hydrolase family 3 (GH3). In some embodiments, the glycoside hydrolase family 3 protein comprises an amino acid sequence having at least 70% identity to SEQ ID NO: 13. In some embodiments, the glycoside hydrolase family 3 protein comprises an amino acid sequence having at least 70% identity to SEQ ID NO: 35 or SEQ ID NO: 36.

The heterologous xanthan-utilization gene or gene locus may further comprise additional genes encoding proteins and enzymes involved in xanthan-utilization including, but not limited to, glucokinases, mannose-6-phophate isomerases, phosphoglucomutases, other glycoside hydrolases (e.g., other glycoside hydrolase family 5 proteins), environmental sensors, and signaling proteins (e.g., response regulators). For example the gene locus may further comprise a glucokinase protein having an amino acid sequence having at least 70% identity to SEQ ID NO: 18 or 20, a transporter protein having an amino acid sequence having at least 70% identity to SEQ ID NO: 26-29, a transcriptional regulator having an amino acid sequence having at least 70% identity to SEQ ID NO: 25, a response regulator having an amino acid sequence having at least 70% identity to SEQ ID NO: 24, an isomerase having an amino acid sequence having at least 70% identity to SEQ ID NO: 22 or 23, a kinase having an amino acid sequence having at least 70% identity to SEQ ID NO: 21, a carbohydrate-binding module protein (e.g. Carbohydrate-binding module family 11 protein) having an amino acid sequence having at least 70% identity to SEQ ID NO: 19, and/or an environmental sensor (e.g. hybrid two-component system (HTCS) protein) having an amino acid sequence having at least 70% identity to SEQ ID NO: 30 or 40.

The heterologous xanthan-utilization gene locus may comprise a nucleic acid sequence having an amino acid sequence having at least 70% identity to SEQ ID NO: 31, 32, or 45.

The bacteria may be from the genus Bacteroides, Clostridium, Faecalibacterium, Eubacterium, Ruminococcus, Peptococcus, Peptostreptococcus, Bifidobacteria, Escherichia, and/or Lactobacillus.

In some embodiments, the genetically modified bacterium is in the genus Bacteroides, including but not limited to, B. acidifaciens, B. amylophilus, B. asaccharolyticus, B. barnesiae, B. bivius, B. buccae, B. buccalis, B. caccae, B. capillosus, B. capillus, B. cellulosilyticus, B. chinchilla, B. clarus, B. coagulans, B. coprocola, B. coprophilus, B. coprosuis, B. corporis, B. denticola, B. disiens, B. distasonis, B. dorei, B. eggerthii, B. endodontalis, B. faecichinchillae, B. faecis, B. finegoldii, B. fluxus, B. forsythus, B. fragilis, B. furcosus, B. galacturonicus, B. gallinarum, B. gingivalis, B. goldsteinii, B. gracilis, B. graminisolvens, B. helcogenes, B. heparinolyticus, B. hypermegas, B. intermedius, B. intestinalis, B. johnsonii, B. levvi, B. loescheii, B. macacae, B. massiliensis, B. melaninogenicus, B. merdae, B. microfusus, B. multiacidus, B. nodosus, B. nordii, B. ochraceus, B. oleiciplenus, B. oralis, B. oris, B. oulorum, B. ovatus, B. paurosaccharolyticus, B. pectinophilus, B. pentosaceus, B. plebeius, B. pneumosintes, B. polypragmatus, B. praeacutus, B. propionicifaciens, B. putredinis, B. pyogenes, B. reticulotermitis, B. rodentium, B. ruminicola, B. salanitronis, B. salivosus, B. salyersiae, B. sartorii, B. splanchnicus, B. stercorirosoris, B. stercoris, B. succinogenes, B. suis, B. tectus, B. termitidis, B. thetaiotaomicron, B. uniformis, B. ureolyticus, B. veroralis, B. vulgatus, B. xylanisolvens, B. xylanolyticus, B. zoogleoformans, and any combination thereof.

In some embodiments, the genetically modified bacterium is a gram-positive gut commensal bacteria. The gram-positive gut commensal bacteria may be from the genus Enterococcus, Staphylococcus, Lactobacillus, Clostridium, Peptostreptococcus, Peptococcus, Streptococcus, Bifidobacterium, and/or Faecalibacterium. In some embodiments, the gram-positive gut commensal bacteria may be Lactobacillus reuteri or Clostridium scindens.

In some embodiments, the genetically modified bacteria may comprise the polynucleotide on a plasmid, a bacterial artificial chromosome or integrated into the genome of the bacterium.

Also provided are compositions comprising the genetically modified bacteria described herein. In some embodiments, the composition is a pharmaceutical composition (e.g., probiotic composition) further comprising excipients and/or pharmaceutically acceptable carriers. The excipients and/or pharmaceutically acceptable carriers may facilitate delivery of the genetically modified bacteria to a subject, for example a subject's gastro-intestinal tract, in a viable and metabolically-active condition, for example in a condition capable of colonizing and/or metabolizing and/or proliferating in the gastrointestinal tract.

The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, and isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995). The composition can comprise additional components, such as vitamins, minerals, carbohydrates, and a mixture thereof.

The composition may take on many forms. In some embodiments, the composition comprises encapsulating (e.g., in tablets, caplets, microcapsules) the genetically modified bacteria for enhanced delivery and survival in the gastric and/or gastrointestinal tract of a subject. In some embodiments, the composition is a foodstuff including liquids (e.g., drinks), semi-solids (e.g., jellies, yogurts, puddings, smoothies, and the like) and solids.

The disclosure also provides, a method of treating a disease or disorder comprising administering a therapeutically or prophylactically effective dose of the genetically modified bacteria or compositions thereof to a subject in need thereof. The specific dose level may depend upon a variety of factors including the age, body weight, and general health of the subject, time of administration, and route of administration. An “effective amount” is an amount that is delivered to a subject, either in a single dose or as part of a series, which achieves a medically desirable effect. For therapeutic purposes, and effect amount is the quantity which, when administered to a subject in need of treatment, improves the prognosis and/or state of the subject and/or that reduces or inhibits one or more symptoms to a level that is below that observed and accepted as clinically diagnostic or clinically characteristic of the disease or disorder. For prophylaxis purposes, an effective amount is that amount which induces a protective result without significant adverse side effects.

The frequency of dosing the effective amount can vary, but typically the effective amount is delivered daily, either as a single dose, multiple doses throughout the day, or depending on the dosage form, dosed continuously for part or all of the treatment period.

The genetically modified bacteria may be administered at about 104 to about 10¹⁰ cfu per dose, about 10⁵ to about 10⁹ cfu per dose, about 10⁵ to about 10⁷ cfu per dose, or about 10⁹ cfu per dose.

The disease or disorder may comprise a gastrointestinal disease or disorder including diseases and disorders that cause inflammation in the gastrointestinal system including, but not limited to, Irritable Bowel Syndrome, diarrhea, Crohn's disease, ulcerative colitis, and gluten intolerance or Celiac's disease. The treatment may be combined with gluten-free or low carbohydrate diets that are high in xanthan gum.

In some embodiments, the administration is oral. The genetically modified bacteria may be administered with food (e.g., concomitantly with food, within an hour of before or after consuming food).

### 5. Examples

### Materials and Methods

Culturing and phylogenetic analysis of xanthan degrading cultures Xanthan degrading cultures were grown in Defined Medium (DM), which was generally prepared as a 2×stock then mixed 1:1 with 10 mg/mL carbon source (e.g., xanthan gum). Cultures were grown in an anaerobic chamber (10% H₂, 5% CO₂, and 85% N₂) maintained at 37° C. Each liter of prepared DM medium (pH=7.2) contained 13.6 g KH₂PO₄, 0.875 g NaCl, 1.125 g (NH₄)₂SO₄, 2 mg each of adenine, guanine, thymine, cytosine, and uracil, 2 mg of each of the 20 essential amino acids, 1 mg vitamin K3, 0.4 mg FeSO₄, 9.5 mg MgCl₂, 8 mg CaCl₂, 5 μg Vitamin B12, 1 g L-cysteine, 1.2 mg hematin with 31 mg histidine, 1 mL of Balch's vitamins, 1 mL of trace mineral solution, and 2.5 g beef extract.

Each liter of Balch's vitamins was prepared with 5 mg p-Aminobenzoic acid, 2 mg folic acid, 2 mg biotin, 5 mg nicotinic acid, 5 mg calcium pantothenate, 5 mg riboflavin, 5 mg thiamine HCl, 10 mg pyridoxine HCl, 0.1 mg cyanocobalamin, 5 mg thioctic acid. Prepared Balch's vitamins adjusted to pH 7.0, filter sterilized with 0.22 μm PES filters, and stored in the dark at 4° C.

Each L of trace mineral solution was prepared with 0.5 g EDTA (Sigma, ED4SS), 3 g MgSO₄·7H₂O, 0.5 g MnSO₄·H₂O, 1 g NaCl (Sigma, S7653), 0.1 g FeSO₄·7H₂O (Sigma, 215422), 0.1 g CaCl₂, 0.1 g ZnSO₄·7H₂O, 0.01 g CuSO₄·5H₂0, 0.01 g H₃BO₃ (Sigma, B6768), 0.01 g Na₂MoO₄·2H₂O, 0.02 g NiCl₂·6H₂O. Prepared trace mineral solution was adjusted to pH 7.0, filter sterilized with 0.22 μm PES filters, and stored at room temperature.

Samples that showed growth on xanthan gum, as evidenced by loss of viscosity and increased culture density, were subcultured 10 times by diluting an active culture 1:100 into fresh DM-XG medium. For the original culture, multiple samples were stored for gDNA extraction and analysis while for the larger sample set, samples were stored after 10 passages; samples were harvested by centrifugation, decanted, and stored at −20° C. until further processing.

Frozen cell pellets were resuspended in 500 μL Buffer A (200 mM NaCl, 200 mM Tris-HCl, 20 mM EDTA) and combined with 210 μL SDS (20% w/v, filter-sterilized), 500 μL phenol:chloroform (alkaline pH), and ˜250 μL acid-washed glass beads (212-300 μm; Sigma). Samples were bead beaten on high for 2-3 minutes with a Mini-BeadBeater-16 (Biospec Products, USA), then centrifuged at 18,000 g for 5 mins. The aqueous phase was recovered and mixed by inversion with 500 μL of phenol:chloroform, centrifuged at 18,000 g for 3 mins, and the aqueous phase was recovered again. The sample was mixed with 500 μL chloroform, centrifuged, and then the aqueous phase was recovered and mixed with 0.1 volumes of 3 M sodium acetate (pH 5.2) and 1 volume isopropanol. The sample was stored at −80° C. for ≥30 mins, then centrifuged at ≥20,000 g for 20 mins at 4° C. The pellet was washes with 1 mL room temperature 70% ethanol, centrifuged for 3 mins, decanted, and allowed to air dry before resuspension in 100 μL sterile water. Resulting samples were additionally purified using the DNeasy Blood & Tissue Kit (QIAGEN, USA). Illumina sequencing, including PCR and library preparation, were performed by the University of Michigan Microbial Systems Molecular Biology lab as described by Kozich et al (Appl. Environ. Microbiol. 79, 5112-5120 (2013), incorporated herein by reference in its entirety). Barcoded dual-index primers specific to the 16S rRNA V4 region were used to amplify the DNA. PCR reactions consisted of 5 μL of 4 μM equimolar primer set, 0.15 μL of AccuPrime Taq DNA High Fidelity Polymerase, 2 μL of 10× AccuPrime PCR Buffer II (Thermo Fisher Scientific, catalog no. 12346094), 11.85 μL of PCR-grade water, and 1 μL of DNA template. The PCR conditions used consisted of 2 min at 95° C., followed by 30 cycles of 95° C. for 20 s, 55° C. for 15 s, and 72° C. for 5 min, followed by 72° C. for 10 min. Each reaction was normalized using the SequalPrep Normalization Plate Kit (Thermo Fisher Scientific, catalog no. A1051001), then pooled and quantified using the Kapa Biosystems Library qPCR MasterMix (ROX Low) Quantification kit for Illumina platforms (catalog no. KK4873). After confirming the size of the amplicon library using an Agilent Bioanalyzer and a high-sensitive DNA analysis kit (catalog no. 5067-4626), the amplicon library was sequenced on an Ilumina MiSeq platform using the 500 cycle MiSeq V2 Reagent kit (catalog no. MS-102-2003) according to the manufacturer's instructions with modifications of the primer set with custom read 1/read 2 and index primers added to the reagent cartridge. The “Preparing Libraries for Sequencing on the MiSeq” (part 15039740, Rev. D) protocol was used to prepare libraries with a final load concentration of 5.5 μM, spiked with 15% PhiX to create diversity within the run.

MPN/Dilution to extinction experiment An overnight culture was serially diluted in 2× DM. Serial dilutions were split into two 50 mL tubes and mixed 1:1 with either 10 mg/mL xanthan gum or 10 mg/mL monosaccharide mixture (4 mg/mL glucose, 4 mg/mL mannose, 2 mg/mL sodium glucuronate), both of which also had 1 mg/mL L-cysteine. Each dilution and carbon source was aliquoted to fill a full 96-well culture plate (Costar 3370) with 200 p L per well. Plates were sealed with Breathe-Easy gas permeable sealing membrane for microtiter plates (Diversified Biotech, cat #BEM-1). Microbial growth was measured at least 60 hours by monitoring OD₆₀₀ using a Synergy HT plate reader (Biotek Instruments) and BIOSTACK2WR plate handler (Biotek Instruments).

Maximum OD for each substrate was measured for each culture. Full growth on substrates was conservatively defined as a maximum OD₆₀₀ of >0.7. For each unique 96 well plate of substrate and dilution factor, the fraction of wells exhibiting full growth was calculated. Fractional growth was plotted against dilution factor for each substrate. Data were fit to the Hill equation by minimizing squared differences between the model and experimental values using Solver (GRG nonlinear) in Excel. For each experiment, a 50% growth dilution factor (GDF 50) was calculated for each substrate at which half of the wells would be predicted to exhibit full growth.

Neutral Monosaccharide analysis. The hot-phenol extraction method originally described by Massie & Zimm (Proc. Natl. Acad. Sci. 54, 1641-1643 (1965), incorporated herein by reference) and modified by Nie (ProQuest Diss. Theses 136 (2016), incorporated herein by reference) was used for collecting and purifying the polysaccharides remaining at different timepoints. Samples were heated to 65° C. for 5 mins, combined with an equal volume of phenol, incubated at 65° C. for 10 mins, then cooled to 4° C. and centrifuged at 4° C. for 15 min at 12,000 g. The upper aqueous layer was collected and re-extracted using the same procedure, dialyzed extensively against deionized water (2000 Da cutoff), and freeze-dried. Neutral monosaccharide composition was obtained using the method described by Tuncil et al. (Sci. Rep. 8, 1-13 (2018), incorporated herein by reference). Briefly, sugar alditol acetates were quantified by gas chromatography using a capillary column SP-2330 (SUPELCO, Bellefonte, PA) with the following conditions: injector volume, 2 μl; injector temperature, 240° C.; detector temperature, 300° C.; carrier gas (helium), velocity 1.9 meter/second; split ratio, 1:2; temperature program was 160° C. for 6 min, then 4° C./min to 220° C. for 4 min, then 3° C./min to 240° C. for 5 min, and then 11° C./min to 255° C. for 5 min.

Thin Layer Chromatography for Localization of Enzyme Activity Overnight cultures were harvested at 13,000 g for 10 minutes. Supernatant fractions were prepared by vacuum filtration through 0.22 μm PES filters. Cell pellet fractions were prepared by decanting supernatant, washing with phosphate buffered saline (PBS), spinning at 13,000 g for 3 mins, decanting, and resuspending in PBS. Intracellular fractions were prepared by taking cell pellet fractions and bead beating for 90 s with acid-washed glass beads (G1277, Sigma) in a Biospec Mini Beadbeater. Lysed culture fractions were prepared by directly bead beating unprocessed culture.

Each culture fraction was mixed 1:1 with 5 mg/mL xanthan gum and incubated at 37° C. for 24 hours. Negative controls were prepared by heating culture fractions to 95° C. for 15 mins, then centrifuging at 13,000 g for 10 mins before the addition of xanthan gum. All reactions were halted by heating to ≥85° C. for 15 mins, then spun at 20,000 g for 15 mins at 4° C. Supernatants were stored at −20° C. until analysis by thin layer chromatography.

Samples (3 μL) were spotted twice onto a 10×20 cm thin layer chromatography plate (Millipore TLC Silica gel 60, 20×20 cm aluminum sheets), with intermediate drying using a Conair 1875 hairdryer. Standards included malto-oligosaccharides of varying lengths (Even: 2, 4, 6, Odd: 1, 3, 5, 7), glucuronic acid, and mannose. Standards were prepared at 10 mM and 3 uL of each was spotted onto the TLC plate. Plates were run in ˜100 mL of 2:1:1 butanol, acetic acid, water, dried, then run an additional time. After drying, plates were incubated in developing solution (100 mL ethyl acetate, 2 g diphenylamine, 2 mL aniline, 10 mL of ˜80% phosphoric acid, 1 mL of ˜38% hydrochloric acid) for ˜30 seconds, then dried, and developed by holding over a flame until colors were observed.

Proteomic analysis Approximately 1 L of xanthan gum culture was grown until it had completely liquified (˜2-3 days). Supernatant was collected by centrifuging at 18,000 g and vacuum filtering through a 0.2 μm PES filter. 4M ammonium sulfate was added to 200-400 mL of filtrate to a final concentration of 2.4M and incubated for 30-60 mins at RT or, for one sample, overnight at 4° C. Precipitated proteins were harvested by centrifugation at 18,000 g for 30-60 mins, then resuspended in 50 mM sodium phosphate (pH 7.5). Three different fractionation protocols were followed, but after every fractionation step, active fractions were identified by mixing ˜500 μL with 10 mg/mL xanthan and incubating at 37° C. overnight; active-fractions were identified by loss of viscosity or production of xanthan oligosaccharides as visualized by TLC.

1. Resuspended protein was filtered and applied to a HiTrapQ column, running a gradient from β-100% B (Buffer A: 50 mM sodium phosphate, pH 7.5; Buffer B: 50 mM sodium phosphate, 1 M NaCl, pH 7.5). Active fractions were pooled and concentrated with a 10 kDa MWCO centricon and injected onto an S-200 16/60 column equilibrated in 50 mM sodium phosphate, 200 mM NaCl, pH 7.5. The earliest fractions to elute with significant A280 absorbance were also the most active fractions; these were pooled and submitted for proteomics.

2. Resuspended protein was filtered and applied to an S-500 column equilibrated in 50 mM sodium phosphate, 200 mM NaCl, pH 7.5. Active fractions eluted in the middle of the separation were pooled and submitted for proteomics.

3. Resuspended protein was filtered and applied to an S-500 column equilibrated in 50 mM sodium phosphate, 200 mM NaCl, pH 7.5. Pooled fractions were applied to a 20 mL strong anion exchange column running a gradient from β-100% B (Buffer A: 50 mM sodium phosphate, pH 7.5; Buffer B: 50 mM sodium phosphate, 1 M NaCl, pH 7.5). Active fractions were pooled and applied to a 1 mL weak anion exchange column (ANX) running a gradient from β-100% B (Buffer A: 50 mM sodium phosphate, 10% glycerol, pH 7.5; Buffer B: 50 mM sodium phosphate, 1 M NaCl, 10% glycerol, pH 7.5). Active fractions were pooled and submitted for proteomics.

Cysteines were reduced by adding 50 ml of 10 mM DTT and incubating at 45° C. for 30 min. Samples were cooled to room temperature and alkylation of cysteines was achieved by incubating with 65 mM 2-Chloroacetamide, under darkness, for 30 min at room temperature. An overnight digestion with 1 μg sequencing grade, modified trypsin was carried out at 37° C. with constant shaking in a Thermomixer. Digestion was stopped by acidification and peptides were desalted using SepPak C18 cartridges using manufacturer's protocol (Waters). Samples were completely dried using vacufuge. Resulting peptides were dissolved in 8 ml of 0.1% formic acid/2% acetonitrile solution and 2 μls of the peptide solution were resolved on a nano-capillary reverse phase column (Acclaim PepMap C18, 2 micron, 50 cm, ThermoScientific) using a 0.1% formic acid/2% acetonitrile (Buffer A) and 0.1% formic acid/95% acetonitrile (Buffer B) gradient at 300 nl/min over a period of 180 min (2-25% buffer B in 110 min, 25-40% in 20 min, 40-90% in 5 min followed by holding at 90% buffer B for 10 min and re-equilibration with Buffer A for 30 min). Eluent was directly introduced into Q exactive HF mass spectrometer (Thermo Scientific, San Jose CA) using an EasySpray source. MS1 scans were acquired at 60K resolution (AGC target=3×10⁶; max IT=50 ms). Data-dependent collision induced dissociation MS/MS spectra were acquired using Top speed method (3 seconds) following each MS1 scan (NCE˜28%; 15K resolution; AGC target 1×10⁵; max IT 45 ms).

Proteins were identified by searching the MS/MS data against a database of all proteins identified in the original culture metagenomes using Proteome Discoverer (v2.1, Thermo Scientific). Search parameters included MS1 mass tolerance of 10 ppm and fragment tolerance of 0.2 Da; two missed cleavages were allowed; carbamidomethylation of cysteine was considered fixed modification and oxidation of methionine, deamidation of asparagine and glutamine were considered as potential modifications. False discovery rate (FDR) was determined using Percolator and proteins/peptides with a FDR of ≤1% were retained for further analysis.

Kinetics of GH5-30 Lyase-treated xanthan gum was generated by mixing 5 mg/mL xanthan gum with 0.5 U/mL of Bacillus sp. Xanthan lyase (E-XANLB, Megazyme) in 30 mM potassium phosphate buffer (pH 6.5). After incubating overnight at 37° C., an addition 0.5 U/mL of xanthan lyase was added. Both lyase-treated and native xanthan gum were dialyzed extensively against deionized water, heated in an 80° C. water bath to inactivate the lyase, and centrifuged at 10,000 g for 20 mins to remove particulate. Supernatants were collected and stored at 4° C. until use. Kinetic measurements were conducted using a slightly modified version of the low-volume bicinchoninic acid (BCA) assay for glycoside hydrolases used by Arnal et al (Protein-Carbohydrate Interactions. Methods and Protocols (eds. Abbott, D. W. & Lammerts van Bueren, A.) 1588, 209-214 (2017), incorporated herein by reference). Briefly, AEX and SEC purified GH5 was diluted to a 10× stock of 5 μM enzyme, 50 mM sodium phosphate, 300 mM sodium chloride, and 0.1 mg/mL bovine serum albumin, pH=7.5. Reactions were 20 μL of enzyme stock mixed with 180 μL of various concentrations 37° C. xanthan gum. Negative controls were conducted with heat-inactivated enzyme stock. Timepoints were taken by quenching reactions with dilute, ice-cold, BCA working reagent. Reactions and controls were run with 4 independent replicates and compared to a glucose standard curve. Enzyme released reducing sugars were calculated by subtracting controls from reaction measurements.

Growth curves of isolates on XG oligos Pure isolates from the xanthan culture were obtained by streaking an active culture onto a variety of agar plates including LB and brain heart infusion with the optional addition of 10% defibrinated horse blood (Colorado Serum Co.) and gentamycin. After passaging isolates twice on agar plates, individual colonies were picked and grown overnight in tryptone-yeast extract-glucose (TYG) broth medium, then stocked by mixing with 0.5 volumes each of TYG and molecular biology grade glycerol and storing at −80° C. DM without beef extract (DM^{-BE}), with the addition of a defined carbon source, was used to test isolates for growth on xanthan oligosaccharides. Some isolates (e.g., Parabacteroides distasonis) required the inclusion of 5 mg/mL beef extract (Sigma, B4888) to achieve robust growth on simple monosaccharides; in these cases, beef extract was included across all carbon conditions. Unless otherwise specified, carbon sources were provided at a final concentration of 5 mg/mL. Isolates were grown overnight in TYG media, subcultured 1:50 into DM^{-BE}-glucose and grown overnight, then subcultured 1:50 into DM^{-BE} with either various carbon sources. Final cultures were monitored for growth by measuring increase in absorbance (600 nm) using 96-well plates.

Extended metagenome analysis/comparison methodology Individual MAGs in each sample were searched by BlastP for the presence of proteins similar to those encoded by the XG-degrading PUL of R. UCG13 and B. intestinalis. This was done using the amino acid sequences of the proteins in the R. UCG13 and B. intestinalis PULs as the search homologs; both BlastP probes were searched against all the individual MAGs in the different samples with the default threshold e-value of le-5.

R. UCG13 and B. intestinalis/cell. XG Loci in Metagenomes Available cohorts of human gut metagenomic sequence data (National Center for Biotechnology Information projects: PRJNA422434, PRJEB10878, PRJEB12123, PRJEB12124, PRJEB15371, PRJEB6997, PRJDB3601, PRJNA48479, PRJEB4336, PRJEB2054, PRJNA392180, and PRJNA527208) were searched for the presence of xanthan locus nucleotide sequences from R. UCG13 (92.7 kb) and B. intestinalis (17.9kb) using the following workflow: Each xanthan locus nucleotide sequence was used separately as a template and then magic-blast v1.5.0 was used to recruit raw Illumina reads from the available metagenomic datasets with an identity cutoff of 97%. Next, the alignment files were used to generate a coverage map using bedtools v2.29.0 to calculate the percentage coverage of each sample against each individual reference. Metagenomic data sample was considered a to be positive for a particular xanthan locus if it had at least 70% of the corresponding xanthan locus nucleotide sequence covered.

The R. UCG13 locus and B. intestinalis XG locus were used as the query in a large-scale search against the assembled scaffolds of isolates, metagenome assembled genomes (bins), and metagenomes included into the Integrated Microbial Genomes & Microbiomes (IMG/M) comparative analysis system. Within the LAST software package, version 1066, the ‘lastal’ tool was used with default thresholds to search the 2 loci against 72,491 public high-quality isolate genomes, and 102,860 bins from 13,415 public metagenomes, and 21,762 public metagenomes in IMG/M. Metagenome bins were generated using the binning analysis method described in Clum, A. et al. The DOE JGI Metagenome Workflow. bioRxiv (2020), incorporated herein by reference.

Ruminococcaceae UCG13 —Glycosyl Hydrolase 5 (aka XGD26-15, aka GH5-30) Following 16s rDNA gene content determination and metagenomic sequencing of a multi-species xanthan-degrading community, sequence-specific oligonucleotide primers were designed and used to amplify the GH5 sequence from genomic DNA isolated from the multi-species culture. The PCR product for the protein was inserted into a C-terminal His-tagged expression construct using the Lucigen Expresso™ T7 Cloning and Expression System. The engineered plasmid containing the GH5-30 His-tagged sequence was transformed into BL21 (DE3) chemically competent cells. Seed cultures were grown overnight, followed by inoculation of 1 L of either LB or TB media, grown at 37° C. to an OD of ˜0.6-0.8, then induced with 250 μM IPTG and cooled to 18° C. for overnight (12-18 hr) expression. Cells were harvested by centrifugation, lysed with sonication, and recombinant protein was purified using standard His-tagged affinity protein purification protocols employing sodium phosphate buffers and either nickel or cobalt resin for immobilized metal affinity chromatography.

In general, pentameric xanthan oligosaccharides were produced by incubating ≥0.1 mg/mL GH5 with 5 mg/mL xanthan gum in PBS in approximately 1L total volume. For xanthan tetrasaccharides, ˜0.5 U/mL of Xanthan lyase (E-XANLB, Megazyme) was included. After incubating 2-3 days at 37° C. to allow complete liquefication, reactions were heat-inactivated, centrifuged at ≥10,000 g for 30 mins, and the supernatant was vacuum filtered through 0.22 μm PES sterile filters. Supernatants were loaded onto a column containing ˜10 g of graphitized carbon (Supelclean™ ENVI-Carb™, 57210-U Supelco), washed extensively with water to remove salt and unbound material, then eluted in a stepwise fashion with increasing concentrations of acetonitrile. Fractions were dried, weighed, and analyzed by LC-MS and fractions that contained the most significant yield of desired products were combined.

Highly pure products were obtained by reconstituting samples in 50% water:acetonitrile and applying to a Luna® 5 μm HILIC 200 Å LC column (250×10 mm) (OOG-4450-NO, Phenomenex). A gradient was run from 90-20% acetonitrile, with peaks determined through a combination of evaporative light scattering, UV, and post-run analytical LC-MS (Agilent qToF 6545) of resulting fractions.

NMR spectra were collected using an Agilent 600 NMR spectrometer (¹H: 600 MHz, ¹³C: 150 MHz) equipped with a 5 mm DB AUTOX PFG broadband probe and a Varian NMR System console. All data analysis was performed using MestReNova NMR software. All chemical shifts were referenced to residual solvent peaks [¹H (D₂O): 4.79 ppm].

Enzyme Reaction Analysis All enzyme reactions were similar to preparative methods. carried out in 15-25 mM sodium phosphate buffer, 100-150 mM sodium chloride, and sometimes included up to 0.01 mg/mL bovine serum albumin (B9000S, NEB) to limit enzyme adsorption to pipettes and tubes. All R. UCG13 or B. intestinalis enzymes were tested at concentrations from 1-10 μM. Cellobiose reactions were tested using 1 mM cellobiose at pH 7.5, while all other reactions used 2.5 mg/mL pentasaccharide (produced using RuGH5a) and were carried out at pH 6.0. Reactions were heat-inactivated and centrifuged incubated overnight at 37° C., halted by heating at ≥95° C. for 5-10 minutes, and centrifugation at ≥20,000 g for 10 mins. Supernatants were mixed 1:1 with 4 parts acetonitrile and to yield an 80% acetonitrile solution, centrifuged for 10 mins at ≥20,000 g, and transferred into sample vials. 15 μL of each sample was injected onto a Luna® Omega 3 μm HILIC 200 Å, LC column (100×4.6 mm) (00D-4449-E0, Phenomenex). An Agilent 1290 Infinity II HPLC system was used to separate the sample using solvent A gradient was run from 90-20(100% water, 0.1% formic acid) and solvent B (95% acetonitrile, 5% water, with 0.1% formic acid added) at a flow rate of 0.4 mL/min over the course of ˜10-40 mins. Products were detected by collecting mass spectra. Prior to injection and following each sample the column was equilibrated with 80% B. After injection, samples were eluted with a 30 minute isocratic step at 80% B, a 10 minute gradient decreasing B from 80% to 10%, and a final column wash for 2 min at 10% B. Spectra were collected in negative mode on a MS Detector info, using an Agilent 6545 LC/Q-TOF.

Metagenomics analysis Seven samples (15-mL) were collected at four time points (referred to as T1, T2, T3 and T4) during growth of two biological replicates of the original XG-degrading culture. Cells were harvested by centrifugation at 14,000×g for 5 min and stored a −20° C. until further use. A phenol:chloroform:isoamyl alcohol and chloroform extraction method was used to obtain high molecular weight DNA. The gDNA was quantified using a Qubit™ fluorimeter and the Quant-iT™ dsDNA BR Assay Kit (Invitrogen, USA), and the quality was assessed with a NanoDrop One instrument (Thermo Fisher Scientific, USA). Samples were subjected to metagenomic shotgun sequencing using the Illumina HiSeq 3000 platform at the Norwegian Sequencing Center (NSC, Oslo, Norway). Samples were prepared with the TrueSeq DNA PCR-free preparation and sequenced with paired ends (2×150 bp) on one lane. Quality trimming of the raw reads was performed using Cutadapt v1.3, to remove all bases on the 3′-end with a Phred score lower than 20 and exclude all reads shorter than 100 nucleotides, followed by a quality filtering using the FASTX-Toolkit v.0.0.14 (hannonlab.cshl.edu/fastx_toolkit/). Retained reads had a minimum Phred score of 30 over 90% of the read length. Reads were co-assembled using metaSPAdes v3.10.1 with default parameters and k-mer sizes of 21, 33, 55, 77 and 99. The resulting contigs were binned with MetaBAT v0.26.3 in “very sensitive mode”. The quality (completeness, contamination, and strain heterogeneity) of the metagenome assembled genomes (MAGs) was assessed by CheckM v1.0.7 with default parameters. Contigs were submitted to the Integrated Microbial Genomes and Microbiomes system for open reading frames (ORFs) prediction and annotation. Additionally, the resulting ORF were annotated for CAZymes using the CAZy annotation pipeline. This MAG collection was used as a reference database for mapping of the metatranscriptome data, as described below. Taxonomic classifications of MAGs were determined using both MiGA and GTDB-Tk.

Human fecal samples (20) from a second enrichment experiment (unbiased towards the cultivation of Bacteroides) as well as two enrichments with mouse fecal samples were processed for gDNA extraction and library preparation exactly as described above. Metagenomic shotgun sequencing was conducted on two lanes of both Illumina HiSeq 4000 and Illumina HiSeq X Ten platforms (Illumina, Inc.) at the NSC (Oslo, Norway), and reads were quality trimmed, assembled and binned as described above. Open reading frames were annotated using PROKKA v1.14.0 and resulting ORFs were further annotated for CAZymes using the CAZy annotation pipeline and expert human curation. Completeness, contamination, and taxonomic classifications for each MAG were determined as described above. AAI comparison between the human R. UCG13 and the R. UCG13 found in the two mouse samples was determined using CompareM (github.com/dparks1134/CompareM).

Extracted DNA from a second enrichment experiment on XG using the original culture was prepared for long-reads sequencing using Oxford Nanopore Technologies (ONT) Ligation Sequencing Kit (SQK-LSK109) according to the manufacture protocol. The DNA library was sequenced with the ONT MinION Sequencer using a R9.4 flow cell. The sequencer was controlled by the MinKNOW software v3.6.5 running for 6 hours on a laptop (Lenovo ThinkPad P73 Xeon with data stored to 2Tb SSD), followed by base calling using Guppy v3.2.10 in ‘fast’ mode. This generated in total 3.59 Gb of data. The Nanopore reads were further processed using Filtlong v0.2.0 (github.com/rrwick/Filtlong), discarding the poorest 5% of the read bases, and reads shorter than 1000 bp.

The quality processed Nanopore long-reads were assembled using CANU v1.9 with the parameters corOutCoverage=10000 corMinCoverage=0 corMhapSensitivity=high genomeSize=5m redMemory=32 oeaMemory=32 batMemory=200. An initial polishing of the generated contigs were carried out using error-corrected reads from the assembly with minimap2 v2.17-x map-ont and Racon v1.4.14 with the argument —include-unpolished. The racon-polished contigs were further polished using Medaka v1.1.3 (github.com/nanoporetech/medaka), with the commands medaka_consensus--model r941_minfast_g303_model.hdf5. Finally, Minimap2-ax sr was used to map quality processed Illumina reads to the medaka-polished contigs, followed by a final round of error correction using Racon with the argument —include-unpolished. Circular contigs were identified by linking the contig identifiers in the polished assembly back to suggestCircular=yes in the initial contig header provided by CANU. These contigs were quality checked using CheckM v1.1.3 and BUSCO v4.1.4. Circular contigs likely to represent chromosomes (>1 Mbp) were further gene-called and functionally annotated using PROKKA v1.13 and taxonomically classified using GTDB-tk v1.4.0 with the classify_wf command. Barrnap v0.9 (github.com/tseemann/barmap) was used to predict ribosomal RNA genes. Average nucleotide Identity (ANI) was measured between the short-reads and long-reads MAGs using FastANI v1.1 with default parameters. Short-reads MAGs were used as query while long-reads MAGs were set as reference genomes. Short-reads MAG1 showed an Average Nucleotide Identity (ANI) of 99.98% with the long-reads ONTCirc01, while short-reads MAG2 showed an ANI of 99.99% with the long-reads ONT_Circ02. Phylogenetic analysis revealed that ONT_Circ02 encoded four complete 16S rRNA operons, three of which were identical to the aforementioned R. UCG13 OTU.

Temporal metatranscriptomic analysis of the original XG-degrading community. Cell pellets from 6 mL samples collected at T1-T4 during growth of two biological replicates of the original XG-degrading culture were supplemented with RNAprotect Bacteria Reagent (Qiagen, USA) following the manufacturer's instructions and kept at −80° C. until RNA extraction. mRNA extraction and purification were conducted as described in Kunath et al. (ISME J. 13, 603-617 (2019). Samples were processed with the TruSeq stranded RNA sample preparation, which included the production of a cDNA library, and sequenced on one lane of the Illumina HiSeq 3000 system (NSC, Oslo, Norway) to generate 2×150 paired-end reads. Prior to assembly, RNA reads were quality filtered with Trimmomatic v0.36, whereby the minimum read length was required to be 100 bases and an average Phred threshold of 20 over a 10 nt window, and rRNA and tRNA were removed using SortMeRNA v.2.1b. Reads were pseudo-aligned against the metagenomic dataset using kallisto pseudo-pseudobam. Of the 58089 ORFs (that encode proteins with >60 aa) identified from the metagenome of the original XG-degrading community, 7549 (13%) were not found to be expressed, whereas 50540 (87%) were expressed, resulting in a reliable quantification of the expression due to unique hits (reads mapping unambiguously against one unique ORF).

Plasmid Design and Protein Purification Plasmid constructs to produce recombinant proteins were made with a combination of synthesized DNA fragments (GenScript Biotech, Netherlands) and PCR amplicons using extracted culture gDNA as a template. In general, sequences were designed to remove N-terminal signaling peptides and to add a histidine tag for immobilized metal affinity chromatography (IMAC) (in many cases using the Lucigen MA101-Expresso-T7-Cloning-&-Expression-System). Plasmid assembly and protein sequences are described in source and supplemental data.

Constructs were transformed into HI-Control BL21(DE3) cells and single colonies were inoculated in 5 mL overnight LB cultures at 37° C. 5 mL cultures were used to inoculate 1 L of Terrific Broth (TB) with selective antibiotic, grown to OD ˜0.8-1.1 at 37° C., and induced with 250 μM IPTG. B. intestinalis enzymes were expressed at RT, while R. UCG13 enzymes were expressed at 18° C. overnight. Cells were harvested by centrifugation and pellets were stored at −80° C. until further processing. Proteins were purified using standard IMAC purification procedures employing sonication to lyse cells. R. UCG13 proteins were purified using 50 mM sodium phosphate and 300 mM sodium chloride at pH 7.5; B. intestinalis proteins were purified using 50 mM Tris and 300 mM sodium chloride at pH 8.0. All proteins were eluted from cobalt resin using buffer with the addition of 100 mM imidazole, then buffer exchanged to remove imidazole using Zeba 2 mL 7 kDa MWCO desalting columns. Protein concentrations were determined by measuring A280 and converting to molarity using calculated extinction coefficients.

qPCR/and RNA-Seq on B. intestinalis and Original Community

For qPCR, B. intestinalis was grown as before but cells were harvested by centrifugation at mid-exponential phase, mixed with RNA Protect (QIAGEN), and stored at −80° C. until further processing. At collection, average OD₆₀₀ values were ˜0.8 and ˜0.6 for glucose- and oligosaccharide-grown cultures, respectively. RNeasy mini kit buffers (QIAGEN) were used to extract total RNA, purified with RNA-binding spin columns (Epoch), treated with DNase I (NEB), and additionally purified using the RNeasy mini kit. SuperScript III reverse transcriptase and random primers (Invitrogen) were used to perform reverse transcription. Target transcript abundance in the resulting cDNA was quantified using a homemade qPCR mix. Each 20 uL reaction contained 1× Thermopol Reaction Buffer (NEB), 125 uM dNTPs, 2.5 mM MgSO4, 1X SYBR Green I (Lonza), 500 nM gene specific or SI 7/8)65 nM 16S rRNA primer and 0.5 units Hot Start Taq Polymerase (NEB), and 10 ng of template cDNA. Results were processed using the ddCT method in which raw values were normalized to 16S rRNA values, then xanthan oligosaccharide values were compared to those from glucose to calculate fold-change in expression.

For RNA-seq, total RNA was used from the B. intestinalis growths used for qPCR. For the community grown on XG or PGA, 5 mL cultures of DM-XG or DM-PGA were inoculated with a 1:100 dilution of a fully liquified DM-XG culture. PGA cultures were harvested at mid-log phase at OD600˜0.85 whereas XG cultures were harvested at late-log phase at OD600˜1.2 to allow liquification of XG, which was necessary to extract RNA from these cultures. As before, cultures were harvested by centrifugation, mixed with RNA Protect (Qiagen) and stored at −80° C. until further processing. RNA was purified as before except that multiple replicates of DM-XG RNA were pooled together and concentrated with Zymo RNA Clean and Concentrator™-25 to reach acceptable concentrations for RNA depletion input. rRNA was depleted twice from the purified total RNA using the MICROBExpress™ Kit, each followed by a concentration step using the Zymo RNA Clean and Concentrator™-25. About 90% rRNA depletion was achieved for all samples. B. intestinalis RNA was sequenced using NovaSeq and community RNA was sequenced using MiSeq. The resulting sequence data was analyzed for differentially expressed genes following a previously published protocol76. Briefly, reads were filtered for quality using Trimmomatic v0.3968. Reads were aligned to each genome using BowTie2 v2.3.5.177. For the Bacteroides intestinalis transcriptome reads were aligned to its genome, while for the community data reads were aligned to either the B. intestinalis genome or the closed Ruminococcaceae UCG-13 metagenome assembled genome (MAG). Reads mapping to gene features were counted using htseq-count (release_0.11.1)78. Differential expression analysis was performed using the edgeR v3.34.0 package in R v.4.0.2 (with the aid of Rstudio v1.3.1093). The TMM method was used for library normalization79. Coverage data was visualized using Integrated Genome Viewer (IGV)80.

### Example 1

### Xanthan Gum Degradation

Xanthan gum (XG) has the same β-1,4-linked backbone as cellulose, but contains trisaccharide branches on alternating glucose residues consisting of an α-1,3-mannose, β-1,2-glucuronic acid, and terminal β-1,4-mannose. The terminal β-D-mannose and the inner α-D-mannose are variably pyruvylated at the 4,6-position or acetylated at the 6-position, respectively, with amounts determined by specific strain and culture conditions (FIG. 1A).

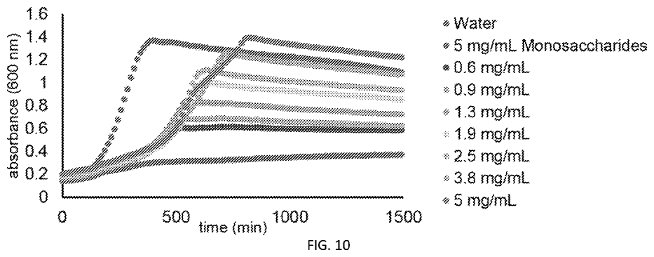

A group of 80 healthy 18-20 year-old adults were surveyed using a bacterial culture strategy originally designed to enrich for members of the Gram-negative Bacteroidetes, a phylum that generally harbors numerous polysaccharide-degrading enzymes. Based on increased bacterial culture turbidity and decreased viscosity of medium containing XG as the main carbon source, the initial survey revealed that just 1 out of 80 people sampled were positive for these characteristics. Growth analysis of a culture from the single positive subject revealed that bacterial growth was dependent on the amount of XG provided in the medium, demonstrating specificity for this nutrient (FIGS. 1B and 10). Attempts to enrich for the causal XG-consuming organism(s) by sequential passaging for 20 days yielded a stable mixed microbial culture with at least 12 distinct operational taxonomic units (OTUs; FIG. 1C). While these cultures had commonalities at the genus level, there was surprisingly only one OTU that was ≥0.5% and common across all 21 enrichment cultures examined. This common OTU was identified as a member of Ruminococcaceae uncultured genus 13 (R. UCG13). Plating and passaging the culture on BHI-blood plates resulted in loss of two previously abundant Gram-positive OTUs (loss defined as <0.01% relative abundance), including one identified as a member of Ruminococcaceae uncultured genus 13 (R. UCG13) in the Silva database. A corresponding loss of the XG-degrading phenotype was also found when plate-passaged bacteria were re-inoculated into XG.

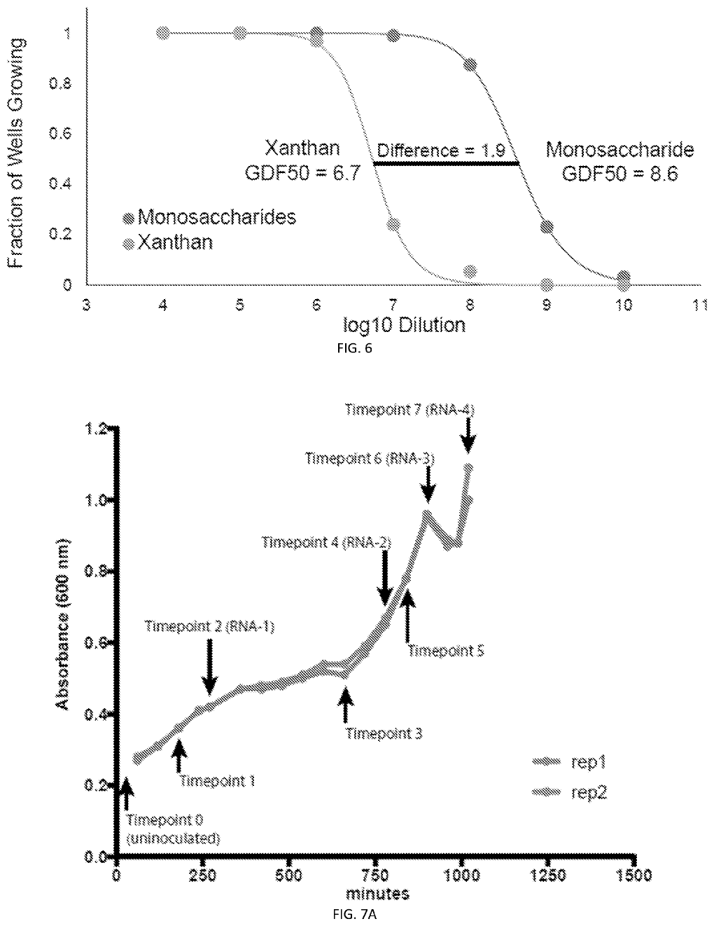

Despite the two most abundant bacteria, including R. UCG13 and a Bacteroides OTU, being present as >20% relative abundance, pure cultures that could degrade XG were unable to be isolated using different solid media effective for Gram-positive and-negative bacteria. Correspondingly, replicate experiments in which the active multi-species community was diluted to extinction in microtiter plates containing medium with either XG, or an equal amount of its component monosaccharides, loss of growth on XG was observed at higher dilutions than simple sugars (growth dilution factor 50 (GDF50): dilution factor at which 50% of wells would grow (FIG. 6). The difference between XG and monosaccharides was an average of 1.8 across n=5 independent experiments (std=0.4; SEM=0.2). Remarkably, when a culture was recovered from the most diluted sample in which XG-degradation was observed and this dilution scheme was repeated again, the twice-diluted culture still contained the 12 original OTUs.

A second survey was completed with a new group of 60 healthy adults. This time, feces were directly sampled within 24 hr after sample collection in anaerobic preservation buffer and using no pre-enrichment or antibiotics. In contrast to the previous results, this experiment revealed that the ability to degrade XG was substantially more frequent, as a greater percentage of people sampled harbored bacterial populations that grew to appreciable levels on XG and decreased its characteristic viscosity. Twenty of these samples were independently passaged 10 times each (one 1:200 dilution per day) and the resulting community structure was analyzed. While all of the passaged cultures contained multiple OTUs (between 12-22 OTUs with relative abundance ≥0.5%) as well as commonalities at the genus level, the only OTU common across all cultures at this threshold was the OTU corresponding to R. UCG13 (FIG. 1D). Collectively, these results suggested that a member of an uncultured Ruminococcaceae genus facilitates XG degradation.

### Example 2

### Xanthan Gum Utilization in R. UCG13 and Bacteroides intestinalis

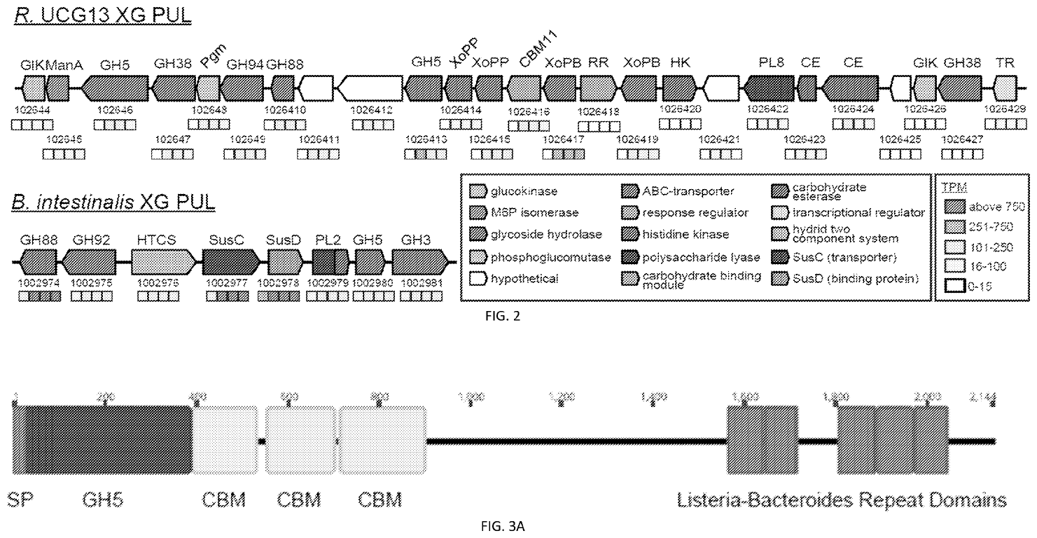

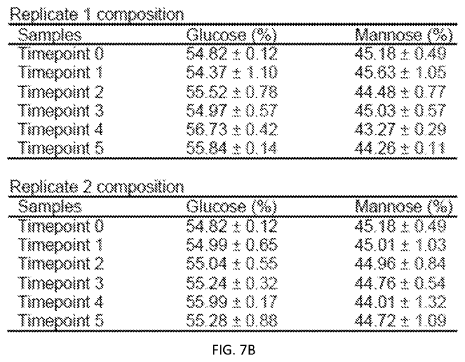

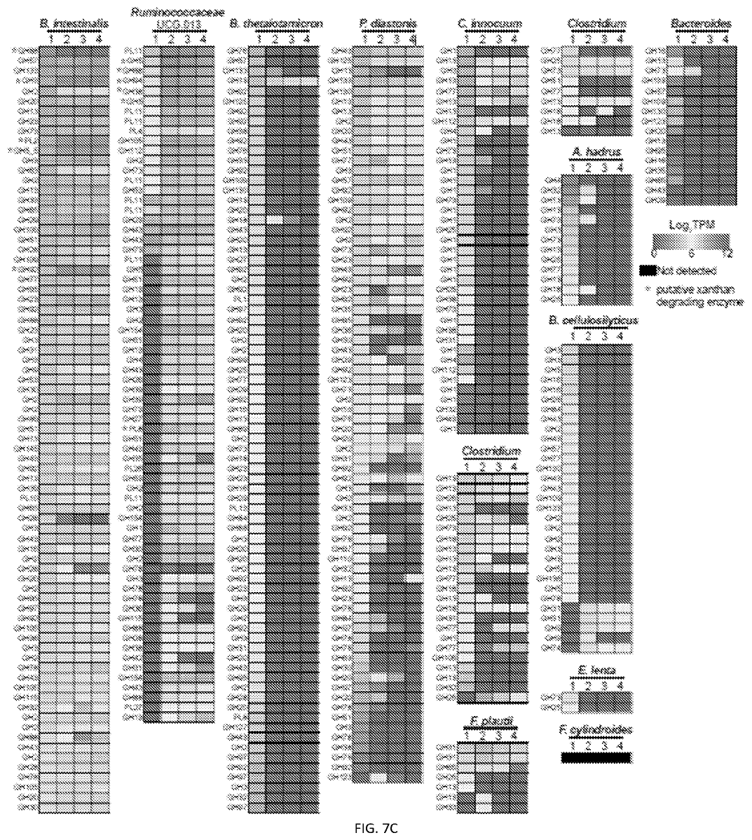

To identify XG-degrading genes within the bacterial consortium, a temporal multi-omic analysis was applied to samples taken from the original XG-degrading culture. Two replicates of the original culture were grown in liquid medium with XG and timepoints were harvested for metagenomic, metatranscriptomic and monosaccharide analysis of residual polysaccharide (FIGS. 7A-7C). From samples harvested at early, intermediate, and late points in growth, metagenome assembled genomes (MAGs) were reconstructed. Taxonomic analysis revealed one specific MAG that was distantly related to the recently cultured bacterium Monoglobus pectinolyticus, which is also the closest relative of the R. UCG13 OTU based on 16S rDNA analysis. Annotation of carbohydrate active enzymes (CAZymes) in this MAG revealed a single locus encoding several highly expressed enzymes that are candidates for XG degradation (FIG. 2, FIGS. 7A-7C). These included a polysaccharide lyase family 8 (PL8) with homology to known xanthan lyases from Paenibacillus nanensis and Bacillus sp. GL1 (FIG. 2).

Xanthan lyases typically remove the terminal pyruvylated mannose prior to depolymerization, leaving a 4,5 unsaturated residue at the glucuronic acid position, although some tolerate non-pyruvylated mannose. This same locus also contained two GH5 endoglucanases with the potential to cleave the xanthan gum backbone, a GH88 to remove the unsaturated glucuronic acid residue produced by the PL8, and two GH38s which could potentially cleave the alpha-D-mannose. Two carbohydrate esterases (CEs) could remove the acetylation from the mannose and possibly the terminal pyruvate, although the latter activity has not been described. SignalP 5.0 predicted SPI motifs for the two GH5s and one of the CEs (1026424, plasmid 13-8D that is an acetylase), while the other enzymes lacked membrane localization and secretion signals. In addition to putative enzymes to cleave the glycosidic bonds contained within xanthan gum, this locus also contained proteins predicted to be involved in sensing, binding, and transporting the released sugars or oligosaccharides.

Colocalization and expression of genes that saccharify a common polysaccharide as discrete polysaccharide utilization loci (PULs) is common in the Gram-negative Bacteroidetes. Although not present in all xanthan gum-degrading cultures, a MAG was obtained for a strain of B. intestinalis, which was the most abundant OTU in the original xanthan culture (up to 51.0% of the original culture, 26.0% and 32.7% in samples 32 and 11 respectively, 8 other samples ranging from 0.3-4.4%). This MAG contained a putative xanthan PUL that was highly expressed during growth on XG (FIG. 2, FIGS. 7A-7C) and encodes hallmark SusC-/SusD-like proteins, a sensor/regulator and predicted GH88, GH92 and GH3 enzymes, which could potentially cleave the unsaturated glucuronyl, α-linked mannose and cellobiose linkages in XG, respectively. Like the candidate gene cluster in R. UCG13, this PUL also contains a GH5 enzyme that could cleave the XG backbone, although such an activity has yet to be described for this family. Finally, a family 2 polysaccharide lyase (PL2) is also present and, while these typically function on galacturonic acid substrates, it may be responsible for removing the terminal mannose. In addition to the lyase domain, this multi-modular protein contains a carbohydrate esterase domain (CE) that could remove the acetyl groups positioned on the mannose. Extensive work has been conducted to characterize the substrate-specificity of PULs, which is demonstrated by hundreds of genomes with characterized and predicted PULs in the PUL database (PUL-DB). However, this database only harbored a single genome with a partially related homolog of the B. intestinalis PUL (B. salyersiae WAL 10018 PUL genes HMPREF1532_01924-HMPREF1532_01938).

Although less dramatic, several microbes showed increased expression of CAZymes over the culture time course, suggesting that other microbes may cross-feed on either XG oligosaccharides released by the primary degraders, or on additional substrates produced by XG consumers (FIGS. 7A-7C). Interestingly, neutral monosaccharide analysis showed a relatively stable 1:1 ratio of glucose:mannose in residual polysaccharide in the culture, suggesting that lyase-digested xanthan gum was not accumulating as growth progressed (FIGS. 7A-7C).

### Example 3

### R. UCG13 Encodes Enzymes with Xanthanase Activity

To investigate the cellular location of the enzymes responsible for xanthan degradation, the original culture was grown in XG medium and separated into filtered cell-free supernatant, cells that were washed to remove supernatant and resuspended or lysed, or lysed cells with supernatant. Incubation of these fractions with XG and subsequent analysis by thin layer chromatography (TLC) revealed that the cell-free supernatant was capable of depolymerizing XG into large oligosaccharides, while the intracellular fraction was required to further saccharify these products into smaller components. Liquid chromatography-mass spectrometry (LC-MS) analysis of the cell-free supernatant incubated with XG revealed the presence of pentameric oligosaccharides matching the structure of xanthan gum.

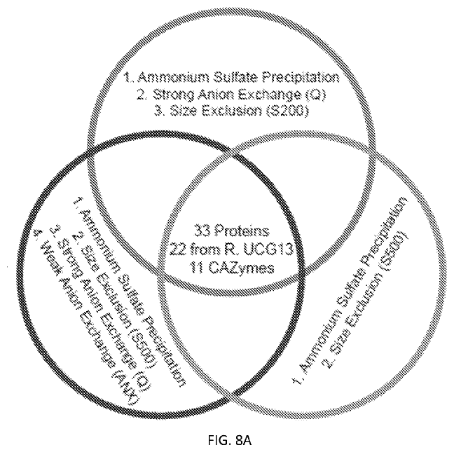

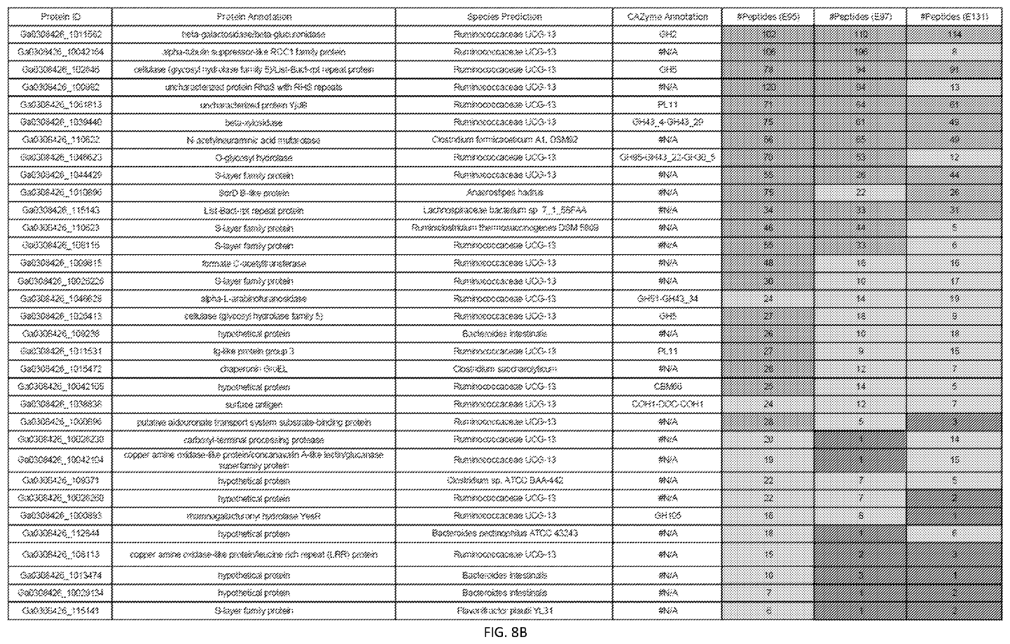

Three independent cultures were grown in liquid medium containing XG and cell-free supernatants were subjected to ammonium sulfate precipitation. Each of the resuspended protein preparations was able to hydrolyze XG as demonstrated by a complete loss of viscosity overnight. Each sample was fractionated with a variety of purification methods, collecting and pooling xanthan-degrading fractions for subsequent purification steps and taking three different purification paths (FIG. 8A). The purest sample obtained ran primarily as a large smear when loaded onto an SDS-PAGE gel, but separated into distinct bands after boiling, indicating possible formation of a multimeric protein complex, which is reminiscent of cellulosomes. Proteomic analysis of the samples from the three different activity-guided fractionation experiments yielded 33 proteins present across all three experiments, including 22 from R. UCG13, 11 of which were annotated as CAZymes (FIG. 8B). While most of the proteins were either detected in low amounts or lacked functional predictions consistent with polysaccharide degradation, one of the most abundant proteins across all three samples was the GH5 previously identified in the R. UCG13 xanthan locus.

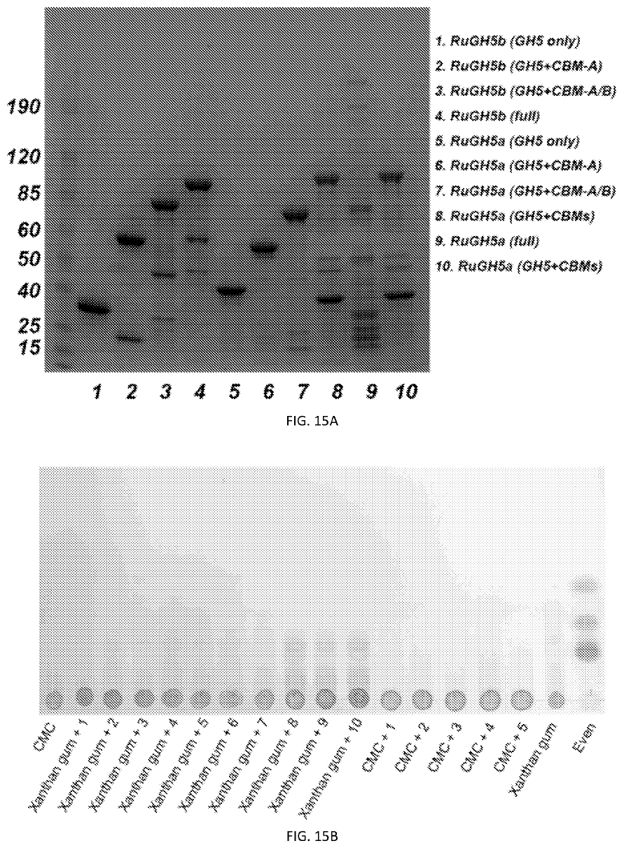

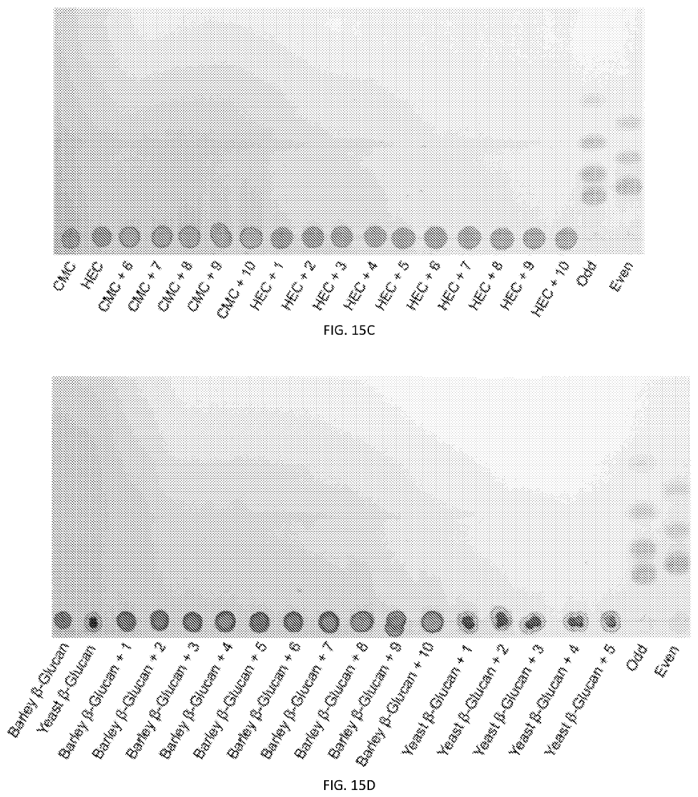

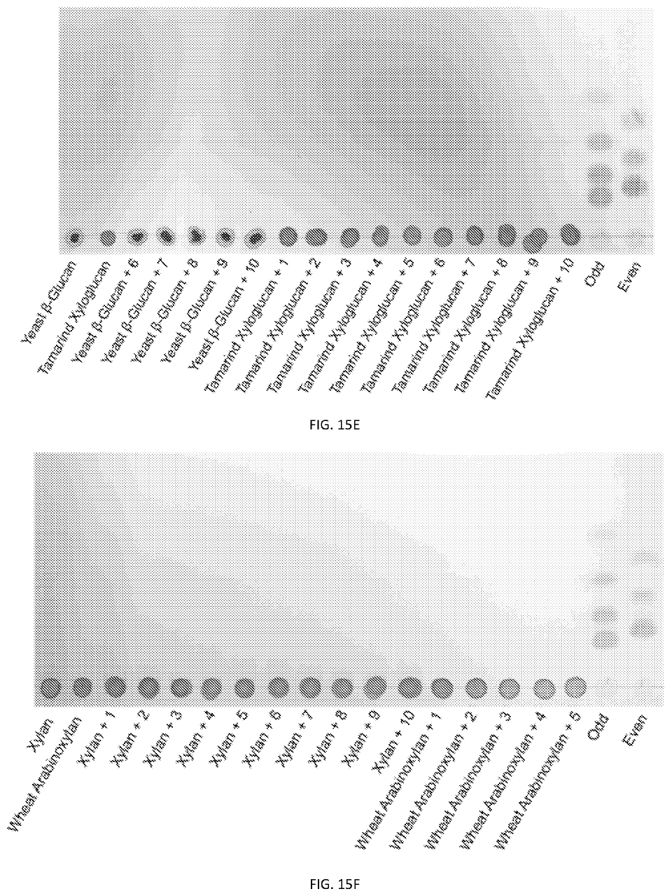

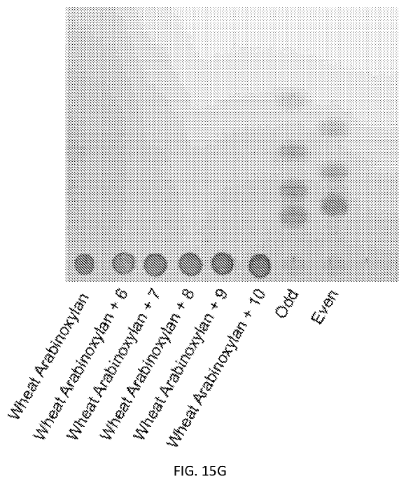

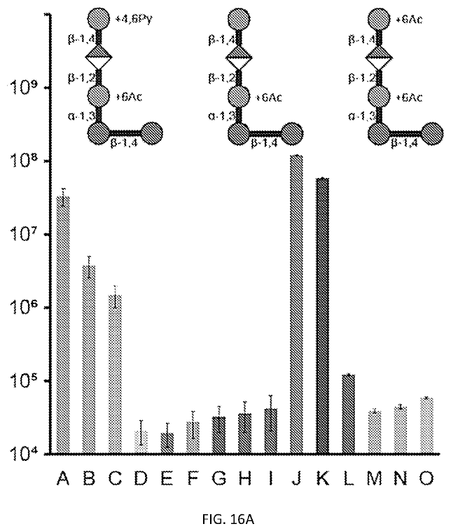

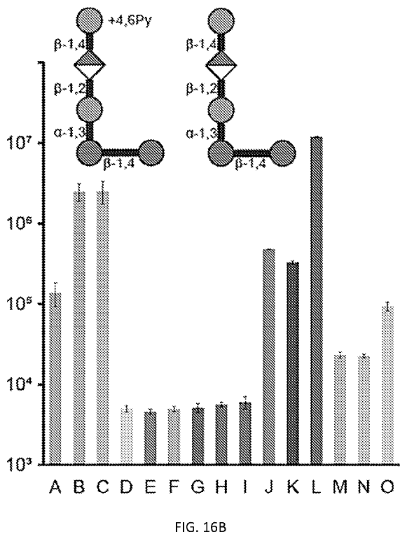

The R. UCG13 GH5 consists of an N-terminal signal peptide sequence, its main catalytic domain which does not classify into any of the GH5 subfamilies, and 3 tandem carbohydrate binding modules (CBMs), which are often associated with CAZymes and assist in polysaccharide degradation (FIG. 3A). The protein also contains a significant portion of undefined sequence and Listeria-Bacteroides repeat domains (PF09479), a β-grasp domain originally characterized from the invasion protein InlB used by Listeria monocytogenes for host cell entry. These small repeat domains are generally thought to be involved in protein-protein interactions and are almost exclusively found in extracellular bacterial multidomain proteins. Recombinant forms of the entire protein, the GH5 domain only, and the GH5 domain with either one (CBM-A), two (CBM-A and CBM-B), or all three of the CBMs (A-C) were expressed. All but the full-length construct yielded reasonably pure proteins, but only the construct with the GH5 and all three CBMs showed activity on xanthan gum (FIG. 15). An alternate GH5 (R. UCG13 GH5b) was also expressed in a variety of forms but did not display any activity on XG (FIG. 15).

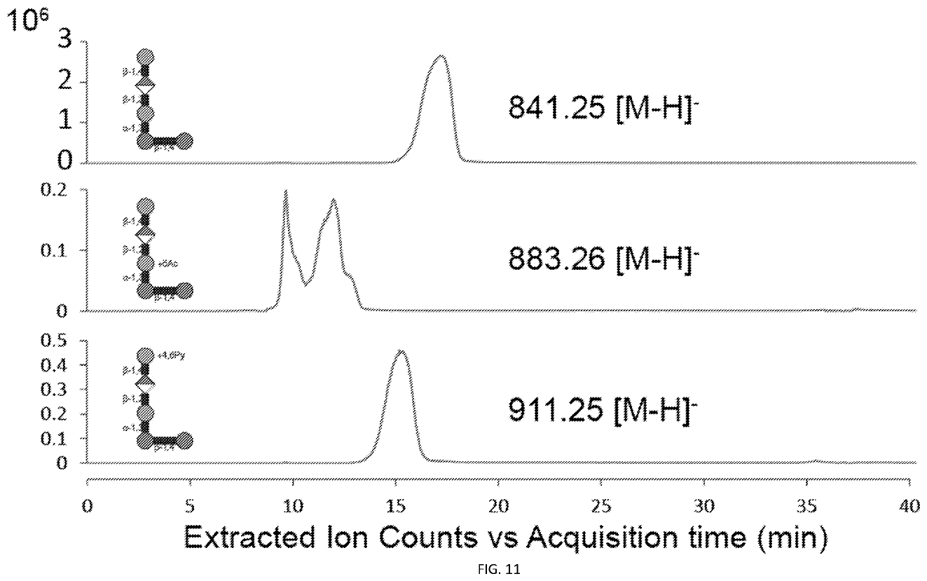

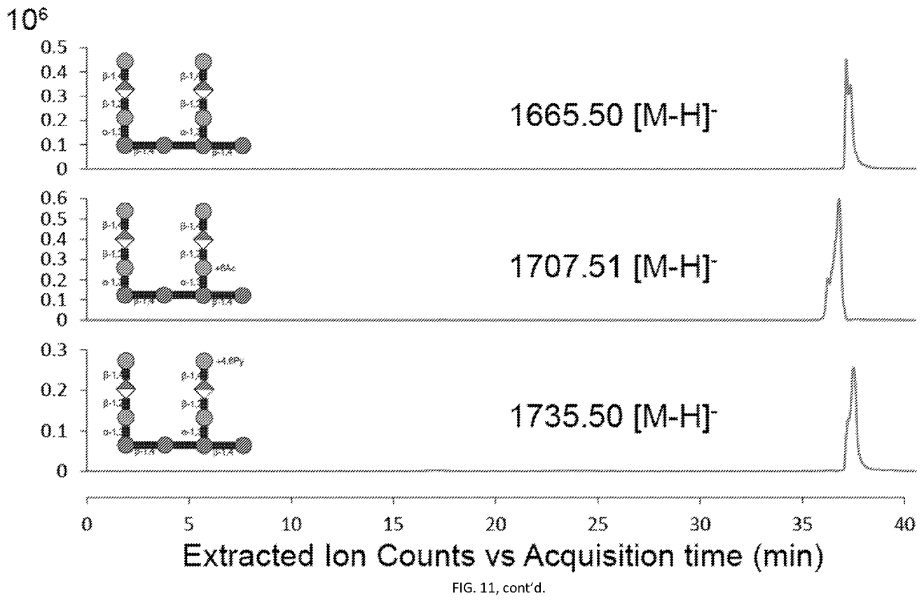

Analysis of the reaction products showed that R. UCG13 GH5 (R. UCG13 GH5a) releases pentasaccharide repeating units of XG, with various acetylation and pyruvylation (including di-acetylation as previously described), and larger decasaccharide structures (FIGS. 3B and 11). While isolation of homogenous pentameric oligosaccharides proved difficult, coincubation of XG with R. UCG13 GH5 and a Bacillus sp. PL8 facilitated isolation of pure tetrasaccharide, followed by in-depth 1D and 2D NMR structural characterization, which was useful in determining the GH5 cut site in the XG backbone. Surprisingly, GH5 cleaved XG at the reducing end of the non-branching backbone glucosyl residue (FIG. 3C). This contrasts with material produced by other known xanthanases (such as the GH9 from Paenibacillus nanensis or the β-D-glucanase in Bacillus sp. strain GL1), that hydrolyze xanthan at the reducing end of the branching glucose. While R. UCG13 GH5 displayed little activity on other polysaccharides (FIG. 15), it was able to hydrolyze both native and lyase-treated XG with comparable specificity, once more in contrast to most previously known xanthanases, which show ≥600 fold preference for the lyase-treated substrate (FIG. 3D). One exception is the xanthanase from Microbacterium sp XT11, which also cleaves native and lyase-treated xanthan gum with similar kinetic specificity; however, this enzyme only produces intermediate XG oligosaccharides, whereas R. UCG13 can cleave XG down to its repeating pentasaccharide moiety.

### Example 4

### B. intestinalis Cross-Feeds on XG Oligos with its Xanthan Utilization PUL

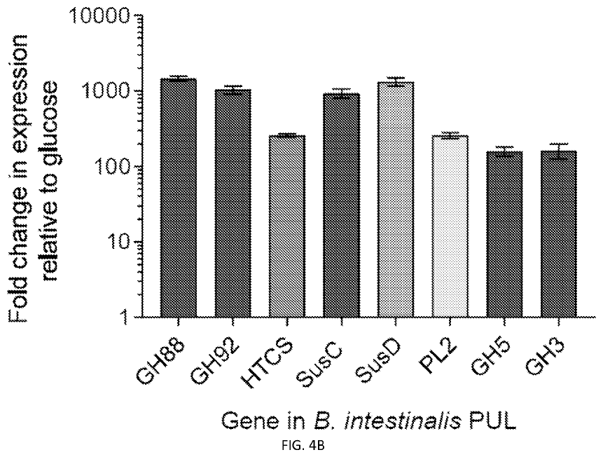

Although R. UCG13 was recalcitrant to culturing efforts, several bacteria were isolated from the original consortium, including the Bacteroides intestinalis strain that was the most abundant (FIG. 1C) and also had a highly expressed candidate PUL for XG degradation (FIG. 2). While this strain was unable to grow on native XG as a substrate, it may be equipped to utilize smaller XG fragments, such as those released by R. UCG13 during growth via its GH5 enzyme. Using the recombinant R. UCG13 GH5, sufficient quantities of mixed XG oligosaccharides (XGOs) (primarily pentameric, but also some decameric oligosaccharides) were generated to test growth of Bacteroides intestinalis. While isolates of P. distasonis and B. clarus from the same culture showed little or no growth (FIG. 17), the B. intestinalis strain achieved comparable density on the XG oligosaccharides as cultures grown on a stoichiometric mixture of the monosaccharides that compose XG, suggesting that it uses most or all of the sugars contained in the oligosaccharides (FIG. 4A) All of the genes in this locus were activated >100-fold (and some >1000-fold) during growth on XG oligosaccharides compared to glucose reference (FIG. 4B). Whole genome RNA-seq analysis of the B. intestinalis strain grown on XGOs revealed that the identified PUL was the most highly upregulated in the genome, validating its role in metabolism of XGOs (FIG. 17). Interestingly, R. GH5 XGOs treated with PL8 continued to support B. intestinalis growth, but tetramer generated from the P. nanensis GH9 and PL8 failed to support any growth (FIG. 17). Growth was rescued in the presence of glucose but not in the presence of Ru GH5a XGOs to upregulate the PUL (FIG. 17), suggesting that either the B. intestinalis transporters or enzymes are incapable of processing this alternate substrate.

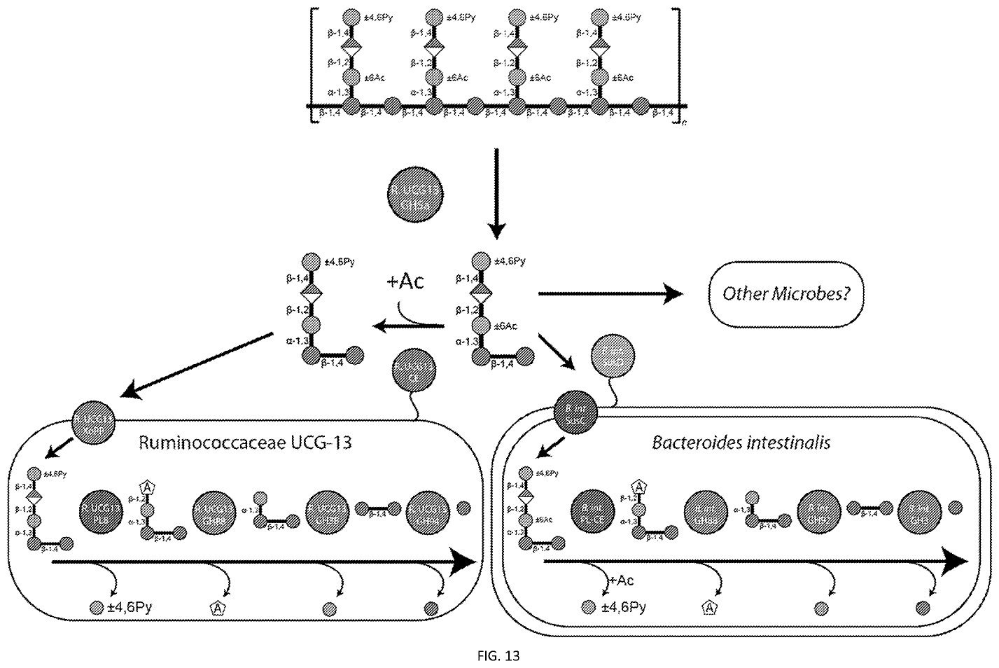

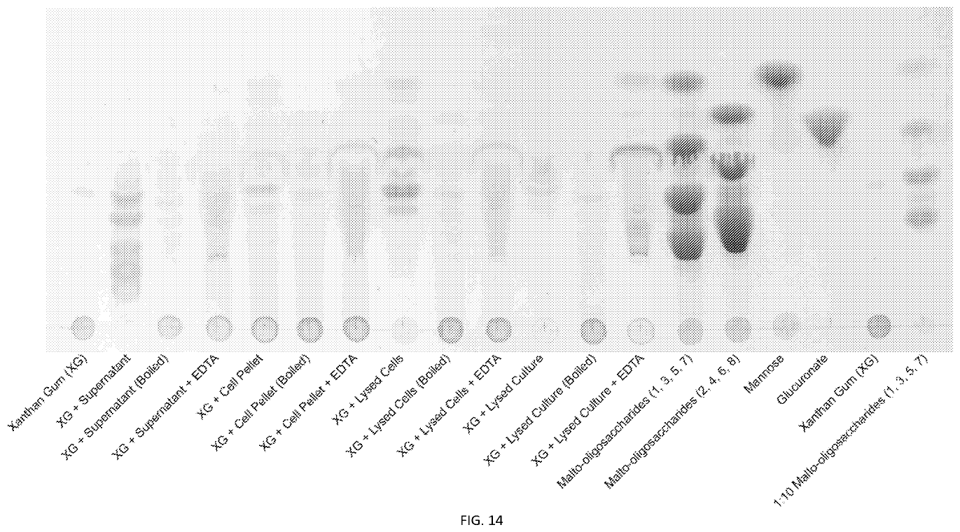

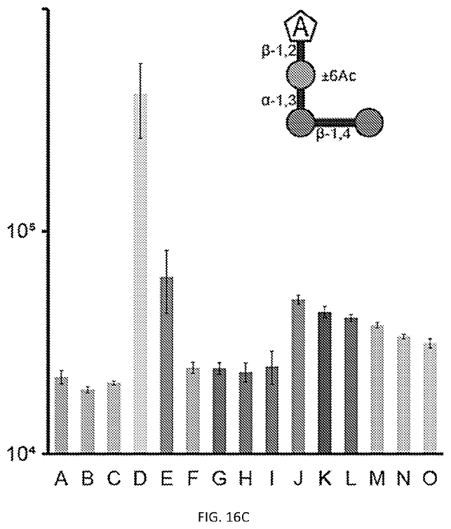

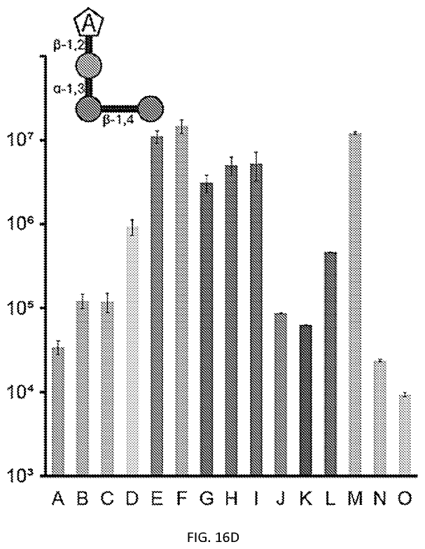

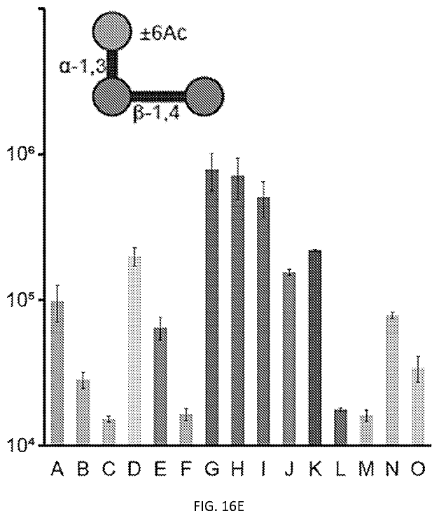

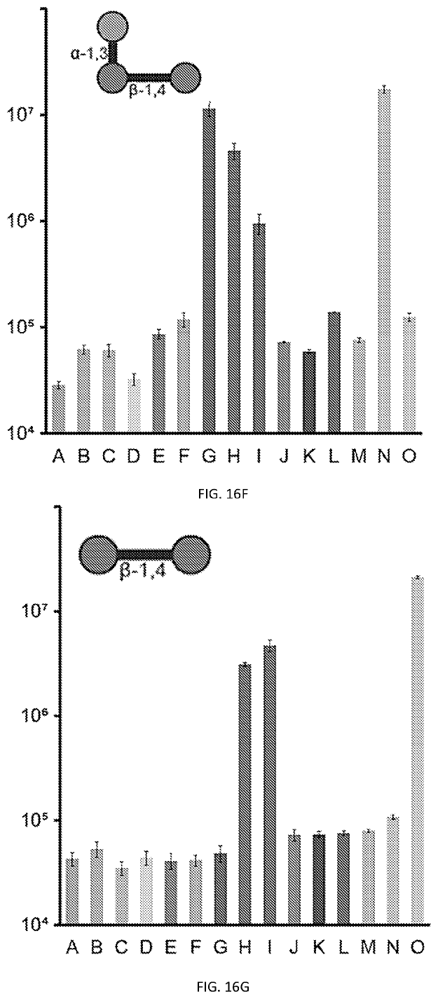

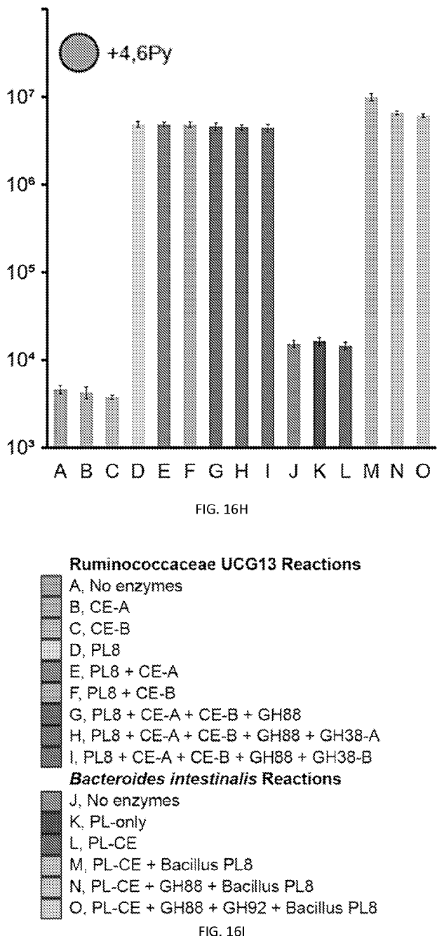

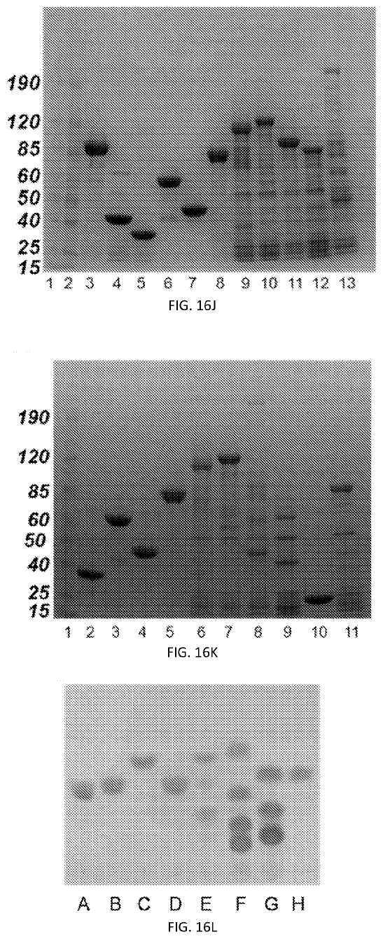

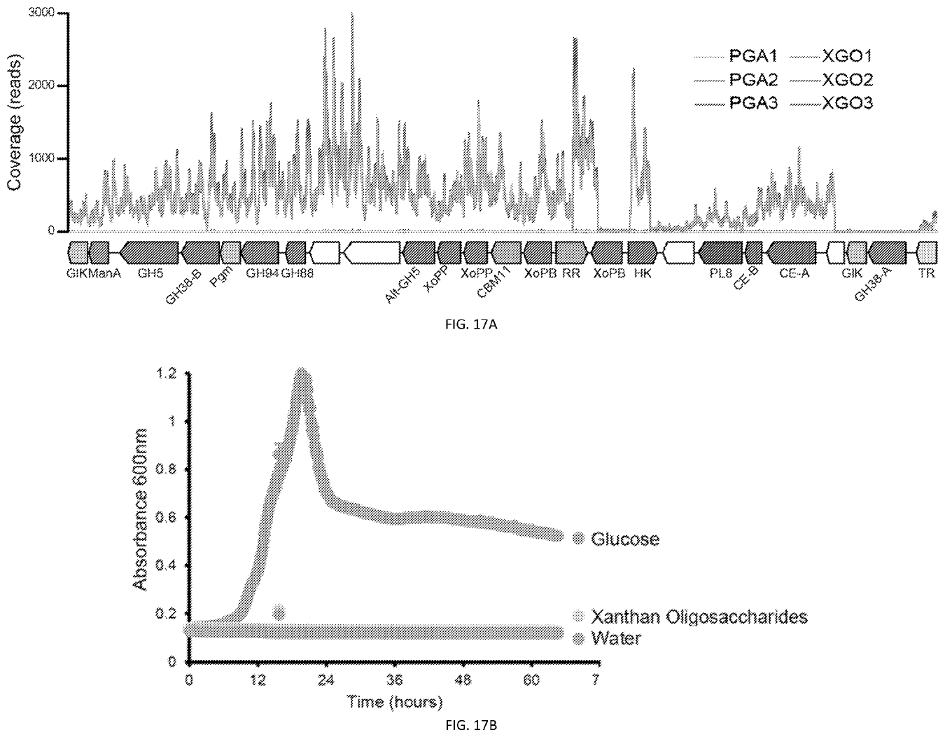

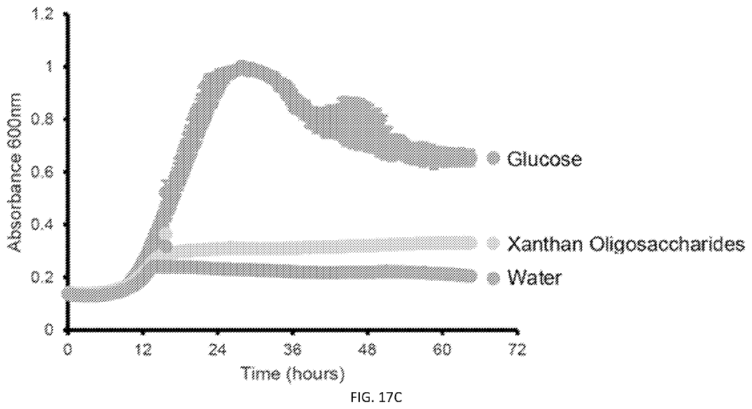

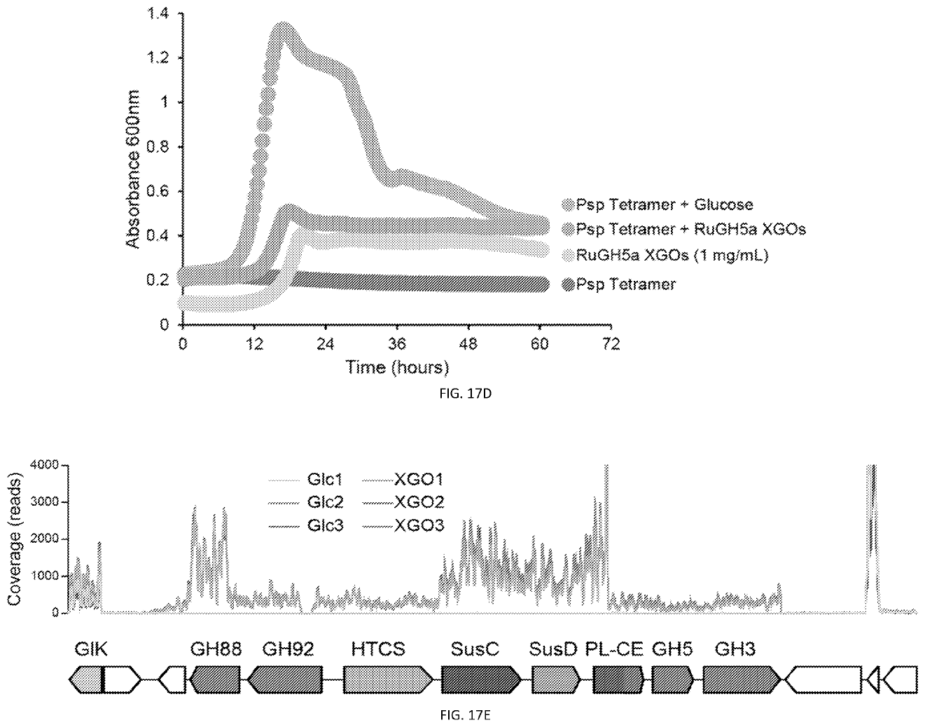

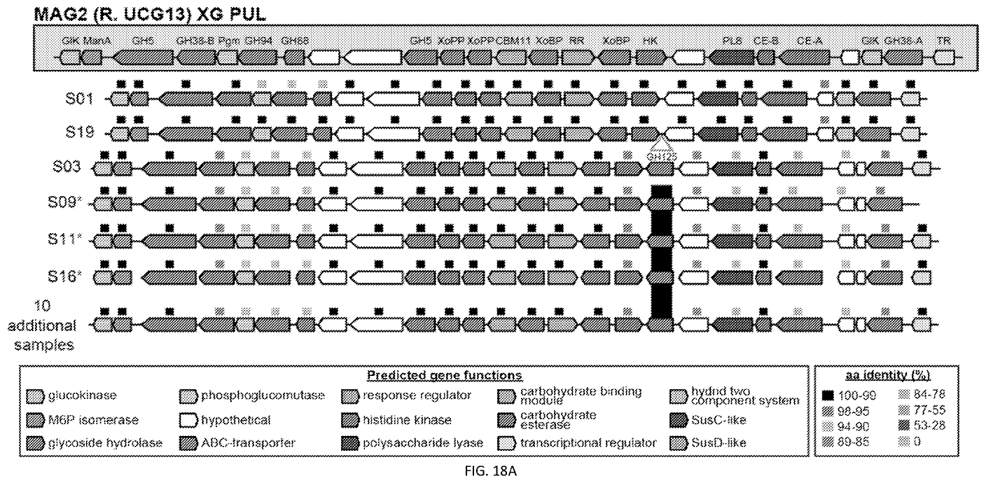

To further test the role of the identified B. intestinalis PUL in XG degradation, the recombinant forms of the enzymes it contains were tested for XG degradation. The carbohydrate esterase domain C-terminal to the PL2 bimodular protein was able to remove acetyl groups from acetylated xanthan pentasaccharides (FIG. 16). Xanthan lyase activity was unable to be detected for the PL2 enzyme on full length XG or oligosaccharides, thus it is likely that this enzyme or another lyase acts to remove the terminal mannose residue since the GH88 was able to remove the corresponding 3,4 unsaturated glucuronic acid residue from the corresponding tetrasaccharide that would be generated by its action (FIG. 16). The GH88 reaction proceeded irrespective of the acetylation state of the mannosyl residue. The GH92 was active on the trisaccharide produced by the GH88 as observed by loss of the trisaccharide and formation of cellobiose in these reactions (FIG. 16). Finally, the GH3 was active on cellobiose, but did not show activity on either tri- or tetra-saccharide, suggesting that this enzyme may be the final step in B. intestinalis saccharification of xanthan oligos (FIG. 16). SignalP 5.0 predicted SPII signals for the GH5, GH3, GH88, and SusD proteins while the GH92, PL2, HTCS, and SusC all had SPI motifs. While signal peptides do not definitively determine cellular location, these predictions and accumulated knowledge of Sus-type systems in Bacteroidetes suggest a model in which saccharification occurs primarily in the periplasm (FIG. 13).

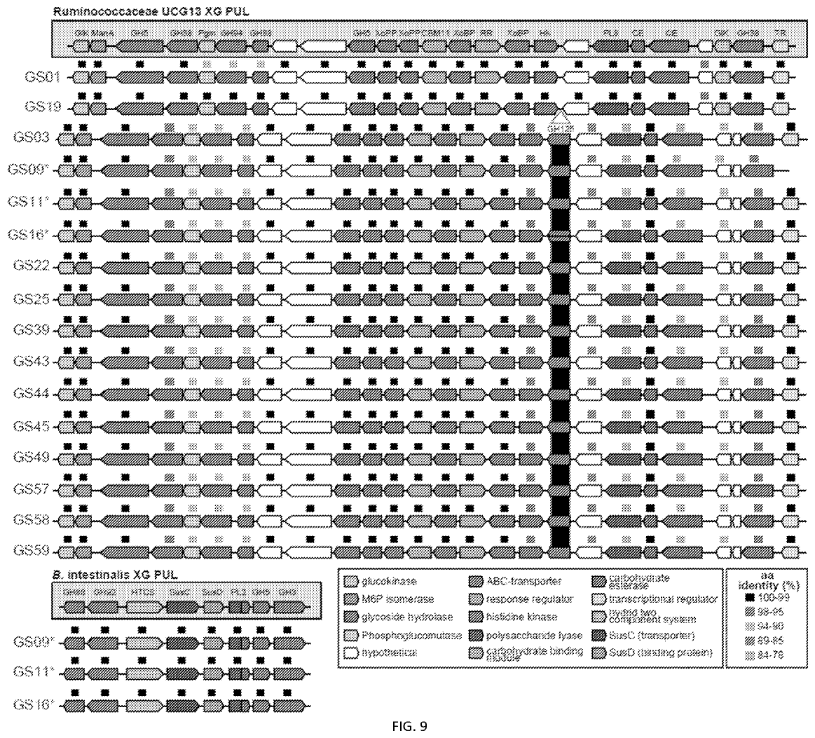

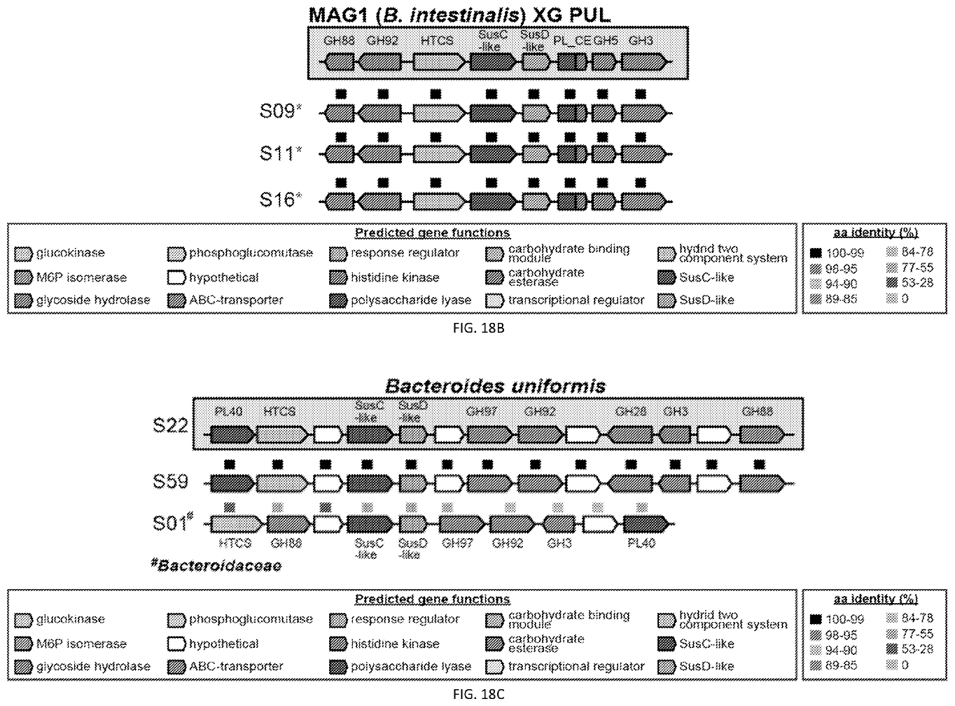

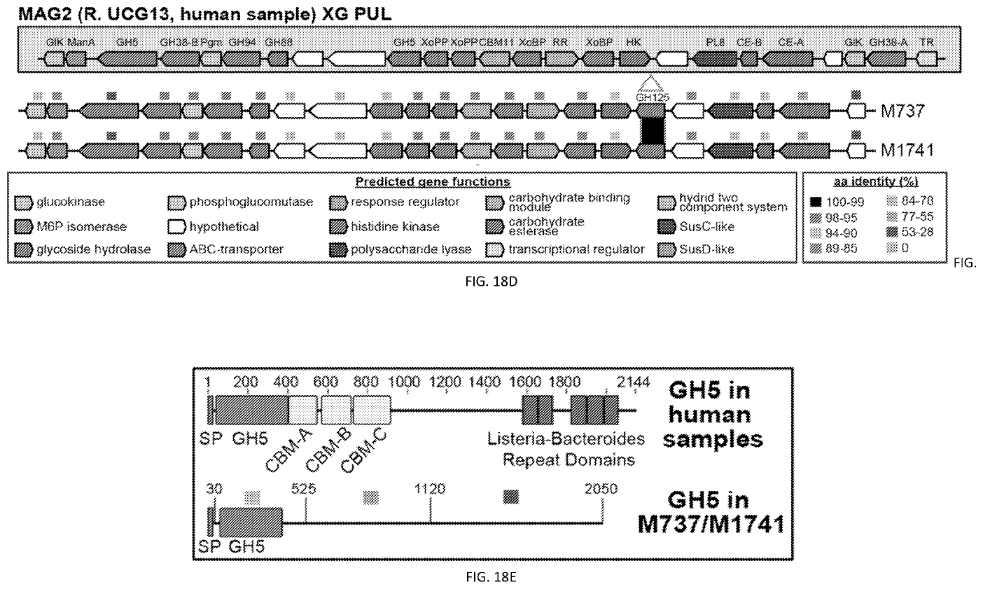

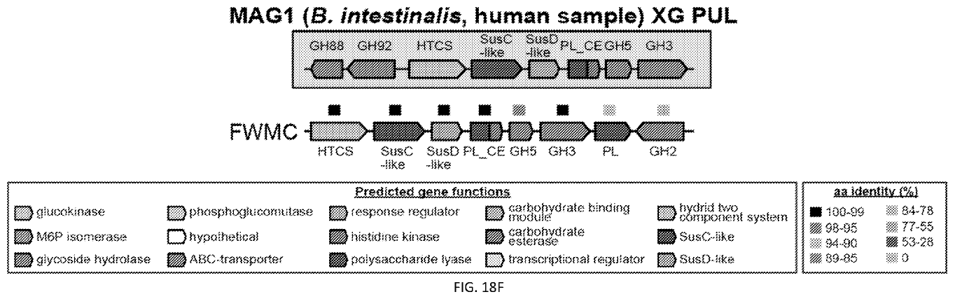

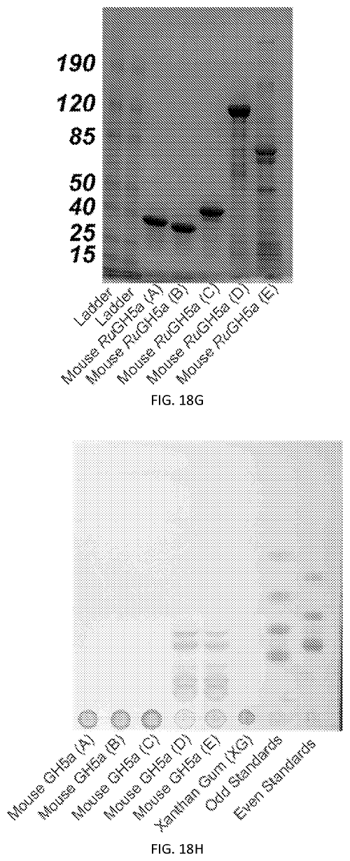

Additional metagenomic sequencing was performed on 20 additional XG-degrading communities and it was found that the R. UCG13 XG utilization locus is extremely well conserved across these cultures with high amino acid identity and only one variation in gene content, insertion of a GH125 coding gene (FIG. 9) (FIG. 18). The additional GH125 gene was observed in most of the loci (14/17), suggesting that this gene provides a complementary, but non-essential function, possibly as an accessory α-mannosidase. In contrast, only a subset of the samples (4/17) contained the B. intestinalis PUL, which showed essentially complete conservation in xanthan cultures that contained this PUL (FIG. 9). Across all these cultures, R. UCG13 accounted for an average of only 23.1%±1.2 (SEM) of the total culture (FIG. 1D), suggesting that additional microbes beyond B. intestinalis have the ability to cross-feed on products released by R. UCG13, either from degradation products of XG or by using other growth substrates generated by R. UCG13. For example, the bacterial communities in samples 1, 22, and 59 contained other microbes belonging to the Bacteroidaceae family that harbor a PUL with a GH88, GH92, and GH3, suggesting that these bacteria can metabolize XG-derived tetramers (FIG. 18).

### Example 5

### Engineering Xanthan Gum Utilization Loci into Other Microbes for Rationally Designed Probiotics

Bacteroides intestinalis Xanthan Gum Utilization Locus. Primers are designed and used to amplify the entire B. intestinalis xanthan gum utilization locus, with overlapping ends to facilitate assembly. PCR fragments of the locus are assembled and circularized into the linearized Bacteroides genomic insertion vector, pNBU2, using Gibson assembly and the NEBuilder HiFi DNA Assembly kit. The pNBU2 vector can be used to insert DNA into one of two tRNA-Serine sites in numerous Bacteroides genomes (Martens, E. C., et al., Cell Host Microbe 4, 447-457 (2008), incorporated herein by reference). After assembly and transformation into Lucigen TransforMax EC100D pir+ electrocompetent E. coli, the plasmid is transformed into S17-1 1 pir E. coli for conjugation into Bacteroides thetaiotaomicron and additional Bacteroides spp by conjugation. B. theta strains with the inserted xanthan utilization locus are tested for the ability to grow on xanthan gum oligosaccharides, indicative of gain of function. Strains that successfully grow on xanthan oligosaccharides with the transferred/engineered locus are tested for their abilities to colonize animal digestive tracts and the pre-existing gut microbiome, the dose (cfu/ml by oral gavage or lyophilized bacteria in capsule) of invading, recombinant B. theta and the dosage of xanthan pentasaccharides administered to the animals can be systematically varied.

The Ruminococcaceae UCG13 GH5-30 enzyme can be transferred into Bacteroides spp. This is accomplished by genetically engineering an insertion of this gene into the B. intestinalis PUL that confers xanthan oligosaccharide metabolism thereby making expression of the GH5-30 gene regulated the same as other xanthan-degrading functions. To adapt this enzyme to be expressed on the surface of the Gam-negative Bacteroides cell, its native secretion signals are removed and recombined with an N-terminal domain of the B. theta surface protein SusF, for which the signal sequence required for secretion and trafficking to the cell surface has been determined. This process results in an active extracellular GH5-30 capable of depolymerizing xanthan gum and engineered Bacteroides that are not only capable of utilizing xanthan oligosaccharides but are fully capable of depolymerizing and growing on native xanthan gum.

Ruminococcaceae UCG13 Xanthan Gum Utilization Locus Gram-positive microbes are potentially superior organisms for production of secreted peptides and proteins. The minimal xanthan gum utilization locus from R. UCG13 may be transferred to Gram positive microbes that are genetically tractable, including but not limited to Lactobacillus reuteri and Clostridium scindens to engineer gram-positive probiotics that can successfully colonize the gastrointestinal tract with co-feeding of xanthan gum.

### Example 6

### R. UCG13 Encodes Enzymes Required for XG Saccharification

In contrast to characterized PL8 xanthan lyases, the R. UCG13 PL8 showed no activity on the complete XG polymer but removed the terminal mannose from xanthan pentasaccharides produced by R. UCG13 GH5 (FIG. 16). This further supports the model in which the GH5 first depolymerizes XG, followed by further saccharification of the XG repeating unit, likely inside the cell. Both R. UCG13 carbohydrate esterases were able to remove acetyl groups from acetylated xanthan pentasaccharides (FIG. 16). The tetrasaccharide produced by the PL8 was processed by the GH88 and both GH38s, which were able to saccharify the resulting trisaccharide (FIG. 16). The GH94 catalyzed the phosphorolysis of cellobiose in phosphate buffer, completing the full saccharification of XG (FIG. 16). Apparent redundancy of several enzymes (CEs and GH38s) could be partially explained by different cell location (e.g., CE-A has an SPI signal while CE-B does not), unique specificities for oligosaccharide variants in size or modification (e.g., acetylation or pyruvylation), additional polysaccharides that the locus targets, or evolutionary hypotheses where this locus is in the process of streamlining or expanding. Additional support for the involvement of this locus in XG degradation was provided by RNA-seq based whole genome transcriptome analysis, which showed the induction of genes in this cluster when the community was grown on XG compared to another polysaccharide (polygalacturonic acid, PGA) that also supports R. UCG13 abundance (FIG. 17).

### Example 7

### Xanthan Utilization Loci are Widespread in Modern Microbiomes

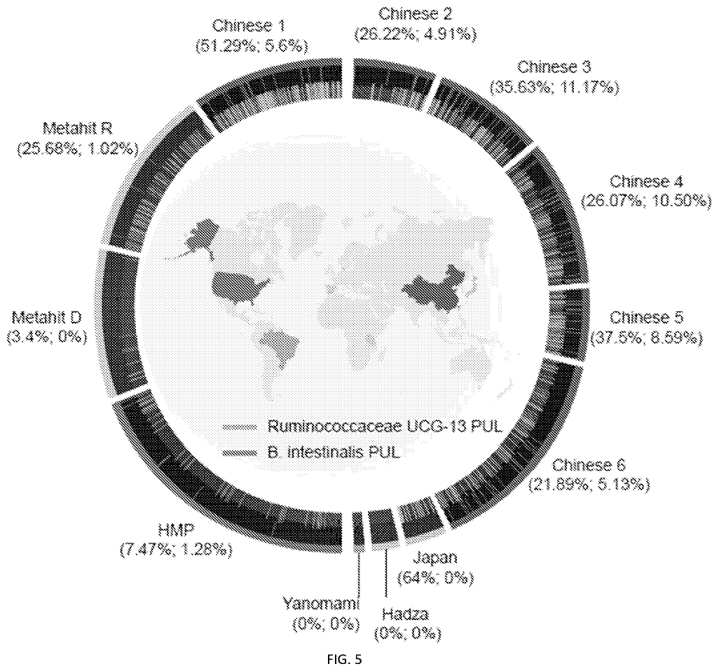

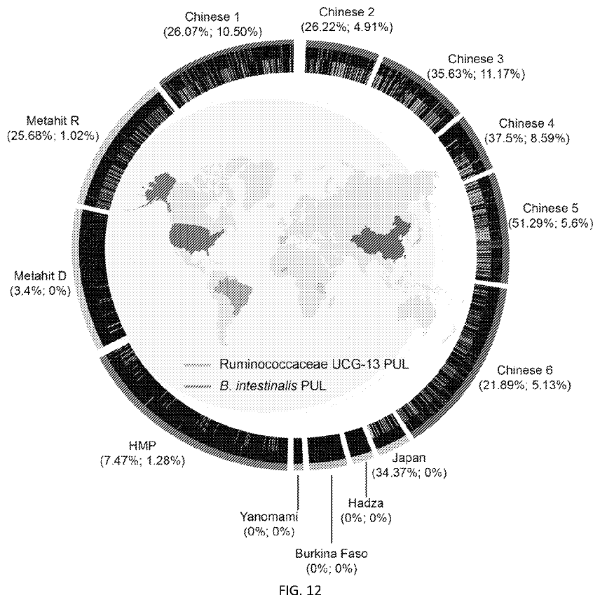

Using each locus as a query, several publicly available fecal metagenome datasets collected from worldwide populations were searched. All modern populations sampled displayed some presence of the R. UCG13 XG locus, with the Chinese and Japanese cohorts being the highest (up to 51% in one cohort) (FIGS. 5 and 12). The B. intestinalis locus was less prevalent, with two industrialized population datasets (Japan and Denmark/Spain) lacking any incidence. Where the locus was present, its prevalence ranged from 1-11%. The three hunter-gatherer or non-industrialized populations sampled, the Yanomami, Hadza, and Burkina Faso had no detected presence of either the R. UCG13 or B. intestinalis locus.

Although the size of the hunter-gatherer datasets is relatively small, excluding the possibility of a false negative suggests several equally intriguing hypotheses. Most obviously, inclusion of XG in the modern diet may have driven either the colonization or expansion of R. UCG13 (and to a lesser extent B. intestinalis) into the gut communities of numerous human populations. This is in concordance with previous observations that found that a set of volunteers fed xanthan gum for an extended period produced stool with increased probability and degree of xanthan degradation. Alternatively, the modern microbiome is drastically different than that of hunter-gatherers and these differences simply correlate with the abundance of R. UCG13, rather than any causal effect of XG in the diet. Another possible hypothesis is that the microbiomes of hunter-gatherer populations can degrade XG but use completely different microbes and pathways.

To further probe the presence of the identified XG utilization genes in other environments, an expanded LAST search of both loci was conducted in 72,491 sequenced bacterial isolates and 102,860 genome bins extracted from 13,415 public metagenomes, as well as 21,762 public metagenomes that are part of the Integrated Microbial Genomes & Microbiomes (IMG/M) database using fairly stringent thresholds of 70% alignment over the query and 90% nucleotide identity. This search yielded 35 hits of the R. UCG13 locus in human microbiome datasets, including senior adults, children, and an infant (12-months of age, Ga0169237_00111). 12 hits of the B. intestinalis XGOs locus were also found, all in human microbiome samples except for a single environmental sample from a fracking water sample from deep shales in Oklahoma, USA (81% coverage, 99% identity) (FIG. 18). XG and other polysaccharides such as guar gum are used in oil industry processes, and genes for guar gum catabolism have previously been found in oil well associated microbial communities. Since most samples searched were non-gut-derived, this demonstrates that XG-degrading R. UCG13 and XGOs-degrading B. intestinalis are largely confined to gut samples and can be present across the human lifetime.

### Example 8

### Mammalian Microbiomes Harbor Xanthan Utilization Loci

To investigate the prevalence of XG-degrading populations beyond the human gut microbiome, a mouse experiment using feed with 5% XG showed increased levels of short chain fatty acids propionate and butyrate, suggesting the ability of members of the mouse microbiome to catabolize and ferment XG43. After culturing mouse feces from this experiment on XG media and confirming its ability to depolymerize XG, the community structure in two samples (M1741 and M737) was metagenomically characterized, revealing a microbial species related to R. UCG13 (AAI values between the human R. UCG13 and the mouse R. UCG13 were 75.7% and 75.2% for M1741 and M737, respectively) as well as a XG locus with strikingly similar genetic architecture to the human XG locus (FIG. 18). Although several genes are well conserved across both the human and mouse isolates, significant divergence was observed in the sequences of the respective R. UCG13 GH5 proteins that, based on data with the human locus, initiate XG depolymerization. Specifically, this divergence was more pronounced in the non-catalytic and non-CBM portions of the protein suggesting that while the XG-hydrolyzing functions have been maintained, other domains may be more susceptible to genetic drift. As with the human R. UCG13 GH5, recombinant versions of the mouse R. UCG13 GH5 were able to hydrolyze XG (FIG. 18H) but did not show significant activity on a panel of other polysaccharides. The GH5-only constructs did not degrade XG but constructs D and E (with regions homologous to the human RuGH5a CBMs) were able to hydrolyze XG. Of note, the engineered, truncated protein, construct E showed similar XG hydrolytic activity as that of the full-length protein, construct D.

An additional targeted search of the R. UCG13 locus in several animal- and plant-associated microbiomes was performed and homologous loci were found in both cow (5 positive samples) and goat (one positive sample) microbiomes. Together, these data show that the R. UCG13 XG locus is more broadly present in mammalian gastrointestinal microbiomes.

### Example 9

### B. salyersiae Cross-Feeds on XG Oligos with its Xanthan Utilization PUL

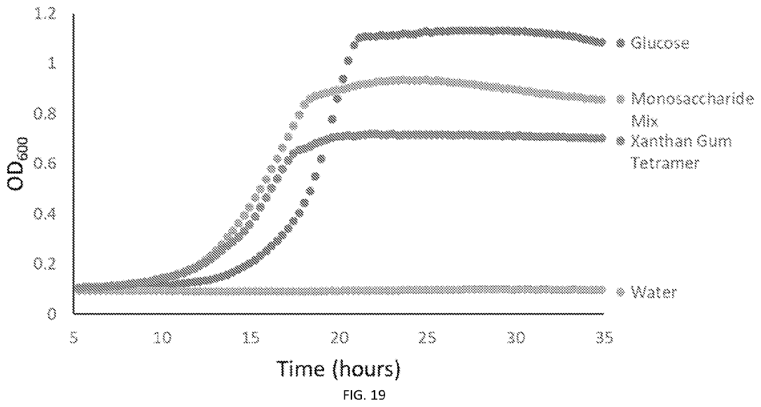

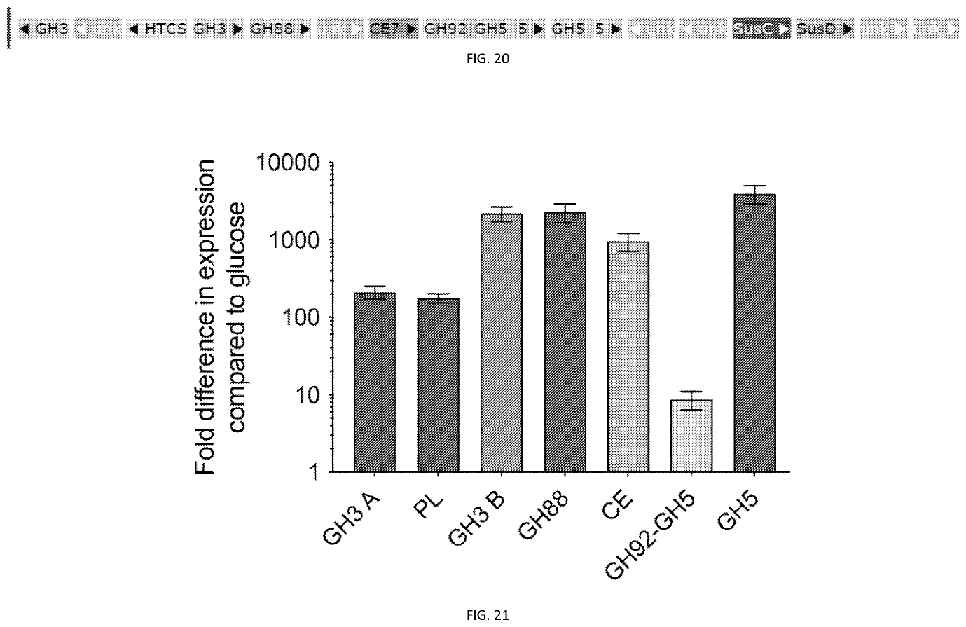

Another strain that had a candidate PUL for XG degradation was B. salyersiae (FIG. 20). Using the recombinant R. UCG13 GH5, as described above for B. intestinalis, sufficient quantities of mixed XG oligosaccharides (XGOs) (primarily pentameric, but also some decameric oligosaccharides) were generated to test growth of B. salyersiae. B. salyersiae utilizes, albeit partially, xanthan gum oligosaccharides treated with xanthan lyase (FIG. 19).

To further test the role of the identified B. salyersiae PUL in XG degradation, the gene expression of the enzymes was tested when grown on XGOs. As shown in FIG. 21, each of the putative enzymes from the PUL was overexpressed when grown on XGOs as compared to glucose, suggestive of a role for these enzymes in catabolizing xanthan gum oligosaccharides.

It is understood that the foregoing detailed description and accompanying examples are merely illustrative and are not to be taken as limitations upon the scope of the disclosure, which is defined solely by the appended claims and their equivalents. Various changes and modifications to the disclosed embodiments will be apparent to those skilled in the art and may be made without departing from the spirit and scope thereof.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

Sequences:
SEQ ID NO: 1. - Rucg13 GH5 domain
KIVKQGTDEMVVLRGVNVPSMDWGMAEHLFESMTMVYDSWGANLIRLPINPKYWKNGSV
WDEKNLTKEQYQKYIDDMVKAAQARGKYIILDCHRYVMPQQDDLDMWKELAVKYGNNS
AVLFGLLNEPHDIKPVGVEKPTTVEQWDVWYNGGQIIVGGEEVTAIGHQQLLNEIRKQGAN
NICIAGGLNWAFDISGFADGYNERPNGYRLIDTAEGHGVMYDSHAYPVKGAKTAWDTIIGP
VRRVAPVIIGEWGWDSSDKNISGGDCTSDIWMNQIMNWMDDTDNQYDGIPVNWTAWNLH
MSS
SEQ ID NO: 2 - Truncated xanthanase
MEEAAADAQNAEINYNRSVPLEVKGNKIVKQGTDEMVVLRGVNVPSMDWGMAEHLFESM
TMVYDSWGANLIRLPINPKYWKNGSVWDEKNLTKEQYQKYIDDMVKAAQARGKYIILDC
HRYVMPQQDDLDMWKELAVKYGNNSAVLFGLLNEPHDIKPVGVEKPTTVEQWDVWYNG
GQIIVGGEEVTAIGHQQLLNEIRKQGANNICIAGGLNWAFDISGFADGYNERPNGYRLIDTAE
GHGVMYDSHAYPVKGAKTAWDTIIGPVRRVAPVIIGEWGWDSSDKNISGGDCTSDIWMNQI
MNWMDDTDNQYDGIPVNWTAWNLHMSSSPKMLYSWDYKTTAYNGTHIKNRLLSYNTAP
EKLDGVYSTDFSTDDVFRSYTAPSGKASIKYSDESGNVAITPAAANWYATLNFPFDWDLNGI
QTITMDISAATAGSVNIGLYGSDMEVWTKAVDVNTEVQTVTIGINELVKQGNPQTDGKLD
AALSGIYFGAATADTGSITIDNVKIVKLATPVYTANTYPHKDMGEESYIDIDTTGFKKQTTA
WNSKFTGTTMQITDANVLNINGETTKTKCVTYTRDATDTEGCRAKFDLNTVPSMDAKYFTI
DIKGNGIAQKLTVSLSGLAYITVNMAEGDTDWHQYIYSLEGNVEYPEDITYVQISADTRTTA
EFYIDNIGFSNTKSERLIPYPEKTFVYDFATYNKNTTKYEAAISTESGSEGDTIVATKEEGGLG
FDSKALEVKYSRNGNTPSKAKVVYSPNDFFKGNVNDDERTANRATLKADMEYMTDFVFY
GKSTSGKNEKINVGVIDTASAMTTYTDTKEFTLTTEWKQFRVPFDEFKILDGGSNLDCARVR
GFIFSSAENSGEGSFMIDNITHTSIKGDIEWGLPHHHHHH
SEQ ID NO: 3 - Full-length Rucg 13 GH5-30 enzyme
MERVIFMKKFLSLVTAIVMTVSLCIMPVYAQTYEEAAADAQNAEINYNRSVPLEVKGNKIV
KQGTDEMVVLRGVNVPSMDWGMAEHLFESMTMVYDSWGANLIRLPINPKYWKNGSVWD
EKNLTKEQYQKYIDDMVKAAQARGKYIILDCHRYVMPQQDDLDMWKELAVKYGNNSAV
LFGLLNEPHDIKPVGVEKPTTVEQWDVWYNGGQIIVGGEEVTAIGHQQLLNEIRKQGANNIC
IAGGLNWAFDISGFADGYNERPNGYRLIDTAEGHGVMYDSHAYPVKGAKTAWDTIIGPVRR
VAPVIIGEWGWDSSDKNISGGDCTSDIWMNQIMNWMDDTDNQYDGIPVNWTAWNLHMSS
SPKMLYSWDYKTTAYNGTHIKNRLLSYNTAPEKLDGVYSTDFSTDDVFRSYTAPSGKASIK
YSDESGNVAITPAAANWYATLNFPFDWDLNGIQTITMDISAATAGSVNIGLYGSDMEVWT
KAVDVNTEVQTVTIGINELVKQGNPQTDGKLDAALSGIYFGAATADTGSITIDNVKIVKLAT
PVYTANTYPHKDMGEESYIDIDTTGFKKQTTAWNSKFTGTTMQITDANVLNINGETTKTKC
VTYTRDATDTEGCRAKFDLNTVPSMDAKYFTIDIKGNGIAQKLTVSLSGLAYITVNMAEGD
TDWHQYIYSLEGNVEYPEDITYVQISADTRTTAEFYIDNIGFSNTKPERLIPYPEKTFVYDFAT
YNKNTTKYEAAISTESGSEGDTIVATKEEGGLGFDSKALEVKYSRNGNTPSKAKVVYSPNDF
FKGNVNDDERTANRATLKADMEYMTDFVFYGKSTSGKNEKINVGVIDTASAMTTYTDTKE
FTLTTEWKQFRVPFDEFKILDGGSNLDCARVRGFIFSSAENSGEGSFMIDNITHTSIKGDIEWG
LPTPTPEPTPTPLPDPVTVTTAEQLAAITSTEGNIILGADIDLGTTGFTTKSVTHLDLNGHTLTS
SGPFVVDPRHEITIVDTGSTKGAIINTGTTQTSYGIRGTTEAATINIDGAEIDAGGQAILINVAG
RKCNIKDAVINGGSYAINVGTNGGEINIDNALINNKADYKGYALYLQGGIAIIDDGTFGYNG
TTNTLLVARSSELTINGGTFTNPNSGRGAIVTDKQFVGTVTINGGVFENTNAGGYSILDSNEG
YQSIDAETSEIIASPVININDGTFKSAIGKTKSTNSSATEISIKGGQFAADPTVLYPNCIDTDIYSI
TKVAEGKYVVTKKGVEPTPEPTPEPVAKIVSSIEEINTLTASDDYVKLGADIDLGTSSIKTKC
AMRLDLNGHTLSGGGSTVIEAMYNLTVVDTGTTKGTIKNVNTSTSYGIKFAVKDAVLTIDG
AKVEAMSQAIMLSGTGSILHLKDSVINGNSYAVNLSNGIINIENTVINDDSEYKGYALSVAN
GTAVINSGIFNYNGNMSSITFSGSSEITINGGTFKNSVSKRGAINTVKGFSGTLTINGGTFENT
AENNGYSILDGDEATTETVPVINITGGTFKSTIGATKPANTTTVITISGGTYSFDPTSYVTDTET
YRVIDNGDGTYKVAPNSQVYSVTLNACGGSEVMVEDFKEENIPDNGIELPIPTKAGYKFDG
WYTEENNGSQVNGITKDNLSDIFRNEATVTLYAHWTLLNYTITYEGLNDATNTNPSNYTVE
TEAITLAAPGTRKGYTFGGWYTDVEYQNKIEIIEQGTTGNKILYAKWDEIASGSITASFVSTG
TIPSDIVQGTINVTEKAYENDEVSFMVTLPKGYTLENVLCTADGENLNTITEENGSYTFIMPG
KNVTITVNVRPIQYTINLDLQEGTGTTTTIYGSVENLPVLPNDNPKKQGYNFKGWFDAPTKG
TVITMDNLNTASNMLALFGNNTELTIYAQYTEVGNFVVIYSAVGADEETIPTDNTQYNIAET
SIIKIPNQEPKKLGYTFEGWKTGTDDTVYKYGTQNDTYTVPNDINGAITFIAQWSINEYEITY
ELNGGINAENAPVSYTIETDTITLPVPTKDGYNFEGWYTDAAFENAVAAIAKGSVGDMVLY
AKWSEKDMAVYKINNYEKGNVSVRKRTDTDDSSSVVIVAFYKTLNNNSVLIKTSIAEIGAIE
KGDDISKTVEEPEDYSYAKVFMWNDLNGMMPRCNSPKMDK
SEQ ID NO: 4 - Rucg13 PL8 (polysaccharide lyase 8 family protein)
MILLIHIKMGGMIMTDFNILRKRYSDVLCGRGYNGKKTADCILQSDERTEQRLVQLGGRIEK
AITSNEPGVINATLKGILDISISFSQNNSQFYHNKNIKNEIFNALNTLEKVYNDTTVPKGNWW
YWEIGIPLSINSIFTLMYDYTDKSQLKRYMAAEKHENDRIKLTGANRIWESVIFAVRGILLSD
NDSIKNAISGIQDVMVITDSGDGFYKDGSFIQHDNIPYNCGYGRSLIQELAPMLYIFKDTEFEN
KNTDIINTWIEKSYLPFIYNGRAMDMVRGREISRYYEQSDLACTHILSAMLILSEMPEFNELK
GTIKTQITDNFFEYASVFTAELAEHLQEDNNIKPKEIKPYFMAFNSMDRVVKHGNGYTIGLA
MHSERTAAYESINDENQNAHHTSDGMMYIYKKNEPKSDFFWQTIDLQRLPGTTVLRGSTVK
PNINAAGDFTGGCGIGENGVCTMKLISNENSLKANKSWFFFDKEVVCLGSCINSEEESEVETI
IENRLVTDNSRFTVHGNEESEGYIIKGAYLDGSHDVGYCFPEEQEVNIFREIRSGDWNNMSIK
SDGKSYKGRYLTMWIKHGRKVKDVSYEYIVIPKCHEEEINDYYRKSGIRIIENSDSIQCVKKN
GTTGVVFLKDKTHSAGGISCDRRCIVMTTQTCGTLELSISDITQKQDKIYIELDYSAQEIISKSE
RINIIQLVPYVCMEIDTCAARGEEQHIKFGGVKNV
SEQ ID NO: 5 - Rucg13 GH94
MENLLVRRTNMKYGYFDDLNKEYVIETPRTPLPWINYLGTNGFFSLISNTSGGYCFYKDAK
HRRILRYRYNNIPADNGGRYFYINDNGDCWTPSYMPMKKELDFYECRHGMGYTKITGERN
GVRVEQTAFVPVDDNCEIHRIKVTNTSGEAKNINLFSFVEFCLWNAQDDMLNYQRNLNTGE
VEIDGSAIYHKTEYRERRNHYAFYSVNTEISGFDTDRDTFLGAFNGLDTPDRVINGKSGNSV
ASGWYPIASHQIDVSLDAGESREYIFVLGYIENEKDEKFESLNVINKTKAKEMIARYESSAQC
DAELDKLKLYWDNLLSVFTLESNDEKLNRMVNIWNPYQCMVTFNMSRSASYYESGIGRGM
GFRDSNQDLLGFVHQIPERARERIIDIASTQFEDGSAYHQYQPLTKQGNNEIGGGFNDDPLW
LILGTVAYIKETGDYGILDEQVPFDCDKNNTATLLEHLNRSFGHVTNNLGPHGLPLIGRADW
NDCLNLNCFSEIPDESFQTTGDDDGRVAESVLIAGMFVYIGREFARLYKTLNNDEMYKYISD
EVEKMTEAVLEYGYDGEWFIRAYDANGNKVGSDECDEGKIFIESNGFCVMAGIGKEDGRA
QKALDSVKKYLECEYGIVLNYPPYSGYRLELGEISSYPPGYKENAGEFCHNNPWVIIGETVM
GNGERAFELYKKIAPAYLEEISEIHKTEPYVYSQMIAGRDAVRAGEAKNSWLTGTAAWNYY
TVSQYLLGIRPDFDGLVIEPCISKDISEFKVTRKFRGKTYNILVKNTGEGTVKITADNGTVNG
TTVSSDAEICNVEVVM
SEQ ID NO: 6 - Rucg 13 GH38-8
MILIYNSDIMYNKYIKPKFIIWYKKEFQMSKNVHIISHSHWDREWYLPFEQHRMRLVELIDK
CMEVFEKDDSFKSFFLDGQTIVLDDYLEIRPENKEKLIKYTKEGKFIIGPWYILQDEFYTSGEA
NIRNLLVGMKEAEKYGAMCKMGYFPDAFGNAGQMPQLLKQAGMDTVTFGRGVRPVGFD
NEVQENGNYESPYSEMMWESPDGTKIFGILFANWYNNGNEVPTDKKIAKEYWDDRLKKVA
TFASTDEYLLMNGCDHQPVQADLGKAIEVASELYPDINFKHSNFPEYIKAIKEKVPNDLAVV
KGELTSQDTDGWSTLMNCASSHIYLKQMNRKCESALENGAEPVRVLSSVLGQNYPSDELEY
SWKKLMQNHPHDSICCCSVDEVQDEMATRFNKSKQVADYLVSEGKRYIADKINTKEYEKY
KNALPFVVFNTAGRERTSVVSVEIDVTRKSGWLKKCAYDLDEINVPNYKLIDSDGNSIPFKIE
DLGVKFGYDLPKDKFRQPYMARRVRVTFEAENISAVGYKTYALVEGDTEKVTDTLVSSEN
CMENDAIRVEINKNGSLNVTDKASGRTYKGVAYYEETGDLGNEYMYKMPEGSKAITTQDT
VAKIELAEDEPYRAMYKITNTITVPKSGDDNFEDEKSHMVFFKERVGGRSNDTVEMKIETFV
SLDKNGKGVKIKTRFDNEVKDHRVRIMVPTGINSDVHKADSVFEVVTRNNRHNAGWNNPS
ACEHEQGFVSIDDGEKGIAVANIGLYEYEMLPDLDNTIAVTILRAVGEMGDWGVFPTPKAQ
CLGISETEIEIVPFKGDLISSGAYEECYQFRTDIITADTDCHDGVMPLDYSMINWQGNGLILTG
IKQKGNGEDIIIRWVNVSDKTTTLTIQKSDVIDNLYISNIIEKKIKKIDSNNNYFNIEVKPYEIM
TVGIAK
SEQ ID NO: 7 - Rucg13 GH38-30
MERKNIKCHFISNTHWDREWKFSAQRTRHMLVTAIDMLLDIFEKEPDYKHFHLDSQTLPIQD
YLEINPEKKEILKKYISEGKLAVGPWFCLPDEFCVGGESLIRNLLLGHKIANEFGKVSKTGYS
PFGWGQISQMPQLYHGFGIDFASFYRGLNTYMAPKSEFYWEGADGTTIYASRLGQRPRYNM
WYIMQRPVFYGKRDGDNRRVSWGAGDGIFRFADPARCEYEYQYSHRKYEYHDEYIAEKTE
QALSEQDDEWTTPNRFWSNGHDSSIPDMRESRLIKDANAVYEGVDVFHSTVYDFEQSVIRD
FDKNSPVLKGEMRYPFTKGSVSALFGWVLSARIKVKQENFETERLLTSYAEPMAVFASVCG
AVYPQAFINKAYNYMLQNHGHDSIGACGRDVVYKDVEYRFRQSREIATCVLERALMDLSG
DIDFAGWDKNDMALVMFNPAPFKRSLTVPCELEIPLEWECDSFEIVDAEGNVCPHQNISSINP
MYQIVQDLADAVDVLPVSRHTIRIFVKDIPSMGYKTLKVVPKYHTRATTPVNMLCGINTME
NEYLKVKINSNGTLKVTEKETGREYDNIGYFKDTGENGSPWEHKTPELDEEYTTVNERAIVS
LVYSGELETKYRIVLNWAIPENIVDGGKKRSSRLAPYRIETLVTLRKGARWVEFETKINNNV
PNHYLQAAFPTDVDAEFVYAQGQFDVVKRPIAKPDYSKYDEIPMTEQPMNSFVDICNENEG
AAILNTGLKAYESDDDYNHTVYLSLLRCFELRIYVTPEEQNYSRIENGSQSFGEHTFRYAFM
PHKGDWEDAQVWKAAEDFNMEILIGQTAPTEHGKNPLEKSFIELENENLHISAVKRSEDGL
GCVVRLFNPSSETVKNRIRFNGGIAEISDKQSPIERQVHSFELPCTENRKWASVKKVTLEELS
ETELSVDTNGWCDVEVTPKQIYTLKYE
SEQ ID NO: 8 - Rucg13 GH88
VNIDKAITYAESIVRKSLNYFYDCFPTEQSENLVFKKFENVSWTTGFYEGILWLMYELTGDK
AFYNSAKHHSEMFHKRLVDRVELEHHDMGFLFTLSSVADYRITGDEQAKQDGIEAAEWLL
KRYQPKGKFIQAWDAMDDSQSYRFIVDCMLNIPLLFWASEVTGYKKYYDAAYNHMQTSIA
NIIRPDASSYHTFFFDPVTNKPLRGETHQGFSDDSSWARGQSWAVYGLALCYHYTKEKSILP
LFERVTHYFIDHLPEDSVPYWDLIFSDGSDEPRDTSAAVVAVCGILEMEKYYHNQEFLDAAE
KMMTSLSEKYTTVDYPQSNGIIKDGMYSRKHGHEPECTSWGDYFYLEALMRMKKSDWKIY
W
SEQ ID NO: 9 - Rucg13 CE (carbohydrate esterase)
MKKIISLMLAVTMICASIGLTAFAATTTTVEAEADGVSAYTLPSSDKSNSKILKNTVSSKESV
TYYIQANNTPRATMFKLAQVNTGDKINVDINFTYLDTATMELEYCLFVSDSEITLTSHSQDL
VKEELEKHTDESNIKNWSTNKSNMKYSLPNGITASKDGFVYLYIGCGDLSEDKTQVTKKIQ
WSIDSFDVNIDSDGGGETEPDTTPTPTTTINPDVTPTPTPTASPTPTPTLEPELTLNAVYSSNM
VLQRKEPITITGTGKSGNTVSVNFNGADEQTTIEHGLWEITLPAMEAVKSATMTVSSGDNMI
TLDNVAVGDVIFCTGQSNMFNRLETFPTLMNEELSEAYEDVRYMNSFDEISEWKVATMENS
KQFSALGFLIGKRMIKKDSDVPIGLISSSLGGSSIMQWIPTYSVNWDSQAKRMMAGASSKGG
LYTQRLLPLKNLKASAVVWYQGEANTTFESGTVYEQALTSLINNWRKTFNDEDLPFVVIQL
PTANFAKIYSTIRIGTGVRAGQWNVSQRMDNVKTVVSNDTGTTNNVHPNDKGPIADRAVA
YIEDFINNTQSNVESPSFDYMERSGDKLILHFKNTYGSLSTDDGGVPLGFELKDDDGIYKDVT
PTINGDTIEIDVTDITNPQVKYAWSDTPGIAKDLVEAQTDTPAVINTFNAAGRPIAPFMTDLT
EKYASKAVNKELSTTEFYNYAPYISKVEQSGDDIVISAYDTDGVVSKVEVYIDEGEIKAGDA
KQRDDGKWVFTPDVTSGVHSVYAIATDNDNINSLTCVDYTTYNIIRPTRYDYVKGYTESPSS
VEYNNGDDMLAKATNDVNGTTTTVTSAIPTGETTKSLKLSATGNKATANATIPISKADNPQ
KTLTIEYDTMFESADDAIGASRGMYAKTKEGNELWLTYFTASSLRTAITNTGGNWCYEQA
MSIKNNQWHHIKLELHPNTGIFSIWLDGTMLQDNVSFVKEGSSFDTCKGAFDTLKEGITDLR
FYHTASNNIENATYIDNVKVTEVSYSEEEIIPPAKIQEATPQISIDYINETLTGFESQEPYTIKVG
EGNAKDITLGEGVTTISLDDEKIGYAGKLLSIEIVKKARNTETYTDSDVQQLTVKARPKAPTT
VQGVNATEIGGKGKLTGMNGMQYKLKRTDEWSSTQLVDTVEVDAGEYNVRKAATDTDFA
SEKTTITVETFIAEKEMTPEIAIDYTTEELINFVEDGTYTINGLDVTLTDNKLSLANYITNEQIT
LSIVKKGNNVTTVASEAQTLIVKARPAAPTKSEIIVTQPSVIGGKGTIAGIADTMEYSTNNGIN
WTTGDGDDIGDIEPGTTYKIRYKAVSADEEAERQFKSAEYSVTIIAYDAMPETQPTISINYVN
EKLTGFTEGCDYIIKIDDGVATDKDNVTEDIDIDNTYFGHTLKIVKKDDGIKTSNSEAFELSIP
KRSSAPNVAAVEEQTYQGNDGKITGVDTTMEYKSLSEPTFTWMQCVGTEITNLAPGSYIVR
VAAVADESFASEVMSVTINAAAKDEPTEPTVNITYDDKNGNVNAIFTNITEEGMVYVAEYN
ENGTLLSIKSDEISDSVIIPFTCVNKSKVKVFIWKNDMKPLFNKVFTLN
SEQ ID NO: 10 - Rucg 13 altCE (carbohydrate esterase)
MFNKKFNLLKEATEYGFMPYMETYILDGKKRPIVVIFPGGGYGMVSEREAERIAMAYNAAG
FHAAVVYYCVEPHTHPLPIQNAANAVAMLRENAEKWNIDTDKVIVCGFSAGGHLAASLSA
LWNDSEIFSEREIELAMHKPNAQILSYPVITSGEFAHKDSFKNLTGTDDESNHLWSSLSLERRI
TDIIPPTFLWHTYEDICVPVENTLMYAAGLRRVGVPFELHIFEKGEHGLSRVSDELIWSKRKF
EREYPWLSLSVDWLNQLF
SEQ ID NO: 11 - B. intestinalis SusD
MKKRHIIGSFLLGLLLTVNTGCEDFLDQKDTSGINENSLFLKPEDGYSLVTGVYSTFHFSVDY
MLKGIWFTANFPTQDFHNDGSDTFWNTYEVPTDFDALNTFWVGNYIGISRANAAIPILQRM
KDNGVLSEKEANTLIGECYFLRGVFYYYLAVDFGGVPLELETVKDEGLHPRNSQDEVFASV
VSDMNIAAGLLPWKAEQGSADRGRATREAALAYQGDALMWLKQYKEAVEVFNQLDSKC
QLEENFLNIHEIANRNGKESIFEVQFTEYGSMNWGAFGVNNHWISSFGMPVAISGFAYAYAD
KKMYDSFENGDLRRHATVIGPGDEHPSPLIDLQDYPKLKDFATKGNGNIPASFYQDEEGNV
LNTCGTVENPWLDGTRSGYYGVKYWRNPEVCGTRGAGWFMSPDNIMMMRYAQVLLSKA
ECLYRLNDSNGAMAIVQKVRDRAFGKLQNSAVEVPAPANTDVLKVIMDEYRHELTGETSL
WELLRRTGEHANYIKEKYGITIPTGKDLMPIPQTQIGLNQNLKQNPGY
SEQ ID NO: 12 - B. intestinalis SusC
MKTKFIATFFLLICGSVMFAQTRTVKGKVVDKANEPLIGVAVNIKNTSQGSITDFEGNYSIQV
NTENAVLVFSYIGYDKQEIKVGARNVIDVVMHEASIALDQVVVVGYGTSKRGDVTGSISSID
AAEIKKVPVVNVGQALQGRMSGVQVTNNDGTPGAGVQVLIRGVGSFGDNSPLYVVDGYPG
ASISNLNPSDIQSIDVLKDASAAAIYGNRAANGVVIITTKRGNADKMQLSVDATVSVQFKPS
TFDVLNAQDFASLATEISKKENAPVLDAWANPSGLRTIDWQDLMYRAGLKQNYNLSLRGG
SEKVQTSISLGLTNQEGVVRFSDYKRYNIALTQDYKPLKWLKSSTSLRYAYTDNKTVFGSG
QGGVGRLAKLIPTMTGNPLTDEVENANGVFGFYDKNANAVRDNENVYARSKSNDQKNISH
NLIANTSLEINPFKGLVFKTNFGISYGASSGYDFNPYDDRVPTTRLATYRQYASNSFEYLWE
NTLNYSNTFGKHSIDVLGGVSIQENTARNMSVYGEGLSSDGLRNLGSLQTMRDISGNQQTW
SLASQFARLTYKFAERYILTGTVRRDGSSRFMRGNRWGVFPSVSAAWRIKEESFLKDVDFIS
NLKLRASYGEAGNQNIGLFQYQSSYTTGKRSSNYGYVFGQDKTYIDGMVQAFLPNPNLKW
ETSKQTDIGIDLGFFNNKLMLTADYYIKKSSDFLLEIQMPAQTGFTKATRNVGSVKNNGFEF
SVDYRDNSHDFKYGVNVNLTTVKNKIERLSPGKDAVANLQSLGFPTTGNTSWAVFSMSKV
GGSIGEFYGFQTDGIIQNQAEIDALNANAHRLNQDDNVWYIASGTAPGDRKFIDQNGDGVIT
DADRVSLGSPLPKFYGGINLSGEYKGFDFNLFFNYSVGNKILNFVKRNLISMGGEGSIGLQN
VGKEFYDNRWTETNPTNKYPRAVWSDVSGNSRVSDAFVEDGSYLRLKNIEVGYTLPANILK
KASISKLRIFASVQNLFTITGYSGMDPEIGQSMSSSTGVAGGVTASGVDVGIYPYSRFFTMGF
NLEF
SEQ ID NO: 13 - B. intestinalis GH3
MKTFILSFLIYAGCSLPLTAQQIKPAIPSDPEIEAKINKLLQKLTLEEKIGQMCEITIDVITDFSD
KENGFRLSESMLDTVIGKYKVGSILNTPFSIAQEKEVWADLITRIQKKSMEEIGIPCIYGVDQI
HGTTYTRGGTFFPQSINMAAAFNRQLTRRGAEISAYETKACCIPWNYAPVMDLGRDPRWPR
MWESYGEDCYVNAEMGVQAVKGLQGENPNHIGENNVAACIKHFMGYGVPVSGKDRTPSSI
SRTDLREKHFAPFLASIQAGALSLMVNSGVDNGVPFHANKELLTGWLKEELNWDGMIVTD
WADINNLCLRDHIAETKKEAIQIAINAGIDMSMVPYEVSFCTYLKELVEEGKVSMARIDDAV
SRVLRLKYRLGLFDNPYWDIRKYDQFASPEFASVALQAAEESEVLLKNEDDILPLAKGKKIL
LTGPNANSMRCLNGGWSYSWQGDKADECAQAYNTIYEAFCNEYGKESVIYEPGVTYKTSA
DALWWEENTPRIAQAVSAAEKADVIIACIGENSYCETPGNLTDLNLSTNQKDLVKALAATG
KPIILVLNEGRPRIIHDIVPLAKAVVHIMLLGNYGADALVNLVSGKANFSGKLPFTYPHLINSL
ATYDYKPCENMGQMGGNYNYDAVMDVQWPFGFGLSYTTYSYSNLKVNRTSFDADNELVF
TVDVTNTGKMAGKESVLLYSRDLVASITPDNIRLRNFEKVDLQPGETKTVTMKLKGSDLAF
VGADGKWRLEKGAFRMTCGTQKLEVHCTTTKIWQTPNISKSGI
SEQ ID NO: 14 - B. intestinalis PL2
MKNTVLPLILFLCMLCLGSHLYAGHSMHPLNQISYVKKKIKEQQEPYFTAYRQLMHYADSI
QEVSQNALVDFAVPGFYDKPEEHRANSLALQRDAFAAYCSALAYQLSGEERYGQKACYFL
NAWSSTNKKYSEHDGVLVMSYSGSALLMAAELMMDTPIWNPQDKDAFKTWVSQVYQKA
VNEIRVHKNNWADWGRFGSLLAASLLDDKEEVARNVQLIKSDLFVKIAEDGHMPEEVVRG
NNGIWYTYFSLAPMTAACWLVYNLTGENLFVWEHNDASLKKALDYMFYFHQHPSEWKW
DTRPNLGAHETWPDNLLEAMAGIYNDASYLQYVESSRPHIYPLHHFAWSFPTLMPVSLKGY
DLTDNNTWANYNRYEVANKTVKKPVAIFMGNSITEGWNRSHPDFFTQNGYVGRGISGQVT
AQMLARFRADVLDLKPQVVCILAGTNDIAQNCMYMSVENIAGNIFSMAELAKANGIKVVIC
SVLPATRYSWRPTVQNPAGQIIQLNKLLQKYAQKNKIPYVDFHSMMKDEQNGLPQKYSKD
GVHPTKEGFSMMEPIIKEAIDKLLK
SEQ ID NO: 15 - B. intestinalis GH5
MKNIYYILILCCLCLFSCDSHPDTKSSLPFGVNLAGAEFFHKKMDGVGQFGIDYHYPTTREF
DYWKSKGLTLIRLPFKWERIQRELYGELNREEIDYIKYLLDEAGARDMKILIDMHNYGRRK
DNGKDRIIGDSVSIDHFASVWKQIAGELKEHSALYGYGLINEPHDMLDSVPWFKIAQAAIEE
SRKVDLKTAIVVGGNHWSSAARWQEISDDLKHLHDPSDNLIFEGHCYFDEDGSGIYRRSYD
EEKAYPTIGIDRTRPFVEWLKTNNLRGFIGEYGVPGDDERWLVCLDNFLDYLSKENINGTY
WAAGAQWNKYILSIHPDDNYQTDKIQLGVLTKYLETKN
SEQ ID NO: 16 - B. intestinalis GH88
MRKQLSLLLVSISLGWVGCAPDKQADTIHLDRQLEYCDAQIRRTLSEADQDSCLMPRSMEA
NQTNWNMSNIYDWTSGFWPGILWYDYEATGDEEIKAQAIRYTECLLPLVTPAHGADHDIGF
QIFCSFGNAYRITGNEEYKTVILKGAQKLAKLYNPKVGTILSWPGMVKRMGWPHNTIMDN
MMNLEILFWAARNGGGQELYDIAVKHAQTTMKYSFREDGGNYHVAVYDTIDGHFIKGVTN
QGYGDSSLWARGQAWAIYGYMMVYRETQDKTFLRFAEKVTELYLENLPEDYIPYWDFDAP
DMIKQPKDASAAAITASALIELSELEDTPSLASRYLNAATRMLGELSSERYQCRDIKPAFLMH
STGNQPGGYEIDASINYADYYYLQALLKYKKAMGL
SEQ ID NO: 17 - B. intestinalis GH92
MKTRTLGICLFLLMNVSFIKGQSLADKVDMWMGTYGAGHCVVGPQLPHGSVNPSPQTAYG
GHAGYVPDQPIRGFGQLHVSGIGWGRYGQIFLSPQVGFNPGETDHDSPKQGEEATPYYYKV
MLSRYDIQVEISPTHHCVAYRFTFPETDOGNILLDIAHNIPQHIVPEVKGLFHGGEINYNPEQQ
TLTGWGEYSGGFGSTDAYKVYFAMKTDTPLKEVKITDQGDKALYACLALNKNPGVVHLN
VGISLKSIENASLFLSEEIADNSFNTVKENAKAIWDNTLSSIKIKSENEAEERLFYTTLYHSFV
MPRDRTGDNPHWDSESAHMDDHYCVWDTWRTKYPLMVLLRESYVAQTINSFIDRFAHNG
VCNPTFTSSLDWTSKQGGDDVDNIIADAIVKNVKGFDYEKAYALMKWNAYHARSKDYLRL
GWEPETGGIMSCSAGIEYAYNDFCTSEIAGIMHDENTQKELYERSGNWSQLFNPLQESHTYK
GFIVPRKANGEWVAIDPAKAYGSWVEYFYEGNSWTYTLFVPHQFDRLIEYCGGKANMIKRL
SYGFENNLISLNNEPGFLSPFIFTHCGRPDLTARYVSQIRKDNFSLLKGYSDNEDSGAMGSW
YIFTSIGLFPNAGQDFYYLLPPAFTDVELTMENGKKISIKVLKDTPDACYIKSVSINGKVLDK
GWIYHREIAEGATLVYELTNKENAWHINE
SEQ ID NO: 18 - Rucg13 GlK Glucokinase A
MKYYIGIDLGGTNIAAGIVDKTGKIIAKDSVPTLNTRPIEAIMLDMTKLCKTLLDKSQMDINK
IEAVGIGCPGTVDNKNGIISYSNNIPMKNVPMRKFMEKQLNISVNLENDANAAALGEYTAN
GHNASSYILITLGTGIGGGAVINSKIYRGFNGVGIEPGHMTLINGGERCTCGKHGCWETYGS
VTALINQTKLKMTDNPDSLMHKISGKFGEVNGRVAFEAAKAGDKAGLEVVEKYTEYVADG
ITSVINIFEPEILVIGGGISKEGEYLLNPIRKFVEINEFNKYRPKTKIEIASLNNDAGIIGAALSAN
R
SEQ ID NO: 19 - Rucg13 CBM11
MKKLVSLIIAMSIFFSINCAIFATNVSYMADFESADAKFGNSTTYSGTKNTAGDYSDFVKPE
WVADGGKENSTGLRITYKAATWYAGEVFFPIPVAWQNGADAEYLNFDYNGKGIVNISLST
GSAATDTLTKGTKYSYKLNADTNGEWQSISIPLSEFKNNGNPVTIANIGCVTFQAGENGGLS
NSASETKAMTAAELEAKARNGSIIFDNMELSNVGENVLNPNATPEPTEKPDNTTRTIDFDTY
TLSHKQTWAGFNNNDKTYSDSIKSEITENGKEGCALELTYKAATWYAGEIFMSIPKEWAINK
NSECLEFDAKGQGKIKISLETGEVVNGIRYGHTVTINTNDEWQKISVPLSEFVNNGNEVPLTD
VVGMAFSAAESGNLDNNAEETKMMSADELEEKAVTGCVVIDNITLAEQDTTSPTAAPEATT
QPTEISYVADFETADTKFASGKTWGGFKNKSNDYQDYIKAKWLQDGGVDGSTAFCVYYQS
ATYYAGEIFVPAPAVWTNNGAKGAEYLNFDYKGKGAVKISFSTGNTVDGTLTSGTRYTRRF
ELDSHGDWAKISVPLSEFVNGENIVNMTEIGTVTFQAAENANLDNNSDDTKAMSADELKEI
ARTGEIIFDNMTLSETEGKTTLFSSVKVTAEIDGKEITNLTNGDIKIKAIASDIEKDTNMVMIV
AVYKENGVIDTVRMAGQKIIGDGELMLDLNVTDAEHQTMKVFIFDDFTNLHPIINVTNFL
SEQ ID NO: 20 - Rucg13 Glk glucokinase B
BMPTIRFVYTYSLLWWAERLCGKMYYIGIDLGGTNIAAGIVTEEGKIVVKDSVPTLSERPTD
EIVTDMANLSKKLVQSIGIEMNEIKGIGIGCPGTIDFETGEIVYSNNIKINHYPLADKFKEHIPL
PVKVDNDANCAALGEYKINRHCASVFALVTLGTGVGGGVIINGKVFRGFNGAAGELGHMTI
VSGGKMCTCGKEGCLESYASATALISQTKDALETHKDTIMHGIVKKEGKISGRTAFEAAKQ
GDEVAKKVVSNYERYLADGIVSIENIFQPEIIAIGGGISKEGDYLIEPIREYVYNTGFNKHMTK
TKIVAAQLFNDAGIIGAAMLAI
SEQ ID NO: 21 - Rucg13 HK histidine kinase
MSEKFNNMSFRTKLLLSYIAVIILCIIIFGLTVFSSISRRFENEITDNNAQITGLAVNNMTNTMN
NIEQILYSVQANSTIEKMLTASNPPSPYEEIAAIEQELSKIDPLKATVSRLSLYIENRTSYPSPFD
SNVTASVYSKNEVWYKNTKELNGSTYWCVMDSSDANGLLCVARAFIDTRTHKILGIIRADV
NLSQFTNDIAHISMNNTGKLFLVYENHIINTWNDSYINNFVNENEFFKAISADSDKPQLVQIN
KEKHIINHSRLKDSSLILVRASKLDDFNSDIHIIEKSMITTGIIALLVALIFIFIFTRWLTAPITKLI
KHMERFENNYERIPIEITSHDEMGKLGESYNSMLNTIDSLITDVEDLYKKQKIFELKALQAQI
NPHFLYNTLDSIHWMARAHHAPDISKMVSALGTFFRHSLNKGNEYTTIENELNQISSYVSIQ
KIRFEDKFDVVYDIDENLLHCTIVKLTIQPLVENSIIHGFDEIEEGGMITIRIYPEDDYIFIDVIDN
GSGADTNELNKAITHELDYNEPIEKYGLTNVNLRIQLYFDKTCGLSFKTNETGGVTATIKIKR
KEPEYKTIDL
SEQ ID NO: 22 - Rucg13 Pgm phosphoglucomutase
MQCRGGNVMNFNIPDLGIIDGSSGFRNLPSTTDGRFTSGEDGVKHIVCTGDGKVEFVAFENQ
TLAYVNSALGYGAYYPLHPVNRNGKIKAVLMDLDGTSVRSEEFWIWIIEKTTASMLDDESF
KLEESDIPFVSGHSVSEHLQYCIDKYCPGESLDKARNFYFDHVNREMKEIMEGRGRKNAFVP
QEGLKEFLLALKAKGIKIGLVTSGLYEKAMPEILSAFRALDMGEPTDFYDAIISAGYPLRKGS
VGTLGELSPKPHPWLYAETCAVGLGVGFDERGSVIAIEDSGAGVCSARIAGYTTIGLAGGNI
KESGTMPMCSRYCNNLAEILDYIEEEA
SEQ ID NO: 23 - Rucg13 ManA M6P Isomerase
MFFSVLHMAIINIKGVKIVSELYPVRLIPVFKDYLWGGTKLKTVFNKKSELNILAESWELSAN
KDGQSIIANGKYQGYGLKEYIDIVGKEIVGTKGLALDDFPILIKFIDAKKNLSVQVHPDDEYA
TCHDGANAKTEMWYILDCNVGAYLYYGFKKDITKQEYQDAIRSNTITDVLNKVPVHKGDV
FFIPAGTVHAIGAGILICEIQQNSNTTYRVYDYDRRDKDGNKRELHIREALESSNLKKSTYSN
SVLDGDDIILTQCDYFTVRRLKVQNRVQLRIDKTSFHSLIITDGSGELYMGGEILKLNKGDSIF
IPAQNNEYTVSGPCEIILSFL
SEQ ID NO: 24 - Rucg13 RR response regulator
MNVKLLICDDEKIIREGLASLDWNTRGIEVVGTAKNGEVAFELFQKMLPDIVISDIKMPTKD
GIWLSEQIHKISPNTKIIFLTGYNDFEYAQSAINNGVCQYLLKPIDEFELYEIVDKLTKEIHLEQ
QKAEKEIELRKTLRNSRYFLLNYLFNRAQYGILDFELFEISKKAAAMTTFVIRLDTDSTNYG
MNFMIFEALIEHLPKTINFIPFFSNSDLVFICCFNEPEGESEQKLFSCCENLGDFIDTEFNVNYNI
GIGIFTSEISELEASYTSALQALDYSDRLGQGNIIYINDIEPKSQLSAYQSKLIETYIKALKNND
EKQSKKSVKELFDVMERSDMNLYNQQRRCMSLILSISDALYDIDCDPTILFKNTDAWSLIRK
TQSPAELKTFVENITDVVISYIESVQKQKAANIITQVKALVEKNYARDASLETVASQVFISPC
YLSVIFKKETNITFKNYLIQTRIEKAKELLEKTDLKIYDIAEKVGYNNTRYFSELFQRICGKTP
SQYRASHNPSMPQDI
SEQ ID NO: 25 - Rucg13 TR transcriptional regulator
MSDKKPLYKQIMDKLKERIKSGDFEYDAPFVTEDRITKEYGVSRITAIRALEELEHDGLINRK
RGSGSFVSKNAMSILGKDKEDNAAVTIHKKNRDISLVALVMPFDIKLGNMFKCFDGINSVLN
KENCFVSIYNANRSVENEEKILRSLLEQGIDGVICYPVRGGRNFEVYNQFLVKKIPLVLIDNYI
ENMPMSYIVSDNSGGGKALCEYALEHGHKKIGFFCRGRVNETISIRDRYMGYAAALEEKGL
GVNLDYVYANIDDKYEMLTEEERQQYGNVENYLKTIVNRMHEQGISCVLCQNDWVAIQVY
NCCKALDISVPNEMCIMGFDNISELDEMDGGNKIITVEQNFFELGVKAGETVLREINGEMPGI
KYIVPVKIAVRN
SEQ ID NO: 26 - Rucg13 XoPP transporter A
MLVVLGASFTSESAISEFGFHAIPKEWSLDAYRYIITSKETILRAYGVTIFVTIVGTLMSTLVV
ALYAYPLSRKDFKYRKLFTFIAFFTMLFSGGTVAGYMVTTGILNLKNSIWVLIFPYVMNAW
HVIVMRSFYSMSIPTAIIEAAKIDGANEYQIYFKIVLHISLPGLATIALFATLTYWNDWWLPLL
YITEPQKYNLQYLLQSMISNIQNLTENSAQMGSANLLANVPKEGARMALCIIATLPILFVYPF
FQKYFIQGLTVGSVKE
SEQ ID NO: 27 - Rucg13 XoPP transporter B
MLSMCIPGLIFFILFNYLPMFGIIIAFKQYRYDLGIWASPWNGLKNFEFMFSSPDAWVITRNTI
AYNLLFIFGGLVFNVAMAIGLSELRNKAVSKLCQTVVIMPHFLSYVIVSFLVLAFLHVENGLI
NRSLIPALGLEGVDWYSNPKYWPWILVIVNFWKTTGYGSVVYLAGIAGIDTSLYEAAKVDG
ASRWQQIRYITLPALVPLMVVLTILNVGKIFNSDFGLFYQVPLNTGALYPATNVISTYVYNM
LMSAGTGSVGMASAAAFYQSIVGFILVMTTNFIVKKISPENALF
SEQ ID NO: 28 - Rucg13 XoPP transporter C
MIMGKDETSEPLSKKKGDKIMRKKIAALLAMLMLGGVLTGCGGGNKVATGGEDPNVVPED
TYEINWYMQGMPQEDVASVEAAVNDYLKDKINATLKMHRLESNQYSKQLNTMIAAGEYF
DIAWTTPGVLTYTANARNGAWLALDDYIDTYIPKTIEQLGEIADNARVDGKLYAIPTYKEM
ADSRGWTYRKDIAEKYNINMDNIKTFDELLPVLKMIKENEPNMQYPIDWGSDRTPEALMKY
EEIAGTAVIFYDTDKYDGKVVNLVETPEYLEACKWDNKLYNEGLVKKDIMTATDFEQRLK
DGKTFCYVDFLKPGKAKETSAKFDFELDQSTVSDIWQDNGAGTGSMLAVSRTSKNPERVLR
FLELLNTDATLSNLINYGIEGKHYTKIDDNTITIPDDTSYTLQGYQWMQGNVFLNYLTEGESP
DKVEALKAFNAEAKKPIDYGFKFDNTAVEAEIAACQTVKSEYRKQVIMGSMDPEPIMKEYA
AKLKAAGIDKIIEEAQKQYDEFLANKNKQ
SEQ ID NO: 29 - Rucg13 XoPP transporter D
MKKLLILFLLASVMLSMCSGCTVEKTVESAQAVTVLKVIKPNYISDFTQNIAEFNEANPDIQV
KFIDAPTSTEKRHQLYVSALSGKDSSIDIYWINDEWTKEFVEQKYIKALDGEILLDNSRYIIDA
QERFSVNDSFYAMPVGMDTDVIFYRSDKIHNVPETWDGIINLCRNSDFGLPIKLGLTTSDIQD
MMYNIIEIKEAIGISYAETLNLYKEFIEEYKDIENYTDTIAAFKIGSAAMLMGNSSLWKKLNG
DTSAVKGNIMVASLPNKNQFVRSYALAINSNSKNQEAAIRFLDFMNGKEQQRRLSRDTSLIP
IIRELYDDEMILDANPHVKGIKQSVQNSSSFATVSINGENLKKLEEALIKFFNNEETSMNTGKI
FEDLMQ
SEQ ID NO: 30 - B intestinalis HTCS
MKQLITTLFIFIFLQPSWASLYRNYQVEDGLSHNSVWAVMQDKQGFLWFGTVDGLNRFDG
NSFKIYKKLQGDSLSIGNNFIHCLKEDSHGHFLVGTKQGFYLFNRESETFSHVRLDNRSRGG
DDTSINYIMEDPDGNIWLGCYGQGIYVLGPDLQVRKHYINKGNPGDIASNHIWCMVQDYNG
VIWIGTDGGGLIRLDPKDERFTSIMHEKDLNLTDPTIYSLYCDMDNTIWVGTSISGLYRCNFR
TGKVTNIVYPHRKILNIKAITAYSNNELVMGSDAGLIKVDCIQETISFINEGPAFDNITDKSIFSI
AHDMEGGLWIGTYFGGVNYYSPYANKFAYYPGSSEEVSKSIISYFTEESSDKIWVGTKNEGL
LLFNPAKISFETTHLQIDYHDIQALMMDNDKLWISVYGKGVSMVDVHSNTLLKRYSNDVGG
PDLLTSNIVNVIFKSSKGQIFFGTPEGVDCLDAETKKINRLERTKGIPVKAIMEDYNGSIWFAA
HMHGLLHLSADGTWESFTHMPEDSTSLMSNNVNCIHQDARYRIWVGSEGEGMGLFNPKTK
KFEYILTENLGLPSNIIYAIQEDADGNIWVSTGGGLARIEPETRSICTFRYIEDLIKIRYNLNCAL
RGRDNHLYFGGTNGFIAFNPKDIQNNEYKPPICLTGFQISGNEVVPGIEGSPLKKSISMTQKIE
LESNQAAFSFDFVCLSYLSPAQNKYAYKLEGFDTDWHYVANGNNKAIYMNIPSGKYTFYV
KGTNNDGVWCDTPIKVTVIVKRHFWLSNMMLLVYAILAISAFTLLIRRYNKRLDSINQDKM
YKYKVEKEKEIYETKINFFTNMAHEIRTPLSLIVAPLENIISSGDGSQQTKSNLEIMKRNANRL
LELVNQLLDFRKIEEDMFRLCFSKQNISEIVRNIHKRYVQYAKLKDIDIRLVEPEKDIACVVD
KEAMEKVIGNLLSNAVKYANSLITINISTDNNLLTISVKDDGPGIKSEFIDKIFESFFQIENNAQ
RTGSGLGLALSKSLVTKHKGNIAASSDYGHGCTLTFTIPMDLPISISQLTEEYPEKEDISVQQT
ALSPVEGKLRIVLAEDNQELRSFLSNYLSDYLDVYEAQNGLEALQLVENENIDIIVSDILMPE
MDGLELCKALKSNPAYSHLPFILLSARTDTATKIEGLNTGADVYMEKPFSSEQLRAQINSIIN
NRNSIRENFIKSPLDYYKQKSAEPNGNTEFIEKLNIIILDNLTNEKFSIDNLSEMFLMSRSNLHK
KIKNIVGMTPNDYIKLIRLNQSAQLLATGKYKINEVCYLVGFNTPSYFSKCFYEHFGKLPKDF
IVIE
SEQ ID NO: 31 - Rucg13_XG
CATTTATCTATATTTTATGTACAAATATTAATATTTGCTTCTATACTATATATTATTTATCTATTCG
CACTTAAGGCAGCACCTATAATTCCTGCATCATTATTCAAAGATGCAATTTCAATTTTTGTCTTTG
GTCTATATTTATTGAATTCATTTATTTCAACAAATTTTCTGATTGGATTCAAAAGATATTCCCCTT
CTTTGCTTATTCCGCCACCAATAACCAAAATCTCAGGTTCAAAAATATTTATGACACTTGTTATA
CCGTCAGCAACATATTCTGTATATTTCTCAACCACTTCCAACCCTGCCTTATCACCTGCTTTTGCC
GCCTCAAAAGCCACTCTGCCATTTACTTCACCGAATTTCCCCGAAATTTTATGCATTAAGCTGTCC
GGATTGTCAGTCATTTTTAATTTAGTCTGATTTATGAGAGCAGTTACAGAACCATATGTTTCCCA
GCAGCCGTGTTTCCCACAAGTACACCTTTCACCACCGTTTATAAGTGTCATATGTCCCGGTTCTAT
TCCTACACCGTTAAATCCTCTATAAATTTTACTGTTAATAACTGCACCACCGCCTATACCTGTACC
AAGTGTTATTAGAATATAGCTTGAAGCATTATGTCCATTTGCCGTATATTCGCCCAAGGCAGCTG
CATTTGCGTCATTTTCAAGATTCACTGAAATATTAAGTTGTTTCTCCATAAATTTACGCATTGGCA
CATTCTTCATCGGGATATTATTTGAATACGATATTATACCATTTTTATTGTCCACCGTTCCCGGAC
ACCCAATGCCAACTGCTTCAATCTTATTAATGTCCATCTGCGACTTATCTAAAAGTGTTTTACACA
ATTTAGTCATATCAAGCATTATCGCTTCTATCGGACGTGTATTCAAAGTAGGAACACTATCCTTT
GCAATAATTTTTCCTGTTTTATCAACAATTCCTGCAGCGATGTTAGTTCCACCTAAATCTATTCCT
ATATAATACTTCATCTATAAATCACTCCATTCCTTAAGTTTGTTTAAAATTTTATAAAAATGATAA
TATAATTTCACAAGGTCCGCTAACAGTATATTCATTATTTTGAGCGGGGATAAATATACTATCTC
CCTTATTCAGTTTAAGAATCTCTCCACCCATATACAATTCCCCGCTTCCGTCTGTAATTATAAGTG
AATGAAAGCTTGTTTTATCAATTCTAAGCTGCACTCTATTTTGTACTTTCAGTCGACGAACGGTG
AAATAATCACATTGAGTCAAAATAATATCATCACCATCAAGCACAGAATTTGAATAAGTAGATT
TTTTCAAATTTGAAGATTCAAGAGCTTCTCTAATATGTAATTCTCTTTTATTCCCGTCCTTATCGC
GTCTATCATAATCATATACACGATAAGTCGTATTGGAATTCTGTTGTATTTCACATATAAGAATT
CCCGCTCCTATCGCATGTACAGTTCCCGCAGGTATGAAAAATACATCTCCTTTGTGAACAGGCAC
CTTATTAAGTACATCTGTTATTGTATTGCTTCTGATTGCATCTTGATATTCCTGCTTTGTAATATCT
TTTTTAAATCCGTAATACAGATATGCACCAACATTGCAATCGAGTATGTACCACATTTCTGTCTT
AGCATTTGCACCGTCATGGCAAGTGGCATACTCGTCATCGGGATGAACCTGCACAGACAAATTC
TTTTTTGCATCTATAAACTTTATAAGTATTGGAAAATCGTCAAGGGCAAGACCTTTTGTACCTAC
AATTTCCTTTCCAACTATATCTATGTACTCTTTCAAGCCATATCCTTGATATTTACCATTGGCTATT
ATACTTTGACCGTCCTTATTAGCCGATAGTTCCCAACTTTCTGCCAGTATATTCAGTTCTGATTTT
TTATTAAACACAGTTTTTAATTTTGTTCCACCCCAGAGATAATCCTTAAAAACAGGAATAAGGCG
AACAGGATAAAGTTCTGACACTATTTTCACTCCTTTGATATTTATAATAGCCATGTGCAAAACAC
TGAAAAACATAGAGTTTATACACTTTTACGGATATGGCTAATCCGTTTTGTCACTATAAATTATA
TTGGGTATATAGAAAAACCACTCTGATTTGGTATAATATTTGCACGTTTTTAATTTATTTTATAAT
AAATAACAAACAGAACATACAAACGACACAAAATTCCATTTAGTTTGACATGGGTAACGTTTTT
TAAGATAAGAATTTACAGTCGGTTATATGTTCTGTTTATAAATTAATATTTAGATGTTTTGTTATA
TTATTTATCCATCTTCGGCGAATTACAACGTGGCATCATTCCATTTAAGTCATTCCACATAAATAC
TTTTGCATAAGAATAGTCCTCCGGTTCTTCCACTGTCTTTGATATATCATCACCTTTTTCAATAGC
TCCTATTTCGGCTATTGACGTCTTAATCAAGACAGAATTATTGTTTAATGTTTTATAAAATGCAAC
TATTACAACGCTTGAGCTATCATCTGTGTCTGTACGCTTTCTTACGCTTACATTTCCTTTTTCATAG
TTGTTTATCTTATATACAGCCATATCTTTTTCCGACCATTTTGCATATAAAACCATATCACCAACG
GAACCCTTGGCAATAGCTGCAACTGCATTTTCAAATGCAGCATCTGTATACCATCCCTCGAAGTT
ATAGCCATCCTTTGTCGGAACTGGCAATGTTATCGTATCTGTTTCAATAGTATACGATACAGGAG
CATTTTCGGCATTTATTCCGCCATTGAGCTCATATGTAATCTCATATTCATTTATTGACCATTGTG
CAATAAATGTAATAGCACCGTTTATATCATTTGGGACTGTATATGTGTCATTCTGTGTACCATATT
TATAAACCGTGTCATCAGTGCCTGTCTTCCAGCCTTCAAATGTATATCCTAATTTTTTTGGTTCTT
GATTAGGAATTTTAATTATCGATGTTTCCGCAATATTATATTGGGTGTTATCAGTAGGAATTGTTT
CCTCATCCGCTCCTACCGCAGAGTATATAACTACAAAATTACCCACTTCTGTATACTGAGCATAA
ATTGTAAGCTCTGTATTATTTCCGAACAGTGCAAGCATATTTGAAGCGGTATTAAGATTATCCAT
CGTAATTACAGTTCCTTTTGTCGGTGCATCAAACCAGCCCTTGAAGTTATATCCTTGTTTTTTCGG
ATTGTCATTCGGTAAAACGGGTAAATTTTCAACACTGCCGTATATAGTCGTTGTTGTACCTGTAC
CCTCTTGCAAATCCAGATTAATTGTATACTGTATCGGACGTACATTCACTGTTATCGTAACATTTT
TACCTGGCATTATGAATGTATAGCTGCCATTTTCTTCTGTAATAGTGTTTAAATTTTCTCCGTCAG
CAGTACACAAAACATTTTCCAACGTATATCCCTTAGGAAGCGTAACCATAAATGAAACTTCATCA
TTTTCATATGCCTTTTCTGTCACATTTATCGTACCTTGTACTATATCTGACGGAATTGTTCCCGTTG
ATACAAAGCTTGCGGTAATACTTCCTGACGCTATTTCATCCCATTTTGCATATAATATTTTATTTC
CCGTCGTGCCTTGTTCGATTATTTCTATCTTGTTTTGGTATTCAACATCAGTGTACCAACCGCCAA
ATGTATATCCCTTTCTTGTACCGGGAGCGGCAAGAGTTATTGCCTCAGTTTCAACCGTATAGTTT
GACGGATTGGTGTTTGTTGCATCATTAAGTCCCTCGTAAGTTATCGTATAATTTAACAGAGTCCA
GTGTGCATATAATGTGACTGTTGCTTCGTTTCTAAAGATGTCAGACAGATTGTCTTTTGTTATCCC
ATTAACCTGACTGCCATTGTTTTCTTCAGTGTACCAGCCGTCAAATTTGTATCCGGCTTTTGTTGG
TATAGGAAGTTCAATACCATTATCAGGGATATTTTCTTCCTTAAAATCCTCAACCATTACTTCACT
TCCGCCGCACGCATTAAGTGTGACAGAATACACCTGTGAATTCGGAGCTACTTTGTATGTACCAT
CGCCGTTATCAATAACTCTGTATGTTTCCGTGTCGGTAACATAGCTTGTCGGGTCAAATGAATAT
GTTCCGCCGGAAATAGTGATGACCGTTGTTGTGTTAGCGGGCTTTGTCGCACCAATAGTAGATTT
AAACGTTCCGCCCGTGATATTTATTACCGGCACTGTTTCTGTAGTGGCTTCATCACCATCAAGAA
TACTGTAACCGTTATTCTCTGCGGTATTTTCAAAAGTGCCTCCGTTTATTGTAAGAGTTCCGGAAA
AGCCCTTTACAGTGTTAATCGCACCTCTTTTACTAACAGAATTCTTAAAAGTTCCGCCGTTTATTG
TAATTTCGCTTGAACCCGAGAAGGTAATACTGCTCATATTCCCGTTATAATTGAATATTCCACTA
TTTATCACCGCGGTACCATTGGCAACTGACAGAGCATAGCCCTTATATTCAGAATCATCATTTAT
AACGGTATTTTCAATATTTATAATACCGTTTGAAAGGTTTACCGCATAGCTATTTCCGTTAATTAC
AGAATCTTTTAAATGAAGTATTGAACCCGTACCGCTTAACATTATCGCTTGACTCATTGCCTCAA
CTTTTGCTCCGTCAATGGTAAGAACTGCATCTTTTACTGCAAATTTTATACCATAAGAAGTTGAT
GTATTAACATTCTTGATTGTGCCTTTTGTTGTACCTGTATCAACAACTGTAAGGTTATACATTGCT
TCTATGACCGTTGAGCCGCCTCCACTCAATGTATGTCCATTAAGGTCAAGACGCATTGCACACTT
TGTCTTTATACTTGATGTTCCCAAATCAATATCTGCACCCAACTTTACATAATCATCAGACGCTGT
AAGAGTGTTAATTTCCTCAATACTCGATACAATTTTTGCTACCGGCTCCGGTGTCGGTTCCGGTGT
TGGTTCCACCCCTTTTTTCGTCACAACATACTTGCCTTCGGCAACCTTTGTTATACTATATATATC
TGTATCTATACAATTTGGATACAGCACAGTCGGGTCTGCGGCAAACTGCCCACCTTTTATAGATA
TTTCAGTTGCTGATGAGTTTGTTGACTTTGTTTTTCCTATTGCCGATTTGAATGTTCCATCATTAAT
ATTTATTACCGGTGACGCTATAATCTCACTTGTTTCTGCGTCTATAGACTGATAGCCCTCATTGCT
ATCGAGGATACTGTACCCTCCGGCATTTGTATTCTCAAAAACACCTCCATTAATCGTAACAGTAC
CTACAAATTGTTTATCTGTAACTATAGCACCTCTTCCACTGTTTGGATTTGTGAATGTGCCGCCAT
TAATTGTCAACTCACTTGAACGTGCTACAAGAAGAGTGTTTGTGGTTCCGTTATAGCCAAATGTT
CCGTCATCTATAATGGCAATACCGCCCTGCAAATAAAGTGCGTAGCCTTTATAATCCGCTTTATT
ATTTATAAGAGCATTATCAATATTAATCTCACCGCCGTTCGTTCCTACGTTTATGGCATAGCTGCC
CCCGTTAATTACTGCGTCCTTTATATTGCACTTTCGTCCTGCAACATTAATCAATATCGCTTGACC
TCCCGCATCAATTTCTGCACCGTCAATATTTATTGTCGCTGCTTCTGTTGTACCTCTTATTCCATA
AGAGGTTTGCGTTGTACCTGTATTTATAATGGCACCTTTGGTGCTTCCTGTGTCAACAATCGTTAT
TTCGTGTCTTGGGTCCACTACGAACGGTCCCGAAGATGTCAATGTATGACCGTTAAGGTCAAGAT
GTGTCACACTTTTTGTCGTAAAGCCTGTTGTGCCAAGGTCTATATCTGCTCCAAGAATTATATTTC
CCTCAGTGCTTGTAATCGCTGCAAGCTGCTCTGCCGTTGTAACAGTTACAGGGTCCGGTAGCGGA
GTAGGCGTCGGTTCAGGAGTAGGAGTTGGTAAGCCCCATTCTATATCACCTTTTATGCTTGTATG
AGTTATATTATCAATCATGAATGAACCTTCCCCACTGTTTTCAGCAGAAGAAAATATAAAACCTC
TTACGCGTGCACAATCAAGATTGCTTCCTCCATCAAGTATCTTAAACTCATCAAACGGAACTCGA
AATTGTTTCCACTCCGTCGTCAAAGTGAATTCCTTAGTGTCTGTATAAGTAGTCATCGCACTTGCT
GTGTCAATCACCCCAACATTTATCTTTTCATTCTTGCCGCTTGTAGATTTTCCATAAAACACAAAG
TCTGTCATATATTCCATATCAGCTTTTAAAGTAGCACGGTTAGCAGTACGCTCATCGTCATTTACA
TTTCCTTTAAAGAAATCATTCGGTGAATAAACCACCTTTGCCTTTGACGGTGTGTTTCCGTTTCTT
GAATATTTCACCTCAAGTGCTTTTGAATCAAATCCAAGCCCGCCTTCTTCTTTAGTCGCAACGATT
GTATCTCCCTCACTTCCGGATTCTGTGGATATTGCTGCCTCATATTTTGTGGTGTTCTTATTATAA
GTAGCAAAATCATAAACAAATGTCTTTTCCGGATACGGAATCAGACGTTCCGGCTTTGTGTTTGA
GAAGCCAATATTATCAATGTAAAATTCGGCAGTTGTTCGCGTGTCTGCTGAAATCTGAACATATG
TTATATCTTCAGGGTATTCAACATTACCTTCTAAACTGTATATATATTGATGCCAATCGGTATCGC
CCTCTGCCATATTTACAGTAATATATGCCAGTCCGCTTAAACTTACTGTCAGCTTCTGTGCAATAC
CATTACCTTTGATATCGATAGTAAAATACTTGGCATCCATAGACGGAACAGTATTAAGGTCAAAT
TTTGCTCTGCAACCCTCAGTATCAGTTGCATCTCTTGTATATGTCACACATTTTGTTTTTGTTGTCT
CACCATTTATGTTTAAGACATTCGCATCCGTAATTTGCATTGTTGTTCCCGTAAATTTGGAATTCC
ACGCCGTAGTCTGTTTTTTAAAGCCAGTCGTATCTATATCAATATAGCTTTCCTCTCCCATATCCT
TATGAGGATATGTATTGGCAGTATAAACAGGTGTAGCGAGTTTTACAATTTTTACATTATCAATA
GTTATAGATCCCGTATCTGCCGTAGCGGCTCCAAAATATATGCCGGAAAGAGCGGCGTCAAGCT
TACCGTCAGTCTGCGGATTACCCTGTTTCACAAGTTCATTTATGCCTATTGTTACTGTTTGAACTT
CAGTGTTTACATCTACAGCTTTAGTCCATACTTCCATATCAGAACCATAAAGACCTATATTTACA
CTTCCAGCAGTTGCTGCGGAAATATCCATTGTTATTGTCTGAATTCCATTTAAATCCCAATCAAA
CGGGAAATTCAGTGTTGCATACCAATTCGCCGCCGCAGGTGTTATCGCTACATTTCCGCTTTCAT
CAGAATATTTAATCGAAGCCTTACCCGATGGAGCAGTATAGCTTCTGAATACATCGTCTGTGCTG
AAATCTGTTGAATATACACCGTCAAGCTTTTCCGGTGCAGTGTTGTATGAAAGTAAGCGATTCTT
TATATGAGTACCGTTATATGCCGTAGTCTTATAATCCCATGAATAGAGCATTTTCGGCGATGAAC
TCATATGCAGATTCCATGCTGTCCAGTTTACAGGAATTCCGTCATACTGATTGTCAGTATCGTCC
ATCCAGTTCATAATCTGATTCATCCATATATCGCTTGTGCAGTCTCCGCCCGATATGTTCTTGTCG
GATGAGTCCCATCCCCATTCACCTATAATCACCGGTGCAACACGTCTTACCGGACCTATTATCGT
ATCCCATGCGGTTTTTGCACCCTTAACCGGATATGCGTGAGAGTCATACATAACGCCATGGCCTT
CCGCCGTATCTATTAGTCTATATCCATTTGGGCGTTCATTATAGCCATCAGCAAAACCGCTTATAT
CAAATGCCCAGTTCAGACCGCCGGCTATACAGATATTATTTGCACCTTGTTTGCGAATTTCGTTT
AGAAGTTGTTGATGACCTATAGCAGTTACTTCTTCACCGCCAACTATAATTTGTCCGCCATTATA
CCACACGTCCCATTGTTCCACTGTAGTTGGTTTTTCAACTCCTACCGGTTTTATATCATGTGGTTC
ATTCAAAAGACCGAAAAGTACCGCACTGTTATTGCCGTACTTAACAGCAAGTTCTTTCCACATAT
CAAGATCGTCTTGTTGTGGCATAACATATCTGTGACAATCAAGAATTATATACTTACCTCTCGCC
TGTGCAGCTTTAACCATATCATCAATATATTTCTGATATTGTTCCTTTGTTAAGTTTTTTTCGTCCC
ATACGCTGCCGTTTTTCCAATATTTAGGATTTATTGGCAAACGTATCAGATTAGCTCCCCAGCTAT
CATAGACCATTGTCATAGACTCAAATAGATGTTCTGCCATACCCCAGTCCATACTCGGAACATTT
ACACCACGAAGAACTACCATTTCATCTGTTCCCTGCTTAACTATTTTATTTCCCTTGACCTCAAGA
GGCACAGAACGATTATAGTTAATTTCCGCATTTTGAGCGTCTGCTGCAGCTTCCTCATATGTCTGT
GCATAAACAGGCATAATGCAAAGCGACACTGTCATTACAATAGCCGTTACAAGACTTAAGAATT
TTTTCATGAATATCACCCTTTCGATTTAATTATATAAAAAGGACTGTTTTTTTGTAGGGAGAAAG
AGAGAGAGAATATTGGAAGGTATCCATTGTCCAATAAATAAAACCTATTCTAAACAGTCCTTTA
CTATATTAACATTATAAAAGAAAAGTTCACCTCAAAAAACGAGGTAAACTTTTTCTCACTACCGA
TTAATTATGTTTTCGGCACATTATTCATATTTTAATGTATAAATTTGTTTTGGAGTAACTTCAACA
TCGCACCAGCCATTTGTATCCACTGAAAGCTCTGTCTCTGAAAGTTCCTCAAGCGTGACTTTTTTA
ACACTTGCCCACTTCCTATTTTCCGTACACGGAAGTTCAAACGAGTGTACCTGTCTTTCTATTGGC
GATTGTTTATCTGATATCTCCGCTATGCCACCATTAAATCTAATTCTATTTTTAACTGTTTCACTTG
ACGGATTAAATAATCTCACCACACACCCTAAGCCGTCCTCACTGCGTTTTACGGCACTTATGTGT
AGATTTTCATTTTCAAGTTCAATAAACGACTTTTCAAGAGGGTTCTTGCCATGTTCTGTTGGTGCT
GTCTGTCCAATTAGTATTTCCATATTAAAATCTTCAGCAGCTTTCCATACTTGAGCATCTTCCCAA
TCACCCTTATGAGGCATAAAAGCATATCGGAAAGTATGTTCACCAAAAGACTGTGAACCGTTCT
CTATTCTCGAATAGTTCTGCTCCTCGGGAGTTACATATATGCGAAGCTCAAAGCAGCGCAATAAT
GACAAATACACAGTATGGTTATAATCATCATCCGATTCATACGCTTTAAGTCCCGTATTCAAAAT
CGCCGCACCTTCATTTTCATTGCATATATCAACAAACGAGTTCATCGGCTGCTCTGTCATCGGAA
TTTCGTCATACTTCGAATAATCCGGTTTCGCAATCGGACGCTTTACCACATCAAACTGTCCCTGA
GCGTATACAAACTCTGCATCCACGTCTGTGGGGAAAGCAGCCTGAAGATAGTGATTAGGAACAT
TGTTATTAATTTTCGTTTCAAATTCAACCCATCTTGCGCCTTTTCTGAGTGTTACAAGCGTTTCTAT
TCTGTAAGGCGCGAGGCGGGAACTTCTTTTCTTGCCGCCGTCAACTATATTTTCGGGAATTGCCC
AATTAAGAACTATTCTGTACTTAGTTTCAAGCTCACCGCTGTATACAAGGCTTACAATCGCCCGT
TCATTAACAGTTGTATATTCCTCATCAAGCTCCGGTGTCTTGTGTTCCCACGGGCTGCCATTTTCA
CCGGTGTCCTTAAAATATCCGATATTATCGTATTCCCTGCCGGTTTCCTTTTCAGTAACCTTAAGC
GTACCGTTTGAATTTATCTTAACCTTCAGATATTCATTCTCCATAGTATTTATGCCGCAAAGCATA
TTAACCGGCGTTGTAGCCCGTGTATGATACTTAGGTACAACCTTGAGTGTTTTATAGCCCATCGA
CGGAATATCCTTAACAAATATTCTTATTGTATGTCTTGACACAGGAAGAACATCGACCGCGTCTG
CCAAATCCTGAACAATTTGATACATAGGATTAATTGACGATATGTTCTGGTGCGGGCATACATTA
CCTTCAGCATCGACAATCTCAAAGCTGTCACACTCCCACTCAAGAGGAATTTCAAGCTCACACGG
AACCGTCAGACTTCTTTTAAACGGAGCCGGATTAAACATTACAAGCGCCATATCATTCTTATCCC
AGCCCGCAAAGTCAATGTCGCCAGACAAATCCATAAGAGCTCTTTCAAGCACGCAGGTTGCAAT
CTCTCTTGACTGCCTGAACCGATATTCTACGTCCTTATATACAACATCTCTGCCGCACGCGCCAAT
CGAATCATGCCCGTGGTTTTGAAGCATATAGTTATAGGCTTTATTAATAAACGCCTGCGGATATA
CAGCTCCGCATACAGACGCAAAAACCGCCATCGGCTCAGCATATGATGTGAGCAATCGTTCTGT
TTCAAAGTTCTCCTGCTTAACCTTAATTCTTGCAGAAAGAACCCAGCCAAACAGCGCACTTACGC
TGCCTTTTGTAAACGGATAGCGCATTTCGCCCTTTAAAACAGGTGAATTTTTATCAAAATCTCTG
ATCACGCTCTGCTCAAAGTCATAAACTGTACTGTGAAAAACATCAACACCTTCATAAACAGCATT
AGCATCCTTGATAAGCCTTGATTCTCTCATATCCGGTATTGATGAATCATGACCGTTCGACCAGA
AGCGGTTAGGCGTTGTCCACTCATCGTCCTGTTCAGAGAGTGCCTGCTCCGTCTTTTCAGCTATAT
ATTCGTCATGATACTCATATTTTCTATGCGAATACTGATATTCATATTCACATCTTGCCGGATCCG
CAAAACGGAATATTCCATCTCCAGCTCCCCATGAAACACGACGATTGTCACCGTCACGCTTGCCA
TAAAACACAGGGCGCTGCATTATATACCACATATTATATCTCGGTCTCTGCCCCAGTCTTGAAGC
ATAAATTGTTGTACCGTCGGCGCCCTCCCAGTAGAACTCCGATTTCGGTGCCATATATGTATTAA
GCCCTCTGTAAAAGGACGCAAAATCTATTCCGAAGCCATGATATAGCTGTGGCATTTGTGATATT
TGTCCCCAGCCAAAGGGCGAATAGCCTGTCTTTGAAACTTTGCCAAATTCATTTGCAATTTTATG
TCCCAGGAGAAGATTTCTTATAAGAGACTCTCCACCCACGCAAAACTCATCCGGCAAACAAAAC
CACGGACCCACAGCAAGCTTTCCCTCACTGATATACTTTTTCAGAATTTCCTTCTTTTCAGGGTTT
ATTTCGAGATAATCCTGAATGGGAAGTGTCTGCGAGTCAAGATGAAAATGTTTGTAATCCGGCTC
TTTCTCAAAAATATCGAGCAGCATATCAATCGCAGTCACAAGCATATGTCTTGTTCTTTGAGCGC
TGAATTTCCACTCCCTGTCCCAGTGGGTATTGGATATAAAATGACATTTTATATTTTTTCGCTCCA
TTTACGCTTCCTCCTCTATGTAATCAAGTATTTCCGCAAGATTATTGCAGTAGCGGCTACACATA
GGCATCGTTCCGCTTTCTTTTATATTGCCTCCCGCAAGTCCTATTGTTGTATATCCGGCAATCCTT
GCCGAGCATACTCCTGCGCCGCTGTCCTCAATAGCGATTACGCTACCGCGCTCATCAAAACCAAC
TCCGAGTCCAACCGCGCAGGTTTCAGCATAAAGCCACGGATGAGGTTTCGGCGATAGCTCACCG
AGCGTTCCAACGCTACCCTTGCGGAGCGGGTATCCTGCTGAAATTATTGCGTCGTAAAAATCCGT
AGGCTCGCCCATATCAAGAGCTCTGAACGCCGAAAGTATTTCCGGCATCGCCTTTTCATATAATC
CCGATGTTACAAGTCCTATCTTAATCCCTTTGGCTTTTAGTGCGAGCAAAAATTCTTTTAATCCCT
CCTGCGGAACAAAAGCATTTTTTCTGCCTCTGCCCTCCATAATTTCTTTCATTTCACGATTAACGT
GGTCAAAATAGAAATTCCGCGCTTTGTCAAGCGATTCACCTGGACAGTATTTATCTATACAATAC
TGTAAATGCTCCGATACGCTATGACCTGATACAAACGGTATATCGCTCTCTTCAAGCTTGAAGCT
TTCATCATCAAGCATACTGGCGGTTGTTTTTTCGATTATCCAAATCCAGAACTCCTCGCTTCTTAC
CGATGTTCCATCCAAATCCATAAGCACAGCCTTGATTTTACCGTTTCTGTTCACAGGATGAAGCG
GATAGTACGCACCATAGCCAAGAGCTGAATTAACATAGGCAAGAGTCTGATTTTCAAAAGCCAC
AAATTCAACCTTTCCGTCTCCTGTGCATACTATATGTTTTACTCCGTCCTCACCGGAGGTAAATCT
TCCGTCTGTAGTGGAGGGAAGGTTGCGAAATCCTGAGCTGCCGTCTATAATTCCCAAATCGGGTA
TATTGAAATTCATTACATTACCACCTCTACATTGCATATTTCCGCATCCGATGAAACGGTTGTACC
GTTTACCGTTCCGTTGTCGGCTGTTATTTTAACAGTTCCCTCTCCTGTGTTCTTTACTAAAATATTA
TATGTTTTTCCTCTGAATTTTCTTGTTACCTTAAATTCAGATATATCTTTTGAAATACACGGCTCTA
TTACAAGTCCGTCAAAATCAGGACGTATACCCAAAAGATATTGTGACACCGTATAATAATTCCA
CGCCGCCGTGCCTGTAAGCCATGAATTCTTCGCTTCTCCCGCTCTTACCGCATCACGTCCAGCAA
TCATCTGTGAATATACATACGGCTCGGTTTTGTGAATTTCACTTATCTCCTCTAAATATGCCGGCG
CAATCTTTTTATATAGTTCAAACGCTCTTTCGCCGTTACCCATTACCGTTTCACCGATTATTACCC
ATGGGTTATTATGGCAGAACTCTCCCGCATTTTCCTTATACCCCGGCGGATACGACGAGATTTCG
CCAAGCTCAAGTCGGTATCCCGAATACGGCGGATAATTGAGTACAATGCCATATTCACATTCAA
GATATTTTTTAACGCTGTCAAGCGCTTTTTGCGCCCTGCCGTCCTCCTTGCCTATCCCAGCCATTA
CACAGAAGCCGTTCGATTCAATAAAGATTTTACCCTCGTCACATTCGTCAGAGCCAACCTTATTG
CCGTTAGCGTCGTAAGCTCTTATGAACCACTCACCGTCATAGCCATACTCTAAAACTGCTTCGGT
CATTTTTTCAACCTCATCCGAAATATATTTATACATTTCATCATTGTTGAGCGTCTTATAAAGCCT
TGCAAATTCTCTGCCAATATACACGAACATTCCTGCAATAAGCACAGACTCTGCTACTCTGCCGT
CATCGTCTCCCGTAGTCTGGAAAGATTCGTCAGGAATTTCGGAAAAGCAGTTCAAATTCAAGCA
ATCGTTCCAGTCCGCACGTCCGATAAGCGGAAGCCCGTGAGGGCCAAGGTTGTTTGTAACGTGT
CCGAATGAGCGATTAAGATGTTCTAAAAGCGTTGCCGTATTGTTTTTGTCACAGTCAAACGGCAC
CTGCTCGTCTAAAATACCGTAGTCGCCTGTTTCCTTGATGTACGCAACCGTGCCTAAAATCAGCC
ACAGCGGATCATCATTAAATCCGCCTCCGATTTCATTATTGCCCTGCTTTGTAAGCGGCTGATAC
TGATGATACGCGCTTCCGTCCTCAAACTGCGTTGAGGCAATGTCAATAATCCTCTCTCTCGCCCTT
TCGGGTATTTGATGTACAAAGCCAAGCAAATCCTGATTTGAATCGCGAAATCCCATTCCCCGGCC
GATACCGCTTTCGTAATATGATGCACTTCTTGACATATTAAAGGTCACCATACACTGATACGGAT
TCCATATGTTTACCATGCGGTTAAGCTTTTCATCGTTTGACTCAAGAGTAAACACCGAAAGGAGA
TTATCCCAGTACAATTTAAGTTTATCAAGCTCCGCGTCGCATTGTGCTGATGATTCATATCTTGCA
ATCATTTCCTTTGCCTTTGTCTTATTGATTACATTAAGGCTTTCAAATTTCTCGTCCTTTTCATTCT
CAATATATCCAAGAACAAATATGTATTCGCGGCTTTCTCCCGCGTCAAGTGACACGTCAATCTGA
TGAGATGCGATAGGATACCAGCCCGAAGCTACTGAATTGCCGCTCTTGCCATTTATTACTCTGTC
AGGAGTGTCAAGACCGTTAAAGGCTCCAAGGAAAGTGTCTCTGTCTGTGTCAAAACCACTTATTT
CGGTATTTACTGAATAGAAAGCGTAATGGTTTCTTCTCTCACGGTATTCTGTTTTATGGTATATAG
CGCTGCCGTCAATTTCCACCTCGCCCGTATTGAGGTTACGCTGATAGTTAAGCATATCGTCCTGA
GCGTTCCAAAGACAGAATTCAACGAATGAAAACAGATTTATGTTTTTAGCCTCACCGCTTGTATT
TGTCACCTTAATTCTATGTATTTCGCAGTTATCGTCCACAGGAACAAAAGCTGTCTGCTCAACGC
GGACGCCGTTTCGCTCGCCGGTGATTTTTGTGTAACCCATACCGTGACGACACTCATAAAAATCA
AGCTCTTTTTTCATCGGCATATACGAGGGTGTCCAGCAATCGCCATTATCGTTTATGTAAAAATA
GCGTCCACCGTTATCCGCAGGGATATTGTTGTATCGGTATCTCAGAATTCTTCTGTGCTTTGCGTC
CTTGTAGAAGCAATAGCCGCCGGAGGTGTTTGAAATTAATGAGAAAAATCCGTTTGTTCCAAGA
TAATTTATCCATGGAAGCGGCGTTCTCGGAGTCTCTATTACATATTCCTTATTCAAATCGTCAAA
ATATCCATATTTCATATTTGTTCTCCTTACCAATAAATTTTCCAATCGCTTTTTTTCATGCGCATTA
ATGCTTCAAGATAGAAGTAGTCTCCCCAACTTGTACATTCCGGCTCATGACCGTGTTTTCTGCTGT
ACATACCGTCTTTTATAATTCCGTTGCTTTGAGGATAATCTACTGTCGTGTACTTTTCCGAAAGAC
TTGTCATCATCTTTTCCGCCGCATCAAGGAATTCCTGATTGTGATAATATTTTTCCATTTCAAGAA
TTCCGCACACCGCAACCACTGCTGCGGAGGTATCCCGTGGTTCATCGCTACCATCGGAGAAAATC
AAATCCCAATACGGAACAGAATCCTCCGGAAGATGGTCAATAAAATAATGCGTAACCCGTTCAA
ACAACGGCAAAATCGACTTCTCTTTAGTATAGTGGTAACATAGAGCAAGACCATATACTGCCCA
TGATTGTCCTCTCGCCCAACTGCTGTCATCGGAAAATCCCTGATGAGTTTCTCCCCGAAGCGGCT
TATTGGTTACAGGATCAAAGAAAAAGGTGTGATATGAAGACGCATCAGGACGGATAATATTTGC
GATTGATGTCTGCATATGATTATAGGCGGCGTCATAATACTTTTTGTAGCCTGTCACTTCGCTCGC
CCAGAATAGAAGCGGAATATTTAACATACAGTCAACAATAAATCTATAACTTTGCGAATCATCC
ATAGCATCCCACGCCTGAATAAATTTACCTTTTGGCTGATAACGCTTCAAAAGCCATTCTGCCGC
CTCAATTCCATCCTGCTTCGCCTGTTCATCTCCTGTTATTCGATAATCGGCGACGCTTGAAAGTGT
AAACAAAAAGCCCATATCATGATGCTCCAGTTCAACTCGATCAACCAACCTCTTATGAAACATTT
CACTGTGATGCTTTGCCGAATTATAAAAAGCTTTATCACCTGTAAGCTCATACATAAGCCATAAT
ATCCCCTCATAAAATCCGGTAGTCCATGAAACGTTCTCAAATTTTTTGAACACAAGATTTTCACT
TTGTTCTGTAGGAAAACAATCATAGAAATAATTTAAGCTTTTTCTTACGATGCTCTCCGCATATGT
AATCGCTTTATCAATATTCACTTTTTATCACTCCTCCTACTATAATAAATTTATGCGTAAATCACA
CGACAGTCATTCGTGTATATATCCATCTTGTTGCTTAAACTGATATAATGAAAGTGCAATTTCTG
ACGTATCACGCCTCAGATTTTCTGTCTGTAGATAAGTCAGTCTCTCATTTGTTGTGTTCGATTTAT
GCAGTAGATAAAAGGAGCTTTTATCCAGCCCTTTTTATTATAATTAGTTGACGCTGTTTACCGCCT
TCGGTATGAAGGATTACCTTTGATGCTGCCTCTCCTGCACTCTGCTCTGCGAGCAAATCATCCTCT
GTGGGAACATATATTCTATTTGTAGTCTCATCAATAAACAACACATTATATCCCGAGAAATCAGC
TATTTCAATTTTGTCTGTCTGTCCCAGAGCAGTATCAACCTTTGTTTTCAGAACAAGATTTGTTCC
GTTTCTGTAAAGCAACGTACCATATACCAATCTATACATTGCACCGTAATTATAGCTACCAATGG
CATTGTATATATGTGATGTATTATGGCCATTTTCAGCATACTTTCCGTCTGACACAGCAATCATTG
TTTTATTTAGTGTTGACGTCTCAACAGTTTTTCCTTCTTCATTCCCTGTTTTTACATAATCAGGATT
GTCTTCATCCTTCAAAGAGAATACCTTAACATAATCCACAAGCTCGCCTTTACTGTTTGTAACAT
AACGAATTATATCACCATGCTTTACTTCCGACATAATTTTTGAACCGTTAGGTCCATTATAGTAA
CGTTTCAGATAGACATCTTCTGCAAGGTCAACCTGCATTTGAGCATTACACTGCCAGCCGATTAT
TCGAGTTACACTCTCATCATTATCATTAACTGCCTTTACTACTTCGTCCACTACTGTAAGGGGTAC
TTTTTTATCTATCTCAGGTATTACGTCACTTGTATAATATACTATTACACCTGCTGTCCGAGATTC
ATCCACGTTATATGCTTCAATGCGATTTTCGGTTGTGTATTGTGTCCCGGTCTGCGGATAATATAC
CCAATCATCGAAATAGGTTAAATTTGTCTTAATATACTGTGATGCATCTCCGATATCCCCTCTATC
GTTTGAAACGGGAACTACAAAAACCTTTGTCTTTGAATTTATCGCCACATTATTTCCGTTATCACT
ATTAAATGCAAGTGTATTTCTAAGATACATGCCTCCCGGTTTTATGGAAGATGTCCTTTTAGAAA
AATCGTTATACATTGTCATAGAATTGTCATGGAATACATCATTCCACTCCGGATTTTCATTGTCGC
TATGATATGGAGTATCGATATATTTAATCTCTCCCTCATCGTTGAGCTTATACATAATTGGGACTC
TTACATACACATAACTGTCATCACTATAATCAGCCATTATCTTATATGACGAATCCAGTGTTGCA
AGCGTAGACCTCTGATAATCCGCTGCTATTTTTAATGCAGTTATTATTTTTTCTGCATCTTTGCAG
GTTAAACCATCAATTTTTGTACTTCTTGCAAGTTTGAATTCATTCATATCACCAGTATAAGGAAGT
AGTTTGGCTATAACATCGCTCCTGTTATCCTCAATCCAAGCCTGTATTAAGAATCCATATTGTATA
TCGTCAGTTTTAAGCAAATCATAACCGGCTATCCTACCTCGGTAATCAAGATACACCGTATATTT
ATTTCCATTTTTAATTGCTTTTGCGTCTTTATAGTATTCAGCATTTGTTGAATACTTATATGTCCTG
TCCTCAAATTTAATTTTAGTGCCATTAGTGCTCTTTACTACACCGGTATACATCAAATTGCTTACT
ATAATCTTTGTCTTGTTTGTTTCAATATCTTTAGCAACTGATAGAATTGAATCCTCTGTTATATAA
TAAGCATCAACAGGATTGCCCATACGGTCAGTCATGTCGCAGTCAGCAAAATTTACCTTTAATGA
CCCGTTTGGGTCATCATTGTACAGACCATAAATCATTCCCTGCGACTCTACAATACGTTTTACAA
CCATTATGTCATATTTGTATACAAAAACAACATCGTACTTACCATTGTTATCGTTATCCACTAATC
TGATATAATCGCAGTTATAAAAATCTTCCTCGTCTAACAATTTTGGCGAAAATCCGTTAAGTATC
TCTTTCGCTGATGAATTAAGAGATACTTTTTTTGTTTTATTGTCGCTATAGTATCGTATCATGTTAT
TTTCAACACTTTTAATATCTTCGCCGTCTATCGTTATTATGCTGTTATTTTTGTGATAAGAGATAT
ATAAAAGAGTATTTCCTCCGGAATCTTCCTTTCGATAATATGCCTCCACCTTACACGCAAGGTAA
TCATATAAATCACTGCGGTCACTGGCAAGAAGATAATCTCCTATTCTAATACCTTTATATATTGT
ATCACCACTCGTTATTGCATGCTCTGAAACAGATTCTACAACTCCGGTGATTTTATAAGCATCCTT
GAAATACTCTAAAGTATTAATGCTTTTATGACCGTTATTTCCATATGAAATGTATACATCGGCCTT
TATAGAATCATTAAGAAGAGATGCCGCATCAGTTCTTCTGATAGAAGCACTTCGGTTATCCAGAA
AGTTCTTTGTTATATCATAATTTGCGGCAATTTGTATATACGCATTATCATTGCCTCCATACATAT
AAGCAATCTGCTTATACCCCAAAGCATTTACAAGTGTTCTGAGTGCATTATCAAAACTTATTTCA
TCCGTACAATCTATATTGAAATCTACATATCCCATACTTTTAAGCATATCATGGGCTTTTTCCCAC
ATGTCTTCTTTGTTTGCATCTTCGTCATAAAATGAGTCACAGTTTATGTAGCGGCACACTGACAG
AAAAAATTCACCTGCCTTGATTGGCTGAAGATATGAACCATCATCAATAGGAACCCACATTTCA
AAAGCTGTAAGCTTTTTTACTGTTTCATCATATTCATTCTTACCCATAATGTACGTATCATATTCA
ATATCCTCTATGTATGCGTCATCGGGGTTATCCAGTTCATGCTTAGGGTGCGAGTCTTGAATGAG
AACATTTTGTTCCTCATCGCTTAGTCCCGCATTATTATCTCTGCTGCCGTCATCATCAAAGTTTTC
AGCTGAAAACACTGATATTGCCGCTGTAAATATTACACTCAGAGAAAGCATCAACGCAATTATT
TTTCTTAATTTATTCATGTTACTTCCCCCTTTATCGTATTACTATTGCCGCAATCGATGGCACATC
GTTATGTCTTGAAATAATCACTCTCGAAGCTTTTGCCTCTCCTGCAAGATTAGATGGAATTATATC
ATCAAGACTTCCCAAATGAACTCCTTCGCGACTGTCCTCCAAAATATATACACGATTATTTTTAA
GATTGAAGTATCTTTCGCATTTCAGACTGTTGCTTCCTGCTGTTCCCGGATATGTCTGTATAATCA
TTGATGAACCTGTTATTGATTTTACAATGCCAAATTCCGTTGAATATTGAACACCGGTGAACCAG
TATGGATTTTTGCCTTGCCATATACGTCCCGATACAACATCGGTATCAGGATTAGCAGCCATGTC
ATCAAATGCGATAAGTGTCTTTGCAGAACCGATATATGACTTTGTTTCTCCGTATTCATTGCCTCT
GATTACCACATCGGGATTATCATTGTTATCAAAATCAAATATTTTATGATAGTCAACAATTTCAT
TATTAGCATTCGTTGCAATCCTTATAAGGTCTCCCTCGTCAATGGTTGATGTAACAGGTCCTTCGC
TGCTATTATACTGTCTTTCAAGTTCTACTCCCTCAGCGGTCACAAATTCCTGTTCTGTACCATTGT
ATAAAAGAGTTAGTAAATAACGCTCGTCATCATCAACAGAAGTGAGCGAAACATTCTTAACCGC
CGCCATAACAGAACTGTAAGTGAGCTCATTTCCTCCTGCATTATCCGAACGTATTACGGCAATTC
CCGCTACCATGTCCTTAGACATATTATATAGTTCCACCGTACCAAAAGTTGCAGACTTCAAATCT
TTAAATTGCTTTATCTCATAATATTGGGAGTCGTCCATTAATGATTTGCTCGCCGGAACATACATT
ATTTTTGTCTTCTCCGAAAGTCTTGCATAACCCGGGAAACTCCTATAGTTTGCATTATAGTATGTG
TCAGAAAGGTCTGTGCTGCGGGTGAATACACTTGCCGTTGTATAAAGCTCGTCAGCATCTACATA
TTGTGCAGTTTTTACTGACTGAATTTCACCATTATTATTCAGCTTGTATCTTATAAGCTGATTAAC
TGAATCCGGGTCTGTATTTACATCCTTGAATAGCTTTTCAATGTCTGTATATTCATTTACTTTTTTG
TTATTTACCTTTGCATTCCGTGCGAACTCTAATGTTTCCATATTTCCGGATGTAGTATATACTTTT
AGGTATGGTCCATCGCTATGCTCTGCCGGAAAAGCTTGTGCAAGATATGCAAAGTTATTTACCTC
ATCATTGTCATCAAATTCGGCATATGCTATTCTGCCACGATGGTCAAGCAATACACTGACAGCAT
TTCCTACTGACAATAATGAATATGTTGTATTTTGTGCCGCAAGAATATCTGTCAAGTCATACTCC
GCATCATCAATACTAACTGTTAAATTCCCGTTGTTTCGAGCAGTCCTCTTAACAACTCCGTCAGCT
TCTTCCCTCGATATGTAAATTACAGCATTTTCTTTTTGTTTATCCTCGATAACTGAAAGAATATCA
TAAATCTTTAAATTTCCTATCGATGTAAACTTTTCGTCAGTGTCATATACAATTACAGTTTCCAAA
TCATTAAGTTTTATCGACGGCTGATTATAATAATCATATAATGTATTTTCATACGGTGTAAGCTG
ATTTATACAGTATATTGCTTCTCTGATTACATTTACTGTATCATACATTCCATCATTGTTACTGTCT
ATCAGAATAACCTCATCAGCGTTTTTAATGTCTTCTGTATCATATTCAGCAACATAATTGTAATTA
TACAAGCGATTTATTGTCTTCGGAAGTGTTTCTTTCTTCTCCGAGTTTGTTGTTTCATTTTTATAAT
ATTTCAATACAGAACCATCAAAGTCTGAAATCAAGTCCTGCTCTAATGAAAGGACATTATTTTTC
TGGTTCGGCTCAATAAATTTTAGAGTTTCATCATCTTCAACGTAAAAAGCGTTTACTCTGTAACC
AAGGTATCTGTATACATCTTTTATATCCGTACTGAAACTATAAGAACCTATTCTCACTTCATCTGC
ACTAAGACCTCCACTTCCGTCTAATGTGCGAGTTCCACATACATATACTATATCATCAAGAGTAA
GTATTCTATGATAATAATACAAAGGTGTTATATCAGAAATAGAGTATATTGACGATTTATCCAAA
TCATCCACAACCATATATGCCTTGGACGCCGAATCAAATATCTCCAATATATCCATAAAAGAAA
GCGTATCATCAATAGTCTTTCTCAAATTCGGAATAATATCGTTGCTTACCGCTACACTGTAGTAT
GAACTGATATTACCGCCGTTCTCGATAGCATAAACATCATATCCCAGTACACGGCACATGATAAT
AGCAACCTCACCCAGAGTTATCGGATTGTCAGCCCCAAATACTGAGTTGTTCTTATCAATATAAC
CGTTATCATAAAGAAATCGAATTTCATTGTAATATGTACTTCCGGGCACCACATCACTGAATATG
GTCTTGTCATCGCTTTTCTCATAATTTTTAGCATTCACAAAACCTGCCAGATATTTTGCAAACTGA
CCTTTCGTTACAATACCGTTTTCCTCGTTAGGAAAAGGAATTATATCCAGTGCCATAAACTTTCC
GGCAAGAAGCATATATCTGTCACTGCCTGTCAAATTCGTATACGATATGTTTCTTGATTTCATAA
ACAGAGATACAGAGTAAAGTATCTGAGCAGTCTGTGCTCTTGTTGCATTTGCCCTCGGCATAAAG
CTTCCATCACCCATACCGTTTATAAGTCCAAGATTTTTTAGTGCATACACGCTATCTTTAGCATAA
TCAGAAATATCACCATCATCTGTAAAGCTATCTATCGCAGCACCGTTCAATTCACAGTTTTCTTG
CTTAAGATATCGTACTATAAGAACTGACATATCTTGACGTGTAATTTTTTCTCCCACACCGAATA
TATCTTCCTCAATTCCACTTACGATACCCATATCGGCAGCAGTATTTACATATGGAGCATACCAC
TTATTCTCATCCACATCACTGAACTTATTATCAACATTTTCCGTTTCCTTTCCGAAAATTCCAAGA
AGCATCTTAACAAACTCCTCACGGGTTACGGAATTATCTGTGCCAAAGCTTCCGTCTCCGCGTCC
GCTGATAACTCCAAGATTTTTTAATGAATTTATCGCAGTATACGCCCAATGGTCCGTATTTACAT
CTGTAAATATTTTATTGTCTTTTTCAGATGACACAGTATCATTTCCGGTCACTTGCGAAACTATCA
TAGATGTACTGCCTTTTCCCGAACCGTTTCCTGATACTGAGCCTATCCCGTTCGTTTTATTTGGAT
TTTTACTGATAGAATTCATGTACTCATCAAGCAATGTCCCTAAAGATGACAACGACTCCACTCTT
TTTTCAGTTACATATTTATAATATCCTGTACGATTACTGGCACTAAGTCCCTCAAGGCTGCTATAA
CTGATATAAGGTGCAAGTATATTCTTATTTTCTGAAATAAGATTGCCAATCTCACCATAACCATT
TACGCATCCAAAAGCCACAAGCAGAGCATTTTCATTAAATGCACTTCTCAACGATTCTATAGAAT
CATAATCATTTTTTGCCGTAGCCGAAAGAACATTGCTGAGTAAACTATCAGATGTTATATACTTT
TTAAAATAATCGGTTCCTCCATCAAGTTCAGCCAAGTCATTATATGAGGAATATATCTCTTTTAA
AGTTTGAATTGAATTTGTTTTGTTTAAAAGCTTAACTACAAGTTCTTCACCGATGTTTTTTCGTAT
ATCGCTTACTGCTGATACGTTTCTGTCGGCAACCGACACGGCTTTTGCAATTTCAGACAATGTTA
ATTCCTGTGACAAATAATTGTCAAGATTTATTATTGCCTTATATTTTTCAAAATAATTTGCACAAT
CAGATTCGGTAGTGCCGAGAGTTTTATACTCGCTTATACATGAGTTTATTTCTGTCTGCGAGGCA
TTATAAAATTTTCTCGATACTGTATTTCCATACGATAAAACCGCATATATTTGTCCATATGCCATT
CCACCGGTATGAATAGCACTGTTTATTGTATTATTTTCAGAATTAATAGTATACGCACCTATATA
CTTATCATCTTTTTTAAACACAACAAATGCAGTTCCATCTCCTGTGACGGTTCCGTTTATGTTTAT
AGTTTCACCACCGTCAGAAGCTATTATATGACTTTCAAAAATCGTTCCGGAAGGTGCAGTGTATT
GCATTACTCCGCTTCCCACTTCTATAGGCGGGCATAAGAGTTTATAATCGGTTTCATTATTAAAA
ATGTATATCTCTACCGAACTATCACTTTCTTTTTTGATGTTAAAATTAAATGTTGTTGTATCACCC
GCACCAATACTGCGTTGCTCAGCAAAAGACGCACTTGTACATACTCCATCGTTGTTTTTTACAAC
TGCGAGAGCACATGCTTCAACGGAAACACTCGATGATTCATTTGTCACCGGTACACTTATATTAT
TTCCGTTTTCTTCAATATCGCTCACTTCCGGTTGTATACCGCTTCTGGCAGTGAAGTTTACATTAT
ATGTTTTCTTTACTTTTCTGCTGCCTGAAGTGATTGTAATCATTCCGTTTCCGGGAACAGACTCAG
GAGCGGTTATTTCATATGATGCACCCGTAAGTTCTTGGCGTGTATTGGTCTTTGGAATAGTAGGT
GTCTCAACATCTACGGTTACATTTTTCTTAATATCCTCCGCAGTATATGACGCCGGCACAGGAAT
ATTATTTGATGACGCGTTTTTGTCAAAGTCCTCGACTTTTACTCCCTTTACAAAAACTTCTGAAAT
ATTTGCATTTTCAAAATACGCTTCTTCTCCGGTTGACGGCGGGGTTATTGCCTTTACTTCATCTGT
AGAACGGTCAAAAAGTATTTCGTCAATCTTCAATGGTTCTTCCCACGGTGTTTCAAGTGTATCTG
AAGTCGGATTAGCACCAAAATAATCTTTATACGGGAAAATTATTGTCATTTGATTAAGTGCCGTA
TAAATATCGCTCTGTATTCCTTGGTCCATCGTTACTGACCCGGACTTAAACGCAGATGTCGGTAT
TGTTATGTATTCCCATTCGCCGTTGTTTGGAAGTTGGAACTTTTTTGAATACTTCTTGTTCCCCTCT
GTACTTGAATACTCCATGGCGATTTCAAGAACACGATGCTCTGCTGCACCATTCCCGTGGTCAAC
TGTCTGAGGTGTATGTACCCACATGGAAATATTTTTTGTATCTCTCATAAGGTCAAGCATTGAAA
TACTTTCATTAGCAAGTTCTATCGGCTCGCTCTTAAACTGCATTACAAAGCCATTGTATCTTTTTG
ACGGGTCGGAAATCTCATGTCCCGGATATGTTATCTGCATAGCATTTCCATACTTACCTGATACA
TAGCTTTTTTTAGTGCTTATAGTACCTCCGTTATAACTGTAGACCATACGAAAGTCAGAGTCACTT
TCCATTGAAAGCTTATAGTTATATTTGATTTGTGATTCTGTATCCTGTGCGGTTACAACAATGTTT
ACACCACCAAGAGCAATTACAGCGGCACATATAACCGAACAGATTTTTTTAATCATCCTCATTCT
CTGCCTCCATTCTATTTTACTATCAGTTTATAGGTCTGCGGCTCAATACCGTTATTCGATACATCC
GATACGGTTATTGTATATTCTCCCTTTTTATCCTTAGGAATGGTAAATTTAAAGTTACTATACGAA
TAATTTCTCCACGGCCAATGCGGATATGAAACCCATTTCGGATAAACCTTGTAAAGTGCATTATC
AACATTGTCAACAGGCAATCCCTCAACCTTTAGTTCACCATTTATCTCATGCTCCGTTTTATTCAT
AATATGAATTGAAATATGTTCTTCAATTCCTGCCTCAACCTCAAATGTCTTTGGAATTTGAATTGC
TTTGTTTGCAGGATATTTTGTAACCAGTTCACCTATCGGTTCCGATTTGACCGGCGTTGCATGTCT
TACTGATTGTCCTGTGAGTTTATCACCAATATTCGTAAAAGCAAAATCGTCAATCCAAAGTCGTC
CCGAAGAGTTTAGTGTCATATTAAAAAGACTGATATATCTTACCTTGTTAAGATGAAGCATATCG
CTCATACCGAGGTCGCTTATCCTAAATTTATACTGTTTCCATTCCGTGCTGTTGACATCGAATGCG
ATACAAAATCTTTCAAACTCACGTTTCTTGAAATTTGAACGCTGTTCATTTTGATTTGTACCTTTT
TCCTGTTCAAAACGCAGTATAAAGCGTTGCTTAGAACCATCGCCCTTTGCCCAAAATGTAAAATA
TTCAGCATCGCCTATATCCCAAGACTTAGGGATTGTTGCCCTCGCCTGCCCTCCATACGAACCTT
CAGGGCAATTATAGCTAAACATAACAGGATTCGAGCCTTGTCCCCTGCTTTCTTCAACTGCATTA
GTAACAGTAAACGAGTCTGTTTCTGTGTTTTCATCGTCTTCCCATGCCTTCCATGTCATACTTTCC
TTTTGGGTAACGCTCGATACCGTAAAAGGCTTTATTTCTTCGTATGGCAGTCCTCCCACTTTTATA
TTATCAATTATAAAACTGCCGGGACCGTCATCAAGAGATGTAAAGAGTACAGACTTAATTTGTGT
TGTATCAAGTATCCCTTGCTCATTCTTAAACAAGCTTAATGGAAAAAACCAGTCCTTCCAGTTAT
CATGTAAATCCATCTGCATATAAGTTGTAAATAATCTACCATCTTTCAACTCAAGTCCAATTCCG
ATTTTACGCATATCACCGTCGCCTTTTATTCTCACGGCAAGCCATTTTGCACCGCTCAAATCAGTA
TCTTTATCAAAATCAGCAATCGCAGCACTGTCCCAACTATCAACACAGCTTCTGTCAAAATCAAT
AAGCTGACCTCTGCCTTCATAACCACTATCTGTTCGTTCTGTCCTTACTTCTTTGCCATACACTTT
AAAGTCCACGGTATTATCGTCAAAATCAATAATATGTTCCCAAGTCGAAAGTGGTGATGGGTCG
ACATACTGTCTATCTTTTTCCTCCGGACGTGTTTCAGGCTCTTGAAGCATTTTATATTTCATATAT
ATACCCGAATAATTTGATGGTGTAAATGTGTCATAGTCCATAAACATATTTGGATTTGCACCCGT
GTGAAAAGACCATGCCGTATAGTTAAGTTCATTACGGTCAATAAAATCATGGATTTCGTTCATCC
ATACATGTGGATATTCACAAAGATAATTGTCATACCAATCAAAAAGTCTTTCTCCCCAGTGTCCA
TATTCACCCACAAGTATAGGAGCTTCATCTACAAGACACTCTACGGCTTTTTCAACCGGATTATA
TTCCGGCTTCATTGGATAAATGTGTGTGTCATAGATTACACCGTTACCCGTTTTATCCTCTAATTT
ATAGCCATGTTCCATGCCGTTATATCCATCGCACAGACCGTCAAAATAGTATGACCAATCAAGAC
CGCCCGCAATTACAATATTTTTTGCACCCTTGTTGCGTATCATATCCAGAATTTCTTGATGACCAT
ATACACGCTGTGTTTTCTTTCCATAGCTGTCGGTAGTTTCAAGCATACCGCCGTTACGCCACATTT
CCCAGTCAATATCATGAGGTTCATTGAGCAAACCGAATATAACTCCCGGATTATTTCCATAAACC
TCTACTGCATCATTCCAGAAATCCTTCTGCTGTTCTGTAATAGCATAAAATTCATGCAGATTAAG
TATTACATACTTTCCGCGTGATGTAATTTGATTAATTACATTATCAACCATTTTGCGGTAATCACC
ATAACTCTTACCCTGATACCATTCTTGTCCAAACCAAAATTTAGAATGAACGCACAAACGAACAC
AATTTGCATTCCAACTGTCACAAAGAAGTCCTACTCTCTTCATAAGGTCATTATCTCCGCCTGTG
GCCCACTGCAAATCCGGAATATTCGCTCCGGTAAGCCTAACCTTTTCGCCTTCAGCATTATATAT
GTATCTGCCTTCAACATGAAGCTCCGAGGGTAATATCTTTTGTACCGTTCCAATTTTAGCTTGTGC
TGATTCCTGTACAAGCAACAATGACATGCTAAAAACTGCAACAAGTAAGCAACTGATAACTCTT
TTTATTTTCATAGTAAACTCCTTATCCGAATAAATATAAAATTATTCTTTTACTGAACCTACCGTC
AATCCTTGAATAAAATATTTCTGGAAAAACGGATAAACAAACAATATAGGTAAAGTCGCTATTA
TACAAAGTGCCATTCTCGCACCTTCCTTAGGAACATTAGCGAGAAGATTTGCCGAACCCATCTGA
GCTGAATTTTCGGTCAAATTCTGAATATTTGAAATCATACTTTGTAGAAGATACTGGAGATTATA
TTTTTGAGGCTCTGTTATATAAAGAAGCGGAAGCCACCAGTCATTCCAGTAAGTAAGTGTTGCAA
ACAGTGCAATCGTTGCCAATCCAGGTAAAGATATGTGCAGAACAATCTTAAAATATATCTGATA
CTCATTTGCCCCGTCTATCTTCGCCGCCTCTATTATTGCAGTCGGTATTGACATTGAATAGAATGA
CCTCATTACAATTACGTGCCATGCATTCATAACATAAGGAAAAATAAGTACCCATATACTGTTCT
TCAGATTAAGAATGCCTGTTGTCACCATATATCCTGCCACTGTTCCACCACTGAAAAGCATTGTA
AAAAACGCTATAAATGTAAACAGTTTTCTGTATTTAAAATCCTTTCTGGAAAGCGGATATGCATA
TAGTGCAACCACCAATGTACTCATAAGAGTTCCTACAATCGTAACAAAAATAGTTACACCATAT
GCTCTTAAAATCGTTTCTTTAGATGTAATTATATATCTATAAGCATCAAGACTCCATTCTTTAGGA
ATAGCATGAAAACCAAACTCTGAAATTGCCGATTCACTTGTAAACGATGCTCCAAGAACCACGA
GCAAAGGATAAACACAAGCTACTACAATAAGAAAAAATATAAAATAAAGAATAATGTCCGAAA
CTTTGATTTTACGTCTTTTTTTCTTAGCCGGAGCTTTAACTTCCGGCTGTTTGATACTATCTATGAT
ATTAATTTCTTGTTTCTTCACTGTTATACCTCCTTTTAGAATAATGCGTTTTCGGGGCTTATTTTCT
TCACAATAAAATTAGTTGTCATAACAAGTATAAATCCAACAATTGATTGATAAAAAGCTGCCGC
TGAAGCCATTCCTACACTTCCCGTACCCGCTGACATCAACATATTATATACGTATGTACTGATAA
CATTTGTAGCCGGATAAAGGGCACCGGTATTTAACGGCACTTGATAGAATAAACCAAAATCGGA
ATTGAATATCTTTCCGACATTTAAAATTGTAAGAACTACCATAAGAGGAACAAGTGCCGGCAAT
GTTATGTATCGTATTTGCTGCCATCGAGATGCTCCGTCAACCTTAGCGGCTTCATAAAGCGAGGT
GTCAATTCCTGCTATACCTGCAAGATATACAACACTGCCATATCCCGTTGTTTTCCAAAAGTTTA
CTATAACAAGAATCCATGGCCAGTATTTCGGATTTGAGTACCAGTCTACACCTTCTAATCCAAGT
GCCGGAATAAGACTTCTATTTATCAGACCGTTCTCAACGTGAAGAAATGCCAGCACAAGAAAGC
TTACAATTACGTATGATAAGAAATGCGGCATAATTACTACTGTCTGGCACAGCTTTGAAACTGCT
TTATTCCTCAATTCAGAAAGTCCGATTGCCATTGCCACATTAAATACAAGACCGCCGAAAATAAA
CAACAAATTATATGCTATCGTATTTCTCGTAATAACCCATGCGTCCGGACTGCTAAACATGAACT
CAAAATTTTTGAGTCCATTCCACGGGCTTGCCCATATTCCAAGATCATAACGATACTGCTTAAAC
GCAATTATAATACCAAACATTGGCAAATAATTAAACAGTATAAAAAATATTAATCCTGGAATGC
ACATTGAGAGCAAGGAACCATTTTCTTTTAAATCTCTTATAAGGCTTTTCTTTTTTTTCACGTCTC
ATCCACTCCTTAAAAACTTAATAATTGCAAGGATTAAAGAGATATATAAATCTCTTTATCTTGCA
ATTATTATAACATGTACTATTTTTCATTAAAATGAGGTAAACTTTAAGCTAGGTGGTTTAATTTAT
AAAAAGTTTGTCACATTGATTATCGGGTGAAGATTTGTAAAATCATCAAATATAAACACTTTCAT
AGTTTGATGCTCAGCATCTGTAACATTCAAGTCAAGCATCAATTCACCGTCTCCGATTATTTTCTG
CCCAGCCATACGAACTGTATCAATCACACCGTTTTCTTTATACACAGCTACAATCATTACCATAT
TTGTATCTTTTTCAATATCAGATGCTATCGCTTTTATTTTTATATCTCCATTTGTAAGATTAGTGAT
TTCCTTTCCGTCAATTTCCGCTGTTACTTTAACAGATGAAAACAGAGTTGTTTTTCCTTCTGTTTCC
GACAGTGTCATATTATCAAAGATAATCTCACCAGTTCTCGCTATCTCTTTGAGTTCATCTGCACTC
ATTGCCTTTGTGTCGTCTGAATTATTATCAAGATTGGCATTCTCAGCCGCTTGAAATGTAACAGT
GCCTATCTCCGTCATATTAACAATATTTTCACCATTTACAAACTCCGAAAGCGGAACACTGATTT
TCGCCCAATCGCCATGAGAATCAAGTTCAAATCTCCTCGTATAACGTGTACCACTTGTAAGCGTT
CCATCGACAGTGTTGCCCGTTGAAAAACTTATTTTTACAGCTCCTTTACCCTTATAATCAAAGTTT
AGGTACTCAGCCCCCTTTGCACCATTATTTGTCCACACTGCAGGAGCCGGCACAAAAATTTCGCC
GGCATAATAAGTTGCCGATTGATAGTAAACACAGAAAGCCGTACTTCCATCAACTCCGCCGTCCT
GCAGCCATTTTGCTTTTATATAGTCTTGATAATCATTCGACTTATTTTTGAATCCTCCCCATGTCTT
ACCGCTTGCGAACTTAGTATCCGCTGTTTCAAAATCCGCAACATACGATATTTCTGTCGGTTGAG
TTGTCGCTTCCGGAGCAGCTGTCGGGGAAGTTGTATCCTGTTCCGCAAGCGTGATGTTATCTATT
ACTACACATCCTGTTACAGCTTTTTCTTCAAGTTCATCCGCACTCATCATCTTTGTTTCTTCAGCAT
TGTTATCGAGGTTTCCACTTTCTGCCGCAGAAAAAGCCATTCCTACCACATCTGTCAACGGCACT
TCATTACCGTTATTTACAAATTCAGAAAGTGGCACACTGATTTTTTGCCATTCATCATTTGTGTTT
ATAGTCACTGTATGACCATATCGTATTCCGTTTACAACCTCGCCCGTTTCAAGAGATATTTTTATT
TTTCCTTGTCCCTTAGCATCAAATTCAAGACACTCTGAATTTTTATTTATTGCCCATTCCTTTGGTA
TTGACATAAATATCTCTCCGGCATACCATGTGGCAGCTTTGTAAGTCAGTTCAAGAGCACAACCC
TCTTTACCGTTTTCAGTTATTTCTGACTTTATACTGTCACTGTAGGTCTTATCATTGTTATTAAATC
CTGCCCAGGTCTGCTTATGACTGAGAGTATAAGTATCAAAATCTATGGTTCTTGTTGTATTATCC
GGTTTCTCTGTAGGTTCCGGTGTTGCATTTGGATTAAGAACATTCTCTCCGACATTGGACAACTCC
ATATTGTCAAAGATTATACTTCCGTTTCTCGCTTTCGCTTCAAGCTCCGCTGCCGTCATTGCTTTC
GTTTCAGAGGCACTGTTACTCAAACCGCCGTTTTCGCCCGCTTGGAATGTCACGCAGCCTATATT
GGCTATTGTTACAGGATTTCCGTTATTTTTAAATTCCGAAAGTGGAATACTTATACTTTGCCATTC
ACCGTTTGTATCGGCATTAAGTTTATAACTGTACTTTGTACCTTTCGTAAGAGTGTCTGTTGCGGC
ACTTCCTGTTGACAGGCTGATATTTACAATACCTTTGCCATTGTAATCAAAATTAAGGTACTCAG
CATCCGCACCGTTTTGCCAAGCTACAGGAATTGGGAAGAAAACCTCGCCTGCATACCATGTCGC
AGCTTTGTAGGTTATGCGAAGTCCTGTTGAATTTTCCTTTCCACCATCGGCTACCCATTCTGGTTT
GACAAAATCACTATAATCGCCGGCTGTGTTTTTAGTTCCGCTATATGTTGTGCTATTACCAAATTT
CGCATCTGCAGACTCAAAATCAGCCATATATGACACGTTTGTCGCAAAGATAGCACAGTTTATTG
AAAAAAATATTGACATTGCGATAATTAATGAAACTAATTTCTTCATAACTAAATCTCCTAAATAT
CCCTCATATAAGTATAGCGGTCAAAGACCGCTATACTTATACCTAATTATTTGTTCTTGTTATTGC
TTGTTCTTGTTCGCAAGAAATTCATCATACTGTTTCTGTGCTTCTTCAATAATTTTGTCAATGCCG
GCAGCCTTAAGTTTAGCGGCATATTCTTTCATAATCGGCTCAGGGTCCATTGAACCCATAATTAC
CTGTTTACGATACTCGCTCTTAACTGTCTGACATGCCGCTATTTCCGCTTCTACCGCAGTATTATC
GAATTTAAAGCCATAATCGATAGGTTTCTTAGCCTCTGCGTTAAATGCTTTCAGAGCCTCAACCT
TATCAGGGCTTTCTCCTTCTGTAAGATAATTTAGGAATACATTTCCCTGCATCCACTGATAACCTT
GTAACGTATACGATGTATCATCCGGAATTGTTATAGTATTATCATCAATCTTGGTGTAATGTTTTC
CTTCAATACCATAGTTGATAAGGTTGCTGAGAGTTGCATCAGTGTTAAGTAGTTCAAGGAAACG
AAGAACTCTTTCCGGATTCTTTGATGTTCTTGAAACAGCAAGCATCGAACCTGTTCCGGCTCCAT
TATCCTGCCATATATCCGATACTGTTGATTGGTCAAGTTCAAAATCAAACTTAGCGGAAGTTTCC
TTGGCCTTACCTGGTTTTAAGAAGTCCACATAACAGAAAGTTTTTCCGTCCTTTAATCTCTGCTCA
AAATCCGTAGCAGTCATAATGTCTTTCTTTACAAGACCTTCGTTATAAAGCTTATTATCCCATTTG
CATGCTTCAAGATATTCCGGAGTTTCTACAAGATTTACAACCTTGCCGTCATATTTGTCTGTATCG
TAGAAAATAACGGCTGTTCCTGCGATTTCCTCGTACTTCATAAGAGCTTCAGGTGTTCTGTCACT
GCCCCAGTCAATCGGATATTGCATATTTGGTTCATTTTCCTTAATCATTTTAAGCACCGGTAACAG
TTCGTCAAATGTCTTAATATTATCCATATTGATATTGTATTTTTCGGCAATATCTTTGCGATATGT
CCAGCCTCTTGAATCTGCCATTTCCTTATATGTCGGTATCGCATAAAGCTTGCCGTCCACTCTTGC
ATTGTCGGCTATTTCTCCAAGCTGCTCAATTGTCTTTGGAATATATGTGTCAATGTAATCATCAAG
TGCAAGCCATGCACCGTTTCTCGCATTTGCCGTATAAGTAAGAACTCCGGGTGTTGTCCATGCAA
TATCAAAATATTCACCCGCCGCTATCATTGTGTTTAGCTGCTTGCTGTACTGATTTGATTCCAGTC
TGTGCATTTTCAGTGTAGCGTTAATTTTATCCTTAAGATAATCATTAACAGCAGCCTCTACGGAA
GCAACATCCTCCTGTGGCATACCTTGCATATACCAGTTGATTTCATATGTATCCTCCGGTACAAC
ATTAGGATCTTCACCGCCTGTTGCAACCTTGTTTCCTCCGCCACAGCCTGTAAGCACACCTCCAA
GCATTAGCATGGCCAATAATGCCGCAATTTTCTTTCTCATAATTTTATCTCCTTTCTTTTTTGATAA
CGGTTCAGACGTTTCGTCCTTTCCCATTATCAGAACATTTTATATTGGTCTTTCTTTATATTTAACA
TTATACACCATAGTATTTCAAATATAAATAGCGATAAACTTTAAAATGTGCATATTTTTTTAATA
AAATTTATATCATTTCCTACTTGAAATAATATAAAAATATGTTAAAATGTTATATAGTTTAATAT
AAAGATAAGATAGGATGGAGAATTATGAATGTAAAGTTGTTAATTTGTGACGATGAGAAGATAA
TACGTGAAGGACTTGCTTCACTGGATTGGAATACCAGAGGTATTGAGGTAGTAGGAACAGCAAA
AAATGGTGAGGTAGCATTTGAGCTTTTTCAGAAAATGCTTCCCGATATTGTTATATCAGATATAA
AAATGCCAACAAAAGACGGCATATGGCTTTCAGAACAAATTCATAAAATTTCACCTAATACAAA
AATTATATTTCTTACAGGATACAATGATTTTGAATATGCACAAAGTGCTATTAATAACGGCGTAT
GTCAATATCTTTTAAAACCGATAGATGAATTTGAACTTTATGAAATAGTTGACAAATTAACAAAA
GAAATACACCTTGAGCAACAAAAAGCAGAAAAAGAAATTGAATTACGCAAAACGCTTAGAAAT
AGCCGTTATTTTCTATTGAATTATTTATTTAATCGTGCACAGTACGGTATTCTTGATTTTGAACTA
TTTGAGATATCTAAAAAAGCTGCGGCAATGACGACATTTGTAATACGTCTTGATACAGACAGTA
CCAACTACGGAATGAATTTTATGATTTTTGAGGCACTAATCGAACATCTTCCTAAAACAATAAAC
TTTATTCCCTTTTTTAGTAATTCGGACCTCGTATTTATTTGCTGCTTTAACGAACCGGAAGGAGAA
TCCGAGCAAAAACTTTTCTCTTGTTGTGAGAATTTGGGAGACTTTATTGATACTGAATTTAACGTT
AATTATAATATAGGAATAGGTATCTTTACTTCGGAAATATCCGAACTTGAGGCAAGCTATACCTC
TGCATTGCAAGCACTCGATTACAGTGACAGACTTGGACAAGGAAACATTATTTATATTAATGATA
TTGAACCAAAATCACAGCTTTCGGCATATCAGTCAAAACTGATAGAAACCTACATAAAAGCACT
TAAAAACAACGACGAAAAGCAAAGCAAAAAAAGCGTCAAGGAACTTTTCGACGTTATGGAACG
TTCTGATATGAATCTTTATAATCAGCAGCGACGCTGTATGTCACTTATTCTTTCAATTTCAGATGC
ACTCTATGATATTGACTGCGATCCAACAATCCTCTTTAAAAATACGGATGCGTGGTCATTAATCA
GAAAAACTCAATCTCCTGCAGAACTTAAAACCTTTGTTGAAAATATTACAGATGTTGTAATATCA
TATATTGAAAGTGTTCAAAAACAAAAGGCTGCAAATATAATCACTCAAGTAAAGGCTCTGGTCG
AAAAAAATTATGCAAGGGATGCCTCGCTTGAAACGGTTGCTTCGCAAGTGTTTATTTCACCTTGC
TATTTAAGCGTTATATTTAAAAAAGAAACTAATATAACTTTCAAAAATTATCTCATACAGACAAG
AATTGAAAAGGCCAAAGAACTTCTTGAAAAAACTGATTTAAAAATATATGATATTGCCGAAAAA
GTAGGATACAACAACACCCGCTATTTCAGCGAGTTATTTCAGCGTATCTGTGGTAAAACTCCATC
GCAGTATAGAGCGAGTCACAATCCATCTATGCCTCAAGACATATAGGAAAAACACCAACTACGT
TCAATGTCATTAATTTAAGTCAAACAAAAAGAATTAAGTTTAGAAATATACTTGAATTTAAAATA
ATTAAAAAAGTGCTTGACTGAGGTGAACTCGAATCAATGGTGTGGTCGAACTATTTTATTATGAT
AATCTAAGACTGTAACCACAATCACTATTTAAGTATCCGTACAACTTTTAACTTAAATTCATAAC
TATATTTTGCAACAAAAAAACCGACCTCCAAAAGTTAGATTTGTGGTCTAACTTTTGGAAATCGG
TTCAATACCTACATGTTTCCAAACCATTTTCTCATTTTGTGCCAAGCAATATCATATTAACTAATG
ACATTGCGGCTATGGTGGTTTTCCTGTTAAATATCACATTAGTTTATTCTTATTGCATCAAATCCT
CAAATATTTTTCCTGTATTCATTGACGTTTCTTCATTATTAAAGAATTTAATAAGTGCTTCCTCCA
GCTTTTTTAGATTCTCTCCATTAATACTTACTGTTGCAAAAGAGCTTGAATTTTGTACACTTTGCT
TTATTCCCTTCACATGTGGATTTGCATCTAATATCATTTCATCGTCATAAAGTTCTCTTATTATCG
GGATAAGCGATGTATCTCTTGATAGTCTTCTCTGCTGCTCTTTTCCATTCATAAAATCAAGAAACC
TTATTGCTGCCTCTTGATTTTTTGAATTTGAGTTTATTGCAAGGGCATAACTTCTTACAAATTGAT
TTTTATTAGGCAGACTTGCTACCATTATATTTCCCTTAACTGCCGAAGTATCACCATTAAGCTTTT
TCCATAAAGAGCTGTTTCCCATCAACATCGCTGCACTGCCAATTTTAAAGGCGGCTATTGTATCA
GTGTAATTTTCAATATCTTTGTATTCTTCAATAAATTCCTTATATAGATTAAGCGTTTCTGCATAG
CTTATGCCTATCGCCTCTTTTATTTCTATAATATTATACATCATATCTTGAATATCTGAGGTAGTC
AGTCCCAGTTTAATAGGTAGTCCGAAATCAGAATTACGGCAAAGATTTATAATTCCATCCCATGT
TTCAGGAACGTTATGAATCTTATCGCTCCTATAAAATATAACATCAGTATCCATTCCCACAGGCA
TAGCATAAAAAGAATCATTAACAGAAAATCTTTCCTGTGCGTCAATTATATATCTGCTATTATCT
AATAAAATCTCTCCATCAAGAGCCTTTATGTATTTCTGCTCAACAAACTCCTTAGTCCACTCATCA
TTAATCCAGTATATATCGATCGAACTGTCTTTACCACTTAACGCCGAAACATATAGCTGATGCCT
CTTTTCTGTTGAAGTGGGTGCATCAATAAATTTTACTTGAATATCTGGGTTTGCTTCATTAAATTC
AGCTATATTTTGCGTGAAATCTGAGATATAGTTTGGTTTTATTACTTTTAAAACAGTAACAGCCT
GTGCGGATTCCACTGTTTTCTCCACTGTGCATCCTGAACACATTGATAACATCACAGAAGCAAGC
AAAAACAAAATAAGTAATTTTTTCATTAAAAACACTTCCTTTATAAGTCTTGCTAATAATATTAT
ACCCTAAATACTTTATTTAGTAAATAGCGGAAAACTTTAAATAGTGCATATTTTTTTGTATTACTC
TGCCAAATATTTTTATGACTTGAAATTTAATATGGTTATGGTATAATTATATCATAGAAGTGTTTT
TATATATACCAATTTAACTTTATAATGATAAAACGGATTAGCTATATCCATAAAAATTCATAAAC
TTTATATTTTCAGTATTTTGTAATGGCTATTATATTATAAAAAGGAGGTGTTTGTCATGTCTGAAA
AGTTCAATAATATGTCATTTCGTACAAAATTGTTGCTTTCATATATTGCGGTTATTATATTATGTA
TCATTATTTTTGGTTTAACCGTATTTTCGAGCATTTCAAGAAGATTTGAAAATGAAATAACTGAC
AATAACGCACAGATAACAGGCCTCGCTGTCAATAATATGACAAATACCATGAATAATATTGAGC
AAATTCTGTATAGTGTTCAAGCCAATTCGACAATTGAAAAAATGCTGACTGCGTCCAATCCTCCG
TCTCCTTATGAAGAAATTGCCGCCATAGAACAAGAACTTTCAAAAATAGACCCCTTAAAAGCAA
CAGTATCACGTCTTAGTCTATATATTGAAAACCGTACATCATACCCATCTCCGTTTGATTCGAAT
GTGACCGCTTCCGTTTATTCCAAAAATGAGGTATGGTATAAAAACACAAAGGAACTGAACGGAA
GCACATACTGGTGCGTTATGGATTCCTCTGATGCCAATGGCTTGTTGTGCGTTGCTCGTGCGTTTA
TAGATACGAGAACCCATAAAATACTCGGAATAATCCGTGCAGATGTTAATCTTTCGCAATTTACA
AATGATATTGCACATATAAGTATGAACAATACAGGTAAGCTTTTTTTGGTATATGAAAATCACAT
AATAAACACGTGGAATGATAGCTACATAAACAACTTTGTAAACGAAAATGAATTTTTTAAAGCA
ATAAGTGCTGATTCCGATAAGCCTCAGCTTGTTCAGATAAACAAAGAAAAACATATTATAAACC
ATAGCCGGCTCAAGGACAGCTCTCTTATCCTTGTACGTGCTTCAAAACTTGATGATTTTAACAGT
GATATACATATAATCGAAAAATCAATGATAACTACAGGAATTATAGCATTACTTGTCGCTCTAAT
ATTCATTTTTATTTTTACACGTTGGCTTACAGCTCCTATAACAAAGCTTATAAAGCATATGGAAC
GCTTTGAAAATAACTATGAGCGTATACCGATAGAAATAACCTCTCATGACGAGATGGGCAAACT
GGGTGAGTCCTACAACTCTATGCTCAACACCATAGATTCTCTCATAACCGATGTTGAAGATTTAT
ATAAAAAACAAAAGATATTTGAACTTAAGGCTTTGCAAGCTCAAATTAACCCTCACTTTTTATAC
AATACCCTTGACTCAATTCATTGGATGGCACGTGCTCATCATGCACCGGATATAAGTAAAATGGT
GTCCGCATTGGGAACTTTTTTCCGTCATTCTCTTAATAAAGGCAACGAATATACTACAATAGAAA
ACGAATTAAATCAAATATCAAGCTATGTATCTATACAAAAGATACGCTTTGAGGATAAATTTGA
CGTTGTATATGACATTGACGAAAATCTTCTGCACTGTACAATCGTAAAATTAACAATTCAGCCTC
TTGTTGAAAATTCTATCATCCATGGTTTTGATGAAATTGAAGAAGGCGGTATGATAACAATTCGT
ATCTATCCCGAAGATGATTATATATTTATTGATGTTATTGATAATGGTAGCGGCGCAGACACTAA
TGAGTTAAATAAAGCTATTACTCATGAATTGGACTACAACGAACCAATCGAAAAATATGGACTT
ACAAATGTAAATCTGAGAATTCAGTTATATTTTGATAAAACCTGCGGCTTATCATTTAAAACCAA
TGAAACCGGCGGTGTAACAGCCACAATAAAAATAAAGCGAAAAGAACCGGAATATAAAACTAT
TGATTTATAATTTTGTATATGAGGATTGACTGCAATTCAAATACCGAAAATAATATAACTCAATA
CTGCCGCAAACTTAATGCTTATTCAGCTTTGCACACTGTTATGAAGAATCTTTCATAACAGTGCT
ATCTTTGCCATGTTCCGGCAATACCGAGATTATATTCAACAGAATGTAACGAATGTCAAAAATAA
TTGACCGTTCATTATATTTATATAAAAGCACCTTGCTTTTTAAGTTCTTTTCTTAATTCTTCAATGT
CAATGTCTTGCACGTTTATATTATTTACAGCACATATTCCGGCGGCAACACCTCCGCCATGACCT
ATAGCTCCTGCAATAGGTGATACACGTATTGCCGCTTGTGCCTCGAAATTTGCACTGATGCAGCG
TCCGACTGTAATCAAATTTTTCACATTGTCAGAAATAAGCGAACGGTACGGAATATGATATATAT
CCCCATATTCTAACGAAAGTTTTGTTTTTTCATACATTTGGGTATCATTTCCCTCCGGAGCGTGAA
TATCTATTGGATAACCGCCGCAAGCAATGGTATCATCAAAATCTACACAGGATATAATATCCTCA
GCTGTAAGCACATAGCTACCTTTCAGCTGACGTGAACCTCGTATGCCTATAAATGGACCCGTAAA
CTCAAGCTCTGCATTTTCAAAGCCCTGTACCTCTGTTTTAAGCAATTCAAGCACTTCCCAAGCCTG
CTTTCTTCCGAGAATTTCAGCACGAGTTAAATCCTTCGGTTCGGTGGGGTCTGCATTAATTATGC
GTGTGGTATTTACAATAAACTCTCCTACCCTGTCTGTTTCAAAAAACAGAATATCCTCCCTCTGA
AATGAAATCTTGCCTGTCTTTTGTGCTTTTTTCAATGTATTTACATACCCGCCTATAGATAGCTTT
GGAGCACGGGTAACTTTGCTTAAATCACTCTTAAGCCGAGGAAATTCATCATTATTATTCATTAT
ATATTCCTTGACTCTATCCGTATCAACATTAATAACCTTAAAATTCATTGTCATAGGCTGGCATTT
CCCATCTGCCTCACGACCTTTGTTTGTTTCAAGCCCTGCAAGAAAAGCAAGGTCACAATCTCCGC
TTGCATCAACAAACATTCTAGACTCTATACATCGTTTACCGTTTTTCCCATATACCTCAACAGAGT
TTATTACGGCATTTTCGTATTCAACATCACAGACCACACTATGATAGAGAAGTTCAACACCGGCT
TCTGCCATCATATCCTCAAGTTCAAGTTTCATTCCTTCAGCCGAAAACGGAGTAACTGTATATGT
ATAGCCCGTAGTATCAAAAATATGCCCCGGAGAGAACCCTTTATTCATTAATCTTTCTATAAGTT
CATTCGTTACACCCCGAACAACTTGAACATCACCGGCATGAAACGTCATCATAGGTCCCGTACCA
CATGCTGTAAGTGTACCGCCTAAAAAGCCATATTTCTCAATCAATATAACACTTGCTCCACATCT
TGCAGCTGCTATTGCTGCCATACAACCGGATATTCCACCTCCGATTACCGCTACATCATACATTTT
TTACACCTCCAAATTTTATATGTTGTTCCTCGCCCCTTGCAGCACAAGTATCAATCTCCATACATA
CATACGGTACAAGTTGTATTATATTAATTCGTTCGCTCTTACTTATTATTTCTTGTGCACTGTAGT
CAAGCTCTATATAAATTTTATCTTGTTTCTGTGTAATATCAGATATAGAAAGTTCAAGCGTTCCAC
AAGTCTGCGTGGTCATCACAATACAACGCCTATCACAACTTATCCCCCCTGCACTGTGCGTCTTA
TCTTTCAAAAACACCACTCCGGTTGTTCCATTCTTTTTTACACACTGTATTGAATCAGAATTCTCA
ATTATCCTTATTCCACTTTTTCTGTAATAATCATTTATTTCTTCTTCATGGCATTTTGGAATAACTA
TATATTCGTAAGAAACGTCTTTTACCTTTCTTCCGTGTTTAATCCACATTGTAAGATACCTTCCCT
TGTAACTCTTACCATCTGATTTTATGCTCATATTATTCCAATCTCCGCTTCGTATCTCTCGAAAAA
TATTTACCTCCTGTTCCTCCGGGAAACAGTAGCCCACATCATGACTACCATCAAGATAAGCTCCT
TTTATAATATAACCTTCACTTTCTTCATTACCATGTACTGTAAATCTCGAATTATCTGTTACAAGT
CGATTTTCAATTATCGTTTCTACTTCACTTTCTTCTTCACTGTTTATACAACTTCCAAGACAAACC
ACTTCTTTATCGAAGAAAAACCACGATTTATTCGCTTTCAGACTATTTTCATTTGAAATCAACTTC
ATTGTGCATACACCGTTTTCCCCTATGCCGCATCCTCCTGTAAAATCACCTGCGGCATTAATATTA
GGTTTTACTGTCGAGCCTCGTAAAACAGTTGTCCCTGGTAGGCGTTGTAAATCTATTGTTTGCCA
AAAAAAGTCCGACTTTGGCTCATTTTTTTTATAAATGTACATCATACCGTCCGAGGTATGATGAG
CATTTTGATTTTCATCGTTAATTGATTCATATGCTGCAGTCCGTTCTGAATGCATGGCAAGACCTA
TCGTATAACCATTTCCGTGTTTAACAACTCTATCCATACTGTTAAATGCCATAAAATATGGCTTG
ATTTCTTTCGGTTTAATATTATTATCTTCTTGTAAATGTTCCGCAAGTTCTGCCGTAAATACTGAA
GCATATTCAAAAAAATTATCTGTAATCTGTGTCTTGATCGTACCCTTCAATTCATTAAACTCAGG
CATTTCACTTAATATAAGCATTGCCGAAAGTATGTGCGTACAAGCAAGGTCACTCTGCTCATAAT
AACGTGAAATTTCTCTGCCACGTACCATATCCATAGCTCTGCCATTATATATGAAAGGAAGATAG
GATTTTTCAATCCATGTATTAATTATATCTGTATTTTTATTTTCAAATTCTGTATCTTTAAAAATAT
ATAACATCGGTGCAAGTTCTTGTATAAGCGAGCGTCCGTAACCACAATTATATGGTATATTATCA
TGTTGAATAAATGAACCATCCTTATAAAAACCGTCACCGCTGTCTGTTATAACCATTACATCCTG
TATACCGGATATAGCATTCTTTATACTATCGTTGTCTGAAAGTAATATTCCACGAACAGCAAAAA
TAACTGACTCCCAAATTCTGTTAGCACCGGTAAGCTTTATCCTATCATTAAAATGTTTTTCCGCCG
CCATATATCGTTTGAGTTGTGATTTGTCAGTATAATCATACATCAAGGTAAAAATACTGTTGATA
CTAAGAGGGATACCTATTTCCCAGTACCACCAGTTACCTTTAGGCACAGTAGTGTCATTATATAC
TTTTTCTAAAGTATTTAGTGCATTAAATATTTCATTTTTTATATTTTTATTATGATAGAATTGTGAG
TTATTTTGAGAAAACGATATAGAAATGTCCAGTATTCCTTTCAGTGTTGCATTTATAACTCCCGG
CTCATTTGATGTAATTGCCTTTTCTATACGCCCACCAAGCTGTACAAGCCTTTGTTCTGTGCGTTC
ATCTGATTGTAGTATGCAATCAGCCGTTTTTTTGCCGTTGTATCCTCTACCGCATAAAACATCAGA
ATATCGCTTTCTTAGTATGTTAAAATCCGTCATAATCATTCCTCCCATTTTTATATGTATCAATAA
TATCATATATGTCTATGCATATTAAATGGGAGAATCTTTAATTAATGCGTTAAATTTTTTATATTT
CAATTTAAAAGGACGCTTTAAAATAGAGCGTCCCTTTAAATTTTATAACAGTTCCCATTCTTTTAT
TCATTCTCTTTTTATAGGCTCATCTCTTGCCTAATCAATATACACGTTTTAAAACAACTGATTTAG
CCAATCAACTGACAGACTCAACCACGGATACTCTCTTTCAAACTTTCGTTTGGACCATATTAACT
CATCACTCACCCTTGACAAACCATGCTCGCCTTTCTCAAATATATGTAATTCAAACGGTACTCCA
ACCCTCCTCAAGCCAGCTGCATACATCAGTGTATTTTCAACAGGAACACATATATCCTCATATGT
ATGCCATAAAAACGTTGGTGGAATTATATCCGTAATACGCCTCTCAAGCGACAGACTGCTCCATA
GATGATTTGACTCGTCATCTGTACCTGTAAGATTTTTAAATGAATCTTTATGAGCAAATTCACCC
GATGTTATTACCGGATAACTTAAAATTTGAGCATTTGGCTTATGCATTGCTAACTCAATCTCGCG
CTCGCTGAATATTTCAGAATCATTCCACAAGGCACTTAGTGATGCTGCAAGATGTCCACCGGCAG
AAAATCCGCATACAATTACCTTATCCGTATCTATATTCCATTTTTCTGCATTTTCTCTAAGCATGG
CAACTGCATTAGCTGCATTTTGTATAGGAAGCGGATGTGTGTGCGGTTCAACACAATAATACACT
ACTGCCGCATGGAATCCTGCTGCGTTATATGCCATAGCAATACGCTCTGCCTCACGCTCAGATAC
CATTCCATATCCACCTCCGGGAAATATCACAACTATTGGACGTTTCTTACCGTCCAGAATATACG
TTTCCATATACGGCATAAATCCATATTCTGTCGCTTCTTTCAACAAGTTAAATTTCTTATTAAACA
TAACATTCACTCCGTCTTAATTTTTGTCAGTTAAACGGGCATATCCTATCACAGAATATGCCCGA
TATGCTCATCTTAATTCAATGTAAATACTTTATTAAATAATGGTTTCATATCATTTTTCCATATAA
AGACCTTCACCTTACTCTTATTTACGCAAGTAAATGGAATAATTACACTGTCTGAAATTTCATCT
GATTTTATCGAAAGCAATGTACCATTCTCATTATATTCTGCAACATAAACCATGCCTTCTTCTGTT
ATATTGGTAAATATAGCATTAACATTTCCATTTTTATCATCATACGTTATATTCACTGTCGGTTCT
GTAGGCTCATCCTTTGCTGCCGCATTAATTGTCACACTCATAACTTCACTGGCAAAGGATTCATC
TGCAACTGCCGCCACACGAACAATGTAAGACCCCGGTGCAAGATTTGTGATTTCCGTGCCTACAC
ACTGCATCCATGTGAATGTAGGTTCTGACAATGATTTATATTCCATTGTAGTGTCAACACCTGTG
ATTTTGCCGTCATTGCCTTGATAAGTCTGTTCTTCAACCGCTGCTACATTTGGTGCGGATGAACGT
TTAGGAATAGAAAGTTCAAACGCTTCACTGTTGCTTGTTTTTATGCCATCATCTTTTTTTACGATT
TTTAGGGTATGACCGAAATATGTGTTGTCAATGTCGATATCCTCTGTTACATTATCTTTATCCGTC
GCTACGCCATCATCTATTTTTATTATATAGTCACAGCCTTCCGTAAAGCCTGTTAGCTTTTCGTTT
ACATAGTTAATCGAGATTGTAGGCTGCGTTTCCGGCATTGCATCATACGCAATAATTGTAACCGA
ATATTCTGCACTCTTAAACTGCCTTTCGGCTTCCTCGTCTGCACTTACAGCTTTATAACGGATTTT
ATATGTGGTTCCAGGCTCAATATCACCTATATCATCTCCATCGCCCGTAGTCCAATTAATACCATT
ATTCGTACTATATTCCATAGTGTCTGCGATACCTGCAATAGTACCTTTGCCGCCAATCACACTCG
GTTGTGTTACTATGATTTCCGATTTTGTCGGTGCTGCAGGACGTGCCTTAACTATTAGAGTCTGTG
CTTCACTCGCAACCGTTGTCACATTATTACCCTTCTTTACTATCGAAAGCGTTATCTGTTCATTTG
TTATATAATTAGCTAATGATAACTTGTTATCTGTTAATGTAACATCTAAACCATTAATTGTATATG
TTCCATCCTCAACAAAGTTTATAAGTTCCTCTGTTGTATAATCAATTGCAATTTCAGGCGTCATTT
CCTTTTCAGCTATAAACGTTTCAACTGTAATTGTTGTCTTCTCACTTGCAAAGTCTGTATCTGTTG
CAGCTTTGCGGACATTGTATTCGCCTGCATCAACCTCAACTGTATCAACCAACTGCGTACTACTC
CATTCATCTGTTCTCTTGAGTTTGTACTGCATGCCATTCATACCTGTGAGTTTACCTTTGCCGCCT
ATTTCCGTAGCATTTACACCTTGAACGGTTGTAGGTGCCTTAGGACGTGCTTTAACTGTCAACTG
TTGCACGTCACTGTCGGTATATGTTTCGGTATTGCGTGCTTTTTTTACAATTTCAATAGACAATAA
TTTGCCGGCATAACCAATTTTTTCATCATCAAGCGATATTGTCGTCACGCCCTCGCCAAGCGTTAT
ATCTTTAGCATTACCTTCACCCACCTTTATCGTATACGGTTCCTGTGACTCAAAGCCGGTAAGTGT
TTCATTTATATAATCAATGCTTATCTGTGGAGTTGCTTCCTGTATTTTTGCCGGTGGTATAATTTC
CTCCTCCGAATATGATACTTCTGTTACTTTAACGTTATCTATATACGTCGCATTTTCAATATTATTT
GAAGCTGTATGATAGAATCGCAAGTCTGTTATTCCTTCCTTTAAAGTGTCAAAGGCTCCTTTACA
TGTATCAAATGAACTTCCTTCTTTTACAAAACTCACATTATCTTGAAGCATTGTTCCATCTAGCCA
TATTGAAAATATTCCTGTGTTTGGATGAAGTTCAAGTTTTATATGGTGCCATTGGTTATTCTTTAT
AGACATTGCTTGTTCATAGCACCAGTTTCCTCCCGTATTAGTAATAGCTGTTCTGAGTGACGATG
CGGTGAAATATGTAAGCCATAGTTCATTTCCTTCCTTTGTTTTAGCATACATACCTCTTGATGCAC
CAATCGCATCATCAGCACTTTCAAACATCGTGTCGTATTCTATTGTTAATGTTTTCTGCGGATTAT
CTGCCTTACTTATAGGAATAGTTGCATTAGCCGTTGCCTTATTGCCGGTGGCAGACAACTTCAAC
GACTTTGTTGTTTCTCCTGTAGGTATTGCCGACGTTACCGTCGTTGTTGTCCCATTAACGTCATTC
GTTGCCTTTGCCAGCATATCATCGCCGTTGTTATACTCAACAGATGATGGACTTTCTGTATATCCC
TTCACATAATCATATCTTGTAGGTCTTATAATATTATAAGTTGTATAGTCAACACAGGTAAGACT
GTTAATGTTATCATTGTCTGTTGCAATTGCATAAACACTGTGCACACCCGATGTAACATCAGGTG
TAAATACCCATTTACCGTCGTCACGTTGTTTAGCATCACCCGCCTTGATTTCTCCCTCGTCAATAT
ATACCTCAACTTTACTGACAACACCATCTGTGTCATAAGCACTTATTACAATGTCGTCGCCTGAC
TGCTCTACTTTTGATATATACGGTGCATAATTATAGAACTCTGTCGTTGACAACTCTTTATTTACA
GCTTTAGAAGCATATTTCTCTGTCAAATCAGTCATAAACGGTGCTATAGGTCTGCCTGCAGCATT
AAATGTATTAATCACGGCTGGGGTGTCAGTTTGTGCTTCAACTAAATCCTTTGCAATACCCGGAG
TATCTGACCATGCATACTTAACCTGTGGATTTGTTATATCTGTTACATCTATTTCTATAGTATCGC
CGTTAATTGTCGGTGTAACATCTTTGTATATTCCGTCATCATCTTTAAGTTCAAAGCCCAACGGCA
CACCTCCATCATCGGTTGAAAGAGAGCCGTATGTATTCTTGAAATGAAGAATAAGTTTATCGCCA
CTGCGTTCCATATAATCAAAAGATGGGCTTTCAACATTTGATTGTGTATTGTTAATAAAATCTTCT
ATATAAGCAACTGCTCTGTCAGCAATAGGTCCTTTATCATTTGGGTGAACATTATTTGTAGTTCCT
GTATCATTGCTCACAACTGTTTTTACGTTATCCATTCTTTGTGATACATTCCATTGTCCCGCTCTTA
CTCCGGTGCCAATGCGTATTGTCGAATAAATTTTTGCAAAATTCGCAGTAGGAAGTTGAATGACT
ACAAATGGTAAGTCCTCATCATTAAATGTTTTTCTCCAGTTGTTAATAAGCGAAGTCAATGCTTG
CTCATACACTGTGCCGCTTTCAAAGGTAGTATTTGCCTCTCCTTGATACCATACAACAGCAGATG
CTTTCAAATTCTTTAGCGGCAGAAGTCTTTGTGTATATAGTCCGCCTTTTGAGCTTGCTCCCGCCA
TCATTCTCTTTGCTTGTGAATCCCAATTTACTGAATATGTCGGAATCCACTGCATTATTGACGACC
CACCCAAACTTGAACTTATAAGACCTATAGGAACATCAGAATCTTTTTTTATCATCCTCTTACCTA
TCAAGAATCCAAGTGCAGAAAATTGCTTTGAATTTTCCATAGTAGCAACCTTCCACTCTGAAATC
TCGTCAAATGAATTCATATATCTCACGTCTTCGTAAGCTTCACTTAATTCTTCGTTCATCAACGTT
GGAAAAGTCTCAAGGCGATTAAACATATTTGATTGTCCCGTACAGAATATAACATCACCGACAG
CCACATTATCGAGAGTAATCATGTTATCACCGCTTGATACTGTCATAGTTGCTGATTTTACAGCTT
CCATAGCAGGAAGGGTTATCTCCCATAAACCATGTTCTATTGTTGTTTGCTCATCAGCCCCATTA
AAATTTACTGAAACCGTATTCCCCGATTTTCCTGTACCTGTAATAGTAATAGGTTCTTTTCTCTGG
AGTACCATGTTTGAGGAGTACACCGCATTCAAAGTTAACTCCGGTTCAAGAGTAGGCGTTGGGG
TTGGACTTGCCGTTGGGGTTGGCGTTGGTGTCACATCCGGATTGATTGTTGTGGTTGGCGTTGGT
GTTGTATCCGGCTCTGTTTCGCCACCTCCATCACTGTCTATATTCACATCAAATGAGTCAATAGAC
CACTGTATTTTCTTAGTGACTTGAGTTTTATCCTCACTTAAATCTCCACAGCCTATATATAAATAT
ACGAATCCATCCTTTGATGCCGTAATTCCGTTAGGCAGGGAATACTTCATATTTGATTTGTTTGTT
GACCAGTTTTTTATATTACTTTCATCTGTATGCTTTTCAAGTTCCTCTTTTACCAAATCCTGCGAGT
GTGATGTAAGTGTTATTTCGCTATCAGAAACAAAAAGACAATATTCCAATTCCATTGTTGCAGTA
TCAAGATATGTAAAATTAATATCGACATTAATCTTGTCGCCGGTATTTACTTGTGCCAACTTAAA
CATTGTCGCTCTTGGTGTATTGTTTGCTTGTATATAATAAGTGACACTTTCTTTACTGCTTACAGT
ATTTTTTAATATTTTACTATTACTTTTGTCTGAACTTGGCAGTGTATACGCTGATACTCCGTCCGC
TTCCGCTTCTACTGTAGTTGTAGTAGCCGCAAATGCCGTCAAACCGATAGATGCACATATCATCG
TCACAGCTAACATCAATGAAATAATTTTTTTCATTTTTTTCCCCTCTTTCCGTTTTTATATACTTTT
ACTCATAATAACTATATCTTGTTATTAATCTTACCACGGGAATTTTTTTATTAAAATGCGGTAAAC
TTTAAGCTGTGCGGTTAAATTTATTAAAACCAACCATGTTTTTCGGTACAAAAGTCATATAAATC
GTAAAATATGCTGTTAAAAGCTTGACTTGCCATAACATTATCCATAACCCTATTGACGCAAAAAA
ACAGTGCCATTAGAGTTGATTTTCAAACCAACCTAACGACACTGTTTTATTATAAAGTATAAATA
ATTTCTTCTAAAATTTAATCGCTATATCATTCCAAAATGTTTGAGTGCGTATTCTATGCCATTATC
GAGCAAATCTTTTGTCACAAAAGATGCCGACTTTTTAAGTTCTTCACTGCCATTACCCATAACGA
TACTGTTAGGCACTGCCGTAAGCATAGGCAAATCATTCATACTGTCGCCTATTGCATACGCATTA
TCAATCGGAATATTATGGTATTCAAGAACTTTTTTTATTCCTGTACCCTTTGAAAATCCTTTTACC
GTCATCTCACAAAAGCCGTCACCACGCACAATAAAATCAAAATCTTTTTCGATTTCTCTTTTAAA
TCGTTCTATATTGCTTTTTTCATCATACCAAGCCAAGAATTTGTCAAAAGCAAAACCATCGTCGC
TGACATCAGGTGACAAATCTTTACCTTGCATTTCAAAACGTCTTTTCAGTTTTACAAAACCGTCTA
AATTTCTACTGCGTTTATCACAATAAAATGACTTAGAATGTTCATACATTGGTGTCATATTACATT
CAAACACAAGTTTAGCAACATTTTTGCAAAGCTCCGATGACAATGTATGCTGATATATCACTTTG
CCATCACATTCTATATACATTCCGCAACCACATATATATCCGTCAAAGCCTATATCTCTTATTCTC
TGTTCAACGTTCATAACGGTTCTGCCCGTATCAACATACATCAAATGTCCGTTTTTCTGTGCCTTA
TGTATTGCATTTACCGCACTGTCGGGTATTATATATGCCTCGTCATCAGATATAAGTGTACCGTCC
AAATCAAAAAATACTATCATAAAAATCACTCCAATAAGTATATTATATATGGTGCAAAAAGATT
TTTCAAGTCGGACATATTTAATTTATATCGCAAGCATTGCCGCACCTATAATTCCCGCATCATTG
AAAAGCTGAGCCGCAACAATCTTTGTTTTTGTCATATGCTTATTAAAGCCCGTATTATACACATA
TTCACGAATCGGCTCAATAAGATAATCGCCCTCTTTGCTTATTCCTCCGCCGATTGCAATAATTTC
GGGTTGAAAAATATTTTCTATACTGACAATTCCGTCTGCCAAATATCTCTCATAATTGCTTACAA
CTTTTTTTGCGACCTCGTCACCTTGCTTAGCCGCCTCGAATGCCGTTCTGCCCGAAATTTTGCCCT
CTTTTTTCACTATACCATGCATAATAGTATCTTTATGAGTTTCAAGTGCATCTTTAGTCTGCGATA
TAAGAGCAGTCGCAGACGCATATGATTCCAAGCACCCCTCTTTACCGCAGGTACACATTTTACCT
CCGCTGACAATAGTCATATGACCGAGTTCACCGGCAGCACCGTTAAAACCTCTGAAAACTTTACC
GTTGATTATTACACCGCCACCCACTCCGGTACCTAAAGTAACCAAAGCAAACACACTTGCACAG
TGTCTGTTTATCTTATATTCACCCAATGCCGCACAATTTGCGTCATTATCCACTTTAACAGGCAAA
GGTATATGTTCTTTGAACTTATCGGCAAGAGGATAATGATTTATTTTTATATTATTTGAATACACT
ATCTCTCCTGTTTCAAAATCAATTGTACCGGGACAGCCTATGCCAATACCCTTAATCTCGTTCATT
TCAATACCAATACTTTGGACAAGTTTTTTTGACAAATTCGCCATATCGGTAACTATTTCATCTGTC
GGACGTTCTGACAAAGTAGGTACAGAATCTTTTACAACAATCTTTCCCTCCTCTGTCACAATACC
TGCCGCAATATTTGTTCCGCCAAGGTCTATTCCTATATAATACATTTTTCCACATAGCCTTTCTGC
CCACCAAAGCAATGAATATGTATATACAAATCTTATCGTGGGCAGATAAGGTTATAATACATTAT
TTTGCAATTCCTACGGTCATTATCTCATACGGTTTTACCTCAATATTGAAATAATTGTTATTACTG
TCAATTTTCTTAATCTTCTTTTCTATGATATTACTGATGTAAAGATTATCGATAACATCACTCTTTT
GAATTGTCAACGTTGTAGTTTTATCGCTTACATTTACCCAACGAATAATTATATCTTCGCCGTTGC
CTTTTTGCTTGATACCCGTAAGAATCAAGCCGTTGCCTTGCCAATTAATCATAGAATAATCAAGC
GGCATAACTCCGTCATGACAATCTGTATCGGCTGTTATAATATCTGTTCTGAACTGATAGCACTC
CTCATAAGCTCCACTTGAAATCAAATCACCCTTAAACGGTACAATTTCAATTTCCGTTTCGCTTAT
ACCCAAGCATTGAGCCTTAGGAGTCGGGAACACGCCCCAATCGCCCATTTCACCGACTGCACGA
AGAATTGTAACTGCGATTGTATTATCTAAATCCGGTAACATTTCATATTCGTACAAGCCTATATTT
GCGACCGCTATACCCTTTTCACCGTCATCAATACTCACAAATCCCTGCTCGTGTTCACACGCACT
CGGATTATTCCAACCTGCATTATGACGATTATTTCTTGTAACAACCTCAAACACGGAATCAGCCT
TATGTACATCGGAATTTATACCTGTCGGCACCATAATTCTAACTCTATGGTCTTTTACTTCATTAT
CAAATCTCGTTTTGATTTTTACACCCTTACCGTTTTTGTCAAGTGAAACAAATGTTTCTATTTTCA
TTTCAACGGTATCGTTACTTCTTCCGCCGACTCTTTCTTTAAAGAATACCATATGACTCTTTTCGT
CCTCGAAATTATCATCGCCCGACTTCGGAACTGTAATTGTGTTTGTGATTTTATACATTGCTCTGT
ACGGCTCATCTTCCGCAAGTTCAATCTTCGCAACTGTATCTTGCGTTGTTATCGCCTTACTTCCCT
CAGGCATTTTATACATATATTCATTGCCCAAGTCACCTGTTTCTTCGTAATAAGCGACGCCTTTAT
ATGTTCTGCCGCTTGCTTTGTCTGTTACATTAAGCGAGCCGTTCTTGTTTATTTCTACACGAATTG
CATCGTTTTCCATACAATTCTCACTGCTGACAAGTGTATCTGTTACTTTTTCCGTGTCACCCTCAA
CAAGGGCATATGTTTTATATCCGACAGCGGATATATTTTCAGCCTCAAATGTTACACGAACACGT
CTTGCCATGTACGGTTGTCTGAACTTATCCTTAGGCAAATCGTAGCCGAATTTAACTCCCAAATC
TTCGATTTTAAACGGTATTGAATTTCCGTCTGAATCTATAAGCTTATAGTTCGGCACATTTATTTC
GTCTAAATCATATGCACATTTTTTAAGCCAGCCCGATTTACGAGTTACGTCAATTTCCACCGATA
CGACCGATGTGCGCTCTCTGCCTGCCGTGTTAAACACAACAAACGGAAGTGCATTTTTGTACTTT
TCGTATTCCTTTGTATTAATCTTATCCGCAATATATCTTTTTCCCTCTGATACAAGATAGTCGGCA
ACTTGCTTACTCTTATTAAAACGTGTTGCCATTTCGTCTTGTACCTCATCAACACTGCAACAGCAG
ATACTGTCGTGAGGGTGATTTTGCATAAGTTTCTTCCATGAATATTCAAGTTCGTCCGACGGATA
ATTTTGTCCCAAAACAGATGATAAAACTCTGACAGGCTCTGCACCGTTTTCAAGTGCCGATTCAC
ATTTTCTGTTCATCTGCTTTAAGTAAATATGCGATGACGCACAATTCATAAGCGTTGACCAACCG
TCTGTATCCTGGCTTGTAAGTTCGCCTTTTACAACTGCCAAATCATTTGGTACTTTCTCTTTAATT
GCCTTAATATATTCCGGGAAATTTGAATGTTTAAAATTAATGTCGGGATAAAGTTCCGACGCAAC
TTCTATTGCCTTGCCTAAATCAGCCTGTACAGGCTGATGGTCACAACCGTTCATCAACAAATATT
CATCAGTTGACGCAAAAGTCGCAACTTTTTTCAATCTGTCGTCCCAATATTCTTTTGCAATTTTCT
TGTCTGTCGGAACTTCGTTGCCGTTATTATACCAATTCGCAAACAGAATACCGAAAATCTTCGTA
CCGTCCGGAGATTCCCACATCATTTCTGAATACGGAGATTCATAGTTTCCATTTTCTTGAACTTCG
TTATCAAACCCGACAGGACGTACACCTCGTCCGAAAGTCACTGTATCCATTCCCGCTTGTTTTAA
AAGTTGGGGCATTTGCCCCGCATTACCGAATGCGTCCGGGAAATATCCCATTTTACACATAGCTC
CGTACTTTTCAGCCTCTTTCATACCGACAAGCAAATTTCTGATATTTGCCTCACCGCTCGTATAAA
ATTCGTCTTGCAAGATATACCAAGGACCGATTATAAATTTACCCTCCTTGGTATACTTAATAAGT
TTTTCTTTATTTTCCGGTCTTATTTCAAGATAATCGTCAAGCACAATAGTCTGACCGTCAAGGAAA
AAGCTTTTGAATGAATCGTCCTTTTCAAAAACCTCCATACATTTATCTATCAATTCAACAAGTCG
CATTCTGTGTTGTTCAAACGGAAGATACCACTCTCTGTCCCAATGAGAGTGTGATATTATATGTA
CATTTTTGCTCATTTGAAATTCCTTCTTATACCATATAATAAATTTGGGTTTGATATATTTGTTATA
CATTATATCGCTATTATATATCAAAATCAATACTCAAGTAAAATTTCGTTGCACCTGTTGCACTAT
TATTACCGTCATAAAGTTCAACCTTTGATGTTGCAATAGATTGATTATCACGAATAAGTTTTATTT
GAATACCGTTAGTGCTTCCCTCCAATATAAACACCATTGCGTTTTACAATGATTTGTACCACGAG
TATAGAACTACTCTACACTGTCTCATTTGTATACGAATAGACTTCTTCATGATATATTTTAACTTC
AAATTATCTCCGTCCATTCCAATTATATATCAATTTTTTATTTGTTATGTATAATTATATTACACA
ATATATCAAAGTTCAGTATTTTTCTGTTTTTACATAATCTAAAATTTAATTTTAACAAAAAAAATA
AACCGTTAGATTGTTTCAAACGGTCTATTTTTAAAATTCAATTGCGAACAGCAATTTTAACAGGC
ACAATGTATTTAATTCCGGGCATTTCGCCGTTTATTTCTCTAAGAACGGTTTCACCTGCTTTCACA
CCCAGTTCAAAGAAGTTTTGCTCCACGGTTATTATCTTGTTTCCGCCATCCATTTCGTCAAGCTCG
CTTATATTATCAAAACCCATTATGCACATTTCATTTGGAACCGATATATCAAGTGCCTTACAACA
ATTATATACTTGAATTGCCACCCAATCGTTTTGGCACAAAACACAAGAAATTCCTTGTTCATGCA
TACGATTTACAATCGTTTTCAAATAATTTTCAACGTTGCCGTATTGTTGTCTTTCTTCTTCAGTCA
GCATTTCGTACTTGTCGTCAATGTTCGCATATACATAATCAAGATTAACTCCTAAACCTTTTTCTT
CAAGTGCCGCCGCATATCCCATATATCTATCCCTTATTGATATAGTTTCATTCACACGACCTCTGC
AAAAAAATCCAATCTTTTTATGTCCATGCTCTAATGCATATTCGCAAAGTGCTTTTCCGCCGCCG
CTGTTATCCGACACAATATAACTCATAGGCATATTTTCTATATAATTATCAATAAGCACAAGAGG
AATTTTTTTAACTAAAAACTGATTATACACTTCAAAATTTCTGCCACCACGTACAGGATAACATA
TAACGCCGTCTATCCCCTGTTCCAAAAGTGAACGAAGTATTTTCTCCTCGTTTTCCACACTTCTGT
TTGCATTATATATACTGACAAAGCAATTTTCCTTATTGAGCACACTATTAATTCCGTCAAAGCATT
TGAACATATTACCAAGCTTTATATCAAACGGCATAACCAATGCCACAAGAGATATATCTCTGTTT
TTTTTGTGTATCGTAACTGCGGCATTGTCTTCCTTGTCCTTGCCAAGAATACTCATCGCATTTTTT
GAAACAAAACTACCGCTGCCTCGTTTTCTGTTAATCAATCCGTCATGTTCAAGCTCCTCAAGTGC
TCTGATTGCGGTTATACGACTCACACCGTATTCCTTAGTAATTCTGTCCTCTGTGACAAACGGTGC
GTCGTATTCAAAATCTCCCGACTTTATACGCTCTTTTAATTTATCCATAATTTGTTTGTACAATGG
TTTTTTATCTGACATTAACGTTATTCCTCCAAGAACAATATATCTTAATTCATTTTCTTTCTTATAT
TTAATATATCACTTTTCGAGTGACTTGTCAAACAATATATCAATTAAAACAAACAATTTGTTTCTC
TCTCGA
SEQ ID NO: 32 - B intestinalis
CCTTGCAAACAACAAGTTAAGTTCTTGTTGACAAGGAATGGACTTCTTGCAAACACCTATAAATC
AAAGTCTTGTTTATTCCACTTACAGGCCCATAGCTTTTTTATATTTAAGCAATGCCTGTAAATAGT
AATAATCTGCATAATTTATAGAAGCATCGATCTCATATCCGCCTGGCTGATTACCAGTACTATGC
ATCAAAAAGGCAGGTTTTATGTCCCGACACTGATATCTTTCGGAAGATAATTCTCCCAACATCCG
TGTGGCTGCATTTAAATAGCGGGAGGCCAATGATGGAGTATCTTCCAGTTCGGATAACTCAATA
AGCGCAGAAGCCGTAATTGCTGCTGCCGAAGCATCTTTAGGTTGTTTGATCATGTCCGGTGCATC
AAAATCCCAATAGGGTATATAATCTTCGGGCAGGTTTTCCAAATAAAGTTCTGTGACTTTTTCGG
CAAATCGCAGAAATGTCTTATCCTGAGTTTCCCGATAAACCATCATATAGCCGTAGATAGCCCAT
GCCTGTCCGCGTGCCCATAAACTGGAGTCACCGTATCCCTGATTGGTTACCCCTTTTATGAAATG
TCCGTCAATCGTATCATAGACTGCAACATGATAATTGCCTCCATCTTCACGGAAGGAGTATTTCA
TAGTCGTTTGTGCATGTTTCACGGCTATATCATACAGTTCCTGTCCGCCACCATTCCTGGCAGCCC
AAAAGAGAATTTCCAGATTCATCATATTATCCATAATGGTATTATGGGGCCACCCCATTCTTTTT
ACCATTCCAGGCCACGAAAGTATGGTGCCTACCTTGGGATTATATAACTTTGCTAACTTTTGTGC
CCCTTTCAGGATGACCGTTTTATATTCCTCATTTCCGGTTATGCGATAAGCATTACCGAAACTACA
GAATATCTGGAAACCGATATCATGGTCGGCACCATGTGCAGGAGTTACCAATGGCAACAAACAT
TCGGTATACCGAATGGCTTGTGCTTTTATCTCTTCATCACCCGTAGCCTCATAATCATACCAAAG
GATACCGGGCCAGAAGCCACTGGTCCAATCATAAATATTGCTCATGTTCCAATTGGTCTGATTGG
CTTCCATGCTCCTGGGCATTAAACAGGAATCTTGATCGGCCTCGCTTAATGTCCGCCTTATCTGG
GCGTCACAGTACTCCAATTGTCGGTCTAAATGGATAGTATCTGCTTGTTTATCGGGCGCACAGCC
CACCCATCCGAGGCTGATGCTAACCAACAACAAACTCAGTTGCTTTCTCATACTTTTAGCTCATT
TAAATTATTATTCTTTACAACACACTCTTTACCTGCTTCAGATTGCCAATGGTTCCGACCCAACCG
CAAACAACCTCTTAGCAGACAGAGATCTTTTTCTTTTTATTCATTAATATGCCATGCATTTTCCTT
ATTTGTCAATTCATAAACAAGGGTAGCTCCTTCTGCTATTTCCCTGTGATATATCCACCCCTTATC
CAGTACCTTACCATTGATAGATACAGATTTTATGTAACAGGCATCAGGCGTATCCTTTAGAACCT
TGATACTTATTTTTTTCCCATTCTCCATAGTCAGTTCCACGTCAGTGAAGGCGGGTGGCAACAGA
TAATAAAAGTCTTGTCCCGCATTAGGGAATAACCCTATGGAGGTAAATATATACCAGGACCCCA
TCGCACCGCTATCTTCATTATCCGAATATCCTTTGAGCAGAGAAAAATTATCTTTTCGTATCTGAG
ACACATAACGAGCTGTCAGATCCGGTCTTCCGCAATGGGTAAATATGAAGGGAGATAAGAAACC
GGGTTCATTATTTAAACTGATCAGATTATTCTCAAATCCATAAGACAGCCGCTTTATCATATTCG
CTTTTCCACCACAATATTCTATCAATCGGTCAAACTGATGTGGAACAAACAAAGTGTAAGTCCAA
GAGTTACCTTCATAAAAATATTCTACCCACGAACCATATGCCTTGGCAGGGTCTATAGCTACCCA
TTCACCATTCGCCTTACGGGGTACTATGAATCCTTTATAAGTATGGCTTTCCTGCAAAGGATTGA
ATAATTGGCTCCAATTGCCGGAACGTTCGTAAAGTTCCTTTTGAGTATTTTCATCATGCATGATTC
CGGCTATTTCAGAAGTACAGAAATCATTGTAAGCATATTCTATTCCGGCACTACACGACATGATT
CCGCCAGTTTCCGGTTCCCAGCCTAATCGCAGATAGTCTTTACTGCGTGCATGATAAGCATTCCA
CTTCATAAGTGCATAAGCTTTTTCATAATCGAATCCCTTTACATTTTTCACGATAGCATCAGCTAT
TATATTATCCACATCGTCACCTCCCTGTTTAGAAGTCCAATCCAATGAACTGGTAAAAGTGGGAT
TGCATACACCATTGTGTGCAAAACGATCAATAAATGAATTTATAGTCTGAGCCACATAGCTCTCG
CGCAACAAAACCATGAGTGGATATTTGGTACGCCACGTATCCCAAACACAATAATGGTCGTCCA
TGTGGGCTGATTCACTATCCCAATGCGGATTATCGCCGGTACGATCTCGAGGCATCACAAAACTG
TGGTAAAGCGTGGTATAAAACAGCCGTTCCTCAGCTTCATTCTCAGATTTGATTTTTATGGAAGA
AAGCGTATTGTCCCATATCGCTTTAGCATTCTCCTTTACGGTATTGAAACTGTTATCCGCAATCTC
TTCTGATAAAAACAGGGAGGCATTTTCTATACTTTTCAATGATATACCTACGTTTAGATGGACTA
CTCCCGGATTCTTATTCAGAGCCAGACAAGCGTATAAAGCTTTGTCACCCTGATCTGTGATCTTC
ACTTCCTTCAAAGGTGTATCTGTTTTCATCGCAAAATACACTTTATAAGCATCTGTACTTCCAAAA
CCACCGCTGTATTCTCCCCATCCCGTCAAAGTTTGCTGTTCGGGATTGTAGTTTATCTCCCCGCCG
TGGAATAATCCTTTTACCTCAGGCACTATATGCTGCGGAATATTGTGCGCAATATCCAGTAGAAT
ATTCCCCTGATCCGTTTCAGGAAAAGTGAAACGATAGGCAACGCAATGATGTGTAGGCGAAATC
TCCACCTGAATATCGTATCGACTTAACATTACCTTATAATAGTAAGGTGTGGCTTCTTCACCTTGC
TTGGGCGAATCGTGATCTGTTTCACCTGGATTAAAACCGACTTGAGGTGACAGAAATATCTGTCC
GTAACGCCCCCAGCCGATTCCTGAAACATGCAGTTGTCCAAAGCCCCGTATCGGCTGATCGGGT
ACATAACCGGCATGTCCACCATATGCAGTCTGGGGAGATGGGTTGACGGAGCCATGTGGAAGTT
GAGGTCCGACAACACAATGCCCAGCTCCATAAGTTCCCATCCACATATCTACTTTGTCGGCTAAA
GATTGTCCTTTTATAAAGGATACATTCATTAATAAGAAAAGGCATATGCCCAATGTTCTGGTCTT
CATGGAATGTTATGTTTAAGGTGATAAATCTATTATTTTCATTGATACACAAAAGTACGCGATAG
TTACCATTGGCAGATACAAAAATTCTCTTAAAGTATACAACAATAACAAGCACTACAGCTTTTTA
CAATATTCTTGCACCCAAAAATTATATATTTGTATGTCGGAAATAAAAATAGGCCTACTTTTGGG
CAGTTAAAATCACTACTATGAAACAGCTTATTACTACCTTATTTATCTTTATATTCCTTCAGCCAT
CCTGGGCTTCGCTCTACAGAAACTATCAGGTGGAAGACGGGCTCTCTCATAATAGCGTCTGGGCT
GTTATGCAAGACAAGCAAGGTTTTCTATGGTTTGGGACGGTAGACGGCCTTAATCGTTTTGACGG
TAATTCCTTCAAGATCTATAAGAAATTGCAAGGGGATTCCTTATCCATAGGCAATAATTTTATCC
ATTGCCTGAAAGAAGATTCTCACGGTCATTTTCTGGTAGGAACCAAGCAGGGATTCTATCTGTTC
AACCGCGAGAGTGAAACATTCAGCCATGTCAGGCTGGACAACCGCTCACGTGGAGGAGATGATA
CCAGCATTAATTATATAATGGAAGATCCCGACGGAAATATATGGTTAGGATGCTACGGACAAGG
TATCTATGTGTTAGGCCCGGACCTGCAGGTCAGAAAACATTATATCAATAAAGGGAATCCGGGT
GACATTGCTTCCAATCATATCTGGTGCATGGTGCAGGATTATAATGGAGTAATCTGGATAGGAAC
AGACGGAGGAGGCTTAATCCGCCTTGACCCCAAGGACGAAAGATTTACTTCGATTATGCACGAA
AAGGACTTAAACCTGACAGATCCCACGATTTACAGTTTATACTGTGATATGGATAATACGATTTG
GGTAGGAACTTCTATCAGTGGACTCTATCGTTGTAACTTCCGGACAGGAAAGGTAACCAATATA
GTATACCCTCACCGTAAGATATTAAATATTAAAGCTATTACGGCATATTCCAATAATGAGTTGGT
GATGGGTTCGGACGCAGGACTGATCAAAGTCGATTGCATTCAGGAAACGATTTCCTTTATTAATG
AAGGACCGGCATTTGATAATATAACAGACAAAAGTATATTTTCCATAGCCCATGATATGGAAGG
CGGCCTATGGATAGGAACCTACTTTGGAGGTGTCAACTACTATTCTCCATACGCCAATAAATTTG
CCTATTATCCAGGATCCAGCGAAGAGGTTTCAAAGAGTATTATCAGTTATTTTACGGAAGAATCT
TCCGACAAGATATGGGTAGGAACCAAGAATGAAGGGCTATTACTATTCAATCCGGCAAAAATAT
CGTTTGAGACTACCCATTTACAGATTGATTATCACGACATCCAGGCATTGATGATGGACAATGAC
AAATTGTGGATCAGTGTATATGGGAAGGGAGTCAGTATGGTCGACGTACATTCCAATACCTTGCT
AAAGCGCTATTCCAATGACGTAGGAGGCCCTGATCTGCTAACATCCAATATTGTGAACGTCATAT
TTAAGTCGTCGAAAGGACAGATTTTCTTTGGAACCCCTGAAGGTGTTGATTGTCTGGATGCTGAA
ACTAAAAAAATCAACCGGCTGGAACGCACAAAGGGCATACCGGTGAAAGCCATAATGGAAGAT
TATAATGGTTCCATCTGGTTTGCCGCTCACATGCATGGACTGCTCCATTTATCGGCTGACGGAAC
CTGGGAATCCTTCACCCACATGCCGGAAGATTCAACCTCATTAATGAGTAACAATGTGAATTGCA
TTCATCAGGATGCCAGATATCGCATCTGGGTAGGTAGTGAAGGAGAAGGAATGGGACTTTTCAA
TCCGAAAACCAAGAAGTTTGAATACATACTTACCGAAAATCTGGGACTTCCCTCGAATATAATCT
ATGCCATCCAGGAAGATGCAGATGGCAATATATGGGTAAGTACCGGTGGCGGTCTGGCCCGGAT
TGAACCGGAAACACGTTCTATCTGTACTTTCAGATACATTGAAGACCTGATTAAGATACGTTACA
ACCTGAATTGCGCCCTGCGGGGTAGAGATAATCATCTATATTTCGGAGGAACAAATGGCTTCATT
GCTTTCAATCCGAAAGATATACAGAATAACGAGTATAAACCGCCCATCTGCCTCACGGGATTCC
AGATTTCAGGGAATGAAGTTGTCCCCGGTATCGAAGGTTCACCATTGAAGAAGTCTATAAGCAT
GACGCAGAAGATAGAACTTGAATCTAACCAGGCTGCCTTTAGTTTCGACTTTGTTTGCTTGAGTT
ATCTCTCGCCTGCACAGAACAAATATGCGTACAAGCTTGAAGGCTTTGATACGGACTGGCACTAT
GTGGCCAATGGTAATAACAAAGCCATCTATATGAATATACCTTCGGGCAAATATACTTTTTATGT
GAAAGGAACCAATAATGACGGAGTTTGGTGTGATACCCCTATAAAGGTGACTGTTATCGTAAAA
CGCCACTTCTGGCTATCCAATATGATGTTACTGGTTTATGCCATTCTCGCAATCTCCGCATTTACT
TTACTTATCCGCAGGTACAACAAGCGTCTGGACTCTATCAATCAGGATAAGATGTATAAGTACA
AAGTAGAAAAGGAAAAAGAAATATATGAAACCAAGATTAACTTTTTCACCAATATGGCCCATGA
AATACGTACTCCGTTGTCATTGATTGTAGCTCCTCTGGAGAACATCATTTCATCGGGCGACGGAA
GTCAGCAAACCAAAAGCAATCTGGAAATAATGAAACGGAATGCAAACCGGCTGCTGGAACTCG
TAAACCAACTATTGGATTTCCGCAAGATAGAAGAAGATATGTTCCGCTTGTGCTTCAGCAAGCA
GAATATTTCAGAGATTGTCCGCAATATACATAAACGGTATGTGCAATATGCAAAACTGAAAGAC
ATAGATATAAGACTGGTAGAACCGGAAAAGGACATTGCTTGTGTGGTAGATAAGGAAGCGATG
GAAAAAGTCATCGGAAACCTGCTCTCCAATGCCGTAAAATATGCCAATAGCCTGATAACTATCA
ACATAAGTACAGACAACAATCTATTAACAATCAGTGTAAAAGATGATGGTCCGGGCATTAAGAG
TGAATTTATAGACAAGATATTCGAATCATTTTTTCAGATAGAGAATAATGCGCAGAGAACAGGT
TCGGGATTAGGGTTGGCATTATCAAAATCACTGGTAACAAAACACAAAGGGAATATTGCAGCCT
CATCCGATTATGGGCATGGATGTACATTGACATTCACAATTCCTATGGATCTTCCAATCAGTATA
TCACAGCTTACGGAAGAATATCCGGAAAAAGAAGATATCTCCGTGCAACAAACTGCGCTATCTC
CTGTAGAAGGGAAGTTAAGAATAGTGTTGGCTGAAGACAATCAGGAACTCCGGAGTTTTTTAAG
TAATTATTTAAGTGACTATCTGGATGTATATGAAGCTCAAAATGGTTTGGAAGCATTACAATTGG
TAGAGAATGAAAACATTGATATCATAGTATCGGATATCCTTATGCCCGAAATGGACGGTCTGGA
GCTTTGCAAAGCCTTGAAGTCTAATCCGGCTTACTCGCATCTGCCGTTTATCTTATTGTCCGCCCG
AACAGATACGGCCACCAAGATAGAAGGACTGAACACGGGAGCCGACGTGTATATGGAAAAGCC
TTTTTCGAGCGAACAGTTGCGTGCACAGATCAACAGTATCATCAATAACCGTAACAGTATCCGTG
AAAACTTCATTAAATCGCCTTTGGATTATTACAAGCAGAAGAGTGCCGAACCCAATGGAAATAC
TGAGTTCATAGAGAAACTGAATATTATTATTTTAGATAACCTCACCAATGAGAAATTCTCCATAG
ACAATCTCTCCGAGATGTTTCTGATGAGCCGGTCCAATCTGCATAAGAAGATAAAGAATATCGT
AGGCATGACGCCTAATGATTATATCAAACTGATTCGTTTGAATCAAAGTGCACAGCTGCTGGCTA
CCGGGAAATATAAGATAAATGAGGTGTGCTATCTGGTAGGTTTTAATACACCTTCTTATTTCTCC
AAATGTTTTTATGAGCATTTTGGAAAGCTGCCAAAAGACTTTATCGTGATAGAATAAATGATTAC
TAACCCAATAATTCAAGAAGGGAGGCTTATGAGGAAAAGATAAAACAGGATGGTAATCGAAGA
AGAAAGCATGAGGCATCCGACCTCCCCATGCTCTCTGCATAATAAGACTTACAGCACAGGATAT
TTGTTTGCAAGCTGATATTATGCCCAAAGATAACCATTTTCACACTGAAGAGAAGTAACCATGAG
AATTTTATATTGGTTTTTATGGTTTGTTTGTAATTTCTAATACTAATAGTATCCATTAAACAAGGA
TTAATAGCATGAAAACAAAATTTATTGCCACATTCTTTTTGCTTATATGTGGTTCCGTCATGTTTG
CTCAAACACGTACGGTAAAGGGCAAGGTTGTCGATAAGGCAAATGAACCGCTGATTGGTGTAGC
AGTTAATATTAAGAATACATCACAAGGCAGTATTACAGACTTTGAAGGAAATTATTCCATACAA
GTGAATACGGAGAATGCCGTACTGGTATTTTCGTATATAGGATATGATAAACAAGAAATAAAAG
TAGGTGCACGCAATGTGATTGACGTGGTAATGCATGAAGCTTCCATTGCGCTGGACCAAGTAGT
GGTAGTAGGCTATGGAACATCCAAAAGAGGAGATGTAACCGGCTCTATCAGTTCCATCGATGCG
GCAGAAATAAAGAAAGTACCGGTGGTAAATGTAGGACAGGCTTTGCAAGGCCGTATGTCGGGTG
TGCAGGTGACCAATAATGACGGAACACCCGGAGCCGGAGTGCAGGTCCTGATACGTGGCGTAGG
ATCATTTGGAGATAACTCACCGCTGTATGTAGTGGATGGATATCCCGGTGCAAGCATTTCCAATC
TGAATCCGAGTGACATACAAAGCATTGACGTACTGAAAGATGCTTCAGCAGCAGCCATTTATGG
GAACCGTGCTGCTAACGGCGTTGTCATCATCACTACCAAAAGAGGAAATGCGGATAAAATGCAG
TTGTCGGTAGACGCAACTGTTTCCGTACAGTTTAAGCCTTCTACTTTTGACGTACTGAATGCACA
GGATTTTGCATCTTTGGCTACGGAAATAAGTAAAAAGGAAAATGCTCCGGTACTGGATGCATGG
GCTAATCCTTCCGGGTTGCGCACCATCGACTGGCAGGATCTGATGTATCGTGCCGGATTGAAGCA
GAACTACAATTTAAGTCTGCGGGGAGGTTCTGAAAAGGTACAGACTTCCATCTCTCTGGGATTAA
CCAATCAGGAGGGTGTAGTGCGGTTCTCTGATTACAAACGCTATAACATAGCATTAACACAGGA
TTACAAGCCGTTGAAATGGTTGAAATCTTCTACCAGCCTGCGCTATGCATATACGGACAATAAGA
CTGTATTCGGTTCCGGCCAGGGCGGCGTAGGAAGATTGGCCAAGCTGATTCCGACCATGACGGG
TAATCCACTCACCGATGAAGTGGAAAATGCAAATGGAGTATTCGGCTTCTATGACAAGAATGCC
AATGCCGTAAGAGATAACGAGAACGTATATGCACGTTCCAAATCGAACGACCAGAAAAACATAT
CCCATAATCTGATAGCCAATACCTCATTGGAAATCAACCCTTTCAAAGGCTTGGTATTCAAGACT
AATTTTGGTATCAGCTACGGTGCTTCTTCCGGTTACGACTTCAATCCTTACGACGACCGTGTTCCC
ACCACACGCCTTGCCACTTACAGACAGTATGCCAGCAATAGTTTTGAGTATTTGTGGGAAAACAC
CCTGAATTACTCTAACACATTCGGCAAACATAGCATCGACGTATTGGGTGGTGTATCTATTCAGG
AGAACACAGCACGCAACATGAGTGTGTATGGTGAAGGATTATCGAGTGACGGTCTGAGAAACCT
GGGCTCTCTGCAAACGATGCGTGATATCAGTGGCAACCAGCAAACCTGGTCTCTGGCTTCACAAT
TTGCCCGTCTGACCTACAAATTTGCCGAACGTTACATCCTGACAGGAACAGTTCGTCGCGACGGT
TCATCCCGTTTTATGCGCGGAAACCGCTGGGGTGTATTCCCTTCCGTATCAGCAGCATGGCGTAT
TAAGGAAGAAAGTTTCCTGAAAGATGTGGATTTCATCAGTAACCTGAAGTTGAGAGCAAGTTAT
GGTGAAGCAGGTAACCAGAATATCGGTCTGTTCCAATACCAGTCATCTTACACTACCGGTAAGC
GCAGCAGCAATTATGGATATGTATTCGGACAAGACAAAACCTATATCGACGGTATGGTTCAGGC
CTTCTTGCCGAACCCTAACCTGAAATGGGAAACTTCCAAACAGACGGATATAGGTATAGACCTG
GGATTCTTCAATAATAAGCTGATGCTTACAGCCGATTATTACATCAAGAAATCAAGTGACTTCCT
ACTGGAAATCCAGATGCCTGCACAAACCGGTTTTACTAAAGCCACACGTAATGTAGGTAGCGTT
AAAAACAATGGTTTTGAATTCAGCGTGGATTACCGCGACAACAGTCACGACTTTAAGTATGGTG
TAAATGTGAATTTAACTACCGTAAAGAACAAGATTGAAAGATTGTCACCGGGAAAAGATGCCGT
TGCGAATCTTCAATCATTAGGCTTCCCAACTACGGGTAACACATCCTGGGCCGTATTCAGTATGT
CGAAGGTAGGTGGTTCTATCGGAGAATTTTATGGATTCCAGACAGACGGTATCATTCAGAATCA
GGCAGAAATTGACGCCTTGAATGCGAATGCCCACAGATTGAATCAAGACGACAATGTGTGGTAC
ATCGCTTCCGGAACAGCTCCCGGAGACCGCAAGTTTATAGACCAGAACGGTGATGGCGTAATTA
CCGATGCCGACCGTGTTTCCCTGGGTAGCCCGCTTCCGAAGTTTTATGGAGGTATCAACCTCTCC
GGTGAGTATAAAGGCTTTGATTTCAATTTATTCTTCAACTACTCCGTTGGAAATAAGATATTGAA
CTTCGTTAAGCGCAATTTGATAAGTATGGGAGGTGAAGGCAGTATCGGTTTGCAGAATGTCGGC
AAAGAATTCTACGATAACCGCTGGACTGAAACGAATCCGACCAACAAATACCCGCGTGCCGTAT
GGTCTGACGTTAGTGGAAACAGCCGTGTGTCGGATGCTTTCGTGGAAGACGGTTCTTATCTTCGC
CTGAAGAATATTGAAGTAGGATATACATTGCCGGCAAACATCCTGAAGAAAGCCAGTATTTCTA
AGCTGAGAATCTTTGCCAGCGTACAGAACCTCTTCACTATTACCGGCTATTCAGGTATGGACCCG
GAAATAGGTCAGAGCATGAGCAGTTCAACCGGAGTTGCCGGTGGAGTTACCGCCTCGGGAGTTG
ATGTTGGCATTTATCCTTACTCACGCTTTTTCACCATGGGATTCAATCTTGAATTCTAAGGAGAGA
CATTTCTGTATGACAAATTATAAATTTACAATACAATGAAAAAAAGACATATAATCGGTTCATTC
CTGCTCGGATTGCTTTTAACGGTAAATACCGGCTGTGAAGATTTTCTTGATCAGAAAGATACATC
GGGTATCAATGAGAATTCTCTTTTCTTAAAACCTGAAGACGGTTACTCTTTAGTCACAGGCGTAT
ACTCTACTTTCCACTTCAGTGTAGACTATATGCTGAAAGGAATCTGGTTTACCGCCAACTTCCCT
ACTCAGGATTTTCACAATGACGGTTCGGATACGTTCTGGAATACGTATGAAGTACCGACTGATTT
TGATGCATTAAACACGTTCTGGGTTGGAAACTATATCGGAATTTCCAGAGCCAATGCTGCTATTC
CTATTTTACAGCGCATGAAAGACAACGGTGTACTGAGTGAAAAAGAAGCTAACACACTGATTGG
CGAATGTTATTTCCTGAGAGGTGTATTCTATTATTATCTCGCTGTTGATTTTGGAGGTGTACCTCT
GGAACTTGAAACAGTAAAAGACGAAGGTTTACATCCGCGCAATTCACAGGATGAAGTATTTGCA
TCGGTAGTCTCGGATATGAACATAGCAGCAGGCTTGCTCCCGTGGAAAGCGGAACAAGGCAGTG
CAGACAGAGGACGTGCTACCCGAGAAGCGGCCTTGGCGTATCAGGGAGATGCTTTGATGTGGCT
AAAGCAATATAAAGAGGCAGTAGAGGTATTCAATCAACTGGACAGCAAATGCCAACTGGAAGA
AAACTTCCTGAATATCCACGAAATTGCCAACAGAAACGGAAAAGAATCTATTTTCGAAGTGCAG
TTTACAGAATATGGTTCTATGAACTGGGGCGCTTTTGGTGTAAACAACCATTGGATCAGTTCGTT
CGGCATGCCGGTTGCCATTTCCGGTTTTGCTTATGCATATGCCGACAAAAAGATGTACGACTCTT
TCGAGAATGGTGACTTACGTAGACACGCCACCGTTATCGGACCGGGTGATGAACATCCGTCACC
ATTGATTGACCTGCAGGATTATCCGAAGCTGAAAGATTTCGCAACGAAAGGGAATGGGAATATC
CCGGCTTCTTTTTATCAGGATGAGGAAGGTAATGTGCTGAATACCTGCGGAACAGTAGAAAACC
CCTGGTTAGACGGTACACGTTCCGGATATTATGGAGTAAAATACTGGCGTAATCCGGAAGTTTGC
GGAACCAGAGGTGCAGGTTGGTTTATGAGTCCGGACAACATTATGATGATGCGTTATGCCCAGG
TACTTTTAAGTAAAGCGGAATGTTTGTATCGCCTGAATGACAGTAATGGTGCAATGGCTATTGTA
CAAAAAGTCAGAGACCGTGCTTTTGGTAAATTACAGAATTCCGCAGTAGAGGTACCGGCACCTG
CCAACACAGACGTACTTAAAGTAATCATGGATGAATATCGTCATGAACTCACCGGTGAAACGTC
TCTTTGGTTCTTGCTGAGAAGGACGGGAGAGCATGCCAATTACATCAAAGAGAAATATGGCATA
ACGATACCTACCGGAAAGGATTTGATGCCAATACCTCAGACACAAATTGGTTTGAACCAGAATT
TGAAACAAAATCCCGGGTATTAATTCTTAAGTAGGTAAGAAGATTGTATTTTTGTGGTGGGTTGC
CATGTGAAAGCCGGCAACCCACCCTATTTCAATACTAATAAAAAAAGATGTAGGATATGAAGAA
CACTGTTTTACCGTTGATACTGTTTTTATGTATGCTTTGTTTGGGTTCACACTTGTATGCCGGCCA
CAGTATGCATCCTCTGAATCAGATATCTTACGTAAAGAAGAAAATAAAAGAACAGCAAGAGCCT
TATTTTACGGCTTATCGCCAGTTAATGCATTATGCAGATTCGATACAGGAGGTTTCACAGAATGC
CTTGGTCGATTTTGCGGTTCCGGGGTTCTATGATAAACCGGAAGAACACCGGGCTAATTCTCTGG
CTTTACAGCGTGATGCTTTTGCGGCCTATTGCTCGGCATTGGCTTACCAGTTATCCGGTGAAGAA
CGCTATGGGCAAAAGGCATGTTACTTTCTGAATGCCTGGTCTTCTACCAATAAAAAATATTCGGA
ACATGATGGTGTCCTGGTAATGAGTTATTCAGGCTCCGCCTTGCTGATGGCGGCAGAGTTAATGA
TGGATACGCCGATATGGAATCCTCAGGATAAAGATGCTTTTAAAACTTGGGTATCCCAAGTGTAT
CAGAAAGCTGTGAATGAGATTCGCGTTCATAAGAATAATTGGGCGGACTGGGGACGTTTCGGTT
CTTTGCTGGCAGCTTCCCTTCTGGATGATAAAGAAGAAGTGGCCCGTAACGTGCAGTTGATAAA
GTCCGATTTATTTGTGAAGATTGCAGAAGACGGACACATGCCGGAAGAAGTGGTCCGGGGAAAT
AATGGAATATGGTATACCTATTTTTCATTGGCTCCGATGACTGCCGCCTGCTGGTTGGTTTACAA
CCTGACCGGTGAGAACTTGTTTGTATGGGAACATAACGATGCGTCATTAAAGAAAGCTCTGGAC
TACATGTTTTACTTCCACCAGCACCCTTCGGAGTGGAAATGGGATACACGGCCAAATTTAGGAGC
CCATGAGACCTGGCCTGATAATTTACTGGAAGCAATGGCAGGAATCTATAATGATGCTTCATATC
TTCAGTATGTAGAAAGCAGTCGCCCGCACATATATCCATTGCATCATTTCGCCTGGTCTTTTCCTA
CTTTGATGCCAGTGTCGCTTAAAGGCTATGACTTAACGGATAATAATACATGGGCTAATTATAAT
CGCTATGAAGTGGCAAATAAAACAGTGAAGAAGCCTGTAGCTATTTTCATGGGAAATTCCATAA
CGGAAGGCTGGAACCGCAGTCACCCGGACTTCTTTACACAGAACGGATATGTGGGACGTGGCAT
TTCAGGACAGGTCACAGCACAAATGCTGGCCCGCTTCCGTGCGGATGTTCTGGATTTGAAGCCTC
AGGTAGTATGTATCTTAGCCGGTACAAACGATATTGCCCAAAACTGCATGTATATGTCGGTTGAG
AATATAGCCGGCAATATCTTTTCTATGGCGGAACTGGCCAAAGCCAATGGAATAAAAGTCGTTA
TCTGTTCCGTACTGCCTGCTACCCGTTATTCATGGCGTCCTACTGTTCAGAATCCTGCCGGTCAGA
TTATTCAATTAAACAAGCTACTGCAAAAGTATGCTCAAAAGAATAAGATTCCTTATGTCGATTTT
CATTCCATGATGAAAGACGAACAGAACGGACTGCCTCAAAAATACTCCAAAGATGGAGTACATC
CAACCAAAGAAGGCTTCAGCATGATGGAACCCATCATAAAAGAAGCAATTGACAAACTGCTGA
AATAAATTCAGCGCACGTAAACCTTTATAGAATGAAGAATATATATTATATCCTGATACTTTGTT
GTTTATGCTTATTCTCATGTGACTCACACCCTGATACTAAAAGCTCACTGCCTTTCGGCGTGAACC
TGGCCGGTGCGGAATTTTTCCATAAGAAAATGGACGGAGTGGGACAGTTTGGAATAGATTACCA
TTATCCAACCACCAGAGAGTTCGATTATTGGAAGTCAAAAGGCCTGACCTTAATACGTCTTCCCT
TCAAATGGGAACGTATTCAACGCGAACTCTACGGCGAATTGAATCGCGAAGAAATTGATTATAT
AAAGTATCTGCTGGATGAAGCCGGAGCACGCGATATGAAAATCCTGATAGATATGCACAACTAC
GGACGCCGGAAAGATAATGGCAAAGACCGTATCATAGGTGACAGTGTTTCTATCGATCATTTTG
CATCGGTCTGGAAGCAAATTGCCGGTGAGCTTAAAGAACATAGTGCCCTATACGGATATGGTCT
GATAAATGAGCCGCATGATATGTTAGATTCCGTGCCCTGGTTCAAGATTGCCCAGGCTGCAATTG
AGGAGAGCAGAAAAGTAGATTTAAAGACAGCGATTGTCGTAGGTGGTAATCATTGGAGTTCCGC
TGCCCGCTGGCAGGAGATTAGCGATGATTTGAAACACTTACATGATCCTTCGGACAATTTGATTT
TTGAAGGCCATTGCTATTTTGACGAGGATGGTTCGGGTATTTATCGGCGCTCCTATGATGAAGAG
AAGGCATATCCTACTATTGGGATTGATCGTACCCGCCCCTTCGTAGAGTGGTTGAAAACAAATAA
TCTACGGGGATTCATCGGAGAATACGGAGTTCCAGGAGATGACGAACGTTGGCTGGTATGTCTG
GATAATTTTCTGGATTACCTGAGTAAGGAAAATATAAACGGTACTTACTGGGCAGCCGGTGCAC
AATGGAATAAATATATATTATCAATCCATCCGGATGATAACTATCAAACAGATAAGATACAGTT
AGGAGTTCTGACAAAGTATTTAGAAACCAAGAATTAAATCAAACAGATTCAACAATGAAAACAT
TCATATTATCTTTTTTAATATATGCCGGCTGTTCATTACCCTTGACGGCGCAACAGATAAAACCC
GCTATTCCTTCTGATCCGGAAATAGAGGCAAAGATTAACAAGCTCTTACAGAAACTGACGCTTG
AGGAAAAGATCGGGCAAATGTGCGAGATCACAATTGATGTAATCACAGATTTCTCGGACAAAGA
AAACGGATTCAGATTGAGTGAGAGCATGCTCGATACCGTAATCGGTAAATATAAAGTAGGTTCT
ATTCTGAACACTCCCTTTAGTATAGCTCAGGAAAAAGAAGTCTGGGCAGACCTGATTACAAGAA
TCCAGAAAAAGTCAATGGAAGAAATAGGTATTCCCTGTATATATGGAGTCGATCAGATTCATGG
CACCACCTACACTCGCGGAGGAACTTTCTTTCCTCAAAGCATCAACATGGCTGCCGCCTTCAACC
GGCAACTCACGCGACGTGGAGCTGAAATCTCGGCTTATGAAACCAAAGCATGCTGTATTCCCTG
GAATTACGCTCCGGTTATGGATCTGGGACGTGATCCCCGCTGGCCGCGTATGTGGGAGAGCTAC
GGCGAAGACTGCTATGTAAATGCAGAAATGGGCGTACAGGCAGTGAAAGGTTTGCAGGGAGAA
AATCCGAACCATATCGGTGAGAATAATGTGGCTGCCTGCATCAAGCACTTCATGGGTTATGGCGT
ACCTGTTTCAGGAAAAGACCGCACTCCTTCCTCTATCTCCCGCACGGATTTACGCGAGAAGCATT
TTGCTCCTTTCCTGGCATCCATCCAGGCCGGTGCTTTATCCCTAATGGTTAATTCAGGCGTAGACA
ACGGCGTACCTTTTCATGCAAACAAAGAATTGCTGACCGGCTGGCTAAAGGAAGAACTGAACTG
GGACGGCATGATTGTAACAGACTGGGCCGATATCAATAACCTTTGCCTGCGTGATCATATTGCCG
AAACGAAGAAGGAGGCAATTCAAATAGCCATTAATGCCGGCATTGACATGTCTATGGTTCCCTA
TGAAGTAAGTTTCTGCACTTATCTGAAAGAATTGGTAGAAGAGGGTAAAGTATCGATGGCTCGT
ATTGACGACGCTGTATCTCGTGTACTCCGGTTGAAATATCGCCTGGGACTCTTTGATAATCCTTAT
TGGGACATCAGGAAATACGATCAATTTGCATCACCGGAATTCGCAAGTGTAGCATTGCAGGCAG
CGGAAGAGTCGGAAGTTCTACTGAAGAACGAAGACGATATTCTTCCTTTGGCGAAAGGAAAAAA
GATATTACTGACCGGTCCTAACGCAAACTCCATGCGTTGCCTGAATGGAGGATGGTCTTATTCCT
GGCAGGGAGACAAAGCGGATGAATGTGCACAAGCATACAATACAATCTACGAAGCTTTCTGTAA
CGAGTATGGAAAAGAATCTGTTATCTATGAACCGGGAGTCACTTATAAGACTTCTGCCGATGCTT
TATGGTGGGAAGAAAATACTCCCCGGATAGCCCAAGCCGTATCGGCAGCAGAAAAAGCTGATGT
TATTATAGCCTGTATAGGTGAGAATTCGTATTGCGAAACTCCGGGGAATCTGACAGACCTGAATT
TATCAACGAATCAGAAAGATTTAGTGAAAGCGCTGGCAGCAACAGGTAAGCCGATCATTTTGGT
TTTAAATGAAGGACGCCCACGAATTATCCATGATATCGTTCCTTTGGCGAAAGCCGTTGTACACA
TCATGTTACTCGGCAACTATGGAGCTGATGCATTGGTCAATCTGGTATCAGGGAAAGCGAACTTC
AGTGGAAAACTTCCTTTTACGTACCCGCATCTCATAAATTCATTGGCTACTTACGATTATAAACC
TTGTGAAAACATGGGGCAGATGGGTGGTAACTACAATTATGATGCTGTAATGGATGTGCAATGG
CCGTTTGGCTTCGGACTGAGTTATACCACTTACAGTTATAGTAATTTGAAAGTGAATCGTACTTC
TTTCGATGCTGATAATGAGTTAGTATTTACTGTGGACGTAACTAATACGGGAAAAATGGCAGGA
AAAGAAAGCGTACTATTGTACTCACGTGATTTAGTGGCAAGCATCACTCCTGATAATATCCGCCT
GCGTAACTTTGAGAAAGTGGATCTTCAACCCGGTGAAACAAAAACTGTTACCATGAAATTAAAG
GGAAGTGACTTGGCCTTTGTCGGTGCTGATGGAAAGTGGAGGCTGGAAAAAGGTGCTTTCCGTA
TGACATGCGGAACACAAAAGCTGGAGGTACATTGTACTACAACAAAGATATGGCAGACGCCTAA
TATTAGTAAATCCGGAATTTGAAAAGACCTGTACTTAAATAAGAAAATATAAAAGAAAGAGTAA
AGTTATCCTTTAGGGAGACTTTACTCTTTCTTTTATATAACCAG
SEQ ID NO: 33 - Mouse R. UCG13 GH5, truncated
GSAEINYNRSVPLEVKGNKIVKQGTDEMVVLRGVNVPSMDWGMAENLYESMTMVYDCW
GANLIRLPIHPKYWKDGSIWDGKNLTKEQYQKYIDDMVKAAQARGKYIILDCHRYVMPQQ
DDLDMLKELAVKYGNNSAVLFGLLNEPHDIKPTDIEKPTMEDQWEVWYNGGQIIVGGEEV
TAIGHQQLLNEIRALGANNICIAGGLSWAFDISGLADGYNGRENGYRLIDTAEGHGVMYDS
HAYPVKGTKSSWDTIIGPVRRVAPILIGEWGWDSSDNNISGGDCTSDIWMNQIMNWMDDTD
NQYDGIPLNWTAWNLHMKSSPRMISSWDYKTTAYNGTYIKNRLQSYGNLPETQDGVYSTD
FSTNDVFRGYKAPSGAASVSYSEANENIVVSHKPADWYATLNFPFDWDLNGIQTITMDISAD
TAETLNIGLYGSDMEEWTAPVKVDSTVKNITLSIDQLVRQGNQQTDGILNGAVSGIYIGSSTT
ETANNTKVVTMADEDNTAEYKINNYENGKVSVRKRTDAEDSASTVIVAFYDKNSVLTGIST
ANIRADEKGDEIIKAVNEPASYSCAEVFMWDSLNGMVPRCNPISNKVNITIDNIKIVKLAEPI
YTATEYPHTDIGAESYIDVDNTDFASQSTTKGAASTSYFTCENAEVVGADGENTQAKYITYD
RREGLYGGTVQFDLETVPSMDTKYFTISLKGSGTAQTINVNLGSEVSYNIALAEGDTDWHQ
YIFDISYGAQYPEDIAFVKLASNTKIESYFYADDFGFSKTKPERVIPNPEKTFIYDFATYNRNT
AKYEAVISTMPGSNDDEIRAEKVDGGLDFETQALEITYSRNGNIPSKTMVVYSPSDFFKGNS
NDDERTANRATLKADMEYMTDLVFYGKSTSDKNEKINVGVIDAANSMMTYTDTKEFTLTS
QWQQFRVPFDEFKVLDGGSELDCSRVRGFVFSSAENSGEGSFMIDNITHTSVADIEWAE
SEQ ID NO: 34 - B salyersiae CE7
MRHRVILFICVLQTLFAYAVGAETHFMLTLNEQWKFSTGDSSAWATTEFDDNQWGTISSRQ
YWEEQGYDGYDGYGWYRQHFMISEDWKPIVTNAGGLYIRYEFADDVDEVFVNGVSVGRM
GEFPPEYKVIYGGMRKYKISPGLLRFGEENLIAIRVYDNGGAGGLKTENILLQSITPMDDLML
DIRCDDSDWVFENTETIDFRVRPKQPLAAGGEFNLVCSVTTDTYLPVDSFVYRVKGDFEQPV
SFVPPAPGFYRITLYGEQQGVKSDFLKFNMGYCPEQIISPVDVEPDFDQFWETTLKELSEVVP
DYRMTLLEEKSQGAKNIYRVEMYSLGNVRIEGYYAVPKQKGKFPSVISFLGYGSGGGFPRP
DNLPGFCEFILSTRGQGIQLPVNTYGKWIVHGLEDKSQYYYRGAFMDLVRGIDFLCSRPEVD
TEKIFAEGGSQGGAFTLAACALDRRICAAAPYIPFLSDFEDYFKIAPWPRSVFEEYLRSHEESS
WDEIYRLLSYFDSKNLAPRITCPIIMGVGLQDNICPPHINFSGYNQVKSPKRYYIYYDKEHTV
GKSWWTIRNNFFRSFCN
SEQ ID NO: 35 - B salyersiae GH3 A
MKKLFKLFAFTCLAMSATAQNKTPIYLDETKPIEQRVEDALQRMTLEEKIKLCHAQSKFSSH
GVPRLGIPELWMTDGPHGIREEVLWDEWKGAAWTSDSCIAFPALTCLAATWDLDMSALYG
KSIGEEARFRGKDVLLGPGVNIYRTPLNGRNFEYMGEDPYLAAKMVVPYIKGVQQNGVAA
CVKHFALNNQEMYRGHINVEVSDRALHEIYLPAFKAAVLEGGTWSIMGAYNQYKGQHCCH
NQYLLNDILKKDWNFDGTVISDWGGVHDTYQSAYYGLDLEMGTWTDGLSWGKTNAYNN
YYMALPLLEKIKNGEIEENTVNDKVRRLLRMMFRTSMNTQKPWGSFGTEEHALAGRTIAEN
GIVLLKNENGLLPVDLSQIKKIAVIGENATKVMTLGGGSSSLKVKYEVSPLEGLKKRVGNAV
ELVYAPGYASPLTDKRDPRYIVLEGYRLPDAEKLTKEALEAAKNADIVLFFGGLNKNEHQD
SEGTDRLNYHLPYGQDELIAQLSKVNKNIAVILISGNAVAMPWIKEVPSVLEAWFSGTESGN
AIASVLVGDVNPSGKLPMTFAVRLEDYPAHTVGEYPGDSINVKYNEGIFVGYRWTDKHKIR
SLFPFGHGLSYTTFQYGKALLSSSEMNEKEILTVTIPIKNTGKVKGKEIVQLYIGDEKSSLERP
VKELKGFQKIELNPGEEKVVEFNITSNDLKFYDEAIQDWKAEQGKFNIFIGSSSTDIRAKTKF
NLK
SEQ ID NO: 36 - B salyersiae GH3 B
MGVSVFAADDGGALYLDAGRPVEQRVKDLMSRMTLEEKVGQMCQWVGLEHMRTASQDL
TVDELSNNTARGFYPGITEEDVRQMTIDGKVGSFLHVLTVKEANQLQELAMKSRLKIPLIIGI
DAIHGNAQVVGTTAYPTSIGQASMFDVGLVEEICRQTALEMRATGSQWTFNPNVEVARDPR
WGRVGETFGEDPYLVSLLGVASVRGYQGDGFGKAENVLACAKHFIGGSQPINGTNGSPTDI
SERTLREVFLPPFKATVDAGVYSFMTAHNELNGIPCHANPWLMEDILRKEWGFDGFIVSDW
MDIEHIHDLHRTAVDNKDAFYQSVDAGMDMHMHGPEFYEKVIELVKEGKLTEARIDESCR
KILAAKFRLGLFEKSFTDEKAAKSVLFNEKHQATALEAARKSIVLLTNDGILPLDEAKYKNV
FVTGMNADNQTILGDWALTQPDENVITVLEGLKLVSPDTKFSFVDLGWNIREMDKNKVEQ
AAKQAAKADLAIVAVGEYSLRTNWYDKTCGEDCDRSDINLAGLQQELVESILATGVPTVV
VLVNGRQLGVEWIAGHANALVEAWEPGSLGGQAIAEILYGKVNPSGKLPVTVPRHVGQIQ
MIYNHKPSMYFHPYAIGESTPLFYFGYGLSYTEYAYSDLTVSSAQMSGDGSVEVSVKVTNT
GTTDGEEIVQLYIRDLYSSATRPVKELKDFRRVPLRVGETKTVSFILPAGKLAFYDKKMDYT
VEPGDYEIMVGASSRDEDLMKRIVNVK
SEQ ID NO: 37 - B salyersiae GH5_5
MEKKTKRIAFVLATMLCGWQMMLAQPVSPAPTPTRAANDVKAMFSDAYPEKFGKFQIDY
DDWNSDKFLTTKTIVTPFGAADEVLKIEGLSTGSLQHNAQIALGTCNLSDMEYLHMDVYSP
SENGIGEFSFYLVSGWSKTVSCNVWYNFDTKQEYDQWISIDIPMSTFKNGGLNLAEINVLRI
ARGKQGAPGTIVYVDNVYAYGKAVEPESDVKIVANGNANLTTDVPLISAPTPKVAAANVFN
FFSDHYGDGKFDYAQSDYGDQKTVKSLITINDTEDQVFKIDNIVNGSKANVSIGSPNLSGVD
MLHLDIFSPGNDQGIGEFDFALTDFGGNGNDAGIWLNITDKGWHGQWISIDIPLSKWTGAAN
MIRFRRGGKGSTGKLLYVDNVYAYKSESDDPKPVPDPTTVPVLTKDKSDVISIFCEQYEEPG
YQDEFGIVSAGNWGQNAKQKDEFVEIVAGNQTLKLTSWDLFPFKVHKNSDVMDLSQMDY
LHLSIYQNGALDENNKPVSVCIWINDKDNKVAQAPLLEVKQGEWTSVSFGMDYFKNKIDLS
RVYVIRLKVGGYPTQDIYVDNIFGYKGDPIRPGQVTEPYVDECDQKIQDSTPGTLPPMEQAY
LGVNLASASGGSNPGTFGHDYLYPKFEDLYYFKAKGIRLLRIPFRAPRLQHEVGGELDYDA
GNTSDIKALAAVVKEAERLGMWVMLDMHDYCERNIDGVLYEYGVAGRKVWDSAKNTWG
DWEAMDEVVLTKEHFADLWKKIATEFKDYTNIWGYDLMNEPKGININTLFDNYQAAIHAIR
EVDTKAQIVIEGKNYANAAGWEGSSDILKDLVDPVNKIVYQAHTYFDKNNTGTYKNSYDQ
EIGGNVEVYKQRIDPFIAWLEKNNKKGMLGEYGVPYNGHAQGDERYMDLIDDVFAYLKEK
QLTSTYWCGGSMYDAYTLTVQPAKDYCTEKSTMKVMEKYIKDFDTSIPSSLVETNADGNAI
VLYPNPVKDNLKITSESGIEQVIVFNMIGQKVSERNEKGTNIELNLEALGKGTYLVTVRLEDG
NVVNRKIVKM
SEQ ID NO: 38 - B salyersiae GH88
MSCVLVCAGVLLLLSGLRETDVVGTKKQLSYCDTQIKKTLDAIEGSGLMPRCIDTDATDWY
KIDIYDWTSGFWPGILWYDYENTQNEEIRKAAIHYTESLVPLLDPEHPGDHDLGFQFYCSFG
NAYRLTKDDKYKQVLLKGADKLAGFYDPRVGTILSWPGMVTEMNWPHNTIMDNMMNLE
LLFWAAKNGGNREYYGMAVSHAKVTKENQFRPDGSCYHVAVYDTIDGRFLKGVTNQGYS
DSSLWARGQAWAIYGYTLVYRETGDKEYLRFAEKITDIYLKRLPEDYVPYWDFDDPAIPDA
PRDASAAAIVASGLLELVQLEDNTEKAEEYRDAAVNMLLSLSSDAYQSGIKKPSFLLHCTGN
LPGGYEIDASINYADYYYIEALTRYKKMQAGRDIVEKYPQATQKQVTIAM
SEQ ID NO: 39 - B salyersiae GH92_GH5
MKSHPLLILLIIIPTCLFAGNPDKVSLVDMFMGVKNSSNCVIGPQLPHGSVNPAPQTPNGGHN
GYDENDVIRGFGQLHVSGIGWGRYGQVFISPQVGFKPGETEHDSPKSDEVATPYYYKVNLD
RYKIKTEITPTHHSVYYRFTYPKSGNKNILLDMKHNIPQHIVPIVKGTFLGGNIEYDKASGLLT
GWGEYAGGFGSAAPYKVFFAMRPDVKLKEVKVTDKGTKALYARLSLPEEAETVHLGIGVS
LRSVENACKYLEQEIGARSFDEVKRVAKSAWEDVFATIDVKGGTQEEQRLFYTAMYHSFV
MPRDRTGDNPRWTSGQPHLDDHFCVWDTWRTKYPLMMLVNESFVAKTVNSFIDRFAHDG
ECTPTFTSSLEWEMKQGGDDVDNIIADAFVKNLKGFDRQKAYELVKWNAFHARDSLYLKK
GWIPETGARMSCSYTMEYAYNDDCGARIARIMKDDETADYLENRSQQWVNLFNPNLESHG
FNGFVGPRKENGEWIGIDPALRYGPWVEYFYEGNSWVYTLFAPHQFSRLIRLCGGKEAMAD
RLTYGFEKELIELDNEPGFLSPFIFSHCDRPGQTAKYVDFIRKNHFSRATGYPENEDSGAMGA
WYIFTSIGFFPNAGQDFYYLLPPAFSEVTLTMENGKKIDIKTVKSTPEVNYIESVSLNGKLLDR
TWIRHAEIAEGATIVYHLTDKPGQWSISPFEASRREPQPFGVNLAGAEFFHKKMEGVGRFNK
DYHYPTTDELDYWKSKGLTLIRLPFKWERIQRKLYGELNREEMDYIKFLLAEADKRDMQILI
DMHNYGRRKDDGKDRIIGDSLSIDHFASAWGSISRELKDCKGLYGYGLINEPHDMLASTPW
VGIAQAAIDSIRKNDAKNAIVVGGNHWSSAERWKLVSDDLKNLRDPSRNLIFEAHCYFDED
GSGIYRRSYEEEKAHPYIGVERMRPFVEWLKENDFRGLVGEYGVPADDERWLECLDNFLAY
LSAEGVNGTYWAAGARWNRYILSVHPENDYRKDKPQMKVLMKYLRTQ
SEQ ID NO: 40 - B salyersiae HTCS
MKHTILVLLGLALSFFPARAYHFRSYQVEDGLSHNSVWAVMQDSKGFMWFGTNDGLNRF
DGKKIKVYRKIQGDSLSIGNNFIHCLKEDSRGRFLIGTKQGLYLFDDKLEKFRHIDLDKNIKD
DVSINAIMEDPSGNIWLACHGYGLYVLTPELTTKKHYLSGSDPYSLPSNYIWSIVQDYYGNI
WLGTVGKGLVHFDPKEEKFTQMTQAKELGIDDPVIYSLYCDIDNNIWIGTATSGLIRYTPRS
QKATHYINHVFNIKSIIEYSDHELIMGSDKGLVKFDRTLESFDLINDDTSFDNMTDKSIFSIAR
DKEGSFWIGTYFGGVNYYSPAINRFQYCYNSPHNSSKKNIISGFAENENGDIWIGTHNDGLY
LFNPKSLSFKKPYDIGYHDVQSILSDQDKLYASLYGKGIHILNIKNGQVSASANDIGINHTINS
IAKTSKGQILFTSEGGVISMDASGTLKTLDYLTNTPVKDIAEDYDGSIWFATHSKGLIRLTSD
NRWEVFVNNPDNPKSLPGNNVNCVFQDSKFHIWAGTEGEGLVRFNAKEQNFEPILNDQSGL
PSNIIYSILDDSDGNLWVSTGGGLVKISSDLKNIKTFAYIGDIQRIQYNLNCALRASDNRLYFG
GTNGFITFNPKEITDNPNKPVVMVTGFQIASKEITLSESSPLKETISATKEITLRHDQSTFSFDF
VALSYLSPEQNRYAYILEGFDKEWHYTSDNKAMYMNIPPGTYVFRVKGTNNDGVWSDETA
DITVKIKPPFWLSNLMIGLYIVLAIGIILYFIRRYHRFIERKNQEKIFKYQTAKEKEMYESKINF
FTNIAHEIRTPLSLIAAPLEKIILSGDGNEQTRNNLGMIERNANRLLELINQLLDFRKIEEDMFH
FKFKRQNVVKIVEKVYKQYYQTAKFNKLEISLEAEKNDIECNVDSEAIYKIVSNLIANAIKYA
KSQILITVKERSGNLEIKIKDDGTGIEKQYMEKIFEPFFQIQDKNNAVRTGSGLGLSLSQSLAM
KHNGKISIESEYGKNCNFTLTIPIADGTEEEVQETEAAIPEKSEMPEQSVVEAGTRIIIVEDNTD
MRTFLCESLNDNYTVFEAENGVQALEMVEKENIDIIISDIMMPEMDGLELCNRLKSDPAYSH
LPLVLLSAKTDTSTKIEGLNQGADVYMEKPFSIEQLKAQISSIIENRNNLRKNFIKSPLQYFKQ
NTENNESADFVKKLNTIILENMSDEDFSIDSLSSQFAISRSNLHKKIKNITGMTPNDYIKLIRLN
ESARMLSTGKYKINEVCFLVGFNTPSYFSKCFFEQFKKLPKDFIQITNE
SEQ ID NO: 41 - B salyersiae NZ_KB905466
MKKQFSTLIALLIVGAAPLLGQETDPLNDPTNIDADLYLHAGFSQDSIRPDYSHTYYDNTNH
KLVKGEDGIYSITVPLKKEQIVNKNMEVGIYTYAYSVIYGGKVNGSGNDAVKGSVGPVIAD
EPRLFELAEDRDVTFYAKKLNTGTADAPWYRTMFICDAQPLYLDGTELPLPGEDGVTRYVV
DRGETSRRWEYKLSPIGRWSKTQDFMEDVIPAKWKSNEAYAFLPNGGWWLGGRFLLAYD
YKKLSLEVGKLVDELQTPLFTVNGESIPENLGIVDELLLNGSVITFLKGYYANGGKDSYDPA
FNTSIATVKLCWQIDELPAASFPLTNGEVVRDDNYNKTTEWTVSEADLFEGTTLPAGIHTLK
VWYESEYLGDVLTSEVQSTSFEIEEIVVIPLENKGTAVDLILEGDWNPETFRTIIEEQAVRITTI
DLTGVAGLTELPEMEGLNPNCLVYVNPDVVIAEGVDNVVVFDNEEGRAANILLTEGSDENN
VRLFTADRISYSHNFTADVWSTICLPFSADKGDVTVEEFTGADGEKVIFTGTSAIEANVPYLA
KTSNSEVKTFTATDVQMSVTAEPAPVVPENGYAFHAGYRAVEGDAVVGLHLMNDVGTAF
VKVADGNPEAAGVSAFHAYMQATVDELLTIVHGDDNPTGLGSTEDTGRLTIISHNGSVEIKT
GKAQMIGLYALDGRLVKMVELSQGSNFVNGLDKGIYIMDCQKVVVK
SEQ ID NO: 42 - B salyersiae putative PL
MKKIITIAFLSFLYFVYGYASNHSMHPLKQIDYVLKQVKAQQEPYYSAYQQLIHDADSILKV
SHHALVDFAVPGFYDKPEEHRANSLALQRDAYAAYCSALAYTLSGQQEYGEKACYFLNAW
ASTNEKYSEHDGVLVMTYSGSAFLMAAELMADDPLWSNKEKKDFRKWVKRIYQHAANTI
RVHQNNWADWGRFGSLLAASFLNEKKEVAENVRLIKSDLFHKIATDGSMPEETRRGGNGI
WYTYFSLAPMTGACWLVYNLTGENLFALEQDGTSIKKALDYMAYYNKHPKEWKWDKNP
NTGKNEVWPENLLEAMANLYNDNSYVEYVKGKRPIIYRNHHFCWTFPTLMPTSFENYQ
SEQ ID NO: 43 - B salyersiae SusC
MISKDENIKRRIIGVLFFLCALSPALWAQSRIIKGEVLDPNGEPLIGVGVMIKNTTAGTITDVD
GRYSIQVPDNNAVLSFSYVGYKRKEVKVGSQSVINISLEEESVLMDQVVIVGYGSQKKVNLT
GAVAAISVDESLAGRSVANVSSALQGLMPGLSVSQSSGMAGNNSAKLLIRGLGTINSADPLI
VVDDMPDADINRLNMNDIESITVLKDATASSVYGSRAANGVILVKTKSGKGLEKTQITFSGS
YGWEKPTNTYDFISNYPRALTLQQISSSTNPGKNGENQNFKDGTIDQWLALGMIDDKRYPN
TDWWDYIMRTGSIQNYNVSATGGSEKSNFYASVGYMKQEGLQINNDYDRYNARFNFDYK
VMKNVNTGFRFDGNWSNFTYALDNGFTSDSNLDMQSAIAGIYPYDPVLDVYGGVMAYGE
DPQAFNPLSFFTNQLKKKDRQELNASFYLDWEPVKGLVARVDYGLKYYNQFYKEADIPNR
SYNFQTNSYGIREYVTENAGVTNQTSTGYKTLLNARLNYHTVFATHHDLNAMFVYSEEYW
HDRYQMSYRQDRIHPSLSEIDAALSGTQSTSGNSSAEGLRSYIGRINYSAYGKYLLELNFRVD
GSSKFQPGHQYGFFPSAALGWRFSEESFVKPYIGKWLASGKLRASYGKLGNNSGIGRYQQQ
EVLYQNNYMLDGSIAKGFVYSKMLNPDLTWESTGVFNLGLDLMFFDGKLAAEFDYYDRLT
TGMLQKSQMSILLTGAYEAPMANLGTLRNRGFEANLTWRDRIADFTYSANFNISYNRTNLE
KWGEFLDKGYVYIDMPYHFVYSQPDRGLAQTWTDSYNATPQGVAPGDVIRLDTNGDGRID
GNDKVAYTNFQRDMPTTNFALNLQMGWKGIDVSLLFQGSAGRKDFWNNKYTEINLPDKR
YTSNWDQWNKPWSWENRGGEWPRLGGLVTNKTETDFWLQNMTYLRMKNLMIGYTFPKK
WTRKCFIENLRIYGTAENLLTITGYKGLDPEKAANSQDLYPITKSYSIGVNLSF
SEQ ID NO: 44 - B salyersiae SusD
MKRVYIKYIGLIAGMMMLFSSCADLLNQEPTVDLPATNYWKTESDAESALNGLVSDIRWLF
NRDYYLDGMGEFVRVRGNSFLSDKGRDGRAYRGLWEINPVGYGGGWSEMYRYCYGGINR
VNYVIDNVEKMIANASSEKTIKNLEGIIGECKLMRALVYFRLIMMWGDVPYIDWRVYDNSE
VENLPRTPLAEVKDHILDDLLDAFKKLPEKATVEGRFSQPAALALRGKVLLYWASWNHYG
WPELDTFTPSEEEARKAYKAAAEDFRTVIDDYGLTLFRNGEPGECDEPGKADKLPNYYDLF
LPTANGDAEFVLAFNHGGTNTGQGDQLMRDLAGRSVENSQCWVSPRFEIADKYQSTITGDF
CVPLVKLNPSSVPDARTRPNSAVNPESYKDRDYRMKASIMWDYEICQGLMSKKVTGWVPFI
YKMWGSEVVINGETYMSYNTDGTNSGYVFRKFVRNYPGEERADGDFNWPVIRLADVFLM
YAEADNAVNGPQPYAIELVNRVRHRGNLPVLASSKTSTPEAFFEAIKQERIVELLGEGQRAF
DTRRWREIETVWCEPGGRGVKMYDTYGAQVAEFYVNQNNLAYERCYIFQIPESERNRNPN
LTQNKPYR
SEQ ID NO: 45 - B salyersiae
TTATTTTAGATTAAACTTGGTTTTTGCGCGTATGTCAGTAGATGAGCTGCCTATGAAGAT
ATTGAATTTGCCTTGTTCGGCTTTCCAGTCCTGTATGGCTTCATCGTAGAATTTCAGGTC
ATTGGAGGTGATGTTGAACTCTACCACCTTTTCTTCGCCCGGATTCAGTTCTATTTTTTGG
AACCCTTTCAACTCTTTGACAGGACGTTCCAATGAGCTTTTTTCATCACCGATATAGAGT
TGCACAATCTCTTTTCCTTTTACTTTTCCGGTGTTTTTTATAGGGATAGTAACGGTCAGTA
TCTCCTTTTCGTTCATTTCGGAGGATGAAAGAAGGGCTTTTCCATATTGGAAAGTGGTGT
AGCTTAAACCGTGTCCGAACGGGAACAAGCTCCGGATTTTATGTTTGTCCGTCCAACGG
TAGCCTACGAATATGCCTTCGTTGTATTTCACGTTGATGCTGTCACCGGGATACTCTCCG
ACGGTATGTGCCGGATAGTCCTCCAGGCGGACAGCGAAAGTCATCGGTAACTTGCCGGA
AGGGTTAACATCTCCAACCAATACGGAAGCAATGGCATTACCGGATTCCGTACCGCTGA
ACCAGGCTTCCAAAACAGACGGCACCTCTTTGATCCACGGCATTGCGACTGCATTTCCC
GAAATAAGAATAACGGCTATGTTCTTATTTACTTTGCTGAGTTGGGCTATCAGTTCGTCC
TGTCCGTAAGGCAAATGATAGTTGAGCCGGTCCGTGCCTTCGCTGTCCTGGTGTTCGTTC
TTATTCAGACCTCCGAAGAACAGTACAATATCCGCATTTTTGGCAGCTTCAAGAGCCTCT
TTCGTTAGTTTCTCCGCATCGGGAAGCCGGTATCCTTCGAGAACGATGTACCGGGGATCT
CTCTTATCGGTGAGCGGGCTTGCATATCCGGGAGCGTAAACCAGTTCGACGGCATTGCC
TACCCGTTTCTTCAGCCCTTCGAGCGGAGAAACTTCGTATTTCACTTTCAATGAAGAAGA
GCCACCTCCCAATGTCATGACTTTCGTGGCATTTTCGCCGATAACGGCTATTTTCTTTATT
TGGGAAAGATCGACAGGCAGCAATCCGTTTTCGTTCTTGAGTAGCACGATGCCGTTTTC
GGCAATGGTGCGTCCTGCCAGTGCATGTTCTTCCGTGCCGAATGATCCCCATGGCTTTTG
AGTGTTCATGCTTGTCCGGAACATCATGCGAAGCAGCCTGCGCACTTTGTCATTCACGGT
GTTTTCTTCGATTTCTCCGTTTTTGATCTTTTCCAGTAGCGGCAATGCCATGTAGTAGTTG
TTGTAGGCATTGGTTTTTCCCCAGCTCAATCCGTCTGTCCATGTTCCCATTTCCAGATCGA
GTCCGTAATAGGCCGATTGGTAAGTGTCGTGTACACCTCCCCAGTCGGAAATCACAGTT
CCGTCGAAATTCCAGTCTTTTTTCAGTATATCGTTCAGCAAATACTGGTTGTGGCAGCAA
TGTTGGCCTTTGTATTGGTTGTAAGCCCCCATAATGGACCATGTTCCCCCTTCCAGTACG
GCCGCTTTGAAAGCAGGCAGGTAAATTTCGTGCAGGGCGCGGTCACTGACCTCCACATT
GATGTGTCCGCGATACATTTCCTGATTGTTCAATGCAAAATGCTTGACACAGGCGGCCA
CCCCGTTTTGTTGTACCCCTTTGATGTATGGCACTACCATTTTAGCGGCCAGATAAGGAT
CTTCTCCCATGTACTCAAAGTTTCGTCCGTTGAGTGGTGTGCGGTATATGTTGACACCCG
GACCCAGCAGAACATCTTTCCCCCGGAAGCGGGCTTCTTCGCCTATCGACTTGCCATAC
AGGGCCGACATATCAAGATCCCATGTGGCGGCAAGGCAGGTCAGTGCAGGGAAAGCGA
TGCAGGAATCACTGGTCCAGGCAGCTCCTTTCCATTCATCCCACAGCACCTCTTCCCGGA
TACCGTGTGGTCCGTCGGTCATCCAAAGTTCCGGTATGCCGAGACGGGGCACGCCATGG
GAACTGAATTTAGATTGTGCATGGCACAATTTTATTTTTTCTTCCAGAGTCATTCGTTGA
AGTGCATCTTCCACGCGTTGCTCTATCGGTTTTGTTTCGTCCAGATAGATCGGAGTTTTG
TTTTGCGCCGTGGCAGACATTGCCAGGCATGTGAATGCGAATAATTTAAAAAGCTTTTTC
ATGTTGTAAGATAATCTTTATTGATAATTTTCAAAAGAAGTGGGCATCAGCGTGGGGAA
TGTCCAGCAGAAGTGATGGTTGCGGTAAATGATTGGACGCTTCCCTTTTACATATTCCAC
ATAAGAGTTGTCATTGTATAGGTTTGCCATTGCTTCGAGAAGGTTCTCGGGCCAGACTTC
ATTTTTCCCGGTATTCGGGTTCTTGTCCCATTTCCATTCTTTGGGATGTTTATTATAGTAG
GCCATATAGTCCAGCGCTTTTTTTATGGAGGTTCCGTCTTGTTCCAGAGCGAACAGATTC
TCTCCGGTGAGATTGTACACTAGCCAGCACGCTCCGGTCATCGGTGCCAGTGAGAAATA
GGTGTACCATATGCCGTTGCCGCCTCTCCGGGTCTCTTCGGGCATGCTGCCGTCTGTTGC
TATTTTATGGAATAAGTCCGATTTTATCAGGCGCACGTTTTCCGCAACTTCTTTTTTCTCA
TTTAGGAATGAAGCCGCCAGCAGCGAGCCGAAACGTCCCCAATCCGCCCAGTTGTTCTG
ATGAACCCGGATGGTATTGGCGGCGTGCTGGTAGATGCGTTTCACCCATTTCCGGAAAT
CCTTTTTTTCTTTATTGCTCCAAAGCGGATCATCTGCCATCAGTTCCGCTGCCATCAGGA
ATGCTGAACCGGAATAGGTCATCACCAACACTCCGTCGTGTTCCGAATATTTCTCGTTGG
TAGATGCCCATGCATTCAGGAAATAGCAGGCTTTTTCTCCATATTCCTGTTGGCCGGATA
GGGTGTAGGCCAATGCCGAACAGTAGGCGGCATATGCATCCCGCTGCAATGCCAGGGA
GTTGGCACGGTGTTCCTCGGGTTTATCATAGAATCCCGGTACGGCAAAATCTACCAGTG
CATGGTGCGACACTTTCAAGATGGAGTCGGCATCGTGAATCAATTGCTGATAGGCAGAA
TAATAGGGCTCTTGCTGTGCCTTGACCTGTTTTAGTACATAGTCGATTTGCTTTAACGGA
TGCATGCTGTGATTGCTGGCGTATCCGTAGACGAAATATAAAAAAGAGAGAAATGCTAT
CGTTATAATTTTCTTCATTTTTAGGATTTATTTATTCGTTTGTTATTTGTATAAAATCTTTA
GGAAGTTTCTTGAACTGTTCGAAGAAACATTTTGAGAAATAAGACGGTGTGTTGAAACC
TACCAGGAAGCATACTTCGTTTATTTTGTATTTGCCGGTGGACAACATCCGTGCGCTTTC
ATTCAGCCTGATCAGCTTGATGTAGTCGTTGGGGGTCATTCCTGTGATGTTTTTAATCTTT
TTGTGCAGGTTTGAACGGCTTATGGCAAACTGGCTGGAAAGGCTGTCGATGGAGAAGTC
CTCATCCGACATGTTTTCCAATATGATGGTATTCAGCTTCTTCACGAAATCTGCGCTTTC
GTTGTTTTCCGTGTTCTGTTTGAAATACTGCAACGGAGATTTGATGAAGTTCTTTCGCAG
GTTGTTCCTGTTTTCAATAATGCTGCTTATTTGCGCTTTTAGTTGTTCGATGGAGAATGGT
TTTTCCATGTAAACGTCGGCTCCCTGGTTCAGACCCTCTATTTTAGTGGAAGTATCCGTT
TTGGCAGATAGCAACACCAAAGGCAGGTGAGAGTAGGCGGGATCGCTTTTCAGCCGGTT
ACATAATTCCAACCCGTCCATTTCCGGCATCATAATATCGGATATGATGATGTCTATGTT
CTCTTTTTCCACCATTTCCAGTGCCTGTACTCCGTTCTCTGCTTCAAAGACGGTGTAGTTG
TCGTTTAGGCTTTCGCAAAGGAAAGTCCGCATATCCGTGTTGTCTTCCACGATGATGATC
CTCGTACCCGCTTCCACGACCGATTGTTCGGGCATTTCACTCTTTTCGGGTATGGCAGCT
TCCGTTTCCTGTACCTCTTCCTCTGTTCCGTCGGCAATGGGGATTGTCAGTGTAAAATTA
CAGTTCTTTCCGTATTCCGATTCGATGGAAATTTTTCCGTTGTGTTTCATGGCCAGCGATT
GCGATAGCGATAACCCCAGGCCGGAGCCTGTCCGCACAGCGTTGTTCTTATCCTGTATCT
GGAAGAAAGGTTCGAATATTTTTTCCATATACTGCTTTTCAATGCCGGTACCATCGTCTT
TAATCTTTATTTCCAGGTTTCCGCTTCTCTCTTTTACGGTTATCAGGATTTGGCTTTTGGC
ATATTTAATGGCGTTGGCAATCAGGTTGCTGACAATCTTATAGATGGCTTCGGAGTCGA
CATTGCATTCTATATCGTTCTTTTCCGCTTCCAAAGAGATTTCCAGTTTGTTGAATTTGGC
CGTCTGGTAATATTGCTTATACACTTTTTCCACAATCTTGACGACATTCTGCCGCTTGAA
TTTGAAGTGGAACATGTCCTCTTCTATTTTACGGAAGTCCAGCAGTTGGTTGATCAGTTC
GAGCAGCCTGTTGGCATTGCGTTCTATCATCCCCAGGTTGTTCCTGGTCTGTTCGTTTCC
GTCTCCGGACAAAATTATTTTTTCCAATGGTGCGGCAATCAGCGAGAGCGGTGTACGTA
TTTCATGGGCAATGTTCGTGAAGAAATTGATTTTCGATTCGTACATCTCTTTTTCTTTGGC
CGTCTGGTATTTGAATATCTTTTCCTGGTTTTTACGTTCGATAAAGCGGTGGTATCTCCG
GATAAAATAAAGGATAATGCCGATGGCAAGGACAATATACAGGCCGATCATGAGGTTG
GACAACCAGAACGGGGGCTTTATTTTCACCGTAATGTCTGCCGTTTCATCGCTCCATACT
CCATCATTATTCGTGCCTTTCACACGGAATACATAAGTTCCGGGCGGGATGTTCATGTAC
ATGGCCTTATTGTCGGAGGTGTAATGCCACTCTTTGTCGAAGCCTTCGAGGATGTAGGC
ATATCTGTTTTGTTCCGGCGAAAGATAGCTCAATGCTACAAAGTCGAAGCTGAAAGTGG
ACTGGTCGTGCCGCAACGTTATCTCTTTGGTTGCGCTGATGGTCTCTTTTAGTGGCGACG
ATTCGGAAAGTGTTATCTCTTTGCTGGCAATCTGGAAACCTGTGACCATGACGACCGGTT
TGTTGGGGTTATCTGTAATCTCTTTCGGATTGAATGTGATGAATCCGTTGGTTCCGCCAA
AGTAAAGTCGGTTGTCGGAAGCTCTCAATGCGCAGTTCAGATTGTATTGTATCCGTTGTA
TATCGCCGATATAGGCAAATGTTTTAATGTTTTTCAAGTCGGAGGATATTTTAACCAACC
CTCCGCCTGTGCTTACCCACAGATTGCCGTCCGAATCGTCCAGTATGGAATAGATGATGT
TGGAAGGTAGGCCCGACTGGTCGTTTAAGATTGGTTCGAAGTTTTGCTCTTTGGCGTTGA
ACCGTACCAGCCCTTCTCCTTCCGTCCCTGCCCAGATGTGGAATTTGGAGTCTTGAAATA
CGCAGTTGACATTATTTCCCGGCAAGGATTTCGGATTATCGGGATTATTTACGAATACTT
CCCATCTATTGTCTGAGGTGAGCCGTATCAGCCCTTTGGAATGGGTGGCAAACCAAATG
GAGCCGTCATAATCTTCTGCAATGTCTTTTACCGGGGTGTTGGTCAGGTAATCGAGGGTC
TTGAGCGTGCCGGATGCATCCATCGAGATCACTCCGCCTTCGGAGGTGAAGAGTATCTG
CCCTTTGGAGGTTTTGGCTATGGAGTTGATGGTATGATTAATTCCTATGTCGTTGGCGGA
GGCGCTGACCTGTCCGTTCTTTATGTTCAGGATATGGATGCCTTTGCCGTAAAGGCTTGC
ATAAAGTTTGTCCTGGTCCGACAGAATGCTCTGTACATCGTGGTAACCGATGTCGTATG
GCTTCTTGAAGCTCAGGCTCTTCGGATTGAAAAGGTATAGTCCGTCGTTGTGCGTTCCGA
TCCATATGTCCCCATTTTCATTCTCGGCGAATCCGCTGATGATATTTTTTTTGGAAGAGTT
GTGTGGAGAGTTATAGCAATACTGGAAACGGTTGATGGCAGGCGAATAATAATTTACGC
CCCCAAAGTAAGTTCCGATCCAGAAAGACCCTTCCTTGTCACGTGCAATGGAGAAAATG
GATTTATCCGTCATGTTGTCAAAAGAAGTATCGTCGTTGATCAGGTCGAAACTCTCCAGC
GTACGGTCGAATTTCACCAGTCCTTTGTCCGATCCCATGATGAGCTCGTGGTCGGAATAT
TCGATGATGGATTTGATGTTGAATACATGATTTATATAATGTGTGGCTTTCTGTGATCTG
GGGGTATAGCGTATCAATCCGCTTGTGGCCGTCCCTATCCAGATGTTATTGTCTATGTCG
CAATACAGGCTGTAAATCACGGGATCGTCGATGCCCAACTCTTTAGCCTGTGTCATTTGC
GTGAACTTTTCCTCTTTAGGATCAAAGTGTACAAGGCCTTTGCCCACTGTGCCCAACCAT
ATATTTCCGTAATAATCCTGAACGATGCTCCAGATGTAATTGGAGGGCAACGAATAGGG
ATCGCTACCGGATAGATAATGTTTCTTGGTCGTTAATTCGGGAGTAAGGACATACAGGC
CATATCCGTGGCAGGCCAGCCATATATTTCCGGAAGGGTCTTCCATAATAGCATTGATG
CTCACGTCGTCTTTTATGTTTTTGTCCAGGTCGATGTGTCTGAACTTCTCTAACTTATCGT
CGAAAAGATAGAGCCCTTGTTTGGTTCCGATGAGGAATCTTCCTCGCGAATCCTCTTTCA
GGCAGTGGATAAAGTTATTGCCGATAGATAAAGAGTCGCCCTGTATTTTGCGGTACACT
TTGATTTTCTTGCCATCGAAACGGTTGAGGCCGTCGTTGGTCCCGAACCACATGAAGCCT
TTGCTGTCCTGCATAACCGCCCAGACACTGTTATGCGACAATCCGTCTTCCACCTGATAG
CTCCTGAAGTGATAGGCGCGTGCAGGAAAAAAAGATAAAGCCAAACCTAATAAAACTA
AAATCGTATGTTTCATAGCCTGATGAAATTAAGATGTTCAAATATAGGGCTTTGCTCTCT
TTGGCGATGCAAATATCTTCTTAAAACCTATAAAAATATGGTATAATTGTGAGAATGCA
GTGTATTTATATCTTTGAAAAGTATATTTCTATCCACTTTGTTTTATCAGTTCTACATTTG
TGTCATTCATATTAGTAATTAAAGTCTAATCTTTAGAAACATGAATAAGTTAGTCAGTAC
TTTTATTATTTCATCCTTTACTGCTGCTATGGGCGTATCGGTTTTTGCTGCTGATGATGGC
GGTGCGTTATATCTGGATGCGGGCCGGCCTGTCGAGCAGAGGGTGAAAGATTTGATGTC
GCGCATGACTCTGGAGGAGAAAGTGGGGCAGATGTGTCAATGGGTCGGCTTGGAGCAT
ATGCGAACCGCTTCACAGGATTTGACGGTAGACGAATTGAGTAATAACACGGCGCGGG
GGTTCTATCCCGGCATCACGGAAGAAGACGTGAGACAAATGACGATAGACGGGAAGGT
GGGCTCTTTCTTGCATGTACTCACAGTCAAGGAGGCCAATCAGTTGCAGGAGCTGGCAA
TGAAAAGCCGTCTCAAAATCCCTTTGATTATAGGCATCGATGCCATTCACGGCAATGCG
CAGGTAGTGGGTACTACGGCGTATCCGACGAGCATCGGGCAGGCATCCATGTTCGATGT
CGGCCTGGTTGAAGAGATTTGCCGGCAAACGGCTTTGGAGATGCGTGCTACAGGTTCGC
AGTGGACATTCAATCCCAATGTAGAGGTCGCCCGCGACCCGCGTTGGGGGCGTGTCGGC
GAAACTTTCGGCGAAGATCCCTACTTGGTATCTTTATTGGGCGTGGCTTCCGTGCGCGGG
TATCAGGGAGACGGGTTTGGAAAGGCGGAAAATGTGTTGGCTTGTGCCAAGCATTTTAT
TGGAGGCAGCCAACCGATAAACGGAACGAACGGCTCTCCCACAGACATTTCGGAACGG
ACACTCCGGGAGGTATTCCTGCCCCCCTTTAAGGCGACCGTAGATGCCGGTGTATATAG
CTTTATGACAGCTCATAATGAACTGAACGGCATTCCCTGTCATGCCAATCCATGGCTGAT
GGAAGATATTCTTCGCAAAGAATGGGGATTCGATGGTTTCATAGTCAGTGATTGGATGG
ACATCGAGCATATACACGACTTGCATCGCACGGCAGTGGATAATAAAGATGCTTTCTAC
CAGTCGGTAGATGCCGGAATGGATATGCACATGCATGGACCGGAGTTTTACGAAAAGGT
GATTGAACTGGTGAAGGAGGGAAAACTCACGGAAGCCCGGATCGATGAGTCTTGCCGG
AAAATATTGGCTGCGAAATTCCGGTTAGGACTGTTCGAGAAATCTTTTACCGATGAGAA
AGCGGCGAAAAGCGTCCTGTTCAATGAAAAGCATCAGGCCACGGCATTGGAAGCGGCG
CGTAAGTCCATTGTGCTATTGACCAATGACGGCATACTTCCGCTGGATGAAGCAAAATA
TAAAAATGTATTCGTAACCGGAATGAATGCCGACAATCAGACGATTCTCGGTGATTGGG
CTTTGACACAGCCGGATGAGAATGTGATTACAGTGCTCGAAGGGCTGAAACTGGTATCT
CCCGACACTAAATTTTCATTTGTGGATTTGGGATGGAACATCCGGGAAATGGATAAAAA
CAAAGTGGAACAGGCCGCAAAGCAGGCTGCCAAAGCCGATTTGGCAATTGTGGCGGTG
GGAGAATATTCCTTGCGGACCAACTGGTACGACAAAACTTGTGGCGAAGACTGCGACCG
TTCGGATATCAATCTGGCAGGGTTACAGCAGGAACTTGTGGAGTCCATTCTGGCAACGG
GAGTTCCTACCGTTGTGGTTTTAGTAAACGGGCGTCAGTTGGGGGTGGAATGGATTGCC
GGTCATGCCAATGCTTTAGTCGAAGCGTGGGAGCCGGGTAGTCTCGGAGGACAGGCCAT
TGCCGAAATATTATATGGAAAAGTAAACCCTTCCGGCAAACTGCCGGTGACGGTTCCGC
GCCATGTGGGACAGATACAGATGATTTATAACCATAAGCCGTCCATGTATTTTCATCCGT
ATGCCATCGGAGAGAGTACGCCTTTGTTCTATTTTGGATACGGCCTGAGTTATACGGAAT
ATGCGTATTCGGATCTCACGGTTTCCTCGGCGCAGATGTCGGGGGACGGCAGTGTGGAA
GTGTCCGTGAAAGTGACGAATACGGGAACAACGGATGGGGAGGAGATTGTGCAGTTGT
ATATCCGCGACCTCTATTCCAGTGCGACGCGTCCGGTGAAAGAGTTGAAGGACTTCAGG
CGCGTGCCCCTTCGTGTAGGCGAAACCAAGACAGTTTCTTTCATCTTACCGGCAGGGAA
ACTTGCTTTCTATGATAAGAAGATGGACTATACGGTGGAACCTGGAGACTATGAAATCA
TGGTGGGAGCTTCGTCGAGGGATGAAGATTTAATGAAGAGAATTGTAAATGTAAAATA
ATAGTTGGGATGAAAAGATTGATGAGCTGTGTGTTGGTTTGCGCAGGAGTATTGCTTTT
GCTGTCGGGACTGAGAGAAACAGATGTAGTCGGAACAAAAAAGCAATTATCGTATTGT
GACACGCAGATAAAGAAAACACTGGATGCCATCGAAGGTTCCGGATTGATGCCCCGTTG
CATCGATACGGATGCCACAGACTGGTATAAAATCGATATTTATGATTGGACGAGCGGTT
TCTGGCCCGGCATCTTGTGGTACGATTATGAGAACACCCAAAATGAAGAGATCAGGAAA
GCAGCCATTCACTATACGGAATCGCTTGTGCCTTTGCTCGATCCGGAGCATCCGGGCGA
CCATGATCTGGGATTCCAGTTTTATTGCAGCTTTGGCAATGCCTATCGACTGACAAAGGA
CGACAAATACAAGCAGGTATTGCTGAAAGGTGCCGATAAACTGGCCGGATTTTATGACC
CCCGGGTGGGGACAATCCTCTCGTGGCCGGGTATGGTGACGGAGATGAACTGGCCACAC
AATACCATCATGGACAACATGATGAATCTTGAACTGCTGTTTTGGGCGGCCAAGAATGG
CGGCAACAGGGAATACTATGGCATGGCGGTGAGCCATGCAAAGGTGACAAAAGAGAAT
CAGTTTCGTCCCGACGGTTCTTGCTACCATGTAGCGGTGTACGATACCATCGACGGGAG
GTTCTTGAAAGGCGTTACGAATCAAGGATATAGTGATAGCTCCCTGTGGGCGCGCGGAC
AGGCATGGGCCATTTATGGGTATACGTTGGTTTACAGGGAAACCGGTGATAAGGAATAC
CTCCGTTTTGCCGAGAAAATAACGGATATATACCTCAAACGTTTGCCGGAAGATTATGT
TCCGTATTGGGATTTCGACGATCCGGCTATCCCGGACGCTCCGAGAGACGCATCTGCAG
CGGCCATTGTAGCTTCCGGATTGCTGGAGCTGGTGCAATTGGAAGATAATACGGAGAAA
GCCGAAGAGTATAGAGATGCGGCTGTTAATATGCTGCTCAGTCTGTCGTCTGATGCTTA
CCAGAGTGGTATCAAAAAACCGTCTTTCCTGCTCCATTGCACGGGCAATTTACCGGGAG
GGTATGAGATCGACGCATCCATTAATTATGCTGACTATTATTACATTGAAGCGCTGACA
CGTTACAAAAAAATGCAGGCTGGGCGTGATATTGTTGAAAAGTACCCACAAGCTACGCA
GAAACAGGTCACTATTGCTATGTAAACAGGATTTTGGTAGTAATAAATAATATTGTTGT
ATTTGTTTATCGCTTGTCGGGCTACTTTTGTGCAGAACAGATTGTTTAAACTTAAAAATA
TTGTATTATGAAAAAACAGTTTTCTACTTTGATTGCATTACTTATTGTCGGAGCTGCTCC
CCTTTTGGGGCAAGAAACCGACCCTCTGAACGATCCGACTAATATTGATGCGGATCTCT
ATCTTCACGCCGGATTTTCTCAGGATTCCATCCGGCCGGATTATTCCCATACTTATTATG
ATAACACCAACCATAAACTGGTAAAAGGGGAGGATGGCATATATTCCATTACGGTTCCT
TTGAAGAAAGAGCAGATTGTGAATAAAAACATGGAGGTTGGTATTTATACCTATGCTTA
CTCTGTTATTTATGGAGGAAAAGTGAACGGTTCAGGCAATGATGCCGTTAAGGGAAGTG
TAGGACCGGTTATTGCCGATGAACCCAGACTCTTTGAACTGGCCGAAGACCGGGATGTC
ACTTTTTATGCAAAGAAACTGAATACAGGAACGGCGGATGCTCCGTGGTACAGAACTAT
GTTCATCTGCGATGCACAACCGCTATATCTGGACGGAACGGAGCTGCCGTTGCCGGGCG
AAGATGGAGTGACGAGATACGTAGTGGATAGAGGTGAAACCAGCAGACGGTGGGAGTA
TAAACTCAGCCCTATCGGGCGTTGGAGCAAAACGCAGGATTTTATGGAAGATGTGATAC
CGGCCAAATGGAAATCTAACGAAGCATACGCTTTTCTGCCCAATGGCGGCTGGTGGCTC
GGAGGGCGTTTTCTGTTGGCGTATGACTATAAGAAGTTGAGTCTGGAGGTCGGCAAATT
GGTTGATGAACTGCAAACTCCCTTGTTTACGGTGAATGGAGAAAGTATTCCGGAGAATT
TGGGAATAGTCGATGAATTGTTGCTGAATGGTTCTGTGATTACATTCCTGAAAGGATATT
ATGCCAATGGCGGCAAAGACTCTTATGATCCGGCATTTAATACAAGCATCGCCACCGTG
AAATTGTGTTGGCAGATAGACGAATTGCCTGCTGCCTCTTTCCCTTTGACAAACGGTGAG
GTGGTCAGAGACGATAATTATAATAAAACGACCGAGTGGACGGTTAGTGAAGCGGATC
TTTTCGAAGGAACAACTTTGCCGGCGGGAATACATACGCTGAAAGTATGGTACGAGTCA
GAATATTTAGGGGATGTACTTACTTCTGAAGTACAATCGACGTCCTTCGAGATCGAAGA
GATTGTGGTTATTCCTCTTGAAAATAAAGGAACGGCTGTCGATCTTATTCTGGAGGGAG
ACTGGAATCCGGAAACGTTCCGTACGATTATCGAAGAACAAGCCGTTAGGATTACTACG
ATTGACCTTACCGGAGTGGCCGGCCTGACGGAACTTCCCGAAATGGAAGGTTTAAATCC
GAACTGCCTGGTTTATGTGAATCCGGATGTTGTTATCGCAGAGGGCGTTGATAACGTGG
TTGTATTTGATAACGAAGAGGGTAGAGCAGCCAATATACTTCTGACGGAAGGTTCCGAT
TTCAATAACGTGAGATTATTTACGGCCGACCGGATCTCCTACTCCCATAACTTTACTGCT
GATGTTTGGTCTACCATCTGCTTGCCTTTCAGTGCGGATAAGGGAGATGTAACCGTAGA
AGAGTTTACGGGTGCCGATGGTGAGAAAGTCATCTTTACGGGAACATCCGCCATCGAAG
CCAATGTTCCCTATTTGGCTAAAACAAGTAATTCGGAGGTTAAGACCTTTACGGCAACA
GATGTACAGATGAGCGTTACGGCAGAACCAGCTCCGGTAGTTCCGGAAAATGGTTACGC
ATTCCATGCCGGTTACCGTGCGGTAGAAGGAGATGCTGTCGTAGGACTCCATTTGATGA
ACGATGTGGGGACTGCTTTCGTAAAAGTAGCCGATGGAAATCCGGAAGCTGCGGGAGTT
TCTGCTTTTCATGCTTACATGCAGGCAACTGTTGATGAACTGTTGACAATCGTCCATGGT
GACGATAACCCTACCGGATTGGGTTCGACGGAAGATACCGGCCGGTTGACGATTATCTC
CCATAACGGTTCTGTCGAAATTAAGACGGGCAAGGCGCAGATGATAGGTTTGTATGCAT
TGGATGGCCGTTTGGTGAAGATGGTTGAACTGAGCCAGGGCAGTAATTTTGTCAATGGA
TTGGATAAAGGTATTTATATTATGGATTGCCAAAAGGTAGTAGTGAAGTAAAAGAAGTC
TCCGTGTCTTGTCCCTTGTACAAGCCGGTAGAATCAGAATAAAGAAAAATTTGAATGGA
TAATAAATAAAAGAGGTATTGTTTTTTTTATGCAGATTCAAGATAATAAGTTCATTGTAT
CACTTTATCTTGAATCTGCTTTTTTTGAAATGACAGCCTCTCCCCAACCCTCTCCGTGGG
AGAGGGAGCAAAAAATGACTTGTAAACAATTGATTAACAGAACTAACTTTAGCTCCCTC
TCCCACGGAGAGGGTTGGGGAGAGGCTTTATAACTTTATAAAAATGAGACATCGGGTTA
TCCTATTTATTTGTGTGTTGCAAACCCTGTTTGCATATGCTGTGGGTGCGGAGACTCACT
TTATGCTCACCTTGAATGAGCAATGGAAATTCTCGACGGGCGATTCATCCGCATGGGCC
ACTACGGAATTCGACGATAACCAATGGGGCACTATCTCTTCCAGGCAATACTGGGAAGA
ACAGGGTTATGACGGCTATGACGGTTATGGTTGGTACAGGCAGCATTTCATGATTTCCG
AGGATTGGAAACCGATCGTAACGAATGCCGGAGGTTTATATATAAGATATGAATTTGCC
GATGACGTGGATGAGGTTTTTGTCAACGGGGTCTCTGTCGGTAGGATGGGAGAGTTTCC
ACCGGAATATAAAGTTATTTATGGCGGTATGCGTAAATACAAGATCAGCCCGGGACTGT
TGCGATTCGGTGAAGAGAATCTCATTGCCATCCGGGTGTACGACAACGGTGGTGCAGGA
GGGTTGAAGACAGAAAATATACTCCTGCAATCCATAACTCCGATGGACGATCTGATGCT
GGATATTCGTTGTGACGATAGCGACTGGGTATTCGAAAATACAGAGACAATCGATTTCC
GTGTACGTCCGAAACAACCGCTTGCGGCGGGAGGGGAGTTTAATCTCGTTTGCAGCGTG
ACGACGGATACCTATCTCCCGGTAGACTCTTTTGTGTACCGGGTGAAAGGAGATTTTGA
GCAACCCGTCTCTTTCGTTCCGCCGGCTCCGGGTTTTTACCGGATTACTTTGTATGGAGA
ACAACAAGGTGTAAAAAGCGATTTTCTGAAATTTAATATGGGATATTGCCCGGAACAGA
TTATTTCTCCCGTCGATGTCGAACCCGATTTCGACCAGTTCTGGGAAACTACGCTGAAAG
AGCTTTCCGAAGTTGTTCCCGATTACCGCATGACTTTACTGGAAGAGAAGTCACAAGGA
GCCAAAAACATCTACCGGGTGGAAATGTATTCGTTAGGAAATGTCCGTATCGAAGGGTA
TTACGCCGTTCCCAAGCAAAAGGGCAAGTTTCCGTCTGTCATCTCTTTTCTGGGCTATGG
TTCCGGGGGTGGTTTTCCTCGTCCGGATAATCTGCCCGGCTTTTGCGAGTTTATCCTTTCC
ACCAGAGGGCAAGGCATTCAGCTTCCTGTCAACACCTATGGCAAATGGATCGTACACGG
GCTGGAAGATAAATCACAATACTATTATCGGGGGGCATTTATGGATTTGGTGCGTGGGA
TCGACTTCCTGTGTTCACGTCCGGAGGTGGACACGGAGAAGATTTTTGCCGAAGGCGGA
AGTCAGGGCGGAGCTTTTACGCTGGCAGCCTGTGCACTGGATAGACGCATCTGTGCGGC
AGCACCTTACATCCCTTTCCTGTCGGATTTTGAGGATTATTTTAAGATCGCACCCTGGCC
GCGTAGTGTGTTCGAAGAGTATCTGCGTAGCCATGAGGAGAGTAGTTGGGACGAAATAT
ACCGGTTGCTTTCCTATTTCGACAGTAAGAATCTGGCACCGCGTATTACGTGTCCCATCA
TCATGGGCGTAGGGTTGCAAGATAATATTTGCCCTCCCCATATCAATTTTTCCGGCTACA
ATCAGGTGAAGTCTCCTAAGCGTTATTATATCTATTACGATAAAGAACATACGGTTGGG
AAGAGTTGGTGGACAATCAGAAATAACTTTTTCCGTAGTTTTTGCAACTGAATCTAATTT
ATGTATACCAAAATATTGTTCTTGTCATATTTTGGTATACATAGATTATATTTTTGCATAA
GCGGATTCTTTTTTGGGCTTATTTTGCTTCTGTCAAGAAAGCTAAATTGTTTAATTAAAG
AATCTGTGAATACAATGAAAAGTCACCCTTTACTCATCTTATTAATAATTATTCCCACTT
GTCTTTTCGCCGGAAATCCGGATAAGGTATCTCTGGTAGATATGTTCATGGGGGTAAAG
AACAGCAGTAATTGTGTAATTGGCCCTCAGTTGCCGCATGGCTCTGTGAACCCGGCGCC
GCAAACTCCCAACGGCGGTCACAACGGATACGATGAAAACGATGTGATTCGCGGATTC
GGACAGCTGCATGTTTCCGGCATTGGGTGGGGACGCTACGGACAGGTGTTTATCTCTCC
GCAGGTCGGTTTCAAACCCGGCGAGACGGAACACGACTCTCCTAAGTCCGATGAAGTGG
CTACGCCCTATTATTATAAGGTAAATTTGGACCGCTATAAGATAAAAACCGAAATAACC
CCCACTCACCACAGTGTGTACTACCGCTTCACCTATCCGAAATCCGGTAACAAGAATAT
CCTTTTGGATATGAAACACAACATTCCGCAGCACATTGTCCCCATAGTGAAAGGTACTTT
TCTGGGAGGGAATATCGAATACGACAAGGCATCGGGCTTGCTGACCGGTTGGGGCGAA
TACGCCGGAGGTTTCGGAAGCGCTGCTCCCTACAAAGTGTTTTTTGCCATGCGTCCGGAT
GTGAAATTGAAGGAGGTGAAAGTCACCGATAAGGGGACGAAGGCTCTGTATGCCCGTT
TGAGTTTGCCGGAAGAGGCTGAAACTGTCCATCTGGGCATCGGCGTTTCACTCAGAAGT
GTGGAGAATGCATGTAAATATCTGGAACAGGAGATCGGTGCGCGTAGCTTCGACGAGG
TGAAGCGTGTGGCGAAATCTGCTTGGGAGGATGTGTTTGCCACTATCGATGTAAAAGGG
GGAACCCAAGAAGAGCAGCGTCTGTTCTATACAGCCATGTATCATAGTTTTGTGATGCC
CCGCGATCGTACGGGCGACAATCCCCGTTGGACGAGCGGACAACCTCATCTTGACGATC
ATTTCTGCGTGTGGGATACATGGCGCACCAAGTATCCTTTGATGATGCTTGTCAATGAGA
GTTTCGTGGCAAAAACGGTGAATTCTTTTATAGACCGTTTCGCTCACGACGGAGAGTGT
ACTCCGACCTTTACCAGCTCTCTGGAATGGGAGATGAAACAGGGCGGAGATGACGTGG
ACAATATCATAGCCGATGCTTTCGTGAAAAACCTGAAAGGATTCGACCGCCAGAAGGCG
TATGAACTGGTGAAATGGAATGCGTTTCATGCCCGTGACAGCCTTTACCTGAAAAAGGG
ATGGATTCCTGAAACGGGAGCAAGGATGAGTTGCAGCTACACTATGGAGTATGCCTACA
ATGACGATTGCGGTGCACGTATTGCAAGGATAATGAAGGATGATGAGACGGCGGACTA
TCTGGAAAACCGTTCCCAACAGTGGGTGAATTTGTTTAATCCGAATCTGGAAAGTCATG
GTTTCAATGGCTTTGTCGGTCCGCGCAAAGAGAACGGCGAATGGATCGGTATCGATCCG
GCGTTGCGCTACGGTCCGTGGGTGGAATATTTCTACGAAGGTAATTCTTGGGTGTACAC
ATTGTTCGCTCCTCATCAGTTCAGTCGTCTGATCCGTCTTTGCGGAGGGAAAGAGGCGAT
GGCAGACAGGCTTACTTATGGATTCGAAAAAGAGTTGATCGAACTGGACAATGAACCG
GGATTCCTGTCTCCCTTTATCTTCAGCCACTGCGACCGTCCCGGTCAAACCGCCAAATAT
GTAGATTTTATCCGGAAAAACCACTTCTCCCGGGCTACCGGTTATCCGGAGAATGAAGA
TAGCGGAGCAATGGGGGCATGGTACATCTTTACATCGATCGGTTTCTTTCCCAATGCCG
GACAGGATTTCTACTATTTGCTTCCTCCGGCTTTTTCGGAGGTGACGCTGACAATGGAGA
ATGGCAAGAAAATAGATATTAAAACCGTTAAGTCGACTCCCGAAGTCAATTATATAGAG
TCTGTCAGTCTGAACGGAAAACTGCTGGACCGGACATGGATACGCCATGCCGAGATTGC
GGAAGGCGCTACGATTGTCTATCACTTGACGGATAAACCGGGACAGTGGAGCATCTCTC
CTTTTGAAGCAAGCAGAAGAGAGCCGCAACCGTTCGGGGTGAATCTGGCAGGGGCGGA
GTTCTTCCACAAAAAGATGGAGGGAGTGGGGCGCTTTAATAAAGATTATCACTACCCGA
CTACGGACGAGCTGGACTACTGGAAGTCCAAAGGACTCACTTTGATTCGATTACCTTTC
AAATGGGAACGCATACAGCGTAAGTTATACGGAGAATTGAACCGGGAAGAGATGGATT
ATATCAAATTCTTATTGGCCGAAGCAGATAAGCGCGACATGCAGATATTGATCGATATG
CACAATTACGGCCGGCGTAAGGACGATGGTAAGGACCGCATCATAGGCGACAGCCTTTC
GATCGATCATTTTGCATCGGCTTGGGGATCGATCTCCAGAGAATTGAAAGACTGCAAAG
GCCTGTACGGTTACGGCCTGATCAACGAACCGCATGATATGCTGGCTTCTACTCCGTGG
GTAGGGATTGCACAGGCAGCCATCGACTCCATTCGCAAAAATGATGCGAAGAATGCCAT
TGTGGTGGGTGGTAATCATTGGAGTTCTGCCGAACGCTGGAAACTGGTCAGTGATGATT
TGAAGAACTTGCGCGACCCGTCACGCAATCTGATATTCGAAGCGCATTGCTACTTTGAT
GAAGACGGATCGGGCATTTACCGCCGTTCGTATGAGGAAGAAAAAGCACATCCGTACA
TTGGCGTGGAGCGTATGCGGCCTTTTGTGGAGTGGCTGAAAGAGAATGATTTTCGCGGG
CTTGTCGGTGAATACGGAGTTCCGGCAGACGATGAGCGCTGGCTGGAATGTCTGGACAA
TTTCCTGGCTTATCTTAGTGCGGAAGGCGTGAACGGTACCTATTGGGCGGCCGGTGCCA
GATGGAACAGGTATATTCTTTCCGTTCATCCGGAGAACGATTACCGGAAAGACAAACCG
CAGATGAAAGTATTGATGAAATATTTGAGAACTCAATAATAGATTGTAAACTAAAATTA
AGTATTATGGAGAAAAAAACAAAAAGGATTGCATTTGTCCTGGCAACCATGCTATGTGG
ATGGCAAATGATGCTGGCCCAACCGGTTAGCCCGGCACCGACGCCAACACGGGCGGCG
AATGATGTGAAGGCAATGTTCAGTGACGCTTATCCGGAGAAGTTCGGAAAGTTCCAGAT
AGACTATGATGACTGGAATAGCGATAAATTTTTGACTACCAAAACGATTGTTACTCCTTT
CGGAGCTGCGGACGAGGTGCTTAAAATAGAAGGTCTGTCCACCGGTTCTTTGCAGCACA
ATGCCCAGATAGCCTTGGGTACATGTAATTTGAGCGATATGGAGTATCTTCATATGGAT
GTATATTCTCCTTCCGAAAACGGAATAGGCGAGTTTAGCTTTTATCTGGTAAGCGGTTGG
AGCAAGACAGTATCTTGCAATGTGTGGTACAACTTTGATACGAAGCAGGAGTACGACCA
GTGGATTTCGATAGACATACCGATGAGCACATTTAAAAACGGAGGATTGAACCTGGCCG
AAATCAATGTGTTACGAATTGCAAGAGGAAAACAGGGAGCACCCGGCACAATTGTCTA
TGTGGACAATGTTTATGCATACGGTAAAGCGGTTGAACCGGAGTCGGATGTGAAGATTG
TGGCCAATGGCAATGCCAACCTGACTACGGATGTTCCTTTGATCTCCGCTCCGACACCG
AAGGTAGCTGCCGCCAATGTATTCAACTTCTTCAGCGATCACTATGGCGACGGTAAGTT
CGATTATGCACAAAGCGATTATGGCGATCAGAAAACAGTGAAATCCCTCATTACCATTA
ATGATACGGAGGATCAGGTATTCAAGATCGATAACATCGTGAATGGAAGTAAGGCGAA
TGTTTCCATCGGCTCACCGAATCTTTCGGGAGTGGACATGCTGCATCTGGATATATTTTC
TCCGGGCAATGATCAGGGAATCGGTGAATTTGATTTTGCCCTGACGGATTTTGGAGGAA
ACGGTAATGATGCCGGTATCTGGCTGAATATTACGGACAAAGGATGGCATGGACAATG
GATCTCCATCGATATACCTCTCAGCAAGTGGACGGGAGCTGCCAATATGATCAGATTCC
GCCGTGGTGGTAAAGGCTCGACCGGTAAGCTGTTGTATGTAGACAACGTTTATGCTTAC
AAGAGTGAATCGGACGATCCGAAACCGGTTCCCGATCCTACTACTGTTCCTGTTCTTACC
AAAGATAAGTCCGATGTTATTTCTATTTTCTGCGAACAGTACGAAGAGCCGGGATACCA
AGATGAATTTGGCATAGTAAGTGCCGGAAACTGGGGGCAAAATGCGAAGCAGAAAGAT
GAATTTGTAGAAATTGTAGCAGGTAACCAAACATTAAAACTTACGTCGTGGGATCTCTT
CCCGTTCAAAGTGCATAAGAACAGTGACGTGATGGATTTATCCCAAATGGACTATTTGC
ACTTAAGCATATATCAGAATGGCGCTTTGGATGAAAACAACAAACCGGTTAGCGTTTGT
ATCTGGATCAACGACAAGGATAATAAGGTGGCACAAGCTCCTTTGTTGGAAGTGAAGCA
AGGCGAATGGACTTCCGTCAGTTTCGGGATGGATTATTTCAAAAACAAGATCGATTTGA
GCCGTGTATATGTGATCCGTTTGAAAGTGGGCGGTTATCCTACCCAGGATATTTACGTAG
ATAATATTTTTGGTTATAAGGGCGATCCTATCCGTCCGGGTCAAGTAACCGAGCCATAT
GTGGACGAGTGCGATCAGAAGATTCAGGATTCCACACCGGGCACTCTGCCGCCGATGGA
ACAGGCCTATCTGGGAGTGAATTTAGCTTCTGCTTCCGGTGGAAGTAATCCGGGCACAT
TCGGACACGATTACTTGTATCCTAAGTTTGAGGATTTGTATTATTTCAAGGCGAAAGGCA
TACGTTTGCTCCGTATCCCGTTCCGTGCTCCGCGTTTGCAACACGAAGTTGGAGGAGAAC
TGGATTATGATGCCGGTAATACGTCGGATATCAAGGCGTTGGCCGCTGTTGTGAAAGAA
GCGGAAAGATTAGGTATGTGGGTTATGCTGGATATGCACGACTACTGCGAACGGAATAT
TGACGGTGTATTGTATGAATATGGAGTTGCCGGACGCAAGGTATGGGACTCTGCCAAAA
ACACCTGGGGAGATTGGGAAGCAATGGATGAAGTGGTGTTGACCAAAGAGCATTTTGC
CGACCTGTGGAAGAAGATTGCTACTGAATTTAAAGATTATACGAATATCTGGGGATACG
ACCTGATGAACGAGCCCAAAGGCATTAACATCAATACGCTGTTTGATAATTATCAGGCT
GCCATTCATGCGATTCGTGAGGTGGATACAAAAGCACAAATAGTAATCGAAGGTAAGA
ATTATGCCAATGCTGCCGGTTGGGAAGGTTCAAGCGACATACTGAAAGATCTGGTCGAT
CCGGTCAATAAGATCGTTTATCAGGCACATACCTACTTTGACAAGAACAATACGGGTAC
CTATAAAAATTCTTACGATCAGGAGATTGGCGGAAATGTAGAGGTCTATAAACAACGTA
TCGATCCTTTTATTGCCTGGTTAGAAAAGAACAACAAAAAAGGTATGTTGGGTGAATAC
GGAGTTCCTTATAATGGACATGCGCAAGGTGACGAGAGATATATGGACTTGATCGATGA
TGTATTTGCTTATCTGAAAGAGAAACAGCTTACCTCTACTTATTGGTGCGGTGGATCGAT
GTACGATGCTTATACGCTGACTGTACAACCTGCCAAGGATTATTGTACAGAGAAATCTA
CCATGAAGGTTATGGAGAAATATATCAAGGATTTTGATACCAGTATTCCTTCTTCCCTGG
TGGAAACCAATGCTGACGGCAATGCCATCGTGCTCTATCCCAATCCGGTGAAAGATAAC
TTGAAGATTACTTCTGAAAGCGGAATCGAACAGGTGATTGTCTTCAATATGATAGGCCA
GAAAGTAAGCGAGCGAAATGAAAAGGGCACTAACATCGAATTGAACCTCGAAGCATTG
GGCAAGGGTACTTACTTAGTAACTGTCCGCTTGGAAGACGGTAATGTGGTGAACCGTAA
GATTGTGAAAATGTAATTGATGATGAAATGAAATACAGCCGGGCAACGGCTGTATTTCC
ATACTTGACAGATAGACAAAAGAGACGCAGCATCTTATTGAAAAGGTGCTGCGTCTCTT
TTTTAATGAAAGATTGATAGAGATAGGAACGACTTATTATTTTTTCGACAGAAGAACAA
AAGAACATATTTCCTGCATAGCCTTTATAGGCGGTTTATTTGTTCTTTTGTTCTTCTGTCG
AAAAATAGATTCGTGACTTGTTTTGAGTTGAAGTTGAACCGTTTTATCGATGATATTGAA
TAAAGGCAGCCAGTGGAATCCCCATCGTAGGATAATTTTTGTAGGGATGAGGCTGATAG
ATCTGCATGCCCTCTTCGTCATAGTAATAGCCGTCTTCGTTGTCTATCGTGATACCGATG
TCTACCAGATAATACCCTTCGGATTCCTTTTTATCGAAATTGTCGATCAGGTATTTCCGG
AAACGCCACATGGCAAAGTTTTGCATGACGGTGAAGTTCCCGTCTTTGTTGTCCATCGTA
CCGCAGGTGCTGCAAGGGATCAGTATCACAAACTTGCCGTTGGGCACCGCTTTCAGATA
CGACTCTTTCACTATTCTCAGTTGTTTGTCGAAAGTGGAGAAGTCGGCGTTGATGTTGTT
TCTGAAATCGTTCAGACCGAGCATTTCCGCTAAGAACTGGGGAGGGGTAATGTTCCACA
TGGCAAGGTATTTGCCATAGTCGAAATTCCATGTGAAATCATCTTTCTGGACATTGACCC
ATTGGTTGCCATCGTACATGACGAATGATCTTTTAGCGTTGTCATATAGTATGTCCCCTT
TTGCGGGCGACTTAAGATACCCGTCTTCGTTGAACTTATACAGGCAACTTCCATATTTAC
CGTTGGTGGCTTCCAGGTTGGGTCTTTCTCCTTTCTCTACAAGGAAACAGAGTTGCCAGA
ATTCGGTCGATCCCCAATAACGGAAATCCCCGTCCGGATGCATGAAACCGTGATAGCGG
TTGTTTCCCGTGAATACCTCAAAGTACCAGCTCATGCAGGCGCCGTTGCGTCCTTCGTCG
TATTGCCCGGTTGTGTACTGCGGATCGTCTTCCGTTTCAACCTTTACGTCTCTTAATCCTA
CGAGCTTGAGGTTCGGTACATATCCTTTTCGCAATAACGCATCTTTGTAAAAGGCACCTT
GTGTATAGCTGTCGCCGATGATTTGTGCCACGACTTCGGAATTACCGGTACCTTTTATTC
CCAGTCTGATTCTGGAAGAGTGAGTCGCCACTCTGGTGAAGTTTTTGAGTTCGTATAAGT
TGGCAATGATTTTCTTGTCATTTTCCGGTTTGTCTACCGATACTACCCGTTCCAACCGGC
GTGAGTAGAAATCTCCATTGAATAGGACACTATAATCGAACGGATACCATCTTTTTATG
AACGGTTCTACAAAAATGTCATTTCTGGTATCCGACAGCATGTACAGATAACTGGGCAG
GCATATTTCATTTACGTCGGACTTATTGACGGCCAGCGTGATCTGTGTACCGTCACTGAA
ACTGAAAGTCATTTGATCGCCATTGGCCGAGATATTTGTAATTTGGCTTCCGTCGGTGCC
GTCGATTCCGTTTTGCAGCACAACGGTGCTCCCATCGGAGAGGGTGATTGTGTAACCAT
CGTCCGTAGTGGCTACATGGGTGATGTAGATATTGTTTTGAGATGCTTCGAGCAGCTGTT
TCTGGACGTTTAGCTCGTTGCGCAATTTTTCTACTTCCTCTTTCCAGTCGTCGTTCTGACA
CGAAGGCAGGAGGAGGGTACAACATAAAAGAATGGTGGTGGTGATGAGGTTTTTCATA
AGCGTTTTTATTAAATAATGATGAGATTAAAAATGAAAATATCCCGAAACTGCTTGAAT
CCCGGGATATTTTAGGTAATGATGGAAACTGGTCTTTTTTACAGTTTTATAATGTGTTTG
CTTACTGTTTTTCCACTAACCAACTTCACGGAAATAATGTATGAACCCGATTGCAGGGCC
GATAGGTTGATTTGATTCTCTCCTGCCATGTTGTAGCTGCCGGCCAATTGACCGCTGATG
GCATACAGGTTGGCCGACTGAACTGCTTCTTCGGAATCGATCGTAATATAGTCTGTTACG
GCAGTAGGATAGATGTTTAAACCGTCGTTGGCTTTAGCAGACTTGATTCCTGTGGGCGA
ACCTTTATAAGCGAATATGTTGGATACATAAATATTAGGTGCATATTGCTTGGAGAGTG
GTTCGTATATACCGTCTCTGCTGCCGAGTCTTAAGCCGTTGATCACATAATTCTTATTCTC
CGCAGTCCAGTCGAAGTTTTCGATAGGAAGATCGATAGAATTCCATTGATTGGCTTTCA
AGTCGAATATTTCGCTGTAAGCATCTGCCATTGCCGGGTAATTCCAGGTTACGCCTACCA
CGAACTGGCAATCCTGATCCGGCCAGAAATCGAAATGCAGATAGTCATAATCGGTAACG
GTGGCGCCGGCATTGGTATAGAATGAGGACCATTCCAGGTTAATCATATGCAGAACCGC
ATCCTTATTAATATAATCGTCTACGAAATTATCCGGACTAAGTCCCCAGTTTGTACGTAG
GATCAGTTTGTGATCTGATGCCGGTTCATAAGTCTTTCCATAGAACGAAATGACGTCTGC
TTCCGGGTAAGTCGGAGTAGGAGCGGCCATTGTCGGTTCTTGTGCATTTGCAAATTGTGT
ACTGCCTAATAATGCCAAGGCTGCAATAAAATAAGTAATCTTTCTCATAATCTTAAAATT
TTAGAGTTTAACGATTTGTTCCCTTTTGGTGTGGGCAAAGTAATGGAAAAGATCATTTTG
GGGATGTAATAATCTTATTTTTTTATAGAAGAATATTGTTTTAACTATTTATTTTTCTGAA
ATTCAACCCCACTAAACTAAGATTATTATATCCTTCTATAAATATGAAATATTCTTCTAT
GGAACAAGCTCCGAGGAAGCTACTTTTGTAGACAGGTAAAAGAAAACTTAGTTTGTCAA
CAAAAGAAAGGAGGACATGTAGAAGAAACGATGAATTCAATAAACTGCACTTGTGATA
GATGATAATCTTCCGGGTCGGAGAGCTTGTGATTTATTTAAAAAAGAATCTAATACTGA
TAATTGTATGATTTCAAAAGACGAAAATATAAAAAGGCGGATCATTGGTGTTTTATTTTT
CTTATGTGCTCTAAGTCCTGCATTATGGGCTCAGTCGCGCATTATAAAAGGTGAAGTGCT
CGATCCCAACGGAGAACCTCTGATAGGTGTAGGGGTTATGATTAAAAATACTACTGCTG
GAACCATCACTGATGTCGATGGAAGATATTCCATTCAGGTTCCCGATAATAATGCTGTTC
TTTCCTTCTCTTATGTAGGCTATAAAAGAAAAGAGGTCAAGGTGGGAAGTCAAAGCGTG
ATTAATATTTCTCTGGAAGAGGAATCCGTATTGATGGATCAAGTTGTCATTGTGGGATAT
GGTAGCCAGAAGAAAGTCAATCTGACGGGAGCCGTAGCTGCAATTTCCGTTGACGAATC
CCTTGCCGGCCGTTCGGTTGCCAATGTCTCTTCCGCTTTGCAGGGGTTGATGCCGGGACT
GTCCGTGAGCCAGAGCTCGGGTATGGCGGGAAATAATTCTGCCAAACTGTTGATTCGTG
GTTTAGGAACGATCAATAGTGCCGATCCGCTGATCGTGGTGGACGACATGCCGGATGCC
GATATTAACCGGCTAAATATGAATGATATAGAAAGTATAACCGTCTTGAAGGATGCAAC
GGCTTCTTCCGTTTACGGTTCTCGTGCAGCCAACGGTGTAATACTTGTTAAAACCAAATC
GGGTAAAGGTTTGGAAAAGACGCAAATAACCTTCTCCGGATCGTATGGATGGGAAAAG
CCGACGAATACTTACGATTTTATATCCAATTATCCACGCGCTTTGACTTTACAGCAAATT
TCCTCTTCGACCAATCCCGGCAAGAATGGAGAAAATCAGAATTTTAAGGATGGAACGAT
CGACCAATGGCTGGCATTGGGAATGATTGACGACAAGCGGTATCCGAACACGGACTGG
TGGGATTACATCATGCGAACGGGTTCCATTCAAAATTATAATGTATCGGCAACGGGTGG
AAGCGAGAAATCGAACTTTTACGCATCTGTGGGATATATGAAGCAGGAAGGATTACAG
ATAAATAATGACTACGACCGCTATAACGCCCGTTTTAACTTTGACTATAAGGTGATGAA
AAATGTGAATACCGGATTCCGTTTTGACGGGAACTGGAGTAATTTCACTTATGCCTTGG
ACAATGGTTTCACGAGCGATTCTAACCTGGATATGCAGAGTGCGATTGCCGGTATCTAT
CCTTATGATCCGGTTCTGGATGTTTATGGCGGTGTAATGGCGTATGGAGAAGATCCACA
GGCTTTCAATCCGTTGAGCTTTTTCACAAATCAGTTGAAGAAGAAAGACAGACAGGAGT
TGAATGCTTCTTTCTATCTTGACTGGGAACCCGTAAAGGGTCTGGTAGCCCGCGTGGATT
ATGGTTTGAAGTATTATAACCAATTTTATAAGGAAGCGGACATCCCCAACCGTTCTTAC
AATTTCCAGACGAACTCGTATGGTATCAGGGAATATGTTACGGAGAATGCCGGAGTTAC
AAACCAGACGAGCACCGGTTACAAAACTCTGTTGAATGCCCGTTTGAATTATCACACGG
TTTTTGCTACACACCATGATTTGAATGCCATGTTCGTATATAGCGAGGAATACTGGCACG
ACCGTTATCAGATGTCCTATAGGCAGGACAGAATTCATCCGTCACTCTCCGAAATAGAT
GCTGCCTTGTCCGGAACACAGTCTACTTCCGGTAATTCTTCGGCAGAAGGACTCCGTTCT
TATATCGGACGTATCAATTATTCTGCTTACGGCAAATATTTGCTGGAACTTAATTTCCGT
GTCGATGGTTCGTCTAAGTTTCAACCGGGACACCAGTACGGCTTTTTCCCGTCGGCAGCT
TTGGGCTGGAGGTTTAGCGAAGAGTCGTTTGTGAAGCCTTATATAGGGAAATGGCTGGC
AAGCGGAAAACTCCGTGCTTCTTACGGTAAGCTGGGTAACAATAGCGGTATTGGCAGAT
ACCAGCAGCAAGAGGTGCTTTATCAGAATAACTATATGCTGGACGGTTCGATTGCCAAA
GGTTTTGTGTATTCTAAAATGTTGAACCCGGATCTGACTTGGGAATCTACGGGAGTATTC
AACCTGGGACTGGACCTGATGTTTTTCGATGGAAAACTCGCTGCGGAATTTGATTATTAC
GACCGTCTGACGACCGGTATGTTGCAAAAGTCGCAGATGTCCATTCTGCTGACCGGTGC
TTATGAAGCGCCTATGGCAAATCTGGGGACGCTCCGTAACCGGGGATTCGAAGCGAACT
TAACCTGGAGAGACCGGATTGCAGACTTTACTTATTCTGCCAATTTCAATATCTCTTATA
ACCGTACGAACCTTGAGAAGTGGGGGGAGTTCCTGGATAAAGGATATGTTTACATAGAT
ATGCCTTATCATTTTGTATACAGCCAGCCGGATCGCGGATTGGCTCAAACCTGGACCGA
TTCCTATAACGCTACCCCTCAAGGAGTGGCTCCGGGAGATGTGATCCGTCTGGATACCA
ATGGCGACGGACGCATTGATGGCAATGACAAAGTGGCCTATACAAACTTCCAGCGCGAT
ATGCCGACTACCAACTTCGCCTTGAACCTTCAGATGGGATGGAAAGGTATCGATGTATC
TTTACTGTTTCAAGGATCGGCTGGTCGTAAAGACTTCTGGAACAACAAATATACGGAAA
TCAACCTGCCGGACAAGCGTTATACCTCCAACTGGGATCAATGGAATAAGCCTTGGTCG
TGGGAGAACAGAGGAGGAGAGTGGCCGCGTTTGGGAGGATTGGTGACTAACAAGACGG
AAACTGATTTCTGGTTGCAGAACATGACTTATTTAAGAATGAAGAACCTCATGATCGGT
TATACCTTTCCGAAAAAATGGACGAGAAAGTGTTTCATAGAGAATCTCCGGATTTATGG
AACGGCGGAAAATCTGCTGACTATTACCGGTTATAAAGGACTCGATCCGGAAAAAGCG
GCTAACTCACAAGATTTGTATCCTATCACCAAATCTTATTCTATTGGCGTTAATCTGAGT
TTTTAATAAATGAAAAGCGGAAATTATGAAAAGAGTTTATATTAAATATATAGGTTTGA
TTGCTGGGATGATGATGCTATTCAGTTCCTGTGCCGACTTGTTGAATCAAGAACCTACGG
TGGATCTGCCGGCTACTAATTATTGGAAAACAGAGTCCGATGCCGAATCAGCATTGAAC
GGGCTGGTATCCGATATACGCTGGCTTTTTAACCGGGACTACTATCTCGACGGAATGGG
AGAATTTGTCAGAGTGCGCGGTAACTCTTTCCTGAGCGATAAAGGACGCGACGGAAGA
GCTTACAGGGGGCTTTGGGAAATCAATCCGGTAGGCTACGGCGGCGGATGGTCCGAAAT
GTACAGGTATTGCTATGGGGGCATCAACCGTGTAAACTATGTAATCGACAATGTCGAGA
AGATGATAGCTAATGCAAGTAGTGAAAAAACGATCAAGAACTTGGAAGGCATAATCGG
TGAATGTAAGCTGATGCGGGCTTTGGTTTATTTCAGATTGATCATGATGTGGGGAGATGT
GCCTTATATCGACTGGAGAGTATACGATAATTCGGAGGTTGAGAACTTACCGCGTACTC
CGCTTGCCGAAGTAAAGGATCATATCCTGGATGATTTGCTGGATGCTTTTAAGAAATTG
CCCGAAAAGGCGACAGTTGAAGGCCGTTTTTCACAACCTGCCGCATTGGCTTTACGCGG
AAAGGTACTGCTTTATTGGGCAAGCTGGAACCATTACGGTTGGCCGGAACTGGATACGT
TTACACCGAGCGAAGAGGAAGCTCGAAAAGCATATAAGGCGGCAGCCGAAGATTTCAG
AACGGTGATTGATGACTATGGTCTGACTCTGTTCAGAAATGGAGAGCCGGGAGAATGTG
ACGAGCCGGGAAAAGCCGACAAGCTGCCCAATTACTATGACCTGTTTTTGCCTACGGCA
AACGGTGATGCCGAATTTGTACTGGCATTTAATCACGGTGGCACGAACACAGGGCAGGG
CGATCAGCTGATGCGGGATTTAGCCGGACGAAGTGTTGAAAACTCACAATGTTGGGTAT
CTCCCCGTTTCGAAATTGCCGATAAATATCAGTCTACGATAACCGGTGACTTCTGTGTAC
CGTTGGTTAAGTTGAATCCCTCTTCTGTGCCCGATGCCCGTACCCGTCCTAATTCAGCCG
TGAATCCGGAGAGTTATAAGGACCGGGATTACCGTATGAAAGCGTCGATCATGTGGGAT
TATGAAATATGCCAGGGACTCATGTCCAAGAAAGTGACAGGATGGGTGCCTTTCATCTA
CAAGATGTGGGGAAGTGAAGTAGTTATTAATGGTGAAACCTATATGTCCTACAATACCG
ATGGTACCAATTCCGGATATGTATTCCGGAAGTTTGTGAGGAACTATCCTGGTGAAGAA
CGGGCTGACGGAGATTTCAATTGGCCTGTCATACGTCTTGCCGATGTGTTTTTAATGTAT
GCTGAGGCGGATAATGCCGTAAACGGTCCTCAGCCTTATGCCATAGAGCTGGTGAACAG
AGTGCGTCACAGAGGTAATCTTCCGGTGTTGGCATCCAGTAAGACATCTACTCCCGAAG
CATTTTTCGAAGCGATAAAGCAGGAGAGAATTGTGGAACTGCTGGGAGAGGGCCAGCG
TGCATTTGATACGCGCAGGTGGAGAGAGATCGAAACAGTCTGGTGCGAACCCGGTGGC
AGAGGAGTAAAGATGTATGATACGTATGGAGCACAGGTTGCCGAATTTTATGTGAATCA
GAATAACCTGGCTTATGAACGTTGCTATATTTTCCAGATACCGGAGTCGGAACGTAACC
GTAATCCGAATTTGACTCAGAATAAACCATACAGATAA

### REFERENCES

1. García-Ochoa, F., Santos, V. E., Casas, J. A. & Gómez, E. Xanthan gum: Production, recovery, and properties. Biotechnol. Adv. 18, 549-579 (2000).
2. Shepherd, E. S., Deloache, W. C., Pruss, K. M., Whitaker, W. R. & Sonnenburg, J. L. An exclusive metabolic niche enables strain engraftment in the gut microbiota. Nature 557, 434-438 (2018).
3. Laudisi, F. et al. The Food Additive Maltodextrin Promotes Endoplasmic Reticulum Stress-Driven Mucus Depletion and Exacerbates Intestinal Inflammation. Cmgh 7, 457-473 (2019).
4. Chassaing, B. et al. Dietary emulsifiers impact the mouse gut microbiota promoting colitis and metabolic syndrome. Nature 519, 92-96 (2015).
5. Etienne-Mesmin, L. et al. Experimental models to study intestinal microbes-mucus interactions in health and disease. FEMS Microbiol. Rev. 43, 457-489 (2019).
6. King, J. A. et al. Incidence of Celiac Disease Is Increasing Over Time. Am. J. Gastroenterol. 1 (2020). doi:10.14309/ajg.0000000000000523
7. Beal, J., Silverman, B., Bellant, J., Young, T. E. & Klontz, K. Late onset necrotizing enterocolitis in infants following use of a Xanthan gum-containing thickening agent. J. Pediatr. 161, 354-356 (2012).
8. Vojdani, A. & Vojdani, C. Immune reactivities against gums. Altern. Ther. Health Med. 21, 64-72 (2015).
9. Hehemann, J. H., Kelly, A. G., Pudlo, N. A., Martens, E. C. & Boraston, A. B. Bacteria of the human gut microbiome catabolize red seaweed glycans with carbohydrate-active enzyme updates from extrinsic microbes. Proc. Natl. Acad. Sci. U.S.A 109, 19786-19791 (2012).
10. Quast, C. et al. The SILVA ribosomal RNA gene database project: Improved data processing and web-based tools. Nucleic Acids Res. 41, 590-596 (2013).
11. Goodman, A. L. et al. Extensive personal human gut microbiota culture collections characterized and manipulated in gnotobiotic mice. Proc. Natl. Acad. Sci. U.S.A 108, 6252-6257 (2011).
12. Kim, C. C. et al. Genomic insights from Monoglobus pectinilyticus: a pectin-degrading specialist bacterium in the human colon. ISME J. 13, 1437-1456 (2019).
13. Hashimoto, W., Nankai, H., Mikami, B. & Murata, K. Crystal structure of Bacillus sp. GL1 xanthan lyase, which acts on the side chains of xanthan. J. Biol. Chem. 278, 7663-7673 (2003).
14. Jensen, P. F. et al. Structure and Dynamics of a Promiscuous Xanthan Lyase from Paenibacillus nanensis and the Design of Variants with Increased Stability and Activity. Cell Chem. Biol. 26, 191-202.e6 (2019).
15. Aspeborg, H., Coutinho, P. M., Wang, Y., Brumer, H. & Henrissat, B. Evolution, substrate specificity and subfamily classification of glycoside hydrolase family 5 (GH5). BMC Evol. Biol. 12, (2012).
16. Jongkees, S. A. K. & Withers, S. G. Unusual enzymatic glycoside cleavage mechanisms. Acc. Chem. Res. 47, 226-235 (2014).
17. Rovira, C., Males, A., Davies, G. J. & Williams, S. J. Mannosidase mechanism: at the intersection of conformation and catalysis. Curr. Opin. Struct. Biol. 62, 79-92 (2020).
18. Kool, M. M. et al. Characterization of an acetyl esterase from Myceliophthora thermophila C1 able to deacetylate xanthan. Carbohydr. Polym. 111, 222-229 (2014).
19. Almagro Armenteros, J. J. et al. SignalP 5.0 improves signal peptide predictions using deep neural networks. Nat. Biotechnol. 37, 420-423 (2019).
20. Grondin, J. M., Tamura, K., Déjean, G., Abbott, D. W. & Brumer, H. Polysaccharide utilization loci: Fueling microbial communities. J. Bacteriol. 199, 1-15 (2017).
21. McLean, R. et al. Functional analyses of resurrected and contemporary enzymes illuminate an evolutionary path for the emergence of exolysis in polysaccharide lyase family. J. Biol. Chem. 290, 21231-21243 (2015).
22. Abbott, D. W., Thomas, D., Pluvinage, B. & Boraston, A. B. An ancestral member of the polysaccharide lyase family 2 displays endolytic activity and magnesium dependence. Appl. Biochem. Biotechnol. 171, 1911-1923 (2013).
23. Artzi, L., Bayer, E. A. & Moraïs, S. Cellulosomes: Bacterial nanomachines for dismantling plant polysaccharides. Nat. Rev. Microbiol. 15, 83-95 (2017).
24. Ebbes, M. et al. Fold and function of the InlB B-repeat. J. Biol. Chem. 286, 15496-15506 (2011).
25. Bleymüller, W. M. et al. MET-activating residues in the B-repeat of the Listeria monocytogenes invasion protein InlB. J. Biol. Chem. 291, 25567-25577 (2016).
26. Kool, M. M., Gruppen, H., Sworn, G. & Schols, H. A. Comparison of xanthans by the relative abundance of its six constituent repeating units. Carbohydr. Polym. 98, 914-921 (2013).
27. Moroz, O. V. et al. Structural Dynamics and Catalytic Properties of a Multi-Modular Xanthanase. ACS Catal. 8, 6021-6034 (2018).
28. Nankai, H., Hashimoto, W., Miki, H., Kawai, S. & Murata, K. Microbial system for polysaccharide depolymerization: Enzymatic route for xanthan depolymerization by Bacillus sp. strain GL1. Appl. Environ. Microbiol. 65, 2520-2526 (1999).
29. Yang, F. et al. Novel Endotype Xanthanase from Xanthan-Degrading Microbacterium Microbacterium sp. Strain XT11. 85, 1-16 (2019).
30. Yang, F. et al. Production and purification of a novel xanthan lyase from a xanthan-degrading microbacterium sp. Strain XT11. Sci. World J. 2014, (2014).
31. Ruijssenaars, H. J., De Bont, J. A. M. & Hartmans, S. A pyruvated mannose-specific xanthan lyase involved in xanthan degradation by Paenibacillus alginolyticus XL-1. Appl. Environ. Microbiol. 65, 2446-2452 (1999).
32. Gregg, K. J. et al. Analysis of a new family of widely distributed metal-independent α-mannosidases provides unique insight into the processing of N-linked glycans. J. Biol. Chem. 286, 15586-15596 (2011).
33. Daly, J., Tomlin, J. & Read, N. W. The effect of feeding xanthan gum on colonic function in man: correlation with in vitro determinants of bacterial breakdown. Br. J. Nutr. 69, 897-902 (1993).
34. Kozich, J. J., Westcott, S. L., Baxter, N. T., Highlander, S. K. & Schloss, P. D. Development of a dual-index sequencing strategy and curation pipeline for analyzing amplicon sequence data on the miseq illumina sequencing platform. Appl. Environ. Microbiol. 79, 5112-5120 (2013).
35. Schloss, P. D. et al. Introducing mothur: Open-source, platform-independent, community-supported software for describing and comparing microbial communities. Appl. Environ. Microbiol. 75, 7537-7541 (2009).
36. Massie, H. R. & Zimm, B. H. THE USE OF HOT PHENOL IN PREPARING DNA. Proc. Natl. Acad. Sci. 54, 1641-1643 (1965).
37. Nie, X. Relationships between dietary fiber structural features and growth and utilization patterns of human gut bacteria. ProQuest Diss. Theses 136 (2016).
38. Tuncil, Y. E., Thakkar, R. D., Marcia, A. D. R., Hamaker, B. R. & Lindemann, S. R. Divergent short-chain fatty acid production and succession of colonic microbiota arise in fermentation of variously-sized wheat bran fractions. Sci. Rep. 8, 1-13 (2018).
39. Arnal, G., Attia, M. A., Asohan, J. & Brumer, H. A Low-Volume, Parallel Copper-Bicinchoninic Acid (BCA) Assay for Glycoside Hydrolases. in Protein-Carbohydrate Interactions. Methods and Protocols (eds. Abbott, D. W. & Lammerts van Bueren, A.) 1588, 209-214 (Springer New York, 2017).
40. Wang, J. et al. A metagenome-wide association study of gut microbiota in type 2 diabetes. Nature 490, 55-60 (2012).
41. Yu, J. et al. Metagenomic analysis of faecal microbiome as a tool towards targeted non-invasive biomarkers for colorectal cancer. Gut 66, 70-78 (2017).
42. Liu, R. et al. Gut microbiome and serum metabolome alterations in obesity and after weight-loss intervention. Nat. Med. 23, 859-868 (2017).
43. Gu, Y. et al. Analyses of gut microbiota and plasma bile acids enable stratification of patients for antidiabetic treatment. Nat. Commun. 8, (2017).
44. He, Q. et al. Two distinct metacommunities characterize the gut microbiota in Crohn's disease patients. Gigascience 6, 1-11 (2017).
45. Zhang, X. et al. The oral and gut microbiomes are perturbed in rheumatoid arthritis and partly normalized after treatment. Nat. Med. 21, 895-905 (2015).
46. Nishijima, S. et al. The gut microbiome of healthy Japanese and its microbial and functional uniqueness. DNA Res. 23, 125-133 (2016).
47. Lloyd-Price, J. et al. Strains, functions and dynamics in the expanded Human Microbiome Project. Nature 550, 61-66 (2017).
48. Le Chatelier, E. et al. Richness of human gut microbiome correlates with metabolic markers. Nature 500, 541-546 (2013).
49. Qin, J. et al. A human gut microbial gene catalogue established by metagenomic sequencing. Nature 464, 59-65 (2010).
50. Smits, S. A. et al. Seasonal cycling in the gut microbiome of the Hadza hunter-gatherers of Tanzania. Science (80-.). 357, 802-805 (2017).
51. Conteville, L. C., Oliveira-Ferreira, J. & Vicente, A. C. P. Gut microbiome biomarkers and functional diversity within an Amazonian semi-nomadic hunter-gatherer group. Front. Microbiol. 10, 1-10 (2019).
52. Boratyn, G. M., Thierry-Mieg, J., Thierry-Mieg, D., Busby, B. & Madden, T. L. Magic-BLAST, an accurate RNA-seq aligner for long and short reads. BMC Bioinformatics 20, 1-19 (2019).
53. Quinlan, A. R. & Hall, I. M. BEDTools: A flexible suite of utilities for comparing genomic features. Bioinformatics 26, 841-842 (2010).
54. Dorotea, I. et al. Polypeptides having Xanthan Degrading Activity and Polynucleotides Encoding the Same United States Patent U.S. Pat. No. 9,458,441B2. 2, (2016).
55. McDonald, Sean A., Exploring protein structure-function relationships in xanthanases from glycoside hydrolase family 9 THESIS SUBMITTED IN PARTIAL FULFILLMENT OF THE REQUIREMENTS FOR THE DEGREE OF Master of Science in THE UNIVERSITY OF BRITISH COLUMBIA (Vancouver) December 2019.
56. O'CONNELL, T. Detergent Compositions Comprising Polypeptides Having Xanthan Degrading Activity. WO 2017/046232 A1. (2014).
57. TJON-JOE-PIN, Robert, M. & CARR, Michelle, Alana. YANG, B. Methods and Materials for Degrading Xanthan. EP0975708B1. (2005).
58. Kim Bruno Andersen; Morten Foverskov; Ole, T. R., Martin, S. K. J. M. & Andersen, N. L. Microencapsulation of detergent enzymes. US 2016/0075976 A1. 1, (2016).
59. Baba Hamed, S. & Belhadri, M. Rheological properties of biopolymers drilling fluids. J. Pet. Sci. Eng. 67, 84-90 (2009).
60. Weaver, J. D., Michael A. McCabe & Ronnie G. Morgan. Wellbore Servicing Compositions and Methods of Making and Using Same. US 2016/0271610 A1. (2016).
61. Kumar, A., Rao, K. M. & Han, S. S. Application of xanthan gum as polysaccharide in tissue engineering: A review. Carbohydr. Polym. 180, 128-144 (2018).
62. Ramburrun, P., Kumar, P., Choonara, Y. E., du Toit, L. C. & Pillay, V. Design and characterization of neurodurable gellan-xanthan pH-responsive hydrogels for controlled drug delivery. Expert Opin. Drug Deliv. 14, 291-306 (2017).
63. García-Ochoa, F., Santos, V. E., Casas, J. A. & Gómez, E. Xanthan gum: Production, recovery, and properties. Biotechnol. Adv. 18, 549-579 (2000).
64. Shepherd, E. S., Deloache, W. C., Pruss, K. M., Whitaker, W. R. & Sonnenburg, J. L. An exclusive metabolic niche enables strain engraftment in the gut microbiota. Nature 557, 434-438 (2018).
65. Casas, J. A., Santos, V. E. & Garcia-Ochoa, F. Xanthan gum production under several operational conditions: Molecular structure and rheological properties. Enzyme Microb. Technol. 26, 282-291 (2000).
66. Sworn, G. Xanthan gum. in Handbook of Hydrocolloids 262, 833-853 (Elsevier, 2021).
67. Mortensen, A. et al. Re-evaluation of xanthan gum (E 415) as a food additive. EFSA J. 15, (2017).
68. Pilgaard, B., Vuillemin, M., Holck, J., Wilkens, C. & Meyer, A. S. Specificities and synergistic actions of novel PL8 and PL7 alginate lyases from the marine fungus Paradendryphiella salina. J. Fungi 7, 1-16 (2021).
69. Zhu, B. & Yin, H. Alginate lyase: Review of major sources and classification, properties, structure-function analysis and applications. Bioengineered 6, 125-131 (2015).
70. Terrapon, N. et al. PULDB: The expanded database of Polysaccharide Utilization Loci. Nucleic Acids Res. 46, D677-D683 (2018).
71. Sun, Z., Liu, H., Wang, X., Yang, F. & Li, X. Proteomic Analysis of the Xanthan-Degrading Pathway of Microbacterium sp. XT11. ACS Omega 4, 19096-19105 (2019).
72. Guillén, D., Sánchez, S. & Rodríguez-Sanoja, R. Carbohydrate-binding domains: Multiplicity of biological roles. Appl. Microbiol. Biotechnol. 85, 1241-1249 (2010).
73. Mistry, J. et al. Pfam: The protein families database in 2021. Nucleic Acids Res. 49, D412-D419 (2021).
74. Glenwright, A. J. et al. Structural basis for nutrient acquisition by dominant members of the human gut microbiota. Nature 541, 407-411 (2017).
75. Kielbasa, S. M., Wan, R., Sato, K., Horton, P. & Frith, M. C. Adaptive seeds tame genomic sequence comparison. Genome Res. 21, 487-493 (2011).
76. Chen, I. M. A. et al. The IMG/M data management and analysis system v.6.0: New tools and advanced capabilities. Nucleic Acids Res. 49, D751-D763 (2021).
77. Liang, R. et al. Metabolic capability of a predominant Halanaerobium sp. in hydraulically fractured gas wells and its implication in pipeline corrosion. Front. Microbiol. 7, 1-10 (2016).
78. Schnizlein, M. K., Vendrov, K. C., Edwards, S. J., Martens, E. C. & Young, V. B. Dietary xanthan gum alters antibiotic efficacy against the murine gut microbiota and attenuates Clostridioides difficile colonization. bioRxiv 5, 1-10 (2019).
79. Katzbauer, B. Properties and applications of xanthan gum. Polym. Degrad. Stab. 59, 81-84 (1998).
80. Team, R. C. R: A language and environment for statistical computing. (2020).
81. Wickham, H. Reshaping Data with the reshape Package. J. Stat. Softw. 21, 1-20 (2007).
82. Neuwirth, E. RColorBrewer: ColorBrewer Palettes. (2014). Available at: cran.r-project.org/package=RColorBrewer.
83. Wickham, H. Elegant Graphics for Data Analysis: ggplot2. Applied Spatial Data Analysis with R (2008).
84. Martens, E. C. et al. Recognition and degradation of plant cell wall polysaccharides by two human gut symbionts. PLoS Biol. 9, (2011).
85. Pope, P. B. et al. Isolation of Succinivibrionaceae implicated in low methane emissions from Tammar wallabies. Science (80). 333, 646-648 (2011).
86. Martin, M. Cutadapt removes adapter sequences from high-throughput sequencing reads. EMBnet.journal 17, 10 (2011).
87. Nurk, S., Meleshko, D., Korobeynikov, A. & Pevzner, P. A. MetaSPAdes: A new versatile metagenomic assembler. Genome Res. 27, 824-834 (2017).
88. Kang, D. D., Froula, J., Egan, R. & Wang, Z. MetaBAT, an efficient tool for accurately reconstructing single genomes from complex microbial communities. PeerJ 2015, 1-15 (2015).
89. Parks, D. H., Imelfort, M., Skennerton, C. T., Hugenholtz, P. & Tyson, G. W. CheckM: Assessing the quality of microbial genomes recovered from isolates, single cells, and metagenomes. Genome Res. 25, 1043-1055 (2015).
90. Chen, I. M. A. et al. IMG/M: Integrated genome and metagenome comparative data analysis system. Nucleic Acids Res. 45, D507-D516 (2017).
91. Lombard, V., Golaconda Ramulu, H., Drula, E., Coutinho, P. M. & Henrissat, B. The carbohydrate-active enzymes database (CAZy) in 2013. Nucleic Acids Res. 42, 490-495 (2014).
92. Rodriguez-R, L. M. et al. The Microbial Genomes Atlas (MiGA) webserver: Taxonomic and gene diversity analysis of Archaea and Bacteria at the whole genome level. Nucleic Acids Res. 46, W282-W288 (2018).
93. Chaumeil, P. A., Mussig, A. J., Hugenholtz, P. & Parks, D. H. GTDB-Tk: A toolkit to classify genomes with the genome taxonomy database. Bioinformatics 36, 1925-1927 (2020).
94. Seemann, T. Prokka: Rapid prokaryotic genome annotation. Bioinformatics 30, 2068-2069 (2014).
95. Koren, S. et al. Canu: scalable and accurate long-read assembly via adaptive k-mer weighting and repeat separation. Genome Res. 27, 722-736 (2017).
96. Li, H. Minimap2: Pairwise alignment for nucleotide sequences. Bioinformatics 34, 3094-3100 (2018).
97. Vaser, R., Sović, I., Nagarajan, N. & Sikić, M. Fast and accurate de novo genome assembly from long uncorrected reads. Genome Res. 27, 737-746 (2017).
98. Seppey, M., Manni, M. & Zdobnov, E. M. BUSCO: Assessing Genome Assembly and Annotation Completeness BT —Gene Prediction: Methods and Protocols. (2019).
99. Jain, C., Rodriguez-R, L. M., Phillippy, A. M., Konstantinidis, K. T. & Aluru, S. High throughput ANI analysis of 90K prokaryotic genomes reveals clear species boundaries. Nat. Commun. 9, 1-8 (2018).
100. Kunath, B. J. et al. From proteins to polysaccharides: lifestyle and genetic evolution of Coprothermobacter proteolyticus. ISME J. 13, 603-617 (2019).
101. Bolger, A. M., Lohse, M. & Usadel, B. Trimmomatic: A flexible trimmer for Illumina sequence data. Bioinformatics 30, 2114-2120 (2014).
102. Kopylova, E., Noé, L. & Touzet, H. SortMeRNA: Fast and accurate filtering of ribosomal RNAs in metatranscriptomic data. Bioinformatics 28, 3211-3217 (2012).
103. Bray, N. L., Pimentel, H., Melsted, P. & Pachter, L. Near-optimal probabilistic RNA-seq quantification. Nat. Biotechnol. 34, 525-527 (2016).
104. Speer, M. A. DEVELOPMENT OF A GENETICALLY MODIFIED SILAGE INOCULANT FOR THE BIOLOGICAL PRETREATMENT OF LIGNOCELLULOSIC BIOMASS. (Pennsylvania State University, 2013).
105. Anders, S. et al. Count-based differential expression analysis of RNA sequencing data using R and Bioconductor. Nat. Protoc. 8, 1765-1786 (2013).
106. Langmead, B. & Salzberg, S. L. Fast gapped-read alignment with Bowtie 2. Nat. Methods 9, 357-359 (2012).
107. Anders, S., Pyl, P. T. & Huber, W. HTSeq-A Python framework to work with high-throughput sequencing data. Bioinformatics 31, 166-169 (2015).
108. Robinson, M. D., McCarthy, D. J. & Smyth, G. K. edgeR: A Bioconductor package for differential expression analysis of digital gene expression data. Bioinformatics 26, 139-140 (2009).
109. Thorvaldsdóttir, H., Robinson, J. T. & Mesirov, J. P. Integrative Genomics Viewer (IGV): High-performance genomics data visualization and exploration. Brief Bioinform. 14, 178-192 (2013).
110. Stewart, R. D. et al. Compendium of 4,941 rumen metagenome-assembled genomes for rumen microbiome biology and enzyme discovery. Nat. Biotechnol. 37, 953-961 (2019).
111. Peng, X. et al. Genomic and functional analyses of fungal and bacterial consortia that enable lignocellulose breakdown in goat gut microbiomes. Nat. Microbiol. 6, 499-511 (2021).
112. Clum, A. et al. The DOE JGI Metagenome Workflow. bioRxiv (2020).

1. A polypeptide comprising a truncated xanthanase, wherein the truncated xanthanase comprises a glycoside hydrolase family 5 endoglucanase domain and three carbohydrate binding domains.

2. The polypeptide of claim 1, wherein the glycoside hydrolase family 5 endoglucanase domain comprises an amino acid sequence having at least 70% sequence identity to SEQ ID NO: 1.

3. The polypeptide of claim 1, wherein the polypeptide comprises an amino acid sequence having at least 70% sequence identity to SEQ ID NO: 2 or SEQ ID NO. 33.

4. (canceled)

5. A polynucleotide comprising a nucleic acid sequence encoding the polypeptide of claim 1.

6-8. (canceled)

9. A composition comprising the polypeptide of claim 1, wherein the composition is a cleaning composition or a well treatment composition a wellbore servicing composition.

10. (canceled)

11. The composition of claim 9, wherein the composition is a laundry detergent, a dishwasher detergent or a hard-surface cleaner.

12-13. (canceled)

14. The composition of claim 9, wherein the composition is a liquid, gel, powder, granulate, paste, spray, bar, or unit dose.

15. A method of cleaning comprising contacting an object or a surface with the polypeptide of claim 1, or a composition thereof.

16. (canceled)

17. The method of claim 15, wherein the object or surface comprises a textile, a glass, a plate, tile, dishware, silverware, a wellbore filter cake, or a wellbore.

18. A method of making intermediate sized xanthan gums and/or pentasaccharide repeating units of xanthan gum comprising:
contacting xanthan gum or a composition comprising xanthan gum with the polypeptide of claim 1 or a composition thereof.

19. A genetically modified bacterium comprising the polypeptide of claim 1 or a polynucleotide encoding thereof.

20. The genetically modified bacterium of claim 19, wherein the bacterium is in the genus Bacteroides, Parabacteroides, Alistipes, Prevotella, Clostridium, Faecalibacterium, Eubacterium, Ruminococcus, Peptococcus, Peptostreptococcus, Bifidobacteria, Escherichia, or Lactobacillus.

21. (canceled)

22. The genetically modified bacterium of claim 19, wherein the bacterium is a gram-positive gut commensal bacteria.

23-24. (canceled)

25. A genetically modified bacterium comprising a heterologous xanthan-utilization gene or gene locus, wherein the heterologous xanthan-utilization gene or gene locus comprises one or more nucleic acids encoding a xanthan or xanthan oligonucleotide degrading enzyme and wherein the xanthan-utilization gene or gene locus comprises a gene encoding a glycoside hydrolase family 5 enzyme having at least 70% sequence identity to SEQ ID NO: 2, SEQ ID NO: 3, or SEQ ID NO: 33.

26-28. (canceled)

29. The genetically modified bacterium of claim 25, wherein the heterologous xanthan-utilization gene or gene locus further comprises one or more nucleic acids encoding at least one or all of: one or more carbohydrate uptake proteins; one or more carbohydrate esterases; a polysaccharide lyase family protein; a glycoside hydrolase family 88 enzyme; a glycoside hydrolase family 94 enzyme; and a glycoside hydrolase family 38 enzyme.

30-34. (canceled)

35. The genetically modified bacterium of claim 25, wherein the heterologous xanthan-utilization gene or gene locus further comprises one or more nucleic acids encoding at least one or all of: one or more carbohydrate uptake proteins; a polysaccharide lyase family protein; a glycoside hydrolase family 88 enzyme; a glycoside hydrolase family 92 enzyme; and a glycoside hydrolase family 3 enzyme.

36-45. (canceled)

46. The genetically modified bacterium of claim 25, wherein the bacterium is in the genus Bacteroides, Parabacteroides, Alistipes, Prevotella, Clostridium, Faecalibacterium, Eubacterium, Ruminococcus, Peptococcus, Peptostreptococcus, Bifidobacteria, Escherichia, or Lactobacillus.

47. (canceled)

48. The genetically modified bacterium of claim 46, wherein the bacterium is a gram-positive gut commensal bacteria.

49-51. (canceled)

52. A method for treating a subject in need thereof comprising administering the genetically modified bacterium of claim 19 or a composition thereof to the subject.

53. The method of claim 52, wherein said subject suffers from a gastrointestinal disease or disorder.

54-55. (canceled)
